SEQUENCE LISTING (94 sequences)

SEQ ID NO: 1 - protein, 436 aa, Inula viscosa
MEIKSITSKLVKPSTPTRSNLQNYDISFFDEQMPNINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKFGETFANNDENFHCFTSWCKFSFYKADFGWGKPVWRSTGHYANHKFVILMDDEEGGGVEAWVHLDEKSMCQLEQDPDIKAYAT

SEQ ID NO: 2 - protein, 446 aa, Erigeron canadensis
MEIKNYASKLVKPSKPTPSTLRRYNISVFDEQVANINTNLILYYTASSQNDNKHFDIFSHNLEISLSKTLTDFYPFSGRYIRNASLVNCSDQGALYVQAKANFRLSEFLGLAWDLKLDMLQDLHPCDFGEAGEIDDPMVSVKVTKFECGGVVIGMSFSHKISDMSTMCIFINNWSARTKSLQIGVNNEVELEKRKYYPDFSLAQLFPKRGLIDNSPRVPRSRIGMKNQVQKFIFTRNAISKIREKINMIDDNNRPSKVQIIVALLWKALVGIDKANGQSKIAYAYQVVNMRNKVVPKLADNSFGNFVTFAIVHINPNERDNVVDLQGFVKLLHESIKDIDNICAGLMTHGDTKYELLSKRLMENSQDSSNKEIYYFTSWCRFSFYGADFGLGKPIWRSPGKFPGQNLVTMMDYDQEGGEGIEAWVHLDENRMSQLEQDPDIMAYAI

SEQ ID NO: 3 - protein, 421 aa, Erigeron canadensis
MEIKIQSTQFIKPSKLTPEVFRDFKLSILDQFAASSYINQVFYYKTSCKVDIPDICGQLVNSLSEILTLFYPLAGRIRETELEVDCSDQGVKYLETQVSISLDKFFELGPKIDHIRRLIRAPDQEKSALLIVQVNIFKCGSLVIGVSGSHKVTDAYNLVRFVNEWASLNRTGQTSGAFSISFDNLDSVFPPKEIESFEDSPVIDKSQTKIVTKRFVFNGSIISKLRAKSGPRNPRHSRVTLVAAIIWKAIISVDQVKSGSLRNSVLAPAINLRGKTRLPISESSFGNVWVPYSIRYLQNEMEPKFDNLVKLIENTTRDVITRLLNATSEEICKEAMACYAEVGEELKQNKFAIFTSWCNFPIYNADFGFGKPCWVSEAGRSLDMVTLMDDKHGDGIEAWVSLNEKDMSVFEQNEDILSVLS

SEQ ID NO: 4 - protein, 443 aa, Inula viscosa
MLNKLLKYGRRQLHTIVSRDIIKPSSPTPSQHKTYNLSLFDQITGNSYSPIVAFYPVPGVRQSSHGKTLELKKSLSQTLTQYYPFAGRVPKSLPTYVDCNDEGVEFIEASNDSSLSDFLQQSEQEDFDQLFPNDLIWCDPHIKGDTEGITCPLSIQVNHFACGGVAVASSLYHKVGDGRTLLNFINYWAAVTAKKDTSSINPHFFPHPHKKTHLIEFLRARSRSGCVTRSFKFPNQKLSDLKAKVTAMIMESGQPLKNPTRVEVLSWLLHKCAMVAAKQTNSGNFMEQSGMLIPIDLRGILVEKLPGTSVGNMNFGIEVPTRNESELAPNVSIGEMRKSKTTFQKIQNLDTATKILVGMSTETSLEMSKRLDPYYIYSSLCRFPTYEIDFGWGKPVKVTVGGTLKNVTMLMSTPDGNGIEALVCLEKQDMKIFQNSPELLAYC

SEQ ID NO: 5 - protein, 428 aa, Inula viscosa
MKIKIRSVQIIKPSKPTPKNHRTFKLSLIDQLAPFGNINIIFYYKSSGEEVNMLDRCSQLGKSLSEVLTLFYPLAGRVAKDGLEVDCSDQGVKFLETQVSMRLDDFLKQGPTIDLLSELIGAPRDQVTTTLLIVQVNVFDCGALVIGVSASHKVTDTCNLLRFINEWASINRTGGIDGAFSPSFDNLDSLFPPMKISSSSDQSPNPNDPIAKIISKRFLFNGTTISKLRAKASSSPNHRYSRVTLVTAIIWKVIISIDRVKSGSLRNCLLYPPMNLRGKAGSPISESSFGNVWAPYPIRLLQNELTEQSSFFDLVALIDGTTRNIIRGIQKASGEDICTQTLACYAEAAEELKQNKFCLFTSWCSFPIYNADFGWGKPFWASPVDSLMIHMVTLMDDKHGDGIEAWVCLQEKDMCLFEQDQDIIDFTS

SEQ ID NO: 6 - protein, 440 aa, Pulicaria dysenterica
MEIKNLTSKLVKPLTPTPSNLQNYDISFFDEQMPNINTPLILYYSTSQESPNDNIFDHLETSLSKTLTDFYPLAGRYMRQGSFVDCSDQGVLYIKSMANFRLEDFLGQAWELKFTMLNDLLPCEVPEAGEVDDPLLCIKVTAFECGGFAIGMCFSHKISDMCTMCTFINNWATRSSQENVNKLELEKYSPIFSVAQDFPKVALDDLSPRFPRSIIGMATNVKVFQFKVDAISKMRENLQISKDERNHHSSKIQLIVALFLKALMAIDKAKIGHSKSSIAQQGVNLRNKVVPKLPENLFGNFITLLHGQIEPEEGENMDLDGFLVILNDSVKKIEGEYAKALMSSHKDYEVLVKPFLKFGETLTNNNVNFYSFTSWCKFSFYKADFGWGKPVWRSTGHYAAEKLVIMMDDEEGGGVEAWIHLDEKSMSQLEQDPYIKAYAT

SEQ ID NO: 7 - protein, 440 aa, Pulicaria dysenterica
MEIKNITSKFVKPLTPTPSNLQNYDISFFDEHIPNINTPLILYYSTSQNSPNNNIFDHFETSLSKTLTDFYPLAGRYMRQGSFVDCSDQGVLYIQSKANFRLEEFLDLAWGLKFKMLNDLLPCEVPEAGEVDDPVLCVKVTAFECGGFAIGMCFSHKISDMCTMCTFINNWATRSSQENVNKLKLEKYSPIFSVARDFPKVALDDLSPRFPRSNIGMKTKVKLFQFKVDAISKMRENLHLSKDKTIHHSSKVQLIVALFLKALMAVDKAKIGHSKPSIAQQGVNLRNKVVPKLPENLFGNFISLLHGQIEPEEGENMDLDGFLVILNDSIKKIEGDYAKALMSSDKDYEVLVKHFLKFGETFANNDVNFYSFTSWCKFSFYKVDFGWGKPVWRSTGHYASQKVVIMMDDEEGDGVEAWVHLDEKSMCQLEQDPYIKAYAT

SEQ ID NO: 8 - DNA, 1311 bp, Inula viscosa
  source 1..1311: mol_type = genomic DNA
atggagataaaaagcattacctccaaactagtgaaaccctcaacaccaactcgatctaaccttcaaaactatgacatctctttctttgatgagcaaatgcccaacatcaacacacctctcattctctactactcagcatcacaaaactcaccagatgataacatctttaatcatttggagacttcactatccaaaaccttaaccgatttttaccccctagccgggagatacaagcgtcaaggttcgtttgttgattgtagtgatcaaggtgttctatatatccaatctatagcaaatttccgacttgaagaatttctaggccaagcgtgggagttaaaattcagaatgctaaacgatcttctcccatgtgaggtacctgaggccggtgaagtcgatgatccgttgttatgtattaaagtcacggctttcgaatgcggtggttttgcaattggtatgtgtttagcacataagatagctgatatgtgtaccatgtgcactttcattaacaattgggctactagaagtagccaagaaattgttaataaacaagagttagaaaaatattctcctatttttagtgtcgcacatgacttcccaaaagtggctctagatgatcttagcccaagattaccaaggtcaaacattgggatggagactaaggtgaagttgtttcaatttaaagtggatgcaatatccaaaatgagggaaaaccttcgggataagagttatcattcctcaaaggtacaacttattgttgcactattctcgaaggccttgatggccatagataaagcaaaattcggacactctaagccgtcgattgttcaacaaggagttaacttgcggaataaggtagtccctaagttacccgaaaatttatttgggaatttctttagcttcctacatggacgaattgagcccgaggatggtgaaaacatggatcttgatggtttcctgatgatcttgaaggactcgatgaagaaattcgaggatgagtatgttaaggcactaatgtctagtgacaaagattacgaggttttagttaagccctttttaaagtttggtgaaacttttgcaaataatgatgaaaatttccactgtttcacttcttggtgcaaattctcattttataaagctgactttggttggggaaagccagtttggagaagcacaggacattatgcaaaccataaatttgtgatactgatggatgatgaagaaggtggtggggtagaagcatgggttcatcttgatgaaaaaagcatgtgtcaattagaacaagatccagatatcaaggcctatgcaacttag

SEQ ID NO: 9 - DNA, 1314 bp, synthetic construct
  source 1..1314: mol_type = other DNA
atggaaatcaagtctattacttctaagttggtcaagccatcaactccaactagatctaatttacaaaactacgatatttcctttttcgacgagcaaatgccaaacatcaacactccattaattttgtactattccgcctcacaaaactccccagatgacaacatttttaaccatttagagacttccttatctaagactttgaccgatttctacccattagctggtagatacaaaagacaaggttcctttgtcgactgctctgatcaaggcgttttgtacatccaatctattgctaacttcagactagaggaatttttaggtcaggcttgggaattgaagttcagaatgttgaacgacttattgccatgtgaagtcccagaagctggtgaagttgacgatccattattgtgtattaaggttaccgctttcgaatgcggcggtttcgccatcggtatgtgtttggcccacaaaattgccgacatgtgtaccatgtgtactttcatcaacaattgggctaccagatcttcccaagaaattgttaacaagcaagagctagaaaagtactccccaattttttctgtcgcccacgattttcctaaggtcgctttggatgacttgtccccaagattgcctagatctaacatcggtatggaaaccaaggttaagttgttccaattcaaagttgatgctatttctaagatgagagagaacttgagagataagtcctaccactcctctaaggttcaattgattgttgccttgttctctaaggccttgatggccattgacaaggctaaattcggtcactctaagccatccattgttcaacaaggtgtcaacttaagaaataaggttgtccctaagttgccagaaaacttgtttggtaacttcttttccttcttgcatggtagaatcgaaccagaagacggtgaaaacatggatttagatggttttttgatgatcttaaaagattctatgaagaaattcgaagacgaatacgtcaaggctttgatgtcctctgataaggactacgaagttttagtcaagccattcttgaaattcggtgaaactttcgccaataacgacgaaaatttccactgtttcacctcatggtgtaaattctccttttacaaggccgacttcggttggggtaagccagtctggagatccaccggtcattacgctaaccataagttcgtcattttaatggacgatgaagagggtggcggtgttgaggcttgggttcatctagatgaaaaatctatgtgtcaattggaacaagatccagatatcaaggcttacgctacttaataa

SEQ ID NO: 10 - DNA, 1311 bp, synthetic construct
  source 1..1311: mol_type = other DNA
atggaaatcaaatctatcacctctaaactggttaaaccgagcaccccaacccgctctaacctgcagaactacgatatctcctttttcgatgaacagatgccgaacattaacaccccactgatcctgtactattctgcttctcagaattctccggacgataacatctttaaccatctggaaaccagcctgtctaaaaccctgactgatttctacccgctcgcaggccgctacaaacgtcagggcagcttcgttgattgcagcgatcaaggtgtcctgtacatccagtctatcgcaaacttccgcctggaggaatttctgggccaagcgtgggaactgaaattccgtatgctgaatgacctcctgccttgcgaggttcctgaggcgggtgaagtggatgacccactcctgtgcattaaagtgactgccttcgaatgcggtggcttcgccattggcatgtgcctggcgcacaagattgcagatatgtgtaccatgtgtacctttattaacaattgggcaactcgctcctctcaggagatcgtaaataaacaggaactggaaaaatacagccctatcttcagcgttgcgcacgatttcccaaaagttgcactcgatgacctgagcccgcgtctgcctcgtagcaacatcggtatggaaaccaaagttaaactgtttcagttcaaggtagacgcgatctccaaaatgcgcgaaaacctgcgcgacaagagctatcactcttccaaggtgcagctgatcgttgcgctgtttagcaaagcactcatggcaatcgacaaagcgaagttcggtcacagcaaaccgagcatcgttcaacagggtgttaacctgcgtaataaagttgtaccgaagctgccggaaaacctgttcggcaacttctttagcttcctgcatggccgtatcgaaccggaggatggtgaaaatatggacctggacggttttctgatgatcctcaaagactctatgaagaaattcgaagatgaatacgttaaagcgctgatgtcctctgacaaagactacgaggtactggtaaaaccgtttctgaaattcggcgaaaccttcgcgaataacgacgaaaacttccactgctttacttcctggtgcaaattctctttctacaaagcagacttcggctggggcaagccggtatggcgcagcaccggccactatgccaaccacaaatttgtgattctgatggacgatgaggaaggtggcggtgtcgaagcgtgggtgcatctggacgagaaaagcatgtgccagctggaacaggatccggacatcaaggcttacgcgacttaa

SEQ ID NO: 11 - DNA, 1461 bp, Erigeron canadensis
  source 1..1461: mol_type = genomic DNA
atggagataaaaaactatgcctcaaagcttgtaaaaccttcaaaaccaactccatccactcttcgtcgctataacatctctgtctttgatgaacaagtagctaacatcaacacaaatctcatcctctactacactgcatcatcacaaaatgataataaacatttcgacatctttagtcataatttagagatttcgttatcaaaaaccttaaccgatttttacccattttctggtagatacattcggaatgcttccttagtcaattgtagtgatcaaggtgctctatatgtccaagccaaagcaaatttcagactctcggaatttttagggctagcatgggacttgaaactcgatatgctacaagatttgcacccgtgtgatttcggtgaggctggtgaaatagatgatcctatggtttctgttaaggttacaaagttcgaatgtggtggtgttgttattggtatgtctttttcacataagatttctgatatgtctacaatgtgtatatttattaataattggtccgctagaacaaaaagcctacaaataggagtcataatgaagtagaattagaaaaaagaaaatattatcctgattttagtttggcccaactttttccaaaaagaggtctaattgataatagcccaagagttccaaggtcaagaattggaatgaaaaatcaagtacaaaaatttatattcacaagaaatgctatatctaaaataagagaaaaaataaacatgattgatgacaataataggccatcaaaagtacaaattattgtagcattgttatggaaggccttggtaggcatagataaagcaaatggacaatcaaagatcgcatatgcataccaagtagttaacatgaggaataaagtagtccctaaattagccgataactcatttggcaactttgttacttttgcaattgtgcatattaatcccaatgaaagagacaatgtcgttgatcttcaaggtttcgttaagctcttgcacgagtcaatcaaagatatagataatatttgtgccggattaatgacacacggtgacacaaaatatgagcttttgtctaagcgcttaatggagaatagccaagattctagcaataaggagatatactactttacttcttggtgcagattctcattctatggagctgattttggtttgggtaagccgatttggagaagcccaggaaagtttccgggccaaaatttagtgaccatgatggactatgatcaagagggtggtgaggggatagaagcatgggttcatttagatgaaaatcgcatgtcccaattagaacaagatcccgatattatggcctatgcaatttaattaggctcatttgtggtatgatcattatacgatttttcttttgtggctccgactgtttttgtgtcggtatgtaataaagatgtctttataaccagatatttatcgaattaattttgattaa

SEQ ID NO: 12 - DNA, 1341 bp, synthetic construct
  source 1..1341: mol_type = other DNA
atggaaattaagaactatgcttccaagttagtcaagccttcaaaaccaaccccatccactttgcgtagatacaacatctccgtctttgacgaacaagtcgctaacattaatacaaacttgattttgtattacacagcctcctctcaaaacgataacaagcacttcgatattttttctcacaacttagaaatctctttgtccaagactttaactgatttctatcctttctctggtagatacattcgtaacgcctctttggtcaactgttcagaccaaggtgctttgtacgttcaagctaaagctaacttcagattgtctgaattcttgggtttggcctgggatttaaagttggatatgttacaagatttacacccatgtgatttcggtgaggctggtgaaattgacgatcctatggtttctgttaaggtcaccaaattcgaatgtggcggtgtcgttatcggtatgtctttctctcacaagatttccgacatgtccactatgtgtattttcatcaataactggtccgctagaactaagtccttgcaaattggtgtcaataacgaagtcgaactagaaaaaagaaagtactatccagacttctctttagctcaattattcccaaagagaggtttaatcgataattccccaagagtcccaagatctagaattggtatgaagaaccaagtccaaaagtttatctttaccagaaacgctatttccaagattagagaaaaaatcaatatgatcgatgacaataacagaccatctaaggttcaaatcattgtcgctttattgtggaaagccttggtcggtatcgataaggctaacggtcaatctaaaattgcctacgcttaccaagttgtcaacatgagaaacaaggttgtcccaaagttggctgacaactcctttggtaattttgttactttcgctattgttcacatcaatccaaacgaaagagacaacgtcgttgacttgcaaggttttgtcaagttgttacatgaatctatcaaggatatcgataacatttgtgccggtttaatgacacacggtgataccaagtacgaactattgtctaagagattaatggaaaattcccaagattcttccaacaaggagatttattacttcacttcttggtgtcgtttctctttctatggtgctgatttcggcttgggtaagccaatttggcgttccccaggtaaattcccaggtcaaaacttggtcactatgatggactacgaccaagaaggcggtgaaggtattgaggcctgggttcacttggacgaaaacagaatgtcacaactagaacaagatccagacattatggcttacgctatctaa

SEQ ID NO: 13 - DNA, 1341 bp, synthetic construct
  source 1..1341: mol_type = other DNA
atggaaattaaaaattacgcttctaaactggtaaaaccgagcaagccgacgcctagcacgctgcgccgttacaacatcagcgtattcgatgaacaggttgctaacatcaatactaacctcatcctgtactataccgctagctctcagaacgataacaagcacttcgatattttctcccacaacctcgaaatctccctgtccaagaccctgactgacttctacccgttctctggccgttatattcgcaacgcatccctggtgaattgcagcgaccagggtgcgctgtatgttcaagctaaggccaacttccgcctgtccgaattcctgggcctggcttgggatctgaagctggatatgctgcaagatctgcatccgtgcgacttcggtgaagcaggtgaaatcgacgatccgatggtttccgtgaaggtcaccaaattcgaatgcggcggtgttgtaatcggcatgtccttcagccacaaaatttctgatatgagcactatgtgtatcttcatcaacaattggagcgcccgtaccaagtccctgcagatcggcgtaaataacgaagtcgaactggagaaacgcaagtattacccggacttttccctggcccagctgttcccgaaacgtggtctgattgataatagcccgcgcgtaccgcgtagccgcatcggtatgaagaaccaggtacagaaattcatttttactcgcaacgctatctctaaaatccgtgaaaaaattaacatgattgacgataataaccgtccgagcaaagttcagattatcgtggcactcctgtggaaagctctggtgggcattgataaggcaaatggccagtccaagatcgcctatgcgtaccaggtagttaatatgcgtaataaagtcgttccaaaactggcggataacagcttcggtaactttgttaccttcgctatcgttcacatcaacccgaacgagcgtgataacgtggttgatctgcagggtttcgtcaaactcctgcacgaatccatcaaagacattgataacatctgcgcgggcctgatgactcatggcgacaccaaatatgaactcctgtccaagcgtctgatggaaaattctcaggattcttccaacaaagagatctattacttcacgtcttggtgccgcttctctttttacggtgcagatttcggcctgggcaagccgatctggcgttctccgggtaaattcccgggccaaaacctcgttacgatgatggattatgaccaggaaggcggtgaaggcatcgaggcatgggtacacctggacgaaaaccgtatgtcccagctcgaacaggacccagacatcatggcgtacgcgatctaa

SEQ ID NO: 14 - DNA, 1266 bp, Erigeron canadensis
  source 1..1266: mol_type = genomic DNA
atggagatcaagattcaatccactcagttcataaaaccatccaagttaaccccggaagtttttcgcgatttcaagctatctattctagatcaatttgctgcatcttcatacataaaccaggtcttctactataagactagttgtaaagttgacatcccagatatatgtggtcaactggtcaactctttatctgagatactaactttgttctacccattagccggaagaatcagagaaaccgagctagaagttgactgtagtgatcaaggagtcaagtatctggaaactcaagttagcataagtttggataaattttttgaactaggtcctaagattgaccatattagacgacttatccgtgcaccagaccaagaaaaaagcgcgttgttaattgtccaggtgaacatctttaaatgtggctcacttgttattggagtgagtggttcacataaagtcactgatgcatacaatttagtcaggtttgttaacgaatgggctagcttgaaccgtacagggcaaacaagtggtgccttttctatttcttttgataatttagattctgttttcccaccaaaagaaattgaatcatttgaggactcacccgttatagataagtcacaaaccaaaatagttacgaaaagatttgtgtttaatgggagtataatatcaaaactaagagcaaagtcgggtcctagaaatcctagacatagtcgagtaaccttggtggctgcaattatatggaaggctattatctccgttgaccaagtcaaaagtgggagccttaggaattctgtcttggcccctgcgattaatttaaggggaaagacaaggttaccgatatctgaaagctcgtttgggaatgtgtgggttccttactcaattcgttatttacagaacgaaatggaacccaagtttgataatcttgtgaaattgatagaaaacacaacaagagatgttatcacacgtcttttaaatgcgactagtgaagagatatgtaaagaggcaatggcatgttatgctgaagttggtgaagaactaaagcagaacaagtttgctatatttactagttggtgtaactttccgatatacaatgctgattttggttttggtaagccgtgttgggtaagtgaagcggggcgttcacttgatatggtaacgttaatggatgacaaacatggggatggaattgaagcatgggtgagtttgaatgagaaagacatgtctgtgtttgaacaaaatgaggacattttaagtgtattgtcttaa

SEQ ID NO: 15 - DNA, 1266 bp, synthetic construct
  source 1..1266: mol_type = other DNA
atggaaattaaaatccaatctacacaattcatcaagccatccaagttgactccagaagtcttcagagatttcaagttgtccatcttggatcaattcgctgcctcctcttacattaatcaagtcttctattacaagacttcttgcaaagtcgacattccagatatttgtggtcaattggttaactctttatctgaaattttaaccttgttctaccctttggccggccgtatcagagaaaccgaattggaagtcgattgttccgaccaaggtgttaaatacttagaaacccaagtttccatttccttggacaagtttttcgaattgggtccaaaaattgaccacattcgtagactaattagagccccagaccaagaaaagtcagctttgttaatcgttcaagttaacatcttcaaatgtggttccttggttatcggtgtctctggttcccacaaggtcaccgatgcttacaacttggtcagattcgtcaatgaatgggcctccctaaacagaactggtcaaacctccggtgccttctctatctccttcgacaatttggactccgtcttccctccaaaggaaatcgaatccttcgaagattctcctgttattgataagtctcaaaccaagattgttactaagagattcgtctttaacggttccatcatttccaagttgcgtgctaaatccggtccaagaaatcctagacactcaagagttaccttggtcgccgctattatctggaaggctatcatttctgttgaccaagttaagtctggttccttgagaaactccgttttagctccagctattaacttgagaggtaaaactagattgccaatctctgaatcctctttcggtaacgtctgggttccatactctatcagatatttgcaaaacgaaatggaacctaaattcgataacttagtcaagctaatcgaaaataccactagagatgtcattaccagattgctaaacgctacctccgaggaaatctgtaaggaagctatggcttgctatgctgaagttggtgaggaattgaaacaaaacaagttcgctatttttacctcctggtgtaacttcccaatctataacgctgactttggtttcggtaagccttgttgggtttctgaagctggtagatcattggatatggttaccttaatggatgacaagcatggtgacggtatcgaggcttgggtttctttgaatgaaaaggatatgtctgttttcgaacaaaacgaagatattttgtcagttttgtcttaa

SEQ ID NO: 16 - DNA, 1266 bp, synthetic construct
  source 1..1266: mol_type = other DNA
atggaaatcaaaatccagtctacgcagttcatcaaaccgagcaaactgaccccggaggtcttccgcgatttcaaactgtctattctggaccagttcgcagctagctcctatattaaccaggtattctactataagactagctgtaaggttgatatccctgatatttgcggccagctggttaactccctgtctgaaatcctgactctgttttacccgctggcaggtcgtatccgcgaaaccgaactggaggtggattgcagcgaccagggtgtgaaatacctggaaacgcaggttagcatcagcctggacaaatttttcgaactgggtccgaaaatcgatcacatccgtcgcctgatccgtgcaccggatcaggaaaaatccgctctcctgatcgtccaggtaaatatttttaaatgcggttccctggtgatcggcgtcagcggctctcataaagtaactgacgcgtacaacctggtgcgtttcgtcaacgaatgggccagcctcaaccgtacgggtcagacgtctggtgcgttctctattagcttcgataacctggattctgtgttcccgcctaaagaaattgagagcttcgaggattccccggttattgacaaatctcagactaaaatcgttactaagcgcttcgtgttcaacggttccatcatttctaaactgcgtgctaaatccggcccgcgcaacccgcgccactcccgtgtcacgctggtggcggcaattatctggaaagcgattatcagcgtcgatcaggtaaaaagcggtagcctgcgtaactccgttctggcgcctgctatcaacctgcgtggtaagacccgtctgccgatcagcgagtcttccttcggtaacgtgtgggtcccgtattctatccgttatctccaaaacgaaatggaaccgaaattcgacaacctggtcaaactgattgaaaacaccactcgtgatgttattacccgcctcctgaacgctacctctgaagagatctgtaaggaagcaatggcatgctatgctgaagtgggtgaggaactgaaacagaacaaattcgcaatcttcaccagctggtgcaactttccgatctataacgcggatttcggcttcggcaaaccgtgctgggtttctgaggcaggtcgctccctggacatggttactctgatggatgacaaacatggtgacggcatcgaagcgtgggttagcctgaacgagaaagatatgtctgttttcgaacagaacgaagatattctgtccgtactgtcctaa

SEQ ID NO: 17 - DNA, 1332 bp, Inula viscosa
  source 1..1332: mol_type = genomic DNA
atgttgaacaagcttctaaaatatggaagaaggcaacttcacacaattgtatctagagatatcatcaaaccctcatctccgaccccttctcaacacaaaacctataatctttcgttgttcgatcaaatcactggaaattcatactcaccaattgttgccttttacccagtccccggtgttcgtcaaagttcacatggtaaaacactcgagctaaagaaatccttatcacaaaccctaactcaatactatccatttgccggtagggtacctaaatctttaccaacttatgttgattgcaatgatgagggggttgagtttattgaagcaagcaatgatagctcattgtcggactttctccaacaatcggaacaagaagattttgatcaactctttccaaatgatcttatatggtgcgacccacatattaaaggtgacactgagggtatcacttgcccgttgtcaattcaagttaaccattttgcatgtggaggtgtagcggtggcgtcatctttatatcataaggttggtgatgggcgaactttgttaaatttcataaattattgggccgctgtgacggccaaaaaagatacatcatccattaatccacatttctttcctcacccgcataaaaagactcatttgatagaatttttaagagccagatcacgtagtggttgtgtcacgagaagttttaagttccctaaccaaaagttaagcgatctgaaagccaaggtcacagccatgataatggaatctggacaaccactcaaaaaccctactcgggttgaagttttatcatggctactacataagtgtgcaatggtagccgctaaacaaacaaattcagggaactttatggaacaaagtggcatgcttattccaatagatttgagaggcattttggtagagaaattgcctggaacaagtgttggaaatatgaactttgggatcgaagttccaacacgaaatgaaagtgaacttgcaccaaatgtgtcaattggtgaaatgaggaaaagtaagacaacatttcaaaaaattcaaaacttggacactgcaactaaaatcctagttggaatgtcaaccgaaacttctttagaaatgtcaaaaagactagatccctattacatatattcaagcttgtgcaggtttccaacatatgagatcgattttggctgggggaagcccgtaaaagtaactgtcggtggaacgctaaagaacgtaacaatgttgatgtccactccagatggtaatggcatcgaagcacttgtgtgtcttgaaaaacaagacatgaagatatttcaaaacagccctgagttgctggcctattgctaa

SEQ ID NO: 18 - DNA, 1335 bp, synthetic construct
  source 1..1335: mol_type = other DNA
atgttgaacaaactattgaagtacggtcgtagacaattacacaccattgtttcccgtgacatcattaaaccatcctctccaaccccatcccaacacaaaacatacaatctatctttattcgatcaaattactggtaactcttactctccaatcgtcgccttttacccagttccaggtgtcagacaatcttcacatggtaaaaccttagaattgaaaaagtccttgtctcaaaccttgacccaatactatccattcgccggtagagtcccaaagtctttgccaacttacgttgactgtaatgacgagggtgttgaattcattgaggcttccaacgactcctctctatctgacttcttgcaacaatccgaacaagaagatttcgaccaattattcccaaacgatttaatttggtgtgacccacacatcaagggtgataccgaaggtattacttgcccattgtctattcaagttaatcatttcgcttgtggcggtgtcgctgttgcttcttccttgtaccacaaggtcggcgacggtagaaccttattgaacttcatcaactactgggccgctgtcactgccaaaaaggacacttcctctatcaacccacactttttcccacacccacacaaaaagactcacttgattgaattcttgagagctcgttcccgttccggttgcgttaccagatctttcaagttcccaaatcaaaagttgtctgatttgaaagctaaggttaccgctatgattatggaatctggccaaccactaaagaacccaactagagttgaagttttatcatggctattgcacaagtgtgctatggttgccgctaagcaaactaattccggtaacttcatggaacaatctggtatgttgattccaattgacctaagaggtatcttggtcgaaaagttaccaggtacttctgtcggtaacatgaacttcggtattgaagttccaaccagaaacgaatccgaattagccccaaatgtctctatcggtgaaatgagaaagtctaagaccactttccaaaagattcaaaacttggatactgccactaagattttggtcggtatgtcaactgaaacttctttggaaatgtctaagagactagatccatactatatctactcctctttgtgtagattcccaacttacgagatcgatttcggttggggtaagccagttaaggtcactgtcggcggtactttgaagaacgtcaccatgttaatgtccaccccagacggtaatggtatcgaggctttggtctgtttggaaaagcaagacatgaaaatctttcaaaactctccagaattattggcttactgttaataa

SEQ ID NO: 19 - DNA, 1332 bp, synthetic construct
  source 1..1332: mol_type = other DNA
atgctgaacaaactcctgaaatacggtcgccgtcagctgcataccatcgtttctcgtgatattatcaaaccatccagcccaaccccatcccagcataaaacctataatctgagcctgttcgaccaaatcaccggcaactcctactccccgattgtggcattttacccggttccgggcgttcgtcagtccagccacggcaagacgctggaactgaaaaagtccctgtcccagaccctgacccagtactatccgttcgctggccgtgtgccgaaatccctgccaacgtacgtggattgcaacgacgagggcgttgaattcatcgaagcgagcaacgacagctctctgtctgactttctgcaacagtccgagcaggaagatttcgatcagctgtttccgaacgacctgatttggtgcgacccgcacatcaaaggtgacaccgaaggtatcacttgcccgctgtctatccaagtaaaccacttcgcgtgtggcggtgtggcagttgcatcctctctgtatcacaaagtcggtgacggccgtaccctcctgaatttcattaactattgggcagctgtgacggctaagaaagatactagctccatcaacccgcactttttcccgcatcctcataagaaaactcacctgatcgaatttctgcgcgcgcgcagccgtagcggctgtgtcactcgtagcttcaagttcccgaaccagaaactgtccgacctgaaagcgaaagtgaccgccatgatcatggaatccggccagccgctgaaaaatccgacccgtgtggaggtgctcagctggctcctgcacaagtgcgctatggtggccgcaaagcagaccaactccggtaacttcatggagcagagcggcatgctgatccctatcgatctgcgtggcatcctggttgaaaaactgccgggcacttccgttggtaacatgaacttcggcatcgaagttccgacccgtaacgaaagcgaactggcacctaacgtttccatcggtgagatgcgtaaaagcaagactaccttccaaaaaatccagaacctggataccgctactaaaattctggttggcatgagcaccgaaacttctctggagatgtccaagcgtctggatccatattacatctactctagcctctgtcgttttccgacctacgaaattgatttcggctggggtaaaccggttaaagttaccgtcggtggcaccctgaaaaacgtgactatgctgatgtctactccggacggtaacggtatcgaagctctcgtgtgcctggaaaaacaggacatgaaaatcttccagaacagcccggagctcctggcgtactgctaa

SEQ ID NO: 20 - DNA, 1287 bp, Inula viscosa
  source 1..1287: mol_type = genomic DNA
atgaagattaagattagatctgtacagatcataaagccatcaaaaccgacccctaaaaatcatcgaacttttaagttatcgttgattgatcaacttgctccatttggaaacataaatataatcttctactataagtccagtggtgaggaggttaacatgttagatagatgctctcaactgggtaagtctttatcggaggtgttaactttgttttacccactagctggcagagttgcaaaagatgggcttgaggttgattgcagcgatcaaggggttaagttcttggaaactcaagtgagtatgaggctcgatgattttctcaaacaaggtcccacaatcgacctccttagtgaactcataggggcaccgcgagatcaggtcaccaccacgttgctaattgtccaggtcaatgtctttgattgcggtgcgcttgttattggagtgagtgcttcacacaaagtcacagacacatgcaacctgctgaggttcattaatgagtgggcgagcataaaccgcacagggggcatcgatggtgccttttctccttcctttgataacttggattctctctttccaccgatgaaaatttcatcatctagtgatcaatctcctaatccaaatgatcccatagcaaaaataatcagcaaaaggtttttgtttaatggaactacaatatcaaagctaagagcaaaagccagtagttcaccaaatcatagatatagtcgagtaaccttggtgactgcaataatatggaaggttattatctccatcgatcgagtcaaaagtgggagcctcagaaactgcttattgtacccacccatgaatttgagaggaaaggcaggctcaccaatatcagaaagctcatttgggaacgtatgggctccttatccaatccgtttgttgcaaaacgaattaacggaacaatccagtttttttgatcttgtagccctgatagatggcacaacaagaaacattatcagggggattcaaaaagcaagtggtgaagacatatgcacacaaacattggcttgttatgccgaggcagcagaagagctaaagcagaacaagttttgcctttttacgagttggtgttcgtttcctatatacaacgctgatttcggttggggtaaacctttctgggcaagcccagtagatagcctcatgattcacatggtcactttaatggatgacaaacatggcgacggaattgaagcatgggtctgtttgcaggaaaaagacatgtgtttgtttgaacaagatcaggatattatagacttcacctcataa

SEQ ID NO: 21 - DNA, 1287 bp, synthetic construct
  source 1..1287: mol_type = other DNA
atgaaaatcaaaattagatctgttcaaattatcaaaccatctaagccaacaccaaagaaccaccgtacattcaagttgtctctaatcgatcaattggctcctttcggtaatattaacattatcttttattacaaatcctctggtgaggaagtcaacatgttagacagatgttcacaattgggtaaatctttatctgaagttttgactttattctacccattggccggtagagttgctaaggatggtttggaagtcgactgttctgaccaaggtgttaaattcttggaaactcaagtttctatgcgtttggacgattttttgaaacaaggtcctaccattgacttgctatctgaattgatcggtgctccacgtgaccaagttactaccactttgctaatcgtccaagtcaatgttttcgattgtggtgctttggttattggtgtctctgcttcccataaggtcaccgacacttgtaacttactaagattcatcaacgaatgggcctccatcaatagaactggcggtattgatggtgctttctctccatccttcgataacttggattctttgttccctccaatgaaaatttcttcctcttccgaccaatccccaaacccaaacgatcctattgctaagatcatttctaagagatttttgtttaacggtactaccatttctaaattaagagctaaggcttcctcttcccctaaccatcgttactctagagttaccttggttacagccatcatttggaaggttattatctctattgacagagtcaagtctggttccttgagaaactgtttattgtaccctccaatgaacttgcgtggtaaggccggttccccaatttccgaatcttccttcggcaacgtttgggccccatacccaattagattattgcaaaacgaattgaccgaacaatcctcttttttcgacttggttgccttgattgatggtaccactagaaatattatcagaggtatccaaaaggcttctggtgaagacatctgtacccaaaccttggcctgttacgctgaagccgctgaggaattgaaacaaaacaagttttgtttgttcacttcttggtgctccttcccaatctacaacgctgactttggctggggtaaaccattttgggcttctccagttgattctttgatgattcacatggttactttgatggacgataagcatggtgacggtatcgaggcttgggtttgtttgcaagaaaaggacatgtgtttgtttgaacaagaccaagatattatcgacttcacttcttaa

SEQ ID NO: 22 - DNA, 1287 bp, synthetic construct
  source 1..1287: mol_type = other DNA
atgaaaatcaaaatccgttccgtgcagattatcaaaccgtctaaaccgactccgaaaaaccaccgtacctttaaactgtctctgattgatcagctggcaccgttcggtaacattaacattatcttttattacaagagctccggtgaggaagttaacatgctggaccgctgctcccagctgggtaaatctctgtccgaagtgctgaccctgttctatccgctggctggtcgcgttgcgaaagacggcctcgaagttgactgtagcgatcagggtgtgaagttcctggaaacccaggtttctatgcgtctggatgactttctcaaacagggccctaccatcgatctcctgagcgaactgatcggtgcgccgcgcgaccaggtgaccacgaccctcctgatcgtgcaggtgaatgtctttgattgcggtgccctggttatcggcgttagcgcgagccacaaggtgaccgatacttgcaatctcctgcgttttatcaatgaatgggcttccatcaaccgtaccggtggcattgacggcgctttctctccgtcttttgacaacctggacagcctgttcccaccgatgaaaatttctagctcttccgatcagtccccaaacccgaacgatccaatcgctaaaatcattagcaaacgcttcctgttcaatggtacgaccatctccaaactgcgcgctaaggccagctctagcccgaaccaccgctacagccgcgttaccctggtcaccgctattatctggaaagttattatctccatcgatcgtgtaaaatccggttctctgcgtaactgcctcctgtacccaccgatgaacctgcgtggcaaagcgggttccccgatttctgaatccagctttggtaatgtttgggcgccgtatccgattcgcctcctgcagaacgagctgacggaacagtcctcttttttcgatctggttgcactcatcgatggtaccacgcgtaacattatccgtggtatccagaaggcatctggcgaggatatctgtacccagaccctggcatgctacgcggaagcggctgaggaactgaaacagaacaaattttgtctgtttacctcttggtgttcttttcctatctacaacgcagatttcggctggggtaaaccgttctgggctagccctgtcgacagcctgatgatccacatggtaacgctgatggatgacaagcatggtgacggcatcgaagcatgggtttgcctgcaggaaaaggatatgtgcctgttcgaacaagaccaggacatcattgactttacctcttaa

SEQ ID NO: 23 - DNA, 1323 bp, Pulicaria dysenterica
  source 1..1323: mol_type = genomic DNA
atggagataaaaaaccttacctccaaactagtgaaacccttaacaccaactccatccaaccttcaaaactatgacatctctttctttgatgagcaaatgcctaacatcaacacacctctcattctctactactccacatcacaagagtcaccaaatgataacatatttgatcatttggagacttcactatcgaaaaccttaactgatttttaccccctagccgggagatacatgcgtcaaggttcgttcgttgattgtagtgatcaaggtgttctatatatcaaatctatggcaaactttcgactagaagattttctaggccaagcgtgggagttaaaattcacaatgctaaacgatcttctcccatgtgaggtacctgaggctggtgaagtcgatgatccgttgttatgtattaaagtcacggctttcgaatgcggcggttttgcaattggtatgtgtttttcacataagatttctgatatgtgtaccatgtgcacattcattaacaattgggcaactagaagcagccaagaaaatgttaataaactagagctagaaaaatattctcctatttttagtgtggcacaggacttcccaaaagtagctctagatgatcttagcccaagatttccaaggtcaatcattgggatggcaactaatgtgaaggtgtttcaatttaaagtggatgcaatatccaaaatgagggaaaaccttcaaatatccaaggatgaaaggaatcatcattcctcaaagatacaacttattgtggcactattcttgaaggccttgatggccatagataaagctaaaatcggacactcgaagtcgtcgattgctcaacaaggagttaacttgcggaataaggtagtccctaagttacccgaaaatttatttgggaatttcattaccttactacatggacaaatagagcccgaggaaggtgaaaacatggatcttgatggtttccttgtaatcttgaatgactcggtcaagaaaatcgaaggcgagtatgctaaggcactaatgtctagtcacaaagattatgaggttttagttaagccatttttaaagtttggcgaaactttaaccaataataatgtaaatttctactctttcacttcttggtgcaaattctcattttataaagctgactttggatggggaaagccagtttggagaagcacaggacattatgcagctgagaaacttgtgataatgatggatgatgaagaaggtggtggggtagaagcatggattcatcttgatgaaaaaagcatgagtcaattagaacaagatccatatattaaagcctatgcaacttag

SEQ ID NO: 24 - DNA, 1323 bp, synthetic construct
  source 1..1323: mol_type = other DNA
atggaaatcaagaacttgacttccaagttggtcaagccattgactccaaccccatcaaacttgcaaaactatgacatctcttttttcgatgaacaaatgccaaacattaacaccccactaattttatattactccacctcccaagaatctcctaacgacaacatctttgatcacttggaaacttctctatctaagactttgaccgacttttaccctttggctggtagatacatgagacaaggttctttcgttgattgttccgatcaaggtgttttgtacattaagtctatggctaacttcagattggaagacttcttaggtcaggcttgggaattgaagttcactatgttgaacgacttattgccttgtgaagttccagaagctggtgaggttgatgacccattattgtgtatcaaagttaccgccttcgaatgtggcggttttgccattggtatgtgcttctcccataagatttccgacatgtgtaccatgtgcactttcatcaataactgggctaccagatcttcccaagaaaatgtcaataagttggagttggaaaagtactctccaatcttctctgttgctcaagactttccaaaggttgctttggatgacttgtcaccaagatttccaagatctatcattggtatggctaccaacgttaaagttttccaatttaaggtcgatgccatttctaaaatgagagaaaacttacaaatttccaaggatgaaagaaatcaccattcttccaaaatccaattaattgtcgctttgttcttgaaggctttgatggctattgataaagccaagatcggtcactccaagtcttcaattgctcaacaaggtgttaacttgagaaacaaggttgtcccaaaattgcctgaaaacttgttcggtaacttcatcactttattgcacggtcaaatcgaaccagaagagggtgaaaacatggatttagatggtttcttggttatcttaaatgattccgtcaagaaaatcgaaggtgaatatgccaaggctttaatgtcttcccacaaggattacgaagtcttggtcaaaccattcttgaagtttggcgaaaccttgaccaacaataacgttaacttctattccttcacctcctggtgcaagttttctttttataaggccgattttggttggggtaaaccagtttggagatccactggtcactacgccgctgaaaagttggttatcatgatggacgatgaggaaggtggcggtgttgaagcctggattcacttggatgaaaagtctatgtcccaattagaacaagacccatatatcaaggcttacgctacttaa

SEQ ID NO: 25 - DNA, 1323 bp, synthetic construct
  source 1..1323: mol_type = other DNA
atggagatcaaaaacctgaccagcaaactggttaagccgctgactccgaccccgagcaacctgcagaactatgacatctcttttttcgatgaacagatgccgaacatcaacaccccgctgatcctgtattactctacctctcaggagtctccgaacgacaacatcttcgaccatctggaaacgtccctctccaaaactctgactgatttctatccgctggccggtcgttatatgcgtcagggtagcttcgttgactgctctgaccaaggcgttctgtatattaaaagcatggcgaactttcgtctggaggatttcctgggccaggcgtgggaactgaaattcaccatgctgaacgatctcctgccgtgcgaagtaccggaagctggtgaagttgatgacccgctcctgtgcatcaaagttactgctttcgaatgcggcggttttgctatcggtatgtgctttagccacaaaatttctgacatgtgcaccatgtgtaccttcatcaataactgggcgacgcgtagctctcaggagaacgttaacaaactggagctggaaaagtattccccaatcttctctgttgctcaggactttccgaaagtggctctggacgatctgtctccgcgttttccacgtagcatcattggcatggcaaccaacgtgaaagtgtttcagtttaaggttgatgctatttctaaaatgcgtgaaaacctgcagatcagcaaagatgagcgtaaccaccatagctctaagatccagctcatcgtggctctgttcctgaaagcgctgatggctattgataaagccaaaatcggccattccaaatccagcattgctcaacagggcgttaacctgcgtaacaaggtggttccgaaactgccggaaaacctgttcggtaacttcatcaccctcctgcacggtcaaatcgagccggaggaaggtgaaaatatggatctggatggttttctggtaatcctgaacgacagcgtcaagaaaattgagggcgaatatgcgaaagctctgatgagctcccataaagactatgaagtgctggtaaaaccgtttctgaaattcggtgaaaccctgaccaataacaatgttaacttctatagcttcaccagctggtgtaagttctccttctacaaagcggactttggttggggcaaaccggtatggcgttccactggtcattatgccgctgaaaaactggttatcatgatggacgatgaggaaggcggtggcgtcgaggcgtggatccacctcgacgaaaaatctatgtctcagctggaacaggatccatacatcaaagcctatgccacctga

SEQ ID NO: 26 - DNA, 1323 bp, Pulicaria dysenterica
  source 1..1323: mol_type = genomic DNA
atggagataaaaaacattacctctaaatttgtgaaacccttaacaccaactccatccaaccttcaaaactatgacatctctttctttgatgagcatattcctaacattaacacacctcttattctctactactcaacatcacaaaactcaccaaataataacatctttgatcatttcgagacgtccctatcgaaaaccttaaccgatttttaccccttagctgggagatacatgcgtcaaggttcgttcgttgattgtagtgatcaaggtgttttatatatccaatccaaagcaaatttccgactagaagaatttctagacctagcttgggggttgaaattcaaaatgctaaacgatcttctcccatgtgaggtacctgaggctggtgaagtcgatgatccggtgttatgtgttaaagtcacagctttcgaatgcggtggttttgcaattggtatgtgtttttcacataagatttctgatatgtgtaccatgtgcacattcattaacaattgggctactagaagcagccaagaaaatgttaataaactaaagttagaaaaatattctccaatttttagtgtggcacgtgacttcccaaaagtagctctagatgatcttagcccaagatttccaaggtcaaacattgggatgaaaactaaggtgaagttatttcaatttaaagtggatgcaatatccaaaatgagagaaaacctgcatttatccaaggataaaacgattcatcattcctcaaaggtacaacttattgtggcactattcttgaaggccttgatggccgtagataaagctaaaatcggacactctaagccgtcgattgctcaacaaggagttaacttacggaataaggtagtccctaagttacccgaaaatttatttgggaatttcattagcttactacatggacaaatcgagcccgaggaaggtgaaaacatggatcttgatggtttccttgtgatcttgaatgactcgatcaagaaaattgagggtgattatgctaaggcactaatgtctagtgacaaagattatgaggttttagttaagcactttttaaagtttggtgaaacttttgcaaataatgatgtaaatttctactctttcacttcttggtgcaaattctcattttataaagttgattttggttggggaaagccagtttggagaagcacaggacattatgcatcccagaaagttgtgataatgatggatgatgaagaaggtgatggggtagaagcatgggttcatcttgatgaaaaaagcatgtgtcaattagaacaagatccatacatcaaggcctatgcaacttag

SEQ ID NO: 27 - DNA, 1323 bp, synthetic construct
  source 1..1323: mol_type = other DNA
atggagattaagaacattacttctaaattcgttaagccattaactccaacaccatccaatttgcaaaactacgatatctcttttttcgatgaacatatcccaaatatcaacaccccattgatcctatattactccacctcccaaaactctccaaacaataacatttttgaccactttgaaacctctttgtctaaaactttgactgacttttacccattagctggtagatacatgagacaaggttccttcgtcgactgttctgaccaaggtgtcttgtacatccaatctaaggccaacttcagattagaggaattcttggacttggcttggggtttgaaattcaagatgttgaacgacttgttaccatgtgaagttccagaagctggtgaagttgatgacccagttttgtgtgtcaaggtcaccgctttcgaatgtggcggtttcgctatcggtatgtgtttttcacacaagatttctgatatgtgtaccatgtgtaccttcattaataactgggctactagatcttcacaagagaatgttaataagctaaaattagaaaagtactccccaatcttctctgttgctagagatttccctaaggtcgctttggatgacttgtccccaagattcccacgttctaacatcggtatgaagactaaagttaagttattccaattcaaagtcgacgctatctctaagatgagagaaaacttacatttgtctaaggacaagaccattcatcactcctctaaggttcaattaattgtcgctttgtttttgaaggccttgatggctgttgacaaagccaagatcggtcactccaagccatctatcgcccaacaaggtgttaacctaagaaacaaggttgtccctaagctaccagaaaatctattcggtaactttatttctttattgcatggtcaaattgaaccagaggaaggtgaaaacatggatttggatggtttcttggttattttgaacgactccatcaagaaaattgaaggtgattacgccaaggctttgatgtcttccgataaggactacgaagttttggtcaagcacttcttgaagttcggtgaaaccttcgctaataacgacgttaacttttattccttcacttcttggtgtaagttctcattctacaaggttgacttcggttggggtaagcctgtttggagatctaccggtcattacgcttctcaaaaagtcgttatcatgatggacgatgaggaaggtgatggtgttgaggcttgggttcatctagatgaaaagtctatgtgtcaattggaacaagacccatatattaaggcttacgctacttaa

SEQ ID NO: 28 - DNA, 1323 bp, synthetic construct
  source 1..1323: mol_type = other DNA
atggagatcaaaaacattacctctaagtttgtaaaaccgctgaccccgaccccgtccaacctgcagaactatgatatctcttttttcgacgaacacatcccgaacatcaacacgccactgattctctactattctacctcccagaacagcccgaacaataacatcttcgaccatttcgaaacctccctgtccaaaactctgactgatttttacccactggcaggccgttacatgcgtcagggcagcttcgtggactgctctgatcagggtgtgctgtatattcagtctaaagcgaacttccgtctggaggaattcctggatctggcttggggtctgaaattcaaaatgctgaacgacctcctgccatgcgaagttccggaagcaggtgaagtagatgaccctgttctgtgcgttaaagttaccgctttcgaatgtggcggtttcgccattggcatgtgttttagccacaaaatctctgacatgtgtaccatgtgtactttcattaataactgggcgactcgctccagccaggaaaatgttaacaaactgaaactggagaaatactctccgattttctctgtggcacgtgatttccctaaagttgctctggatgacctgtccccacgttttccgcgctccaacattggcatgaaaaccaaggtaaaactcttccagttcaaagtcgacgcaatttctaagatgcgtgaaaacctgcatctgtctaaagacaaaacgatccatcacagctctaaagttcagctgatcgtggccctgtttctgaaagctctgatggcggtggacaaggctaaaattggtcactccaaaccgagcattgcgcagcaaggtgtgaacctgcgcaacaaagtcgttccgaaactgccggaaaacctgttcggcaactttatcagcctcctgcacggtcaaatcgagcctgaggaaggcgaaaacatggatctggatggctttctggtgatcctgaatgactctattaagaaaatcgaaggtgactacgcgaaagcgctgatgtccagcgacaaagattatgaggtactggttaaacactttctgaaattcggcgaaacctttgcaaataacgacgtgaacttctattctttcacctcctggtgtaaattcagcttttacaaggtcgatttcggttggggcaaaccggtgtggcgtagcaccggccactacgcttctcagaaggttgtgatcatgatggatgacgaggaaggcgacggtgttgaagcgtgggtgcacctggacgaaaaatccatgtgccaactggaacaggatccgtacattaaggcatacgccacttaa

SEQ ID NO: 29-30 - skipped (2 entries)

SEQ ID NO: 31 - protein, 419 aa, Cynara cardunculus
MEIKIRSIQSIKPSKPTPENLRNFRLSLLDQLASSYINLIFYYKASGEINISDRCTQLVKSLSEVLTLFYPLAGRITEDGLIVDCSDQGIKYLETQVSTRLDDFLEQGPKIDLVNQLIGAPDQVTTTLVIQVNVFDCGALVIGVSAAHKVTDTSNLVRFINEWASMNRTGESSGAFCPCIDNMASLFPAREISSSKYSLIPNDPEAIIVTKRFVFNGYTISKLRAKASSPNRKHSRVTLVASLIWKALISIDHVKSGSFRDCLLAPAINLRGKANSAISESSFGNVWTPYPIRFLQNKMEPEFVDLVNLIEDTTRNFITWLPKASSEEICTQAIACYAEAVEEVKQDKFAIFTSWCRFPIYEADFGWGKPYWASGTGSSIEIVTLMDDKHGDGIEAWVSLNEKDMYLLEQDEDLLAFTS

SEQ ID NO: 32 - protein, 429 aa, Helianthus annuus
MVMAMKIEKQSSKLIKPFVQTPPTQSHYKLGFIDELAPAHDTGIVLFFAANSNHNPNFVARLEKSLGKTLTRLYPLAGRYVEETHSVDCKDQGAEFIHAKVNIKLQDFLVSEENVKFTDEFIPSKIGVARQQSDPLLATQVTTFECGGVAIGASATHKIVDASTLCTFVNEWAVTNREENEIEFKGPGFNSSILFPSRGLSSIPFPFINIEMLNKYTKKKLSFSGSAISKMKAKCSNSTRQRSKVQLVSAIIWKTFMGVDLAIHNHQRHSIHIQAVSLRGKMASSIPKTSCGNLCGACTTECTTLERTEELADRLTDSVKKTVTKYSKERHDCEEGQAMVLNLIMSSMDNISESTNVVFTTSWCKFPFYEADFGFGKPTWVAPGIVPVHQMTYMIDDAEGTGVEAYVYLEVKDVPYFEEALEHAIAFGA

SEQ ID NO: 33 - protein, 420 aa, Arctium lappa
MEIKIRSVQSIKPSKSTPKNLRNFRLSLLDQLASSSYINLIFYYKASAEINVSDRCTQLVKSLSEVLTLFYPLAGRITEDGLIVDCSDQGVKYLETQVSTRLDDFLEQGPKIDLINQLIGAPDQVTTPLVIQVNIFDCGALVIGVSAAHKVTDASNLVRFINEWASTNRTGDSNGAFYPSFDNMASVFPPREISSSKYSLIPNDPEAIIVTKRFVFNGDTISKLRAKASSPNHKHSRVTLVASLVWKALIYIDQVKNGSFRDCLLAPAVNLRGKAGSPISESSFGNVWTPYPIRFLQNKMESKFVDLVTLVEDTTRNIIMWLPKASGEEICTQAMACYAEVVEEVKQNKFSIFTSWCGFPIYEADFGWGKPYWASEAGSSIEIITLMDDKHGDGIEAWVSLNEKDMYVFEQDEDILALTS

SEQ ID NO: 34 - protein, 436 aa, Mikania micrantha
MEITENVSKLVKPSTPTPSTLRNYNVSLFDHVSMNVPLILYYYASHKKQNGIQTNMFIDLEISLSQTLTQFYPLAGRYKRNALFIDCSDEGALYIQAKARFQLSEFLDLKKDLKHDMLQDFLPYDIHKIGEMDDPLLSVKVTTFEDGGVAIGMCISHHFADMTTICTFIDNWATRSRLVDNELELKLELKKYSPVVSSCAHLFPKADALDTKNLDATTVNNNYILRVFSFKGSAIEKLRQQVMSDENSIIRHRPSKVQLIMALLWKAFVDTNKANGQLNASLLHLPVNLRNIVAPKYFCGNFFTAANTRIEASEAINLQVFVNRLNESINKTKVKFAKVLSHPEINWDVLLEPFLEVINSDAKVYTFTSWCKFSFYTADFGWGKPVWRSIANVKVPNAVVMMDDKEGDGMEAWVHLDEKHMCELEKDPNIQAYMDA

SEQ ID NO: 35 - protein, 440 aa, Smallanthus sonchifolius
MEIIEEVSKLVKPSTPTPSTLCNYNISFFDEKPRDMNVPLILYYTTSHEEEKDIQTNIFNHLEISLSKTLTDFYPLAGRYTRHASFIDCRDQGALYVQAKAKFHLSEFLGLEQKLKLDMQQDFLPYEVNKAGETGDPLLSVKVTSFECGGVAIGMCISHRFADMATFCTFIDNWATRSREIRNELELELEKYSRVSSSAHLFPKTTDVLINDHIVVRSHGVSNCVLRVFSFKGTAITKLREKIMSDEDNTTRHRPSKVQLIVALLWKAFMDIDRRNGQSKASFVSQAVNLRNIVVLDNFFGNFITLANARVELSEVINLQVLLKLLHDSIHKIKSNHAKALSHFEKDYEVLSKPYSEVYESISNNVNSYLFTSWCKFSFYTADFGCGKPVWRSTTNTKRPNTVMMMDDEEGNGVEAWVQLEDKQMCELEQDPNIQAYMDV

SEQ ID NO: 36 - protein, 440 aa, Smallanthus sonchifolius
MEIIEIVSKLVKPSKPTPPTLCNYNISFFDDIPESVNVPLILYYSTSHKEQKDIQTNIFNHLEISLSKTLTDFYPLAGRYTHLASFIDCRDQGALYIEAKAKFQLSELLGLEQKLKLEMQQDFLPCEVGNGVKNDDPLFNVKVTSFECGGVAIGMCISHKFADMDTFCKFIDNWTTRSREIGNELEFKFEKYSTISSAAHLFPKPDVLANHQLDPTSFEVNNCVMRLFLFKASAIRKLREEVMSDHGNIIRHRPSKVQLIVALLWKAFVDIDRQDGQSKASFVGQAVNLRNIAVSENFYGNLSSFANARIEYNKVINLQVLVKLLHDSVNEMKNSYAKALSQFEKDYEVLSKPFLECLENISSKDVNSYLFSSWCRFSFHTADFGWGKPVWKSITNEKIPNSVTMMDDEEGDGVEAWVHLDEKQMCELEKDSNLQAYMDA

SEQ ID NO: 37 - DNA, 1260 bp, Cynara cardunculus
  source 1..1260: mol_type = genomic DNA
atggagattaaaattcgatccatacaatccataaaaccatcaaagccaactccagaaaatctacgcaatttcaggttatcattgcttgatcagctggcttcttcgtatataaatttaatcttctactataaggccagtggtgagattaacatttccgatagatgcactcagctggtcaagtctttatccgaggtgctaaccttattttacccattagctggcaggatcacagaagatgggctcatagttgattgtagtgatcaagggatcaagtatttggagactcaagtgagtacaagactggacgattttcttgaacaaggtcccaagatcgacctcgttaatcaactcataggtgcaccagatcaagtcacaaccacgttggtaatccaggtcaacgtctttgattgtggtgcacttgttattggtgttagtgccgcacacaaagtcaccgatacaagcaacctagtaaggttcattaatgaatgggctagcatgaaccgcacaggggaaagcagtggtgccttttgcccttgtattgataacatggcctctctcttcccagcaagggaaatttcatcatccaagtactcactgattccaaatgatcctgaagccataattgtcacaaaaaggtttgtatttaatgggtatactatatcaaagctaagagcaaaagccagttccccaaatcgtaagcatagtcgcgtaacattggtggcttcattaatatggaaggcccttatatctattgatcatgtcaaaagtgggagcttcagggattgcttgttggcacctgcgataaatttaaggggaaaagcgaactcagcaatatcagaaagttcgttcgggaatgtatggactccttatccaatccgctttttgcaaaataaaatggaacccgagtttgtcgatcttgtgaacctgatagaagacacaacaagaaactttatcacgtggcttccaaaagcgagtagtgaagagatatgcacacaggcaattgcatgttatgccgaggctgttgaagaagtaaagcaagataagtttgccatttttacaagttggtgtcggttccctatatatgaagctgattttggttggggcaagccctactgggcaagtggtactggcagttcaatagaaatcgtcacattaatggatgacaaacatggcgatgggattgaagcatgggtgagtttgaatgagaaagacatgtatctgttagaacaagatgaggaccttttagccttcacctcctaa

SEQ ID NO: 38 - DNA, 1260 bp, synthetic construct
  source 1..1260: mol_type = other DNA
atggagattaagatcagatctattcaatccatcaagccatctaagcctactccagaaaacttgagaaacttcagactatctctattggaccaattagcctcatcttacattaacttgatcttctattacaaggcttctggtgaaattaatatttccgacagatgtacccaattggttaagtctttgtcagaagttttgactttgttctacccattggctggtagaattactgaggatggtttgattgttgattgttcagatcaaggtatcaagtatttggaaacccaagtttctaccagattggatgacttcttggaacaaggtcctaagattgatttggtcaatcaattgattggtgccccagaccaagttactaccactttggttatccaagttaacgttttcgattgtggtgccttggttatcggtgtctccgccgctcataaggtcactgacacctctaatttggttcgtttcatcaacgaatgggcttctatgaaccgtactggtgaatcttccggtgctttctgtccatgtattgacaacatggcttctttgttcccagctagagaaatttcttcctctaaatattctttgatcccaaatgaccctgaagctattatcgtcaccaagagatttgtcttcaacggttacaccatctctaagttgagagccaaggcttcctctccaaacagaaagcactctagagttactttagtcgcttcattgatctggaaggctttgatttcaatcgatcacgttaagtctggttcattcagagactgtttattggctccagccatcaatttgagaggtaaggctaactctgctatctctgagtcatctttcggtaacgtttggaccccatacccaattagattcctacaaaacaagatggaaccagaatttgttgacttggttaatttaatcgaagacaccactcgtaacttcatcacttggttgcctaaggcttcctctgaggaaatctgtacacaagctatcgcttgttacgctgaagctgttgaggaagttaagcaagataagttcgctattttcacctcttggtgcagattcccaatctacgaagccgatttcggttggggtaagccatattgggcttctggtactggttcttccatcgaaattgtcaccctaatggacgataagcacggtgatggtatcgaggcttgggtttctttgaacgaaaaggacatgtatttgctagaacaagatgaagacttgctagctttcacctcttaa

SEQ ID NO: 39 - DNA, 1260 bp, synthetic construct
  source 1..1260: mol_type = other DNA
atggaaatcaaaattcgctccatccagtctatcaaaccgtccaagccgacccctgaaaacctgcgtaacttccgcctgagcctcctggaccagctggctagctcctacattaatctgatcttctattacaaggcaagcggcgaaatcaacatctccgatcgttgcactcagctcgttaaatctctgtccgaggtgctgaccctgttctatcctctggcgggccgtattaccgaagacggtctgattgtcgattgtagcgaccagggtatcaaatatctggaaacccaggtttccacgcgtctggacgattttctggaacaaggcccgaaaatcgatctggttaaccagctgatcggtgcaccggaccaggttactacgaccctggtaatccaggtgaacgttttcgattgcggcgcactggtcattggtgtttctgcggcacacaaagtgaccgataccagcaatctggtgcgtttcatcaacgaatgggctagcatgaaccgcaccggcgagagctctggcgctttctgtccgtgcattgataatatggcctctctgtttccggcgcgtgaaatttcttccagcaagtactccctgatcccgaacgacccggaagcaattatcgtgaccaaacgcttcgtgttcaatggttatactattagcaaactgcgtgctaaggcctcctctccgaaccgtaaacactctcgtgtgactctcgttgcgagcctgatttggaaagctctgatctctatcgatcacgtgaagtccggttccttccgtgactgcctcctggctccggccatcaacctgcgtggtaaagcaaactctgcaatttccgagagctctttcggtaatgtatggactccatacccgatccgcttcctgcagaacaaaatggaacctgaattcgttgacctggttaacctgatcgaagacactacccgtaacttcattacgtggctgccgaaagcatctagcgaggaaatctgcactcaggcgatcgcatgttatgctgaagcggtggaggaagtgaaacaggacaaattcgcgattttcacttcctggtgtcgtttccctatttacgaagcggactttggctggggcaaaccgtactgggcaagcggtactggctcttccatcgagattgtgaccctgatggacgataaacacggtgatggcattgaggcgtgggtcagcctgaacgaaaaagacatgtatctcctggagcaggacgaggatctcctggcattcacctcctaa

SEQ ID NO: 40 - DNA, 1290 bp, Helianthus annuus
  source 1..1290: mol_type = genomic DNA
atggtgatggcaatgaagattgaaaaacaatccagcaaattaataaaaccctttgttcaaactcctcctacacaatctcactacaagttgggcttcatcgacgagttagctcctgcccatgatactggcatcgttctgttctttgccgctaatagcaatcacaaccccaactttgttgcccgacttgaaaaatcgcttggaaaaaccttaacacgactctaccctcttgcgggtagatacgttgaagaaactcatagtgttgattgcaaagaccaaggtgctgagtttatacacgccaaagttaatatcaaacttcaagattttcttgtctccgaagaaaacgttaagtttactgacgaattcattccatccaagataggggttgctcgtcaacaaagtgacccgttacttgcaactcaagtaaccacctttgaatgtggaggtgtggcaattggtgcaagtgctacacacaagattgttgatgcttccactctatgcacatttgtaaacgaatgggctgttacaaatcgagaagaaaatgagattgaattcaaagggcctggtttcaattcatccatattgtttcctagtcgtggtttaagctctataccatttccatttataaacattgagatgttaaacaagtatacaaaaaagaaactttcattcagtgggagtgcaatatcaaagatgaaagcaaagtgttcaaatagcacccgccaacggtcaaaggtacaattggtatcagcaatcatttggaaaactttcatgggtgttgatctagcaatacacaatcatcaaagacattccatacacattcaggcagtaagcttgagggggaaaatggcatcctcaatacccaaaacttcttgtgggaatctttgtggggcatgtaccacagaatgtacaactcttgaaagaactgaagaactggcagaccgtttaactgattctgtcaagaaaactgtaactaagtactcgaaggagcgccatgactgcgaagaaggacaagcgatggttttgaatttaataatgtcaagcatggacaatattagtgaatctactaatgttgtcttcacaactagttggtgtaagtttcctttttatgaagctgactttggtttcggaaaacctacttgggttgcccctggtatcgtaccggttcaccaaatgacgtatatgatcgatgacgctgaaggtactggagtagaagcatatgtctatcttgaagttaaagatgtgccttatttcgaagaagctctagaacatgctattgctttcggagcataa

SEQ ID NO: 41 - DNA, 1290 bp, synthetic construct
  source 1..1290: mol_type = other DNA
atggttatggctatgaagattgaaaagcaatcctcaaagttaattaaaccatttgttcaaactcctccaacacaatctcactacaaattgggttttattgacgaattagctcctgcccatgataccggtattgttttgtttttcgccgctaattctaaccacaacccaaactttgttgccagattggaaaagtctttaggtaaaactttgactagattgtacccattggctggtagatacgtcgaggaaacacactctgtcgactgtaaggaccaaggtgctgaatttatccacgccaaggttaacattaaattgcaagatttcttagtctctgaggaaaacgtcaagttcactgacgaattcatcccttctaaaatcggtgttgctcgtcaacaatccgatccattattggctacccaagtcaccactttcgagtgtggcggtgttgccattggtgcctctgctactcacaagattgtcgacgcctctaccctatgtaccttcgttaacgaatgggccgttaccaacagagaggaaaacgaaatcgaatttaaaggtccaggtttcaactcttccatcttgtttccatctagaggtttgtcctctatcccatttccattcatcaatattgaaatgttgaacaaatacaccaagaaaaagttgtcattctccggttccgccatttctaaaatgaaggctaagtgttcaaactctactcgtcaacgttccaaggttcaactagtctccgctatcatttggaagactttcatgggtgttgacttggctattcataaccatcaacgtcattctatccacattcaagctgtctctttgcgtggtaagatggcttcctcaatcccaaaaacttcctgcggtaatttgtgtggtgcttgtactaccgaatgtaccactttggaaagaacagaggaattggccgaccgtttgaccgactctgtcaaaaagaccgtcaccaagtactcaaaagaaagacacgactgtgaggaaggtcaagctatggtcttgaacttgattatgtcctctatggataacatttccgaatctactaacgtcgttttcactacctcttggtgtaagttcccattctacgaagctgatttcggtttcggtaagccaacttgggtcgccccaggtattgttccagttcatcaaatgacttacatgattgatgacgctgaaggtactggtgttgaggcctacgtctacttggaagtcaaggacgttccatactttgaagaggctttggaacacgccattgctttcggtgcctaa

SEQ ID NO: 42 - DNA, 1290 bp, synthetic construct
  source 1..1290: mol_type = other DNA
atggtgatggctatgaagattgaaaaacagtctagcaaactgatcaaaccgttcgtgcaaactccgcctacccaatcccactacaaactgggttttatcgatgaactcgccccagctcatgataccggcatcgttctgtttttcgcggcaaactccaaccacaatccgaacttcgtggcacgcctggaaaagtccctgggcaaaaccctgactcgtctgtatccactggcgggtcgctatgtcgaggaaacccactccgttgactgcaaagatcagggcgcagaatttatccacgctaaagtaaacatcaaactccaagatttcctggtgtccgaggaaaacgttaaatttactgacgagttcattccgtctaagattggcgttgctcgtcaacagagcgaccctctcctggcaactcaggttactacctttgaatgcggtggcgtagctatcggcgcatccgctacccataagatcgtagacgccagcaccctgtgcactttcgtcaacgaatgggcagttaccaaccgtgaggaaaatgaaatcgagtttaagggtccgggtttcaactccagcatcctgttcccttctcgtggtctgtcctctatcccgttcccgttcattaacatcgagatgctgaacaaatatactaaaaagaaactgtctttttccggctccgctatttctaaaatgaaagctaagtgctccaattccacccgccagcgtagcaaagtgcagctggtatctgccattatctggaaaactttcatgggcgtggacctggctattcataaccaccagcgtcacagcatccacatccaggctgtttctctgcgtggtaaaatggccagctccatcccgaaaacgtcctgcggtaacctgtgcggcgcctgcactaccgaatgcactacgctggagcgtaccgaggaactggcagatcgtctgactgattctgtcaagaaaaccgtgaccaaatattccaaagaacgccatgattgcgaggaaggtcaggctatggttctgaacctgattatgtccagcatggacaacatctctgaaagcaccaatgtagtgttcactacctcttggtgtaaattcccgttctatgaggccgacttcggcttcggcaaaccgacctgggtggcgccgggtattgtaccagtacaccagatgacctatatgatcgatgacgccgagggcacgggtgttgaagcgtacgtctacctggaggttaaagacgtaccatacttcgaggaagcgctggagcacgctatcgcattcggtgcgtaa

SEQ ID NO: 43 - DNA, 1263 bp, Arctium lappa
  source 1..1263: mol_type = genomic DNA
atggagattaagattcgatccgtacaatccataaaaccatcaaagtcaactccaaaaaatctacgcaatttcagattatcgttgctggatcaacttgcttcatcttcatacatcaatttaatcttctactataaggccagtgctgagattaacgtttcagatagatgcactcagctggtcaagtctttatcagaggtgctaaccttattttacccattagctggcagaatcacagaagacgggctcatagttgattgtagtgatcaaggggtcaagtatttggagacgcaagtgagtacaagactggatgattttctcgaacaaggtcccaagattgacctcattaatcaactcataggtgcaccagatcaagtcacaaccccgttggtaatccaagtcaacatctttgattgtggtgcacttgttattggtgtgagtgccgcacacaaagtcaccgatgcaagcaacctagtaaggttcattaatgaatgggctagcacgaaccgcacaggggacagcaatggtgccttttacccttcttttgataacatggcctccgtcttcccaccaagggaaatttcatcatccaagtactctttgattccaaatgatcctgaagccataattgtcacaaaaaggtttgtatttaatggggatacaatatcaaagctaagagcaaaagccagttcaccaaatcataagcatagtcgagtaacattggtggcttcattagtatggaaggctcttatctatattgatcaagtcaaaaatgggagcttcagggattgcttgttggcacctgcagtaaatttaaggggaaaggcaggctcaccaatatcagagagttcgtttgggaatgtatggactccttatccaatccgctttttgcaaaataaaatggaatccaagtttgtcgatcttgtgaccctggtagaggacacaacaagaaacattatcatgtggcttccaaaagcaagtggtgaagagatatgcacacaggcaatggcatgttatgccgaggttgttgaagaagtaaagcaaaataagttttccatttttacaagttggtgcggattccctatatatgaagctgattttggctggggtaagccctactgggcaagtgaagctggcagttcaatagagatcattactttaatggatgacaaacatggtgatgggattgaagcatgggtgagtttgaatgagaaagacatgtatgtgtttgaacaagatgaggacatcttggccttaacctcctaa

SEQ ID NO: 44 - DNA, 1263 bp, synthetic construct
  source 1..1263: mol_type = other DNA
atggaaatcaaaattagatccgtccaatctatcaagccatctaagtctaccccaaagaacttgagaaactttagattatccttattggaccaattggcctcctcttcctacattaacttgattttctattacaaggcttctgctgaaatcaacgtttctgatcgttgtactcaattagttaagtccttgtctgaagttttgactttgttttaccctttggctggtagaattaccgaagatggtttaatcgttgactgttctgaccaaggtgttaaatacttggaaactcaagtttccacccgtttagacgatttcttggaacaaggtccaaaaattgatttgattaaccaattgattggtgctcctgatcaagttactaccccattagttatccaagtcaatattttcgactgtggtgctttggttatcggtgtttctgctgcccacaaggtcactgacgcctctaacttggttagattcatcaacgaatgggcctccaccaacagaacaggtgactctaacggtgctttctacccatctttcgataatatggcttctgtcttccctccacgtgaaatttcctcatccaagtactcattgattccaaacgacccagaagccatcattgttactaagagattcgttttcaacggtgacactatctccaagttacgtgctaaagcctcatctccaaaccacaagcattcaagagtcactttggtcgcttctttggtttggaaggctttaatttatattgaccaagttaagaacggttctttcagagactgtttactagctccagctgttaacttacgtggtaaggctggctctccaatttccgaatcctctttcggtaatgtttggaccccatacccaatccgtttcctacaaaacaaaatggaatcaaaattcgtcgacttggttactttggttgaagacacaactagaaatattatcatgtggttgcctaaggcttccggtgaggaaatttgtactcaagctatggcttgttacgccgaagttgtcgaggaagttaagcaaaacaagttctccatttttacatcctggtgtggtttcccaatttacgaagccgatttcggttggggtaaaccatactgggcttctgaagctggttcatctattgaaatcattactttaatggacgataagcacggtgatggtatcgaggcttgggtttccttgaacgaaaaagacatgtacgtctttgaacaagacgaagacatcttggctttaacttcttaa

SEQ ID NO: 45 - DNA, 1263 bp, synthetic construct
  source 1..1263: mol_type = other DNA
atggaaatcaaaatccgtagcgttcagtctatcaaaccaagcaaatccaccccgaaaaacctgcgcaacttccgtctgagcctcctggatcaactggcgtcttcctcttacatcaatctgattttttattacaaagcgagcgctgaaatcaacgttagcgaccgttgcactcagctggtgaagtccctgagcgaagtactgaccctgttttacccgctggcgggtcgcattactgaggatggcctcatcgtggactgcagcgaccaaggcgttaagtacctggaaactcaggtttccacccgtctggacgattttctggaacagggcccgaaaattgacctgatcaaccagctgatcggtgcaccggatcaggttactaccccgctggtgatccaggtaaatatctttgactgtggcgcactggttatcggtgtatctgcggcccacaaagttaccgacgcgagcaacctcgtgcgtttcattaatgaatgggcgagcaccaaccgtaccggcgactccaacggtgccttctatccgtctttcgacaatatggcgtccgtcttccctccgcgtgaaatctcttcctctaagtatagcctgatcccgaacgacccggaagcaattatcgtgactaagcgcttcgtattcaacggtgatactatctctaaactgcgtgccaaggcttccagcccgaaccacaaacacagccgcgtaaccctggtggcgtccctggtgtggaaagcactgatctacatcgaccaggtgaaaaacggcagcttccgtgactgcctcctggcaccggctgttaacctgcgcggcaaagcgggctccccgatctccgaaagctctttcggcaatgtgtggaccccgtacccgatccgttttctgcagaacaaaatggaatctaaattcgtagatctggttaccctggtggaagacactacccgtaacattatcatgtggctgccgaaagcgagcggtgaggaaatctgcacccaggcaatggcctgctatgctgaggtcgttgaggaagtgaaacaaaataaattttctatcttcacttcttggtgcggcttcccgatttatgaagctgattttggttggggtaaaccgtactgggcgtccgaggcgggttcctctattgagattatcacgctgatggatgacaaacacggtgatggtatcgaggcgtgggtaagcctgaacgagaaggatatgtatgtattcgagcaggacgaagatatcctcgccctgaccagctaa

SEQ ID NO: 46 - DNA, 1311 bp, Mikania micrantha
  source 1..1311: mol_type = genomic DNA
atggaaataactgagaatgtctcaaagcttgtgaaaccttcaacaccaactccttccacccttcgtaactataacgtctctttgtttgatcacgtgagcatgaatgtgcctttgattctttactattatgcatcacataagaaacaaaatggcatacaaaccaatatgtttatagatttagagatatcattatcacaaactttaactcagttttacccattggcaggaagatataagcgtaatgctttgtttattgattgtagtgatgaaggtgctctatacattcaagcaaaagcgagattccaactatctgaatttctagacttgaaaaaggatttaaaacatgatatgctacaagatttcctcccgtatgacatccataaaattggtgaaatggacgaccccttgttatccgttaaagtcacgactttcgaggacggtggagttgccataggtatgtgcatttcacatcattttgcagatatgaccaccatttgcacattcattgataactgggcaaccagaagccgactagtagacaatgaattggaactaaaactagagctaaaaaaatattctcctgttgtttcaagctgtgcccatctcttcccgaaagctgatgctttagacacaaaaaacctagacgcgacgacagtgaacaataattatattttgcgagtcttttcgtttaaagggtccgcaatagaaaaattaagacaacaagtcatgagtgatgagaatagtatcataaggcataggccatcaaaggtgcaacttattatggctttgttatggaaggcctttgtggacacaaataaggcaaatgggcaattgaatgcatctttgttgcacctcccagttaacttgaggaacatagtagcccctaaatacttttgtggtaatttttttacggcagcaaacacgcgaattgaggctagcgaagcgatcaatcttcaagtttttgttaaccgcttgaatgaatcaattaacaaaacaaaagtgaagtttgctaaagtgttatcacatcctgagataaattgggacgttttgttagaaccctttttagaagttataaatagcgatgcaaaggtatacacgttcacttcatggtgcaaattctcattctacactgctgattttggttggggtaagcctgtttggagaagcatagcaaatgtcaaagtaccaaacgcggtggtcatgatggatgataaagaaggcgatgggatggaagcatgggttcatttagatgaaaaacatatgtgtgagttggagaaagaccctaatatacaagcctacatggatgcttaa

SEQ ID NO: 47 - DNA, 1311 bp, synthetic construct
  source 1..1311: mol_type = other DNA
atggaaattactgagaacgtttctaagttggtcaagccatctacccctactccttcaaccctaagaaactataacgtttcattattcgaccatgtctccatgaatgttccactaattttgtactattacgcttcccacaaaaagcaaaacggtattcaaactaatatgtttattgacttagaaatttcattgtctcaaaccttgacccaattctaccctttagctggtcgttacaagagaaatgccttgtttatcgattgttctgacgagggtgctttgtacattcaagctaaggctcgtttccaattatcagaatttttggatttgaagaaagacttgaaacacgatatgttgcaagattttttgccatacgacattcacaagattggtgaaatggatgaccctctattgtctgtcaaggttaccactttcgaagacggcggtgttgccatcggtatgtgtatttcacatcacttcgccgatatgaccactatttgtaccttcattgacaactgggctaccagatctagattggttgataacgaactagaattgaagttagagttgaaaaagtattctcctgttgtctcttcctgtgcccatttattcccaaaagccgatgccttagatacaaaaaacttggacgctactacagttaacaataactatattttgagagttttctctttcaaaggttctgctattgaaaagttgagacaacaagttatgtctgatgaaaattccattatcagacacagaccttccaaggtccaattaatcatggctttattgtggaaggctttcgttgacaccaacaaggccaacggtcaattgaacgcctctttattgcatttgccagttaacttgagaaacattgttgctccaaaatacttttgtggtaactttttcaccgccgctaacaccagaattgaggcttcagaagctattaacttacaagtcttcgttaacagattgaatgaatccattaacaagactaaagttaaattcgccaaagttttgtctcacccagaaatcaactgggacgtcttattggaaccattcttggaagtcattaactctgacgccaaggtttacaccttcacctcttggtgcaaattttctttctacaccgctgactttggttggggtaagccagtctggcgttccattgccaacgttaaggttccaaacgccgtcgttatgatggatgacaaagaaggtgacggtatggaggcttgggttcacttagatgaaaaacacatgtgtgaattggaaaaagacccaaacattcaggcttacatggacgcttaa

SEQ ID NO: 48 - DNA, 1311 bp, synthetic construct
  source 1..1311: mol_type = other DNA
atggagatcaccgagaacgtaagcaaactggtcaagccgagcaccccgactccttctactctgcgtaactacaatgtctccctgtttgatcacgtttctatgaatgtgccgctgatcctgtactattacgcgtcccacaaaaagcagaatggcatccagaccaatatgttcatcgacctcgaaatcagcctgtctcagaccctgacgcagttttacccgctggctggccgttacaaacgcaacgctctgttcattgactgctctgacgaaggtgcactgtacattcaggccaaagcccgtttccagctgtctgaattcctggacctgaagaaagacctgaaacatgacatgctgcaggactttctgccttacgacatccataagatcggcgaaatggatgacccgctgctctctgttaaagtaaccactttcgaagatggcggtgtagccatcggtatgtgcatctcccaccatttcgctgatatgaccacgatctgtaccttcatcgacaattgggcaacccgttcccgtctggttgacaacgaactggaactgaaactggaactgaagaaatatagcccggttgtgtcttcctgtgcccacctgttcccaaaagccgatgccctggacactaagaatctggacgccactaccgttaacaataactatattctgcgtgtattctctttcaagggttctgctattgagaaactgcgtcaacaggttatgagcgacgaaaacagcattatccgccaccgcccgtccaaagttcagctcatcatggctctcctgtggaaagccttcgtcgacaccaacaaagctaacggtcagctgaacgcgagcctcctgcacctgccggttaacctgcgtaatatcgtggcgccgaaatacttttgtggcaattttttcaccgcggctaacactcgtatcgaagcaagcgaggcaatcaacctgcaggttttcgttaaccgtctgaacgaatccattaacaagacgaaagtgaagttcgcaaaggtgctgtcccatccggagatcaactgggacgttctcctggaaccgttcctggaagtcatcaactccgacgcgaaagtttacacttttacttcttggtgtaaattctccttctatactgctgacttcggttggggtaagccggtttggcgctccatcgcgaacgtgaaagtcccgaacgcagtggttatgatggatgacaaagaaggcgacggcatggaggcttgggtacatctggatgagaaacacatgtgtgaactggagaaagatcctaacattcaggcttatatggacgcgtaa

SEQ ID NO: 49 - DNA, 1323 bp, Smallanthus sonchifolius
  source 1..1323: mol_type = genomic DNA
atggagataattgaggaagtctcaaagcttgtgaaaccttcaacaccaactccatccaccctttgtaactataacatctctttctttgatgagaaaccccgggacatgaatgtgcctttaattctctactacactacatcacatgaagaagaaaaggatatacaaaccaacatctttaatcatttagagatttcattatcaaaaactttaaccgacttttacccattggccgggaggtatactcgtcatgcttcatttattgattgtagggatcaaggcgctctatacgtccaagccaaagcgaaattccatctctcggaatttctaggcctggagcagaagttaaaactagatatgcaacaagatttccttccgtatgaggtcaataaggctggtgaaaccggcgaccccttgttatccgttaaagtcacgagttttgagtgtggtggagttgccataggtatgtgcatttcacatagatttgcagatatggccactttttgcacattcattgataattgggctactagaagccgagaaatacgcaatgaattagaattagaattagaaaaatattcacgtgtttcaagttcggcccatctcttcccaaaaactactgatgtacttataaatgatcatattgtcgttcgctcacatggagtgagtaattgtgttttgcgagtcttttcatttaaagggaccgcgataacaaaattaagagaaaaaataatgagtgatgaggataatactacaaggcataggccttcaaaggtacaacttattgtggcactattatggaaggcttttatggacatagatagacgaaatggacaatcaaaggcgtccttcgtcagccaagcagttaacttgaggaatatagtagtcctcgacaacttttttggtaattttattactttagctaatgcacgagttgagcttagtgaagtgatcaatcttcaagtattacttaagctcttgcatgactccattcacaaaataaaaagtaaccatgctaaagcattatcacattttgagaaagattatgaggttttatcaaaaccctattcggaggtttatgaaagtataagcaataatgtgaactcctacttgttcacttcatggtgcaaattctcattctacactgctgattttggttgcggtaagccggtttggagaagcacaacaaatacgaaaaggccaaacactgtgatgatgatggatgatgaagaagggaatggggtggaagcatgggttcaattagaggacaaacagatgtgtgagttagaacaagatcctaatatacaagcctacatggatgtttag

SEQ ID NO: 50 - DNA, 1323 bp, synthetic construct
  source 1..1323: mol_type = other DNA
atggaaatcattgaggaagtctctaagttggtcaagccatctacaccaactccatccactctatgtaattacaacatttcctttttcgatgaaaaaccaagagatatgaacgttccattaattttatattacaccacttcccacgaggaagagaaggatattcaaactaacattttcaaccacttagaaatttctttatcaaagactttgaccgatttctacccattggccggtagatataccagacacgcttccttcatcgattgtcgtgaccaaggtgccttgtacgtccaagctaaggccaaatttcatttgtctgaattcttgggtttggaacaaaagctaaagttggacatgcaacaagatttcttgccatacgaagtcaacaaggctggtgaaactggtgatccattattgtctgttaaagttacctctttcgaatgtggtggcgttgccattggtatgtgtatctctcatagattcgctgacatggccactttttgcactttcatcgataactgggctactcgttcaagagaaattcgtaacgaattggaattggaattggaaaaatactcccgtgtttcatcttcagctcacctattcccaaagactaccgatgttttgattaacgaccatatcgttgtcagatcccacggtgtttccaactgtgttttgagagttttctcttttaagggtaccgctattactaagttgagagaaaagattatgtccgacgaagataatactaccagacacagaccttccaaagttcaactaattgttgctttattgtggaaggctttcatggatatcgaccgtagaaatggtcaatctaaggcctctttcgtttctcaagctgtcaatctaagaaacattgtcgttttagataactttttcggtaatttcatcaccttagctaacgctagagtcgagttgtctgaagttatcaatttgcaagtcttattgaaattgctacacgattccattcataaaatcaagtctaaccacgccaaggctttgtctcatttcgagaaggattacgaagtcttgtccaagccatactctgaagtttacgaatcaatttccaataacgtcaactcctacttgttcacctcctggtgtaaattttctttctataccgctgactttggttgtggtaagccagtttggagatccactacaaacactaaaagaccaaacactgtcatgatgatggatgacgaggaaggcaacggtgtcgaggcttgggttcaattggaagacaagcaaatgtgtgaattggaacaagaccctaatatccaagcctacatggacgtctaa

SEQ ID NO: 51 - DNA, 1323 bp, synthetic construct
  source 1..1323: mol_type = other DNA
atggaaattatcgaggaagtaagcaaactggtcaagccgagcactccgaccccaagcaccctgtgcaactacaacatctcctttttcgacgaaaaacctcgtgatatgaacgtgccactgatcctctattacactacctctcacgaagaggaaaaagacatccagaccaacatttttaaccacctggaaatctctctgagcaaaacgctgaccgacttttacccgctggcaggccgctacacccgtcacgcctcctttatcgattgtcgcgatcagggtgcgctgtacgtgcaggctaaggcaaagttccacctgagcgagttcctgggtctggaacaaaaactgaagctggacatgcaacaggatttcctgccgtacgaagtcaacaaagcgggtgaaaccggcgatccgctcctgtccgtcaaggtaacttctttcgaatgcggcggtgtggccatcggcatgtgtatcagccaccgcttcgcagatatggccactttttgtaccttcatcgacaactgggctacccgctctcgcgaaatccgtaacgagctggaactggagctggaaaaatattctcgtgtgagctcctctgctcacctgttcccgaaaactaccgacgttctgatcaacgaccacatcgttgtgcgttctcacggcgttagcaactgcgttctgcgcgtattcagcttcaagggtaccgctatcactaaactgcgtgaaaagattatgagcgacgaagacaatacgacccgtcaccgtccgagcaaagttcaactgatcgtggcactcctgtggaaggcgttcatggacatcgaccgtcgcaacggtcaatccaaagcatctttcgtttctcaggcggtgaacctccgcaacatcgtagttctggacaacttctttggtaacttcatcactctggctaacgcgcgcgtggaactgagcgaagtcattaacctgcaggttctcctgaaactcctgcatgattccatccataaaatcaaatctaaccacgctaaagctctgtctcattttgagaaagactacgaagttctctccaaaccgtacagcgaagtttacgaatccatctccaacaatgtaaacagctacctgtttaccagctggtgcaaattcagcttctacactgcggatttcggctgcggcaaaccagtatggcgctccactacgaacactaaacgcccaaacactgtcatgatgatggacgatgaggaaggtaacggcgtggaagcatgggttcagctggaagacaaacaaatgtgcgaactggaacaggacccgaacatccaggcatacatggacgtttaa

SEQ ID NO: 52 - DNA, 1323 bp, Smallanthus sonchifolius
  source 1..1323: mol_type = genomic DNA
atggagataattgagattgtctcaaagcttgtgaaaccttccaaaccaactccacccaccctttgcaactataacatctctttctttgatgacatacccgagagcgtgaatgtgcctttaattctctactactctacatcacacaaagaacaaaaggatatacaaaccaacatctttaatcatttagaaatttcgttatcgaaaactttaaccgatttttacccgttggccgggagatatacacatcttgcttcgtttattgattgtagggatcaaggtgctctatacatcgaagctaaagcgaaattccaactctcagaacttctaggcttggagcagaagttaaaacttgaaatgcaacaagatttcctcccgtgtgaggtcggtaacggtgtgaaaaacgacgaccccttgtttaacgttaaagtcacgagttttgagtgtggtggagttgccataggtatgtgcatttcacataaatttgcggatatggacactttttgcaagtttattgataattggacgactagaagccgcgaaataggcaatgaattagaatttaaatttgaaaaatattctactatttctagcgcggctcatctcttcccaaaacctgatgtacttgcaaatcatcagcttgatcctacctcatttgaagtgaataattgtgttatgcgactctttttgtttaaagcgtccgcaataagaaaactaagagaggaagtcatgagtgatcatggtaatattataaggcataggccttcaaaggtacaacttattgtggcactattatggaaggcctttgtggacatagatagacaagatggacaatccaaggcgtctttcgttggccaagcagttaacttgaggaatatcgcagtctctgaaaacttttatggtaacttatcgagtttcgctaatgcacgaatcgagtataacaaagtgattaatcttcaagttttagttaagctattgcatgactcagttaatgaaatgaagaatagttatgctaaagcattatcacaatttgagaaagattatgaggttttatcaaaaccctttttggagtgtttagaaaatataagtagtaaagatgtaaactcctacttgtttagttcgtggtgcagattctcattccacaccgctgattttggttggggtaagccagtttggaaaagcataacaaatgaaaaaattccgaactctgtgactatgatggatgatgaagaaggggatggggtggaagcatgggttcatttagacgaaaaacaaatgtgtgagttagaaaaagattccaatttacaagcctacatggatgcttag

SEQ ID NO: 53 - DNA, 1323 bp, synthetic construct
  source 1..1323: mol_type = other DNA
atggaaattatcgaaattgtttctaaattggtcaaaccatctaaacctactcctccaactttgtgtaactacaacatttcttttttcgacgatattcctgagtccgttaatgtccctttgattttgtactattccacttcccacaaggagcaaaaggacattcaaactaacattttcaaccatctagaaatttctttgtccaaaactttgactgatttctacccattggccggtagatatacccacttggcttccttcattgactgtagagatcaaggcgccttgtatatcgaggctaaagccaaattccaactatcagaactattgggtctagaacaaaagttgaagctagagatgcaacaagacttcttaccatgtgaagttggtaacggtgtcaagaacgatgacccactattcaatgttaaagttacttccttcgaatgtggcggtgttgccatcggcatgtgtatctcccacaagttcgctgacatggacaccttctgtaaattcattgataactggactaccagatcaagagaaattggtaacgaattggagttcaagtttgaaaagtactcaactatctcctctgctgcccacttgttcccaaaaccagatgttttggctaaccaccaattggaccctacctctttcgaagttaataactgtgtcatgagattgtttttgttcaaggcttcagctatcagaaaattgcgtgaggaagttatgtccgatcatggtaacatcattagacaccgtccatctaaggtccaattgatcgttgctttactatggaaggcttttgttgatatcgaccgtcaagacggtcaatctaaagcctcctttgtcggtcaagctgtcaatttgagaaacatcgccgtttccgaaaatttctacggtaatttgtcatcttttgctaacgccagaattgaatataacaaggtcatcaatttgcaagttttggtcaagttattgcatgactctgttaacgaaatgaaaaactcttacgctaaggctttatcacaattcgaaaaggactatgaagtcttgtctaaaccatttttggaatgcttggaaaacatctcttccaaagacgtcaactcttacttgttttcctcttggtgtagattttccttccataccgctgactttggttggggtaagccagtttggaaatctattactaacgaaaaaattcctaattccgttactatgatggacgatgaggaaggtgacggtgttgaggcttgggtccatttggatgaaaagcaaatgtgtgagttggaaaaggattccaacttgcaggcttacatggacgcttaa

SEQ ID NO: 54 - DNA, 1323 bp, synthetic construct
  source 1..1323: mol_type = other DNA
atggaaattatcgaaatcgtcagcaaactggttaaaccgtccaaaccgaccccgccaaccctgtgtaattataacatcagctttttcgacgatattccggaatctgtaaacgtaccgctgatcctgtactatagcacctctcataaagaacagaaggatatccagaccaacatctttaaccacctggaaatttccctgtccaagaccctgactgacttctacccgctggctggtcgttacacccatctggcatcctttatcgattgccgtgaccagggtgccctgtacattgaagcaaaggccaagttccaactctccgaactcctgggtctggaacagaaactgaaactggaaatgcaacaggactttctgccatgtgaggtgggcaacggcgttaaaaacgatgacccgctgttcaacgtcaaagttaccagcttcgaatgtggcggtgtggcaatcggtatgtgcatctctcacaaattcgccgacatggacactttctgcaaattcatcgacaattggacgacccgttctcgtgaaatcggcaacgaactggaattcaaattcgagaaatattctactattagctctgccgcgcacctgttcccgaaaccggatgtgctggcgaaccaccagctcgatccgacttctttcgaagtcaataactgcgttatgcgtctgttcctgttcaaagcgtccgctatccgtaaactgcgcgaggaagtcatgtccgaccacggtaacattatccgccatcgtccgtccaaagtacaactgattgtagctctcctgtggaaagcgttcgtagacatcgaccgtcaggatggccagtctaaggcttcctttgttggtcaagcggttaacctgcgtaacatcgctgtttctgaaaacttctacggcaacctgtcctcttttgccaacgctcgtatcgagtacaacaaagttattaacctgcaggttctggtaaaactcctgcacgatagcgttaacgaaatgaaaaactcctacgcgaaagcgctgtcccagtttgagaaagattacgaagttctgtccaagccgtttctggaatgcctggaaaatatctcctctaaagatgtaaacagctatctgttctcttcctggtgccgtttcagcttccataccgccgatttcggttggggtaaaccggtgtggaaatctattaccaacgaaaaaattccgaacagcgtaactatgatggatgacgaggaaggcgacggtgtggaggcgtgggtgcatctcgacgagaaacagatgtgcgaactggaaaaagattccaacctgcaggcttacatggacgcataa

SEQ ID NO: 55 - protein, 420 aa, Artemisia annua
MEVKTQSTKYIIPSKSTPENLRSYKLSRLDQISPPSYTKQIYYYKPSGDVSISGICGHLVMSLSEVLTLFYPLAGRLTKDGLEVDCSDQGVKYIETKVSTRLDDFLELGPTIDQVKRLISIPDPGATTLVTVQVNIFDCGALVIGVSASHKVTDAYSLVRFINQWACINRTGCSDDAFSPSFDKMVTLFPPEVVPSIVQIPVSNPQSKMVSKRYMFSGTILSKLRAKAGSPDCKHSRVTLVTAVIWKALIGVDKLNCGSVRNYIMSPAINLRGKVGLEITESSFGNVWVPYAIRYLQNEMEPNFVNLVRLIEDTNRDFIMELPKASSEEICAQAIACYDEVEEEIKQNKSPIITSWCRFPIYNADFGWGNPYWVSEGGRSRDMVTLMDDKNGDGIEAWVDLKEKDMYKFEKDEDITELAS

SEQ ID NO: 56 - protein, 429 aa, Erigeron canadensis
MEIKIQSTRFIKPSKSTPENRRYFKLSLLDQLAPSAYINLIFFYKASGGVNISDRVGQLVKSLSEVLTSFYPLAGRITEDGLAVDCSDQGVEYFETRVSARLDEFLAHGSKMDHVHRLVATPDQVRNTLVIIQVNVFDCGSLVIGVSASHKVTDACNLVRFINAWASTNRTRPSNGPFSPSFDNLDSLFRPRENLSNEHSPVSVELENITVTKRFVFDRTAIRKLRSKTGLENSTKHSRVTLVASLVWKALILIDKVNCGRFRDCLLAPAMNLRGKVGSPISESSFGNVWAPYPIRLLQKEMEPKFVDLVSMIEDTTKSIIAWLATASGEEICKHAMASYAKVNEELKHNKFCMFTSWCRFPIYEADFGWGKPDWARALERSLELVTLMDDKHGDAIEAWVSLNEKDMYVFEQDRDILAFTTSTISEVA

SEQ ID NO: 57 - protein, 421 aa, Helianthus annuus
MEIKIQSIQFIKPSKPTPENLRHFKLSLLDQLAPCSYINPIFYYSTSGEVENSARCGQLANSLSEVLNLYYPLAGRVTEDGLEVDCNDQGVKYLVTRVSTSLDDFLKQGPRIDHMRQLIAARDQDTTWLVTVQVNVFVCGALAIGVIASHKLTDGCNLVRFINQWARMNHACGGDGAFAPTYDKLDHLFPPVTNSSPGHSSDPVDQEAIVVTKRFLFNGTSISKLRAKAGSAKGEHTRVTLVASLLWKALIAMDRVKSGSFRDCLLTVAMNLRGKASSPVSKSTFGNVWAPYPIRFLQNETEPEFGDLVALIEDTTRNVIKWVQKASGEELCRHAKAGYALVDEELKQNKFCIFTSWCRFPIYEVDFGWGKPCWVTEAGSALEMVSLMDDKDGDGIEAWVSLNQKEMYVFERDHDILDFTS

SEQ ID NO: 58 - protein, 429 aa, Helianthus annuus
MVMAMKIEKQSSKLIKPFVQTPPTQSHYKLGFIDELAPAHDTGIVLFFAANSNHNPNFLARLEKSLGKTLTRLYPLAGRYVEETHSVDCKDQGAEFIHAKVNIKLQDFLVSEENVKFTDEFIPSKIGVARQQSDPLLATQVTTFECGGLAIGASATHKIVDASTLCTFVNEWAVTNREENEIEFKGPGFNSSILFPGRGLSSIPLPFINIEMLNKYTKKKLSFSGSAISKMKAKCSNRTSQRSKVQLVSAILWKTFMGVDLAIHNHQRHSIHIQAVSLREKMASSIPKTSCGNLCGACTTECTTLERTEELADRLTDSVKKTVTKYSKERHDCKEGQAMVLNLIMSSMDNISESTNVVFTTSWCKFPFYEADFGFGKPTWVAPGIVPVHQMTYMIDDAEGTGVEAYVYLEVKDVPYFEEALEHAIAFGA

SEQ ID NO: 59 - protein, 432 aa, Daucus carota
  source 1..432: note = subspecies sativus
MKVQIHSKKLVKPFTPTPSNLNHHNLSFIDELAPKMYAPVILYYPCPENVTDKDLFSSTCLLELETSLSKTLVQFYPLAGRYNKHLQLVDCNDKGVEFVEATVDCHLHEVLPHGERSDPQFLNKFIPCEVLITDEAMHPLLAIQVTVFKCGGFAIGVCISHRIADAATLSMFLQAWGTTAKLNKNGNQQESIQKIFPCFDAAVYFPKRGLPHLNFGIFTSSGCKIVTRRFSFDNKAISTLRANILPGTGQTSKLQMVIAVIWKALLGAEKLKDEHARATHIMQPVNLRDKLTTPFLHKHFFGNLCILASVPMMAAENREIPDLAIQLSSSVKGSIEGWAKMMSLGKDDPLLKNMISSTVNYYMISSWSRFPFYETDFGWGKPVWASSVYFPCKNMVLLMDNKKGDGFEAWVSLDEADMNIFEQDFNIKAFST

SEQ ID NO: 60 - protein, 428 aa, Mikania micrantha
MAKSIKIQKQSSKFIKPFVQTPQTLTHFKLGFIDEFSPDVNVGVVLFFSTNTHQTTNFIARLEKSLEKTLTRFYPLAGRLYVDKNATIDCNDHGAEFIHAKVNIKLQNFLVSEANVKFIDDFIPSKIGVVLQQSDPLLAVQVTIFECGGVAIGVKATHKIVDASTLSTLINEWSVTNRQENDNNNDNDNIFSGTNFVSSLLFPPRGLCPMPVPPMNNDELNKYTRKKLSFSENVISNMKAKGSKVQLISAIIWKAFMGVDIAIHSHQRESMLIQAVNLRGKMASLIPKTSCGNLFGTCTTECRTHETIEEVTDRLSDSVRKCITNFSKVHHDCEEGQTMVLKSLLSLTNIRESTCGVIVTSWCKFPFYQVDFGFGKPIWAAPGTIPINQSAYLMDDVQGNGVEAYVFLQVKDIPLFEEALKHVITMSF

SEQ ID NO: 61 - protein, 429 aa, Tanacetum cinerariifolium
MAMKVEKQSSKFIKPFYPTPSNLGRYRLGFTDEIAPLETIGVVLFFSPNSNHDTKFVAQLEKSLEKTLTHLYPLAGRYVDDVDQTVECNDEGAHFIYAIVNIKLYEFLGLKEKFKMADEFIPLSRGTHQFSDPLLEIQVTMFECGGIAVGVRVAHKIADASTLCTFLNEWACISRDENEIGYVGPSFNSSLLFPPRGVRTLPLPPMSADMLSRSTRINFSLSESEISNMKANAIASGKVSARELSKVQLVSAILWKALRVVDRLVHNYLRDSILLQPVNLRGKMASSIPENSCGNLLSICSTKSENVETMEELVNLLSNSVKKTVKEYSKVYHDSEEGQMMVLNSYLNLANIPESSNVVFVTSWCKFPFYEVNFGLGNPIWVVPGTVLSKNLGCLIDDAQGNGVEAYVFLEVKDVPCFEEALDFNVFGD

SEQ ID NO: 62 - protein, 436 aa, synthetic construct
MEIKSITSKLVKPSTPTRSNLQNYDISFFDEQMANINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKFGETFANNDENFHCFTSWCKFSFYKADFGWGKPVWRSTGHYANHKFVILMDDEEGGGVEAWVHLDEKSMCQLEQDPDIKAYAT

SEQ ID NO: 63 - protein, 436 aa, synthetic construct
MEIKSITSKLVKPSTPTRSNLQNYDISFFDEQMHNINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKFGETFANNDENFHCFTSWCKFSFYKADFGWGKPVWRSTGHYANHKFVILMDDEEGGGVEAWVHLDEKSMCQLEQDPDIKAYAT

SEQ ID NO: 64 - protein, 436 aa, synthetic construct
MEIKSITSKLVKPSTPTRSNLQNYDISFFDEQMANINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKYGETFANNDENFHCFTSWCKFSFYKADFGWGKPVWRSTGHYANHKFVILMDDEEGGGVEAWVHLDEKSMCQLEQDPDIKAYAT

SEQ ID NO: 65 - protein, 436 aa, synthetic construct
MEIKSITSKLVKPSTPTRSNLQNYDISFFDEQMANINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKFGETFANNDENYHCFTSWCKFSFYKADFGWGKPVWRSTGHYANHKFVILMDDEEGGGVEAWVHLDEKSMCQLEQDPDIKAYAT

SEQ ID NO: 66 - protein, 436 aa, synthetic construct
MEIKSITSKLVKPSTPTRSNLQNYDISFFDEQMANINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKYGETFANNDENYHCFTSWCKFSFYKADFGWGKPVWRSTGHYANHKFVILMDDEEGGGVEAWVHLDEKSMCQLEQDPDIKAYAT

SEQ ID NO: 67 - protein, 436 aa, synthetic construct
MEIKSITSKLVKPSTPTRSNLQNYDISFFDEQMPNINTPLILYYSASQNSPDDNIFNHLETSLSKTLTDFYPLAGRYKRQGSFVDCSDQGVLYIQSIANFRLEEFLGQAWELKFRMLNDLLPCEVPEAGEVDDPLLCIKVTAFECGGFAIGMCLAHKIADMCTMCTFINNWATRSSQEIVNKQELEKYSPIFSVAHDFPKVALDDLSPRLPRSNIGMETKVKLFQFKVDAISKMRENLRDKSYHSSKVQLIVALFSKALMAIDKAKFGHSKPSIVQQGVNLRNKVVPKLPENLFGNFFSFLHGRIEPEDGENMDLDGFLMILKDSMKKFEDEYVKALMSSDKDYEVLVKPFLKFGETYANNDENFHCFTSWCKFSFYKADFGWGKPVWRSTGHYANHKFVILMDDEEGGGVEAWVHLDEKSMCQLEQDPDIKAYAT

SEQ ID NO: 68 - protein, 440 aa, synthetic construct
MEIKNLTSKLVKPLTPTPSNLQNYDISFFDEQMPNINTPLILYYSTSQESPNDNIFDHLETSLSKTLTDFYPLAGRYMRQGSFVDCSDQGVLYIKSMANFRLEDFLGQAWELKFTMLNDLLPCEVPEAGEVDDPLLCIKVTAFECGGFAIGMCFSHKISDMCTMCTFINNWATRSSQENVNKLELEKYSPIFSVAQDFPKVALDDLSPRFPRSIIGMATNVKVFQFKVDAISKMRENLQISKDERNHHSSKIQLIVALFLKALMAIDKAKIGHSKSSIAQQGVNLRNKVVPKLPENLFGNFFTFLHGQIEPEEGENMDLDGFLVILNDSVKKIEGEYAKALMSSHKDYEVLVKPFLKFGETFTNNNVNFYSFTSWCKFSFYKADFGWGKPVWRSTGHYANEKFVIMMDDEEGGGVEAWIHLDEKSMSQLEQDPYIKAYAT

SEQ ID NO: 69 - DNA, 1263 bp, Artemisia annua
  source 1..1263: mol_type = genomic DNA
atggaggttaaaactcaatctacaaagtacataataccatctaagtcaacccctgaaaatttacgcagttacaagctatcgaggctcgatcaaatttcaccaccttcatacacaaaacagatctattactataagcctagtggtgatgttagcatttcaggtatatgtggtcacttggtcatgtctttatcagaggtgctaactttgttttatccactagctggaagacttacaaaagacgggcttgaagttgattgtagtgatcaaggggtcaagtacatcgaaaccaaagttagtacaagactggatgattttcttgaactaggtcccactattgaccaggttaaacgacttattagtataccagatccaggcgcaactacgttggtaacagtccaagtcaacatctttgattgtggtgcacttgttattggtgtgagtgcttcacataaggtaactgatgcatatagtctagtaaggttcattaaccaatgggcttgcataaaccggacagggtgctctgatgatgcgttttctccttcttttgataaaatggttactcttttcccaccggaggtagttccgtcaattgtacaaatacccgtaagtaaccctcaatcgaaaatggtctcaaaaagatatatgtttagtggcactatactttcaaagctaagagcaaaagcaggttcaccagattgtaaacatagtcgagtaactctagtgacagcagtaatatggaaggctctaattggtgtcgacaaattaaattgtgggagtgtaaggaattacatcatgtcccctgcaattaacttaagagggaaggtaggtttagaaataacagaaagctcgtttgggaatgtatgggttccttatgcaatcaggtatttgcaaaatgaaatggaacccaactttgttaatcttgtgcgcctgatagaagacacaaatagagattttatcatggagcttccaaaagcaagtagcgaagagatatgcgcacaggcaatcgcttgttatgatgaggttgaagaagaaataaagcagaacaaatctcccatcataacaagttggtgtcggtttcctatttataatgctgattttggttggggtaatccctattgggtaagtgaaggtggtcgttcacgagatatggtaactttaatggatgacaaaaatggcgatggaatagaagcatgggtggatctgaaagagaaagacatgtacaagtttgaaaaagatgaggacattacagaactcgcctcctaa

SEQ ID NO: 70 - DNA, 1263 bp, synthetic construct
  source 1..1263: mol_type = other DNA
atggaagttaaaactcaatctactaagtacattatcccatccaagtccaccccagaaaacttacgttcctacaagttgtcccgtttggatcaaatctcccctccatcttatacaaagcaaatttactattacaagccatccggcgacgtttccatctccggtatctgtggtcacttggttatgtccttgtctgaagtcctaactttgttctacccattggccggtagattaactaaagacggtttggaagttgattgttctgatcaaggtgtcaagtacatcgaaactaaggtttctaccagattggacgatttcttggaattaggtccaaccatcgatcaagttaagagattgatctctatcccagacccaggtgccactaccttggtcactgttcaagttaacatcttcgattgcggtgctctagttatcggtgtctctgcctctcataaggtcactgacgcttactctttggtccgtttcattaaccaatgggcttgtattaacagaactggttgttccgatgacgctttttcaccatcctttgataagatggtcaccttgttccctccagaagtcgttccatctattgttcaaattcctgtctctaaccctcaatccaagatggtttccaagagatatatgttctctggtactatcttgtctaagttgagagctaaggctggttctccagactgcaagcattctagagttaccttggtcactgctgtcatctggaaggctttgatcggtgttgataagttgaactgtggttccgttagaaactatattatgtccccagctattaatttgagaggtaaggttggtttggaaattaccgaatcctctttcggtaacgtctgggttccatacgccatcagatacttgcaaaacgaaatggaaccaaacttcgtcaacctagttagattgattgaggatactaacagagacttcattatggaattgccaaaggcttcttccgaggaaatttgtgcccaagctatcgcctgttatgacgaagttgaagaggaaatcaagcaaaataagtccccaatcattacttcttggtgtagatttccaatttacaacgctgacttcggctggggtaacccttactgggtttctgaaggcggtagatcccgtgacatggtcactttaatggatgacaaaaacggcgatggtatcgaggcttgggttgacttgaaggaaaaggacatgtataagttcgaaaaggacgaagatattactgaattagcttcctaa

SEQ ID NO: 71 - DNA, 1290 bp, Erigeron canadensis
  source 1..1290: mol_type = genomic DNA
atggagattaagattcaatccacaaggttcataaaaccatcaaagtcaacgccagaaaatagacgctacttcaagctatcactgcttgatcaacttgctccatctgcatacataaatctaatcttcttctacaaagccagtggtggggtcaatatttcagatagagttggtcaactggtcaagtctttgtcagaggtgctgacttcattctacccgctagctggaagaatcacagaagatgggttagcagttgattgtagtgatcaaggtgttgaatattttgagactcgagtcagtgcaagactggatgagtttctggcacatggttcaaagatggaccatgttcatcgacttgtagccacaccagatcaagttagaaacacgttggtaatcattcaggtgaatgtatttgattgtggctcacttgttataggtgtgagtgcttcacacaaggttactgatgcatgcaacctggttaggttcatcaatgcatgggctagcacaaaccgcacaaggcccagcaacggccccttttctccttcttttgataatttggattctctctttcggccaagggaaaatttatcaaatgaacactcaccagtatcagttgaactagaaaacataacagtgacaaaaagatttgtgtttgacaggactgcgataagaaagctaagatcaaaaacaggtctggaaaatagtactaaacacagtcgagtaacattggtagcttctttagtatggaaggctcttatcttaattgacaaagttaattgtggaagattcagggactgtttgttggctcctgcaatgaatcttagaggaaaggtaggttcgccgatatcagaaagctcgtttgggaacgtatgggctccttatccaatccgtttgctgcaaaaagaaatggaacccaagtttgttgatcttgtttccatgatagaagatacaacaaaaagcattatcgcttggcttgcaacagcaagtggtgaagagatatgcaaacatgcaatggcttcttatgccaaagtaaacgaagaattgaagcacaacaagttttgcatgtttacaagttggtgtcggtttcctatatacgaggctgattttggttggggtaagcctgactgggcaagagcccttgaaaggtcgcttgagctggtaacattaatggatgacaaacatggggatgcaattgaagcatgggtgagtttgaatgagaaagatatgtatgtgtttgaacaagatcgtgacatcttagcctttaccacttcgaccatctcagaggtggcctag

SEQ ID NO: 72 - DNA, 1290 bp, synthetic construct
  source 1..1290: mol_type = other DNA
atggaaattaagattcaatctactagattcatcaagccatccaagtccactccagaaaaccgtagatacttcaagttatccctattagaccaattggccccatctgcctatatcaatttgatctttttctacaaggcttctggcggtgttaacatctctgacagagtcggtcaattggtcaaatcattgtctgaagttttgacttctttctatccattggctggtcgtattaccgaagacggcttagctgttgactgttctgatcaaggtgtcgaatacttcgaaaccagagtttctgctagattggacgaattcttggctcatggttctaagatggatcacgttcacagattagtcgctacacctgatcaagttagaaatactttggttatcattcaagttaacgttttcgactgtggttccttggttatcggtgtttctgcctctcataaggttaccgacgcttgtaacttggtcagatttatcaacgcctgggcttctaccaacagaactagaccatcaaacggtcctttctccccatcttttgacaatttggactccttgttcagacctagagaaaatttgtccaacgaacactcaccagtctctgttgaattggaaaacattaccgtcactaagagatttgttttcgatagaactgctatcagaaagttaagatctaagactggtttggaaaattccactaagcactccagagttacattagtcgcctctttggtttggaaggctttgattttaatcgacaaggtcaattgtggtagattccgtgattgtttgttagctccagccatgaatttgagaggtaaggtcggctctccaatttccgaatcttcatttggtaacgtctgggctccataccctatcagattgctacaaaaggaaatggaacctaagttcgtcgacttagtttctatgattgaagatacaactaagtccatcattgcttggttggctaccgcttccggtgaggaaatttgtaagcacgctatggcttcatacgccaaggtcaacgaggaattgaagcacaacaagttctgtatgttcacctcttggtgtagattcccaatttacgaagctgacttcggttggggtaaaccagactgggccagagctttggaacgttctttggaattggtcaccttaatggacgataaacacggtgatgctattgaggcttgggtttctctaaacgaaaaggacatgtacgtcttcgaacaagatagagatattttggccttcaccacttctaccatctctgaagtcgcctaa

SEQ ID NO: 73 - DNA, 1266 bp, Helianthus annuus
  source 1..1266: mol_type = genomic DNA
atggagattaagattcaatccattcagttcataaaaccatcaaaaccaaccccggaaaatctacgccacttcaagctatcattgctggatcaactagctccatgttcatacataaatccgatcttctactatagcaccagtggtgaagttgaaaactcagctagatgtggtcagttggccaactctttatcagaggtcctaaatttgtactacccactagctggcagagtcacagaagatggtctggaagtagattgtaatgatcaaggggtcaagtatttggtgactcgtgtgagcacaagcctggatgattttctcaaacaaggtcccaggattgaccacatgagacaactcatagctgcaagggatcaagacacaacttggttggttacggtacaagtcaacgtctttgtttgtggtgcacttgctattggtgtgattgcttcacataaactcactgacggatgcaatctagtcaggtttattaaccagtgggcgagaatgaaccacgcatgtggcggcgatggtgcctttgcacctacttatgataaactagatcatctgttcccaccggtgacaaattcatcacctgggcactcatccgatccggttgaccaagaagctattgtcgtcacaaagaggtttctgtttaatgggacttcaatatcaaagctaagagcaaaagctggttcagcaaaaggagaacacactcgggtgacattggtggcttcgttattatggaaggctctgatagccatggatcgagtcaaaagtgggagtttcagggattgtttgttgacggttgcaatgaatttgagaggtaaggcaagctcaccagtatcgaaaagcacgtttgggaacgtatgggctccttatccaattcggtttttgcaaaacgaaactgaacctgagtttggtgatcttgtggctttgatagaagacacaacaagaaatgttatcaagtgggttcaaaaagcaagtggtgaggagctatgcagacatgcaaaggcaggttatgctttggttgatgaagaactgaagcaaaacaagttttgcatttttacaagttggtgtcggtttccgatttacgaggttgattttggttggggtaagccttgctgggtaactgaagctggaagtgcacttgagatggtgagtttaatggatgacaaagatggtgatggaattgaagcatgggttagtttgaaccagaaagaaatgtatgtgttcgaacgcgatcatgacattttagatttcacctcctaa

SEQ ID NO: 74 - DNA, 1266 bp, synthetic construct
  source 1..1266: mol_type = other DNA
atggaaattaagatccaatccattcaattcattaagccttccaagcctaccccagaaaacctaagacatttcaagttgtctttattggatcaattggctccatgttcttatattaacccaattttctattactccacttccggtgaagtcgaaaactctgctcgttgtggtcaattggctaactctttgtccgaagtcttgaacttgtattaccctttggctggtagagttactgaagacggtttagaggttgactgtaacgaccaaggtgtcaagtatttggttaccagagtctctacctctttagacgatttcttgaaacaaggtccaagaatcgaccacatgagacaattgatcgctgccagagatcaagacactacctggttggttactgttcaagtcaacgtcttcgtttgtggtgctttggctatcggtgttatcgcttctcataagttaaccgacggttgcaacttggttagatttattaaccaatgggctcgtatgaatcacgcttgcggcggtgacggtgctttcgctccaacttacgataagttggatcacttattccctccagttactaattcctctccaggtcactcctctgacccagttgatcaagaagccattgtcgttactaagcgttttttgttcaacggtacttccatttccaagttaagagctaaggctggttccgctaaaggtgaacacactagagtcactttggtcgcctctttgttatggaaggctttgattgctatggacagagtcaaatctggttcttttcgtgactgtttattgactgttgctatgaacttaagaggtaaggcctcctctccagtctctaagtccactttcggtaacgtttgggctccatacccaatcagatttttgcaaaacgaaacagaacctgagttcggtgatttagttgctttgatcgaggacacaactagaaacgttatcaaatgggttcaaaaggcttcaggtgaggaattgtgcagacacgctaaggctggttacgccttggttgatgaggaattgaagcaaaacaaattttgtattttcacttcttggtgtagattcccaatttacgaggttgacttcggttggggtaagccatgttgggttactgaagccggttctgctttggaaatggtttccttgatggacgataaggatggtgatggtattgaggcctgggtttccttgaaccaaaaggaaatgtacgttttcgaaagagatcacgacatcttagacttcacatcctaa

SEQ ID NO: 75 - DNA, 1290 bp, Helianthus annuus
  source 1..1290: mol_type = genomic DNA
atggtgatggcaatgaagattgaaaaacaatccagcaaattaataaaaccctttgttcaaactcctcctacacaatctcactacaaattgggcttcatcgacgagttagctcctgcccatgatactggcatcgttctattctttgccgctaatagcaatcacaaccccaactttcttgcccggcttgaaaaatcgcttggaaaaaccttaacacgactctaccctcttgcgggtagatacgttgaagaaactcatagtgttgattgcaaagaccaaggtgctgagtttatacacgccaaagttaatatcaaacttcaagattttcttgtctccgaagaaaacgttaagtttactgacgaattcattccatccaagataggggttgctcgtcaacaaagtgacccgttacttgcaactcaagtaaccacctttgaatgtggaggtttggcaattggtgcaagtgctacacacaagattgttgatgcttccactctatgcacatttgtaaacgaatgggctgttacaaatcgagaagaaaatgagattgagttcaaagggcctggtttcaattcatccatattgtttcctggacgcggtttaagctctataccattgccatttataaacattgagatgttaaacaagtatacaaaaaagaaactttcattcagtgggagtgcaatatcaaagatgaaagcaaagtgttcaaatcgcaccagccaacggtccaaggtacaattggtatcagcaatcctttggaaaactttcatgggtgttgatctagcaatacacaatcatcaaagacattccatacacattcaggcagtaagcttgagggaaaaaatggcatcctcaatacccaaaacttcttgtgggaatctttgtggggcatgtaccacagaatgtacaactcttgaaagaacagaagaactggcagaccgtttaactgattccgtcaagaaaactgtaactaagtactcgaaggagcgccatgactgcaaagaaggacaagcgatggttttgaatttaataatgtcaagcatggacaatattagtgaatctactaatgttgtcttcacaactagttggtgtaagtttcctttttatgaagctgactttggtttcggaaaacctacttgggttgcccctggtatcgtaccggttcaccaaatgacgtatatgatcgatgacgctgaaggtactggagtagaagcatatgtctatcttgaagttaaagatgtgccttatttcgaagaagctctagaacatgctattgctttcggagcataa

SEQ ID NO: 76 - DNA, 1290 bp, synthetic construct
  source 1..1290: mol_type = other DNA
atggtcatggctatgaagatcgaaaaacaatcctctaaattgattaagccattcgtccaaactccacctacccaatctcattacaagttaggtttcattgacgaattggctccagctcatgacaccggtattgttttgtttttcgccgctaattctaaccataacccaaatttcttggctcgtttagaaaaatctttgggcaaaactttgactcgtttatacccattggccggtagatacgttgaggaaacccactctgtcgactgtaaagatcaaggtgccgagttcattcacgctaaggttaacatcaagttgcaagactttttagtctccgaggaaaacgtcaaattcactgatgaatttattccttctaagattggtgttgccagacaacaatctgacccactattggctacccaagtcaccactttcgaatgtggcggtttggccattggtgcttccgctacccataaaattgttgacgcttctactttatgtaccttcgtcaacgaatgggctgttactaacagagaggaaaacgaaatcgaattcaagggtccaggtttcaactcctctatcctattcccaggtagaggcttatcttccatcccattgccattcatcaatatcgaaatgttgaacaagtacactaagaaaaagttgtccttttctggttccgctatctctaagatgaaggccaagtgttccaaccgtacttctcaaagatccaaggtccaattagtctccgccatcttgtggaagactttcatgggtgttgatttagccattcacaaccaccaaagacattctatccacattcaagctgtctctttgagagaaaaaatggcttcttccattccaaaaacttcttgtggcaacttgtgtggtgcttgtacaactgagtgtaccactttggaacgtactgaggaattggctgacagattaactgattctgttaagaaaactgtcacaaagtactctaaggaaagacacgattgtaaggaaggtcaagctatggttttgaacttaatcatgtcatctatggacaacatttctgaatctaccaatgttgtcttcactacctcttggtgcaagttccctttctatgaggctgacttcggttttggtaaacctacttgggtcgctcctggtattgttcctgtccaccaaatgacctacatgattgatgacgctgaaggtaccggtgtcgaggcttacgtttacttggaagtcaaggatgttccatattttgaagaggctttggaacacgctattgccttcggtgcttaa

SEQ ID NO: 77 - DNA, 1299 bp, Daucus carota
  source 1..1299: mol_type = genomic DNA; note = subspecies sativus
atgaaggttcaaatccattctaagaaattagttaaaccatttactccaactccctccaatctgaaccatcacaacctatctttcattgatgagttagctccaaaaatgtatgcccctgttattctgtattatccatgtcctgaaaatgttaccgacaaggatctcttttcttcgacatgtttgctcgagctagagacatcgttgagcaagactctggttcagttctatccactagcagggaggtataacaaacaccttcagttggttgattgcaatgacaaaggagttgagtttgtcgaagccactgtggactgtcatctccacgaggttttacctcacggggaaagatcagatcctcagtttctcaacaagtttattccctgtgaagttctgattactgatgaagccatgcatccattgcttgccattcaggttaccgtgtttaagtgtggtggattcgcgattggcgtttgcatttcacacaggattgctgatgccgccacgctgagcatgttccttcaagcatggggaaccacagcaaagttaaacaagaatggaaatcaacaagagagtattcagaaaatttttccatgttttgatgcggctgtgtactttccaaaaagagggctaccacaccttaatttcggaatattcacaagttctggctgcaagatcgtcacaagaaggttctcattcgacaacaaggcaatatcaaccctaagagccaatattctacctggaaccgggcaaactagtaagttgcagatggtgattgcagtcatatggaaggcattacttggtgcagagaagttgaaagatgaacacgcgagggctactcatatcatgcagcccgtcaacctgagggataagcttaccactccgtttctacataaacatttctttggaaatctatgcattcttgcatcggtgccaatgatggcagcagagaaccgggagattcctgacttagccattcaactgagcagttctgtgaagggcagtatcgagggatgggcaaagatgatgtctctaggcaaggatgatccgcttctaaagaatatgataagcagtacggtcaattactacatgattagtagctggagtaggtttccattttacgaaactgattttggctgggggaagcctgtttgggcaagtagtgtatacttcccttgtaagaatatggtcctcctgatggataataagaagggtgatggcttcgaagcatgggtgagcttggacgaagcagacatgaacatcttcgaacaggatttcaacatcaaagcgttctctacctag

SEQ ID NO: 78 - DNA, 1299 bp, synthetic construct
  source 1..1299: mol_type = other DNA
atgaaggttcaaatccactctaaaaagttggtcaagccatttactccaaccccatctaatttgaaccaccataacttgtctttcattgacgaattggctcctaagatgtacgccccagtcattttgtactatccatgcccagaaaatgtcaccgacaaggatttgttctcttccacctgtctattggaattggaaacttctttgtctaagaccttggttcaattttatccattggccggtagatacaataagcacttgcaattggttgactgtaacgacaagggtgttgaattcgtcgaggctactgtcgattgtcacttgcacgaggttttgccacacggtgaaagatctgacccacaatttttaaacaagtttatcccatgtgaggtcttgatcactgatgaagctatgcacccattattggccattcaagttactgttttcaagtgtggcggttttgctatcggtgtttgtatctcacaccgtattgctgatgccgctaccttgtcaatgttcttacaagcctggggtacaactgctaagttaaacaaaaacggtaaccaacaagaatctatccaaaagattttcccatgtttcgacgctgccgtttacttcccaaagagaggcttgccacatttgaatttcggtattttcacctcctctggttgtaagatcgttaccagacgtttctctttcgacaacaaggccatctctactttaagagctaacattttgccaggtaccggtcaaacttccaaattgcaaatggttattgctgtcatttggaaggccctattgggtgctgaaaagttgaaggacgaacacgctcgtgctacccatattatgcaaccagtcaacttgagagataagttgaccactcctttcttacacaagcactttttcggtaacttgtgtatcttggcttcagtcccaatgatggccgctgaaaatcgtgaaatcccagacttggctattcaattgtcctcttccgtcaagggttccatcgaaggttgggctaagatgatgtccttgggtaaggatgacccattattgaaaaacatgatctcatctactgtcaactattacatgatctcctcttggtcccgtttccctttttacgaaaccgatttcggttggggtaaaccagtctgggcttcctcagtttactttccttgtaaaaacatggttttattgatggataacaaaaagggtgacggtttcgaggcttgggtttctctagacgaagctgacatgaatatctttgaacaagacttcaacattaaggctttctccacctaa

SEQ ID NO: 79 - DNA, 1287 bp, Mikania micrantha
  source 1..1287: mol_type = genomic DNA
atggccaaatcaatcaagattcaaaaacaatcaagcaaattcataaaaccctttgttcaaacacctcaaacactcactcactttaagttaggcttcatcgatgagttttctcccgatgtaaatgttggtgttgttctattcttctctactaacacccatcaaaccacaaactttattgcgcgacttgaaaaatctctagagaaaaccttaacacgattctaccctcttgcgggtagattatacgttgataaaaacgccaccattgattgcaatgatcatggtgctgagtttatacatgccaaagttaacatcaaacttcaaaattttcttgtttccgaagcgaatgttaagttcattgatgacttcattccatccaaaataggggttgtccttcaacaaagtgacccgttacttgcggttcaagtaaccatctttgaatgtggaggtgttgcaattggtgtaaaggctacacacaagattgttgatgcttccactctatcaacactcataaatgaatggtctgttacaaaccgacaagaaaatgataataataatgataatgataatatattctcagggactaatttcgtttcgtccttattgttccctcctcgtggtttatgtcccatgccggtgccacctatgaacaatgacgaattaaacaagtatacacgaaagaaactttcattcagcgagaatgtgatatcaaacatgaaagcaaaggggtcgaaggtgcaattgatatcagcgatcatttggaaagctttcatgggtgttgatatcgcgatacacagccatcaaagagagtctatgttgattcaggcagtaaacttaagggggaaaatggcatccttaatacctaaaacttcttgtgggaatctttttgggacatgtaccacagaatgtaggactcatgagaccattgaagaagtgactgatcgtttaagtgattctgtcaggaaatgtataaccaacttctcgaaggtgcatcatgattgcgaagaagggcaaaccatggttttgaaatcattgttaagtttgactaatattcgtgagtctacttgtggtgtcattgtaacaagttggtgtaagtttcctttttaccaagttgactttggttttggaaaacctatttgggcggcccctgggaccataccaataaatcaatcggcatatttgatggatgatgtccaaggtaacggagtcgaagcatatgtatttcttcaagtcaaagacatccctctctttgaagaagccctaaaacatgttattactatgtcgttttag

SEQ ID NO: 80 - DNA, 1287 bp, synthetic construct
  source 1..1287: mol_type = other DNA
atggctaagtctatcaaaattcaaaagcaatcctctaaattcattaagccatttgttcaaactccacaaactttgactcacttcaagttaggtttcatcgacgaattctccccagacgttaacgttggtgtcgttttgtttttctccactaacactcaccaaacaactaacttcatcgccagattggaaaagtcattagaaaagactctaactagattttatccattggccggtagattgtatgtcgacaagaacgctactattgattgtaacgatcacggtgccgaattcattcatgctaaggtcaacatcaaattacaaaacttcttagtttccgaagctaacgttaaattcatcgatgactttatcccatccaagatcggtgtcgttttgcaacaatctgacccattattggctgttcaagtcaccatcttcgaatgcggcggtgttgccatcggtgtcaaagctactcacaagatcgttgacgcttctacattgtccactttgatcaacgaatggtctgtcactaacagacaagaaaacgacaataacaatgacaacgataacatcttctccggtaccaactttgtctcttccctattgttccctccaagaggtttgtgtccaatgccagtccctccaatgaataacgacgaattgaacaagtacactagaaagaaattatcattctccgaaaacgttatttctaacatgaaggctaagggttctaaggtccaattgatctccgctatcatttggaaggctttcatgggtgttgacatcgctatccactcccatcaaagagaatccatgttgatccaagctgttaacttaagaggtaaaatggcttccttgatcccaaaaacctcttgtggtaacttgttcggtacttgtactacagaatgtagaacccatgaaacaatcgaggaagtcaccgaccgtttgtctgactctgtcagaaaatgtattactaacttctctaaggtccaccatgattgtgaggaaggtcaaactatggttttgaagtctttattgtctttgactaacatcagagaatctacttgcggtgttattgtcacttcctggtgtaagtttccattctaccaagtcgatttcggtttcggcaaaccaatttgggccgctccaggcaccatcccaatcaaccaatctgcttacttgatggatgacgttcaaggtaacggtgttgaagcctacgtcttcttgcaagtcaaggacattccattgtttgaggaagccttgaagcatgtcatcactatgtcattctaa

SEQ ID NO: 81 - DNA, 1290 bp, Tanacetum cinerariifolium
  source 1..1290: mol_type = genomic DNA
atggcaatgaaagttgaaaaacaatcaagcaaattcataaaacccttttatccaactccttcgaaccttggtcgctatagattaggattcacagatgaaattgctcctctcgaaactattggtgtcgttcttttcttttccccaaatagcaaccacgacacaaagtttgttgcgcaactagaaaaatcccttgagaaaaccttaacacatttataccctcttgccggaagatatgttgatgatgttgatcaaacagttgaatgtaacgatgaaggtgctcattttatatacgcgatagtcaacatcaaactttatgaatttcttggcttgaaagaaaaatttaaaatggctgatgagttcattccgttaagtaggggcactcatcaattcagtgacccgttacttgaaattcaagtgactatgttcgagtgtgggggtatagcggttggtgtacgtgttgcacacaagattgctgatgcgtccaccttatgtacattccttaatgaatgggcttgtattagccgagatgaaaatgagattggatacgttggacctagtttcaactcatccttgttgtttcctcctcgtggtgtacgtacacttccgttaccacccatgagtgctgatatgttaagcaggtctacgagaataaatttctcacttagtgaaagtgaaatatccaatatgaaagcaaacgccattgcgagtggtaaagttagcgcacgtgaattgtcaaaggtacaattggtatcagcgatcctttggaaggctctcagagttgttgatcggctagtacacaattatttgagagattctatactcctccaacccgtaaacctaagggggaaaatggcatcgtcaataccggaaaattcttgtgggaatcttttaagtatttgttcgacaaaatctgagaatgttgaaacaatggaagaattggtaaatcttttaagcaactctgtcaagaaaaccgtaaaagagtactcaaaagtataccatgatagcgaagaagggcaaatgatggttttgaattcctacctgaacttagcaaatattcctgaatctagtaatgtggtcttcgtaactagttggtgtaagttccccttttatgaagttaactttggtctcggaaatcccatttgggttgttcctggtactgtactttcaaagaacttggggtgtttgattgacgatgcacaaggtaatggggtggaagcatatgtctttctcgaagttaaagatgttccttgttttgaagaagctctagatttcaatgttttcggtgattaa

SEQ ID NO: 82 - DNA, 1290 bp, synthetic construct
  source 1..1290: mol_type = other DNA
atggccatgaaggttgaaaagcaatcttccaagttcattaaaccattctatccaaccccatctaacttaggtcgttacagattgggtttcactgacgaaatcgccccattggaaacaattggtgtcgttttgtttttctctccaaactctaaccacgacaccaagtttgttgctcaattggaaaagtctttagaaaaaaccttgacccatctataccctttggctggtagatatgtcgatgacgttgaccaaaccgttgaatgtaacgacgaaggtgctcattttatctacgctattgttaatatcaagctatacgagttcttgggtttaaaggaaaagttcaaaatggctgacgaatttatcccattgtctagaggtactcatcaattttccgatccactattggaaatccaagttactatgttcgaatgcggcggtattgccgtcggtgtcagagttgctcataagatcgctgacgcctccactttatgtaccttcctaaacgaatgggcctgtatttctcgtgatgagaatgaaatcggttacgtcggtccatcctttaactcatctctattgtttccacctagaggtgtccgtaccttgccattgcctccaatgtctgctgacatgttatccagatctaccagaatcaacttctccttgtccgaatctgaaatctctaacatgaaggccaacgctattgcttccggtaaggtttctgctagagaattgtctaaggttcaactagtctctgccattttatggaaggccttgagagttgtcgacagattagtccataactacttgagagattctattctattgcaaccagttaatttaagaggtaagatggcctcttcaattccagaaaactcctgtggtaacttgttatccatctgttccactaagtccgaaaacgttgagactatggaggaattggtcaacttattgtctaattctgttaagaaaaccgttaaggaatactccaaggtttaccacgactccgaggaaggtcaaatgatggttttaaactcctacttaaacttggccaacattccagagtcatccaacgttgtctttgtcacttcttggtgtaaattcccattctatgaggttaacttcggtctaggtaatccaatctgggtcgttcctggtaccgttttgtctaagaacttgggttgtttgattgatgacgctcaaggtaacggtgttgaggcttacgttttcttggaagtcaaggatgttccttgtttcgaagaggctttagatttcaatgtttttggtgactaa

SEQ ID NO: 83 - DNA, 1314 bp, synthetic construct
  source 1..1314: mol_type = other DNA
atggaaattaaatctattacttctaagttggtcaagccatcaacaccaaccagatccaatttgcaaaactatgacatttccttctttgatgaacaaatggctaacattaatactcctttgatcttatattactccgcttcccaaaattccccagacgataacatcttcaaccacttggaaacttccttgtccaagaccctaaccgatttctatccattggctggtagatacaagagacaaggctctttcgttgactgttctgaccaaggtgtcctatacattcaatctatcgctaacttcagattggaggaatttctaggccaggcttgggaattgaagtttagaatgttaaatgacctattgccatgcgaagttccagaagctggtgaggtcgatgaccctttgttatgtatcaaggttactgcctttgaatgtggcggtttcgccattggtatgtgtttggctcataagattgctgatatgtgtactatgtgcacctttatcaataactgggccactagatcctctcaagaaatcgttaataaacaagaattggaaaagtactctccaattttttctgtcgcccacgacttcccaaaagttgctttggatgacttatctccaagattgccaagatctaatattggtatggaaactaaagtcaagttgttccaatttaaagtcgatgctatctctaagatgagagaaaacttgagagataaatcttatcactcctctaaggttcaactaattgttgccttgttttctaaggccttgatggctattgataaagccaagttcggtcattctaaaccatctattgttcaacaaggtgttaacctaagaaacaaagtcgttccaaagttgccagaaaacctattcggtaactttttctctttcttgcatggtagaattgaaccagaagatggtgaaaacatggacttagacggtttcttgatgatcctaaaggactctatgaagaaatttgaggatgaatacgttaaggctttgatgtcctctgataaggattatgaagtcttggttaagccatttttgaagtttggtgaaaccttcgctaataacgatgaaaatttccactgtttcacttcttggtgtaagttctccttctacaaggctgatttcggttggggtaagccagtttggagatctacaggtcattacgctaaccacaagttcgttatcttgatggacgatgaggaaggtggcggtgtcgaggcttgggttcacttggacgaaaagtctatgtgtcaattggaacaagacccagatattaaggcttacgctacttaataa

SEQ ID NO: 84 - DNA, 1311 bp, synthetic construct
  source 1..1311: mol_type = other DNA
atggaaatcaaatctatcacctctaaactggtgaaaccgagcaccccgactcgtagcaacctgcagaattacgacatctcttttttcgacgagcagatggccaacatcaacaccccgctgattctgtattacagcgcttctcagaactctccggacgataacatttttaaccacctggaaacctctctctccaagactctgactgacttctatccgctggcaggtcgctacaaacgccagggtagctttgtcgattgctccgaccagggtgttctgtacatccagtctatcgctaacttccgtctggaggaattcctgggtcaagcatgggagctcaaattccgtatgctgaacgacctcctgccatgcgaagtaccggaggctggcgaggtcgacgatccactcctgtgcattaaagtaaccgcatttgaatgcggtggcttcgctatcggtatgtgcctggcgcacaaaatcgcagatatgtgcaccatgtgtaccttcatcaataactgggccacccgttccagccaggaaatcgtaaacaaacaggaactggaaaaatactccccgatcttctctgtggctcacgacttccctaaagtggccctggatgacctgtccccgcgcctgccgcgttccaatatcggtatggaaactaaagttaaactgtttcagttcaaagttgacgccatcagcaaaatgcgcgaaaacctgcgcgacaaatcttaccattccagcaaggtgcaactgatcgtagccctgttctctaaagccctgatggccatcgataaggcgaaatttggccactctaaaccgagcattgtgcagcaaggcgttaacctgcgcaacaaggttgtgccgaaactccctgaaaacctgttcggtaacttcttttctttcctgcacggtcgtattgaaccggaagacggtgaaaacatggacctcgacggtttcctcatgatcctgaaggatagcatgaagaaattcgaagacgagtacgtcaaagctctgatgtcttccgataaagattacgaggtgctggtcaaaccgttcctgaaattcggtgaaaccttcgcgaataacgatgagaacttccactgtttcacttcctggtgtaaatttagcttctataaggcggattttggctggggtaaaccggtatggcgttctacgggtcactacgctaaccacaaatttgttatcctgatggatgacgaggaaggcggtggcgtggaggcttgggtgcatctggacgaaaaatctatgtgccagctggaacaggatccggatatcaaagcgtacgctacctaa

SEQ ID NO: 85 - DNA, 1314 bp, synthetic construct
  source 1..1314: mol_type = other DNA
atggaaatcaagtctattacttcaaaattagtcaagccatccactccaactcgttctaacttgcaaaactacgacatttcttttttcgacgagcaaatgcacaacattaacactccattgatcttgtactattccgcttcccaaaactccccagacgataacattttcaaccatttggaaacttctttgtccaaaactttgacagatttttacccattagccggtagatataagagacaaggttctttcgtcgactgttccgaccaaggtgttttgtacatccaatccatcgctaactttagattggaggaattcttgggtcaggcttgggaattgaagttcagaatgttgaacgacttattgccatgtgaagttcctgaagctggtgaggtcgacgatccattgctatgtattaaagtcactgctttcgaatgtggcggttttgccatcggtatgtgtttagcccataaaattgccgacatgtgtacaatgtgtaccttcattaataactgggctaccagatcctctcaagaaattgttaacaaacaagaattggaaaagtactctcctatcttctctgtcgctcacgattttccaaaggttgctttggatgacctatccccaagattaccaagatctaacattggtatggaaaccaaggttaagttgttccaattcaaggtcgacgccatttctaagatgagagaaaacctaagagataaatcttaccattcttccaaggtccaattgatcgtcgctttgttctcaaaggctttgatggctattgataaggctaagttcggtcactccaaaccatctattgtccaacaaggtgttaatttgcgtaacaaggtcgttcctaagttgccagagaacctattcggcaacttcttttctttcttgcacggtcgtatcgaaccagaagatggtgaaaacatggacttggatggtttcttgatgattttgaaggactctatgaaaaagttcgaagatgaatacgttaaggctttgatgtcctctgacaaggactatgaagtcttggttaagccattcctaaagttcggtgaaacttttgccaataacgacgaaaacttccattgttttacctcttggtgcaaattttctttttacaaggctgacttcggttggggtaaaccagtctggagatcaaccggtcattacgccaatcataagttcgttatcctaatggatgacgaggaaggtggcggtgttgaagcctgggtccatttggatgaaaagtctatgtgtcaattagaacaagatccagacattaaggcttatgctacctaataa

SEQ ID NO: 86 - DNA, 1311 bp, synthetic construct
  source 1..1311: mol_type = other DNA
atggaaatcaaatccattacttccaaactggtgaaaccgtccaccccgacccgtagcaacctgcagaactacgacatctcctttttcgacgaacaaatgcacaatatcaacaccccgctgattctgtactattctgcgtcccagaactccccggacgataacatttttaatcacctggaaacttccctgtctaaaaccctgaccgatttctacccgctggccggccgctacaaacgtcagggttccttcgtggactgtagcgatcagggtgtgctgtatatccagtctattgcaaatttccgcctggaagagttcctgggccaggcctgggaactgaagttccgtatgctgaacgatctcctgccgtgcgaagttccagaagctggtgaagttgacgatccgctcctgtgcatcaaagtgacggcgttcgaatgcggcggttttgctatcggtatgtgtctggcgcacaaaatcgcggacatgtgtacgatgtgcaccttcatcaataactgggcgactcgttcctctcaggaaattgtaaacaaacaggagctggaaaaatactccccgatctttagcgtcgcacacgatttcccgaaagtcgccctggacgatctgtctcctcgcctcccgcgtagcaacattggcatggaaactaaagtaaaactgtttcagtttaaagttgacgccatttccaaaatgcgtgaaaacctgcgtgataagtcctaccacagctctaaagtacagctgatcgtggcactgttttctaaggccctgatggcgatcgataaagcgaaattcggccacagcaaaccttctattgtacaacagggtgtaaacctgcgtaacaaagtagtgccgaaactgccagaaaacctgttcggtaactttttcagctttctgcatggccgtatcgagccggaggacggcgaaaatatggacctggacggcttcctgatgatcctgaaagacagcatgaagaaattcgaagatgaatatgtgaaagctctgatgagctctgacaaagactacgaagtactggttaaaccgttcctgaaattcggtgaaactttcgcaaataacgacgaaaacttccactgcttcacctcttggtgcaagttctctttctacaaagctgacttcggttggggtaaaccggtatggcgtagcaccggtcattacgcgaaccataaattcgtgatcctcatggatgacgaagagggcggtggcgttgaagcgtgggtacacctggatgaaaagtccatgtgtcagctggaacaggacccggatatcaaagcctatgcgacttaa

SEQ ID NO: 87 - DNA, 1314 bp, synthetic construct
  source 1..1314: mol_type = other DNA
atggaaattaagtctatcacttctaagttggtcaaaccttccactccaaccagatccaacttacaaaactacgacatttcttttttcgacgaacaaatggctaatattaacactccattgattttatattactcagcttctcaaaactctccagatgacaacatttttaaccatttggaaacctccttatccaagactttgactgatttctacccattggccggtagatataagagacaaggttcttttgtcgactgttcagaccaaggtgttttgtacatccaatctatcgccaatttcagattggaggaattcttgggtcaagcctgggaattgaaatttcgtatgttgaacgatttgctaccttgtgaagttccagaagctggtgaagtcgatgacccactattgtgcattaaggttaccgccttcgaatgtggcggtttcgctattggtatgtgtttggctcataaaatcgctgatatgtgtaccatgtgtaccttcatcaacaattgggccacccgttcctctcaagaaatcgttaacaagcaagaattggaaaaatactcccctatcttttccgttgcccacgacttcccaaaggttgctttggacgatttgtccccaagattacctagatccaacatcggtatggaaacaaaggtcaagttgttccaatttaaggtcgatgctatttctaagatgagagaaaatttgagagataagtcctatcactcatctaaggttcaattgattgtcgctttgttctctaaggctttgatggctattgataaggccaagtttggtcattccaagccatccattgttcaacaaggtgttaacttgagaaacaaggttgtcccaaagttgcctgaaaacttgttcggtaatttcttttcattcttgcacggtcgtattgaacctgaagacggtgaaaacatggatttggacggtttcttgatgattttgaaggactccatgaaaaagttcgaagatgaatacgtcaaggccttgatgtcctctgataaggactacgaagttttggttaagccatttttgaaatacggtgaaactttcgccaacaatgatgaaaatttccattgtttcacttcttggtgcaagttttccttctacaaagccgattttggttggggtaaaccagtctggagatccacaggtcactacgctaaccataagttcgtcattttaatggatgacgaggaaggtggcggtgttgaggcttgggttcatttagatgaaaagtccatgtgtcaattggaacaagacccagatattaaggcttacgccacctaataa

SEQ ID NO: 88 - DNA, 1314 bp, synthetic construct
  source 1..1314: mol_type = other DNA
atggaaatcaagtctatcacttcaaagttagttaagccttccaccccaacaagatctaacttgcaaaactatgacatttcttttttcgatgaacaaatggccaacattaacactccattgattttgtattactctgcttcccaaaactctcctgatgacaacattttcaaccatttggaaacatctttatccaagactttaactgatttctacccactagctggtagatataagagacaaggttctttcgttgattgttcagatcaaggtgtcttatacattcaatcaattgctaacttccgtttggaggaattcttaggtcaggcttgggaattgaagtttagaatgttgaacgatttattgccatgtgaagttccagaagctggtgaagttgatgacccattgctatgtatcaaggttactgctttcgaatgtggcggtttcgctattggtatgtgtttggctcacaagattgctgacatgtgtactatgtgcactttcattaataactgggctactagatcctctcaagaaattgtcaacaagcaagaattggagaaatactctccaattttctccgtcgctcacgatttcccaaaggttgctttagatgacttatccccaagattgcctagatcaaacattggtatggaaactaaggttaagttgttccaattcaaggttgacgctatttctaagatgagagaaaatttgagagataaatcttaccattcatctaaggttcaattgatcgttgccttattctccaaggctttgatggctattgataaggctaagttcggtcattctaagccatcaatcgttcaacaaggtgttaacttgagaaacaaagtcgttccaaagttaccagaaaacctattcggtaattttttctctttcttgcacggtcgtattgaacctgaagacggtgagaacatggacttggacggtttcttgatgatcttgaaggactctatgaaaaagttcgaagacgaatatgtcaaggctttgatgtcttccgacaaggactacgaagttctagtcaagccatttttgaagttcggtgaaactttcgctaataacgatgaaaactaccactgtttcacatcctggtgcaaattttctttctacaaagctgatttcggttggggtaaaccagtctggcgttctactggtcattatgccaatcacaaatttgttatcttgatggacgatgaggaaggtggcggtgtcgaggcttgggttcacttggacgaaaagtccatgtgtcaattggaacaagacccagatattaaagcctacgctacctagtaa

SEQ ID NO: 89 - DNA, 1314 bp, synthetic construct
  source 1..1314: mol_type = other DNA
atggaaatcaagtctattacatctaagttggttaaaccatctactccaactagatctaacttgcaaaactacgatatctcttttttcgatgaacaaatggctaatatcaataccccattgattttgtactattctgcttctcaaaactcaccagacgataacattttcaaccatttagaaacctccttgtctaaaacattgacagatttctatccactagctggcagatacaagagacaaggttcctttgttgattgttctgatcaaggtgttttatacatccaatccattgctaacttcagattagaggaattcttgggccaggcttgggaattaaagtttagaatgttgaacgatttattgccttgtgaagtcccagaagctggtgaagttgatgacccactattgtgtattaaagttactgcttttgaatgtggtggcttcgctatcggtatgtgtttagctcataagattgctgatatgtgtacaatgtgtacatttattaataactgggccaccagatcttcccaagaaattgtcaacaagcaagaattggaaaaatattctccaatcttttctgttgcccatgactttccaaaggtcgctttggacgatttgtctccaagattaccaagatccaacatcggtatggaaaccaaggttaagttgttccaattcaaggttgacgctatttctaagatgagagaaaacctaagagataagtcttaccactcatctaaggttcaattgattgtcgctttgttctccaaagccttgatggctatcgacaaggctaagtttggtcattctaagccatctattgttcaacaaggtgttaacttgcgtaacaaagtcgttccaaaattgccagaaaacttgttcggtaactttttctctttcttgcacggtcgtattgaacctgaggacggtgaaaacatggatttggatggtttcttgatgattttaaaagactcaatgaaaaagttcgaagatgaatatgtcaaggctttgatgtcatctgacaaggactacgaagtcttagttaaaccattcttgaagtatggtgaaactttcgccaataacgacgaaaactaccactgtttcacctcctggtgtaagttctctttctacaaggctgactttggttggggcaaaccagtttggagatccactggtcattacgctaaccacaaattcgtcattttgatggatgacgaggaaggtggcggtgttgaagcctgggttcatttagacgaaaagtctatgtgtcaattggaacaagatccagacattaaggcttacgctacataataa

SEQ ID NO: 90 - DNA, 1314 bp, synthetic construct
  source 1..1314: mol_type = other DNA
atggaaatcaagtctatcacctccaagttggtcaagccatctactccaacccgttctaacttacaaaactacgacatttcctttttcgatgaacaaatgccaaacatcaacacaccattgattctatattactctgcttctcaaaactctcctgatgacaacatttttaaccacttagaaacatccttatccaaaactctaaccgacttctatccattggccggtagatacaagagacaaggttccttcgtcgactgttccgaccaaggtgttttgtatatccaatctatcgccaactttagattggaggaattcttgggtcaagcctgggaattaaagttcagaatgttgaacgacctattgccatgcgaagttcctgaggctggtgaagttgacgatccactattgtgtattaaggtcaccgccttcgaatgtggcggtttcgctattggtatgtgtttggctcataagattgccgacatgtgcactatgtgtactttcattaataactgggccactagatcttcccaagaaattgtcaacaagcaagaattggaaaagtactccccaatcttttctgttgctcacgatttcccaaaggttgctttggacgatttgtccccaagactacctagatctaacattggtatggaaactaaggtcaagttgtttcaattcaaggtcgatgctatctccaagatgcgtgaaaacttaagagacaaatcctaccactcctctaaggttcaattgatcgtcgctttgttttctaaggctttgatggctatcgataaggctaaattcggtcactctaagccatctattgtccaacaaggtgtcaacttgagaaacaaagttgtcccaaagttaccagaaaacctattcggtaatttcttttcattcttacacggtcgtattgaaccagaagatggtgaaaacatggacttggatggtttcttgatgatcttgaaggactctatgaaaaagtttgaagatgaatacgttaaggccttaatgtcctctgataaggattatgaagtcttggttaagcctttcttgaaattcggtgaaacctacgctaataacgacgaaaacttccattgctttacctcttggtgtaagttctcattctacaaggccgattttggttggggtaagccagtttggagatctactggccattacgctaatcataagttcgttatcctaatggacgatgaggaaggtggcggtgttgaggcctgggtccatttggacgaaaagtctatgtgccaattggaacaagaccctgacatcaaggcttacgctacttaataa

SEQ ID NO: 91 - DNA, 1311 bp, synthetic construct
  source 1..1311: mol_type = other DNA
atggaaattaaatctatcaccagcaaactggtgaaaccgtccaccccgacgcgctccaacctgcagaattacgacatttccttctttgatgaacagatgccgaatatcaacactccgctcatcctgtattactctgccagccaaaacagcccggatgacaacatttttaaccatctggaaacctccctgagcaaaaccctgaccgacttctatccgctggctggtcgttacaagcgtcagggttctttcgttgactgctccgaccaaggtgtgctgtacatccaatctatcgcaaactttcgtctggaggaatttctgggtcaggcgtgggaactgaaattccgtatgctgaacgacctcctgccttgtgaagtgccggaggccggtgaagtggacgatccgctcctgtgtatcaaagtgaccgctttcgagtgcggcggtttcgccatcggtatgtgcctggcgcataaaattgcggatatgtgtaccatgtgtacttttatcaacaattgggcaacccgttctagccaggaaattgtgaacaagcaggagctggaaaagtacagcccgatcttttccgttgcacacgatttcccaaaagttgctctggatgacctgtctcctcgcctgccgcgctctaacatcggcatggaaaccaaagtaaaactgttccagtttaaagtggatgcgatctctaaaatgcgtgaaaacctgcgcgacaaatcctaccactcctctaaagtccagctgatcgttgcgctgttctccaaggctctcatggcgatcgataaagcaaaatttggtcactctaagccttctatcgtgcaacagggtgttaacctgcgcaacaaagttgtaccaaaactgccagagaatctgttcggcaactttttctctttcctgcacggtcgtatcgagccggaagatggtgaaaacatggatctggatggtttcctgatgatcctcaaagacagcatgaagaaatttgaagacgaatatgttaaagccctgatgtcttccgataaggattatgaagttctggttaaaccgttcctgaaatttggtgaaacttacgccaataacgatgagaacttccactgtttcacctcctggtgcaaattctctttctataaagcggatttcggctggggtaaaccggtgtggcgtagcactggccactacgctaaccacaagtttgtgatcctgatggatgacgaagagggtggcggtgtggaagcgtgggttcatctggacgaaaaatccatgtgtcagctggaacaggacccggacattaaagcctatgcaacctaa

SEQ ID NO: 92 - DNA, 1323 bp, synthetic construct
  source 1..1323: mol_type = other DNA
atggaaattaagaatttgacctctaaattggtcaaaccattgactccaaccccatcaaacttgcaaaattatgacatctcctttttcgatgaacaaatgccaaacattaacactccattgatcttatattactctacttctcaagaatctcctaacgataacattttcgatcacttggaaacttccttgtctaagaccttaactgacttttacccattggctggtcgttacatgagacaaggttcattcgttgactgttctgaccaaggtgttttgtacatcaagtctatggctaacttcagattggaagatttcttgggtcaggcttgggaattaaagtttactatgttgaacgacttattgccatgtgaggtccctgaagctggtgaagtcgatgacccattattgtgtatcaaggttactgctttcgagtgtggcggtttcgctatcggtatgtgtttctctcataagatttccgacatgtgtactatgtgcactttcattaataactgggctacccgttcttcccaagaaaatgttaacaaattggaattagaaaagtattctcctatcttctctgttgctcaagactttcctaaggtcgctttggatgacttatctccaagatttccaagatctatcattggtatggctactaatgttaaggttttccaattcaaggtcgatgctatttcaaaaatgagagaaaacttgcaaatctccaaggatgaaagaaaccaccattcctctaaaattcaattaattgttgccttgttcttaaaagccttgatggctattgataaagctaagatcggtcattcaaagtcttccattgctcaacaaggtgtcaacttgagaaacaaggttgtcccaaagctaccagaaaacttgttcggtaacttctttacattcttgcacggtcaaattgaaccagaggaaggtgaaaatatggacttagacggtttcttagtcatcttgaacgattctgtcaaaaagatcgaaggtgaatatgccaaggctttaatgtcttcccacaaggactacgaagtcttggttaagccatttctaaagttcggtgaaaccttcactaacaataacgtcaacttttactcttttacttcttggtgtaaattctctttttacaaagctgacttcggctggggtaagccagtctggagatccactggtcattacgccaacgaaaagttcgtcatcatgatggacgatgaggaaggtggcggtgttgaagcctggatccacttagacgaaaagtccatgtctcaattagaacaagatccatacattaaggcctatgctacctaa

SEQ ID NO: 93 - DNA, 1323 bp, synthetic construct
  source 1..1323: mol_type = other DNA
atggagatcaaaaacctgacttctaaactggtgaaaccgctgaccccgaccccgagcaacctgcagaactacgatatctcttttttcgacgaacagatgccgaacattaacaccccgctgatcctgtattacagcaccagccaagaatctcctaacgataacatttttgaccacctggaaacctccctgtccaaaaccctgactgacttctacccgctggctggtcgttatatgcgtcagggttccttcgttgactgctccgaccagggcgtgctctacatcaaatctatggcgaacttccgtctggaagatttcctgggtcaggcctgggagctgaaattcacgatgctgaacgatctcctgccgtgcgaggtaccggaggccggcgaagttgatgacccactcctgtgcattaaagttaccgcattcgaatgtggtggcttcgcgattggcatgtgttttagccataaaatctctgacatgtgcacgatgtgcactttcatcaataactgggctacccgttccagccaagaaaacgtgaacaaactggagctggaaaaatatagccctattttctctgtggcgcaggattttccgaaagtagctctggatgacctgtccccacgtttcccgcgttctatcattggcatggcgaccaacgtcaaggtattccagttcaaggttgacgcaatctctaagatgcgtgaaaacctgcagatctccaaagatgaacgtaaccatcactccagcaaaatccaactgatcgttgcgctgttcctgaaagcactgatggcaatcgacaaggcgaagattggtcactctaaatccagcatcgctcaacagggtgtaaacctgcgtaacaaagtcgtgccgaaactgccggagaacctgttcggtaacttctttactttcctgcatggccagatcgaaccggaggaaggcgagaacatggatctggatggcttcctggttattctgaacgattctgttaagaaaatcgagggtgaatatgccaaagcgctgatgagctcccataaagattacgaggttctggtgaaaccgttcctgaaattcggcgaaaccttcaccaacaataacgttaacttttactctttcacctcctggtgcaaattctctttctacaaagcagactttggctggggtaaaccggtgtggcgtagcacgggtcactatgcaaacgaaaaattcgtaatcatgatggatgacgaggaaggtggcggtgtagaagcatggatccacctggatgaaaaaagcatgtcccagctggaacaggacccgtacatcaaggcttatgccacctaa

SEQ ID NO: 94 - skipped

(19) United States
(12) Patent Application Publication
(10) Pub. No.: US 2026/0286408 A1
(43) Pub. Date: Sep. 24, 2026

(54) PROCESS FOR MAKING FLAVANONE DERIVATIVES

(71) Applicant: Firmenich SA, Satigny (CH)

(72) Inventors: Kerstin STEINER, Satigny (CH); Nadine FELDMANN PRETRE, Satigny (CH)

(21) Appl. No.: 19/134,614

(22) PCT Filed: Nov. 28, 2023
(86) PCT No.: PCT/EP2023/083331

§ 371 Date: May 30, 2025

(30) Foreign Application Priority Data
Nov. 30, 2022 (EP) 22210547.0
Dec. 19, 2022 (EP) 22214598.9

(51) Int. Cl. C12P 17/06; A23L 27/00; A23L 27/30; C12N 9/02; C12N 9/10

(52) U.S. Cl. CPC C12P 17/06; A23L 27/30; A23L 27/84; A23L 27/86; A23L 27/88; C12N 9/0071; C12N 9/1025; C12Y 114/11009

(57) ABSTRACT

Disclosed herein is a process for making a compound of formula (I) as defined herein by reacting a precursor compound of formula (Ia) as defined herein with an acyltransferase enzyme. In some embodiments the compound of formula (Ia) is aromadendrin, taxifolin, dihydrotamarixetin, 3′—O-methyltaxifolin, pinobanksin, 5-deoxyaromadendrin, 5-deoxytaxifolin, 5-deoxydihydrotamarixetin, 5-deoxy-3′—O-methyltaxifolin or 5-deoxypinobanksin. In some embodiments the compound of formula (I) is aromadendrin-3-O-acetate, taxifolin-3-O-acetate, dihydrotamarixetin-3-O-acetate, 3′—O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′—O-methyltaxifolin-3-O-acetate or 5-deoxypinobanksin-3-O-acetate. Also disclosed herein are acyltransferase enzymes that can be used in this process and certain compositions that include such flavanone derivatives, such as compositions that include such flavanone derivatives and one or more other sweeteners.

### CROSS-REFERENCE TO RELATED APPLICATIONS

This application is a U.S. National Phase Application of International Patent Application No. PCT/EP23/83331, filed Nov. 28, 2023, which claims priority to European Patent Application No. 22210547.0, filed Nov. 30, 2022, and European Patent Application No. 22214598.9, filed Dec. 19, 2022, each of which is hereby incorporated by reference herein in its entirety.

### REFERENCE TO AN ELECTRONIC SEQUENCE LISTING

The instant application contains a Sequence Listing as part of the International Application in XML format and is hereby incorporated by reference in its entirety. Said XML copy, created on May 4, 2026, is named “51928-037003_Sequence_Listing_5_4_26” and is 175,525 bytes in size.

### TECHNICAL FIELD

The present disclosure generally provides a process for the preparation of a class of flavanone derivatives and their use as sweetness enhancers. In some aspects, the disclosure provides certain compositions that include such flavanone derivatives, such as compositions that include such flavanone derivatives and one or more other sweeteners.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami. Sweetness is the taste most commonly perceived when eating foods rich in sugars. Mammals generally perceive sweetness to be a pleasurable sensation, except in excess. Caloric sweeteners, such as sucrose and fructose, are the prototypical examples of sweet substances. Although a variety of no-calorie and low-calorie substitutes exist, these caloric sweeteners are still the predominant means by which comestible products induce the perception of sweetness upon consumption.

Metabolic disorders and related conditions, such as obesity, diabetes, and cardiovascular disease, are major public health concerns throughout the world. And their prevalence is increasing at alarming rates in almost every developed country. Caloric sweeteners are a key contributor to this trend, as they are included in various packaged food and beverage products to make them more palatable to consumers. In many cases, no-calorie or low-calorie substitutes can be used in foods and beverages in place of sucrose or fructose. Even so, these compounds impart sweetness differently from caloric sweeteners, and a number of consumers fail to view them as suitable alternatives. Moreover, such compounds may be difficult to incorporate into certain products. In some instances, they may be used as partial replacements for caloric sweeteners, but their mere presence can cause many consumers to perceive unpleasant off-tastes including, astringency, bitterness, and metallic and licorice tastes. Thus, lower-calorie sweeteners face certain challenges to their adoption.

Sweetness enhancement provides an alternative approach to overcoming some of adoption challenges faced by lower-calorie sweeteners. Such compounds can be used in combination with sucrose or fructose to enhance their sweetness, thereby permitting the use of lower quantities of such caloric sweeteners in various food or beverage products. But, in addition to enhancing the perceived sweetness of the primary sweetener, such compounds nevertheless alter the perceived taste of the sweetener. Thus, many consumers find that it is less pleasurable to consume such sweetness-enhanced products in comparison to unenhanced alternatives having higher calories.

WO2021043842 discloses natural flavanone derivatives that are particularly useful for enhancing the sweetness of natural sugars.

### SUMMARY

The present invention claims a process for making a compound of formula (I):

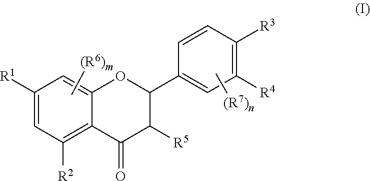

wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A}
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A}
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁵ is —O—C(O)—(C₁₋₂₄ alkyl);
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3;
the process comprising reacting a precursor compound of formula (Ia)

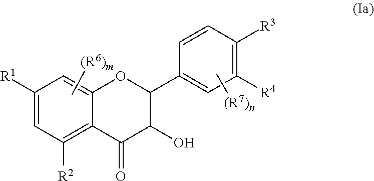

wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A}
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A}
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3;
   with an acyltransferase enzyme to form a compound of formula (I).

A preferred embodiment of the process of the invention is wherein the process is performed in the presence of acyl-CoA.

A preferred embodiment of the process of the invention is wherein the acyltransferase is an acetyltransferase enzyme.

A preferred embodiment of the process of the invention is wherein the acetyltransferase enzyme comprises the amino acid sequence HXXXD (SEQ ID NO: 29) and/or amino acid sequence [DN]FGxG (SEQ ID NO:30). A further preferred embodiment of the process of the invention is wherein the acetyltransferase enzyme comprises the amino acid sequence [ST]S[WL](SEQ ID NO: 94).

A preferred embodiment of the process of the invention is wherein the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68.

A preferred embodiment of the process of the invention is wherein the compound of formula (Ia) is aromadendrin, taxifolin, dihydrotamarixetin, 3′-O-methyltaxifolin, pinobanksin, 5-deoxyaromadendrin, 5-deoxytaxifolin, 5-deoxydihydrotamarixetin, 5-deoxy-3′-O-methyltaxifolin, or 5-deoxypinobanksin.

A preferred embodiment of the process of the invention is wherein the compound of formula (I) is aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, or 5-deoxy-3′-O-methyltaxifolin-3-O-acetate or 5-deoxypinobanksin-3-O-acetate.

A preferred embodiment of the process of the invention is wherein the process is in VIVO.

The present invention also claims a recombinant polypeptide having acyltransferase activity comprising an amino acid sequence having at least 70%, 75%, 80%, 85%, 90%, 95%, 96%, 97%, 98% or 99% sequence identity to any of SEQ ID NOs: 1 to 7, 31 to 36 and 55 to 68 or comprising the amino acid sequence of any of SEQ ID NO: 1 to 7, 31 to 36 and 55 to 68.

The present invention also claims a recombinant cell comprising a compound of formula (I).

In one embodiment, the recombinant cell further comprises an acyltransferase enzyme, preferably an acetyltransferase enzyme. More preferably, the recombinant cell further comprises a recombinant acyltransferase, even more preferably a recombinant acetyltransferase enzyme.

In another embodiment, the recombinant cell further comprises a recombinant nucleic acid sequence encoding an acyltransferase enzyme, preferably an acetyltransferase enzyme. More preferably, the recombinant cell further comprises a recombinant nucleic acid sequence encoding an acetyltransferase enzyme having at least 50%, 55%, 60%, 65%, 70%, 75%, 80%, 85%, 90%, 95%, 96%, 97%, 98% or 99% sequence identity to any of SEQ ID NOs: 8 to 28, 37 to 54 and 69 to 93 or comprising the nucleotide sequence of any of SEQ ID NOs: 8 to 28, 37 to 54 and 69 to 93 or the reverse complement thereof.

In yet a further embodiment, the recombinant cell further comprises the following enzyme(s):
(a) flavanone 3-hydroxylase, (F3H),
(b) chalcone isomerase (CHI),
(c) chalcone synthase (CHS),
(d) 4-coumarate-coenzyme A ligase (4CL),
(e) cytochrome P450 reductase (CPR),
(f) tyrosine ammonia lyase (TAL),
(g) chalcone isomerase-like (CHIL),
(h) cinnamate-4-hydroxylase (C4H),
(i) phenylalanine ammonia lyase (PAL),
(j) flavonoid 3-hydroxylase (F3′H),
(k) 3′-O-methyltransferase (3′-MT),
(l) 4′-O-methyltransferase (4′-MT),
(m) 3-O-methyltransferase (3-MT),
(n) 4-O-methyltransferase (4-MT),
(o) 3-OH specific P450 monooxygenase,
(p) glycosidase, and/or
(q) polyketide reductase (PKR)

A cell may be a prokaryotic, archaebacterial or eukaryotic cell.

A prokaryotic cell may be, but is not limited to, a bacterial cell.

A eukaryotic cell may be, but is not limited to, a fungus (e.g. a yeast or a filamentous fungus), an algae, a plant cell, a cell line.

Preferably the cell is a bacterial, archaebacterial, fungal such as yeast, algal or plant cell.

The present invention also claims a growth medium comprising the recombinant cell of the invention and a compound of formula (I).

The present invention also claims a process for making a compound of formula (I) comprising growing a recombinant cell of the invention under growth conditions suitable for the production of the compound of formula (I).

The present invention also claims a compound of formula (I) obtained or obtainable by the process of any of the previous claims.

The present invention also claims the use of a compound of formula (I) obtained or obtainable by the process of any of the previous claims to (a) enhance a sweet taste, (b) reduce a bitter taste, or (c) reduce a sour taste, of an ingestible composition.

The present invention also claims a method of a) enhancing a sweet taste, (b) reducing a bitter taste, or (c) reducing a sour taste, of an ingestible composition of a product, the method comprising introducing to the product a compound of formula (I) obtained or obtainable by the process of any of the previous claims.

### BRIEF DESCRIPTION OF THE DRAWINGS

FIG. 1. A schematic of flavanone and flavanone derivative biosynthesis.

FIG. 2. Example of negative and positive ESI-MS/MS spectra of aromadendrin-3-O-acetate. Panel A shows negative ESI-MS/MS spectrum of aromadendrin-3-O-acetate. Panel B shows positive ESI-MS/MS spectrum of aromadendrin-3-O-acetate.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### ABBREVIATIONS USED

bp—base pair
kb—kilo base
DNA—deoxyribonucleic acid
cDNA—complementary DNA
DTT—dithiothreitol
GC—gas chromatograph
HPLC—high-performance liquid chromatography
IPTG—isopropyl-D-thiogalactopyranoside
LB—lysogeny broth
MS—mass spectrometer/mass spectrometry
PCR—polymerase chain reaction
RNA—ribonucleic acid
mRNA—messenger ribonucleic acid
miRNA—micro RNA
siRNA—small interfering RNA
rRNA—ribosomal RNA
tRNA—transfer RNA
SMM—supplemented minimal medium
BLAST—basic local alignment search tool
ESI—electrospray ionization
OD—optical density
SDS-PAGE—sodium dodecyl sulfate polyacrylamide gel electrophoresis

### Definitions

As used herein, “solvate” means a compound formed by the interaction of one or more solvent molecules and one or more compounds described herein. In some embodiments, the solvates are ingestibly acceptable solvates, such as hydrates.

As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “halogen” or “halo” means any one of the radio-stable atoms of column 7 of the Periodic Table of the Elements, such as fluorine, chlorine, bromine, or iodine. In some embodiments, “halogen” or “halo” refer to fluorine or chlorine.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted.

As used herein, “substituted alkyl” means an alkyl group substituted with one or more substituents independently selected from C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (=O).

As used herein, “alkoxy” means a moiety of the formula —OR wherein R is an alkyl, as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” means a moiety of the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” means a straight or branched hydrocarbon chain containing one or more double bonds. In some embodiments, the alkenyl group has from 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like. Unless indicated to the contrary, the term “alkenyl” refers to a group that is not further substituted.

As used herein, “alkynyl” means a straight or branched hydrocarbon chain containing one or more triple bonds. In some embodiments, the alkynyl group has from 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. Unless indicated to the contrary, the term “alkynyl” refers to a group that is not further substituted.

As used herein, “heteroalkyl” means a straight or branched hydrocarbon chain containing one or more heteroatoms, that is, an element other than carbon, including but not limited to, nitrogen, oxygen, and sulfur, in the chain backbone. In some embodiments, the heteroalkyl group has from 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain. Unless indicated to the contrary, the term “heteroalkyl” refers to a group that is not further substituted.

As used herein, “alkylene” means a branched or straight chain fully saturated di-radical chemical group containing only carbon and hydrogen that is attached to the rest of the molecule via two points of attachment (i.e., an alkanediyl). In some embodiments, the alkylene group has from 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene. Unless indicated to the contrary, the term “alkylene” refers to a group that is not further substituted.

As used herein, “alkenylene” means a straight or branched chain di-radical chemical group containing only carbon and hydrogen and containing at least one carbon-carbon double bond that is attached to the rest of the molecule via two points of attachment. In some embodiments, the alkenylene group has from 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1, 1-diyl, propenylene, propen-1, 1-diyl, prop-2-en-1, 1-diyl, 1-methyl-ethenylene, but-1-enylene, but-2-enylene, but-1,3-dienylene, buten-1, 1-diyl, but-1,3-dien-1,1-diyl, but-2-en-1,1-diyl, but-3-en-1,1-diyl, 1-methyl-prop-2-en-1,1-diyl, 2-methyl-prop-2-en-1, 1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl. Unless indicated to the contrary, the term “alkenylene” refers to a group that is not further substituted.

As used herein, “aromatic” means a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” means an aromatic ring or ring system (i.e., two or more fused rings that share two adjacent carbon atoms) containing only carbon in the ring backbone. When the aryl is a ring system, every ring in the system is aromatic. In some embodiments, the aryl group has from 6 to 18 carbon atoms, although the present definition also covers the occurrence of the term “aryl” where no numerical range is designated. In some embodiments, the aryl group has from 6 to 10 carbon atoms. The aryl group may be designated as “C₆₋₁₀ aryl,” “C₆-C₁₀ aryl,” or similar designations. Examples of aryl groups include, but are not limited to, phenyl, naphthyl, azulenyl, and anthracenyl. In some embodiments, the term “aryl” refers to phenyl. Unless indicated to the contrary, the term “aryl” refers to a group that is not further substituted.

As used herein, “aryloxy” and “arylthio” mean moieties of the formulas RO— and RS—, respectively, in which R is an aryl as is defined above, such as “C₆₋₁₀ aryloxy” or “C₆₋₁₀ arylthio” and the like, including but not limited to phenyloxy and phenylthio. As used herein “aralkyl” or “arylalkyl” means an aryl group connected, as a substituent, via an alkylene group, such as “C₇₋₁₄ aralkyl” and the like, including, but not limited to, benzyl, 2-phenylethyl, 3-phenylpropyl, and the like. In some embodiments, the alkylene group is a lower alkylene group (i.e., a C₁₋₄ alkylene group).

As used herein, “heteroaryl” means an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. In some embodiments, the heteroaryl group has from 5 to 18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has from 5 to 10 ring members or from 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl. Unless indicated to the contrary, the term “heteroaryl” refers to a group that is not further substituted.

As used herein, “heteroaralkyl” or “heteroarylalkyl” means heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. In some embodiments, the carbocyclyl group has from 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. Unless indicated to the contrary, the term “carbocyclyl” refers to a group that is not further substituted.

As used herein, “(carbocyclyl)alkyl” means a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system, according to any of the embodiments set forth above for carbocyclyl. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic, and according to any of the embodiments set forth above for carbocyclyl. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. In some embodiments, the heterocyclyl group has from 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “(heterocyclyl)alkyl” means a heterocyclyl group connected, as a substituent, via an alkylene group. Examples include, but are not limited to, imidazolinylmethyl and indolinylethyl.

An “acyl” group refers to a —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “glucosyl moiety” is a monovalent moiety in which one of the hydroxyl groups of glucose is replaced by a bond to another atom, functional group, or moiety. Unless otherwise specified, the glucose can have any suitable stereochemistry. Thus, the term includes moieties having D stereochemistry, as well as moieties having L stereochemistry. Further, the term includes moieties having a stereochemistry, as well as moieties having p stereochemistry. The carbon atoms of the glucosyl moiety follow the conventional numbering, as shown below. The diagram is shown for β-D glucose, but applies in an analogous way to glucosyl moieties having a and/or L stereochemistry:

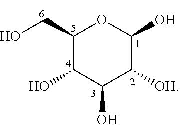 [SMILES: OC[C@H]1O[C@@H](O)[C@H](O)[C@@H](O)[C@@H]1O]

As used herein, a “glucuronyl moiety” is a monovalent moiety in which one of the hydroxyl groups of glucuronic acid is replaced by a bond to another atom, functional group, or moiety. Unless otherwise specified, the glucuronic acid can have any suitable stereochemistry. Thus, the term includes moieties having D stereochemistry, as well as moieties having L stereochemistry. Further, the term includes moieties having a stereochemistry, as well as moieties having p stereochemistry. The carbon atoms of the glucuronyl moiety follow the conventional numbering, as shown below. The diagram is shown for β-D glucuronic acid, but applies in an analogous way to glucuronyl moieties having a and/or L stereochemistry:

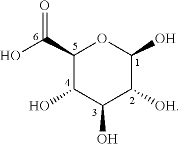 [SMILES: O=C(O)[C@H]1O[C@@H](O)[C@H](O)[C@@H](O)[C@@H]1O]

The term “C₁₋₆ alkyl glucuronyl ester moiety” refers to a glucuronyl moiety (as defined in this paragraph) in which the carboxylic acid group of glucuronic acid has a C₁₋₆ alkyl group in place of the hydrogen atom of the carboxylic acid group. In any of the embodiments below, the C₁₋₆alkyl moiety can have any suitable value, such as methyl, ethyl, isopropyl, propyl, butyl, pentyl, and the like. In some embodiments, the C₁₋₆alkyl moiety is methyl. In some other embodiments, the C₁₋₆ alkyl moiety is ethyl. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (=O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

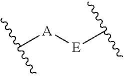

includes the substituent being oriented such that the A is attached at the leftmost attachment point of the molecule as well as the case in which A is attached at the rightmost attachment point of the molecule.

A “sweetener”, “sweet flavoring agent”, “sweet flavor entity”, or “sweet compound” herein refers to a compound or ingestibly acceptable salt thereof that elicits a detectable sweet flavor in a subject,

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the term “flavor-modifying compound(s)” refers to compounds of formula (I) or formula (Ia) or salts thereof, or any embodiments thereof set forth herein.

As used herein, certain substituents or linking groups having only a single atom may be referred to by the name of the atom. For example, in some cases, the substituent “—H” may be referred to as “hydrogen” or “a hydrogen atom,” the substituent “—F” may be referred to as “fluorine” or “a fluorine atom,” and the linking group “—O—” may be referred to as “oxygen” or “an oxygen atom.”

Points of attachment for groups are generally indicated by a terminal dash (-) or by an asterisk (*). For example, a group such as *—CH₂—CH₃ or —CH₂—CH₃ both represent an ethyl group.

Chemical structures are often shown using the “skeletal” format, such that carbon atoms are not explicitly shown, and hydrogen atoms attached to carbon atoms are omitted entirely. For example, the structure

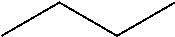

represents butane (i.e., n-butane). Furthermore, aromatic groups, such as benzene, are represented by showing one of the contributing resonance structures. For example, the structure

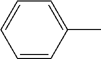

represents toluene.

Positions on the flavanone ring may be referred to by a number, such as the “2-position,” the “3-position,” and the like. These terms refer to specific positions on the fused ring structure of flavanone, even if further substitution of the fused ring structure may otherwise cause the positions to be numbered differently. Thus, for example, the carbonyl-substituted position is referred to as occurring at the “4-position” regardless of whether, in some embodiments, the addition of further substituents would cause it to occur at something besides the 4-position on the fused ring structure. Likewise, the position of the substituted phenyl substituent on the fused ring structure is referred to as occurring at the “2-position” regardless of whether, in some embodiments, the addition of further substituents would cause it to occur at something besides the 2-position on the fused ring structure.

The term “dihydroflavonol” or “flavanonol”, refers to a flavan-4-one comprising an OH group at position 3.

The term “polypeptide” means an amino acid sequence of consecutively polymerized amino acid residues, for instance, at least 15 residues, at least 30 residues, at least 50 residues. In some embodiments herein, a polypeptide comprises an amino acid sequence that is an enzyme, or a fragment, or a variant thereof.

The term “protein” refers to an amino acid sequence of any length wherein amino acids are linked by covalent peptide bonds, and includes oligopeptide, peptide, polypeptide and full-length protein whether naturally occurring or synthetic.

The term “isolated” polypeptide refers to an amino acid sequence that is removed from its natural environment by any method or combination of methods known in the art and includes recombinant, biochemical and synthetic methods.

The terms “biological function,” “function,” “biological activity” or “activity” refer to the ability of the acyltransferase to catalyze the formation of a compound of formula (I).

The terms “nucleic acid sequence,” “nucleic acid,” “nucleic acid molecule” and “polynucleotide” are used interchangeably meaning a sequence of nucleotides. A nucleic acid sequence may be a single-stranded or double-stranded deoxyribonucleotide, or ribonucleotide of any length, and include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference of thymine (T) being replaced by uracil (U). The term “nucleotide sequence” should also be understood as comprising a polynucleotide molecule or an oligonucleotide molecule in the form of a separate fragment or as a component of a larger nucleic acid.

An “isolated nucleic acid” or “isolated nucleic acid sequence” relates to a nucleic acid or nucleic acid sequence that is in an environment different from that in which the nucleic acid or nucleic acid sequence naturally occurs and can include those that are substantially free from contaminating endogenous material. The term “naturally-occurring” as used herein as applied to a nucleic acid refers to a nucleic acid that is found in a cell of an organism in nature and which has not been intentionally modified by a human in the laboratory.

“Recombinant nucleic acid sequences” are nucleic acid sequences that result from the use of laboratory methods (for example, molecular cloning) to bring together genetic material from more than one source, creating or modifying a nucleic acid sequence that does not occur naturally and would not be otherwise found in biological organisms.

“Recombinant DNA technology” refers to molecular biology procedures to prepare a recombinant nucleic acid sequence as described, for instance, in Laboratory Manuals edited by Weigel and Glazebrook, 2002, Cold Spring Harbor Lab Press; and Sambrook et al., 1989, Cold Spring Harbor, NY, Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, exons, and/or a 3′non-translated sequence comprising, e.g., transcription termination sites.

A “chimeric gene” refers to any gene which is not normally found in nature in a species, in particular, a gene in which one or more parts of the nucleic acid sequence are present that are not associated with each other in nature. For example, the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription). The term “chimeric gene” also includes genes obtained through the combination of portions of one or more coding sequences to produce a new gene.

A “3′ UTR” or “3′ non-translated sequence” (also referred to as “3′ untranslated region,” or “3′end”) refers to the nucleic acid sequence found downstream of the coding sequence of a gene, which comprises, for example, a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm. “Expression of a gene” encompasses “heterologous expression” and “over-expression” and involves transcription of the gene and translation of the mRNA into a protein. Overexpression refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

An “expression vector” as used herein includes any linear or circular recombinant vector including but not limited to viral vectors, bacteriophages and plasmids. The skilled person is capable of selecting a suitable vector according to the expression system. In one embodiment, the expression vector includes the nucleic acid of an embodiment herein operably linked to at least one regulatory sequence, which controls transcription, translation, initiation and termination, such as a transcriptional promoter, operator or enhancer, or an mRNA ribosomal binding site and, optionally, including at least one selection marker. Nucleotide sequences are “operably linked” when the regulatory sequence functionally relates to the nucleic acid of an embodiment herein.

“Regulatory sequence” refers to a nucleic acid sequence that determines expression level of the nucleic acid sequences of an embodiment herein and is capable of regulating the rate of transcription of the nucleic acid sequence operably linked to the regulatory sequence. Regulatory sequences comprise promoters, enhancers, transcription factors, promoter elements and the like.

“Promoter” refers to a nucleic acid sequence that controls the expression of a coding sequence by providing a binding site for RNA polymerase and other factors required for proper transcription including without limitation transcription factor binding sites, repressor and activator protein binding sites. The meaning of the term promoter also includes the term “promoter regulatory sequence”. Promoter regulatory sequences may include upstream and downstream elements that may influences transcription, RNA processing or stability of the associated coding nucleic acid sequence. Promoters include naturally-derived and synthetic sequences. The coding nucleic acid sequences is usually located downstream of the promoter with respect to the direction of the transcription starting at the transcription initiation site.

The term “constitutive promoter” refers to an unregulated promoter that allows for continual transcription of the nucleic acid sequence it is operably linked to.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous. The nucleotide sequence associated with the promoter sequence may be of homologous or heterologous origin with respect to the plant to be transformed. The sequence also may be entirely or partially synthetic. Regardless of the origin, the nucleic acid sequence associated with the promoter sequence will be expressed or silenced in accordance with promoter properties to which it is linked after binding to the polypeptide of an embodiment herein. The associated nucleic acid may code for a protein that is desired to be expressed or suppressed throughout the organism at all times or, alternatively, at a specific time or in specific tissues, cells, or cell compartment. Such nucleotide sequences particularly encode proteins conferring desirable phenotypic traits to the host cells or organism altered or transformed therewith. More particularly, the associated nucleotide sequence leads to the production of a compound of formula (I) or a mixture comprising a compound of formula (I) and one or more other compounds in the cell or organism. Particularly, the nucleotide sequence encodes a polypeptide having acyltransferase activity. “Target peptide” refers to an amino acid sequence which targets a protein, or polypeptide to intracellular organelles, i.e., mitochondria, or plastids, or to the extracellular space (secretion signal peptide). A nucleic acid sequence encoding a target peptide may be fused to the nucleic acid sequence encoding the amino terminal end, e.g., N-terminal end, of the protein or polypeptide, or may be used to replace a native targeting polypeptide.

The term “primer” refers to a short nucleic acid sequence that is hybridized to a template nucleic acid sequence and is used for polymerization of a nucleic acid sequence complementary to the template.

As used herein, the term “host cell” or “transformed cell” or “recombinant cell” refers to a cell (or organism) altered to harbor at least one nucleic acid molecule, for instance, a recombinant gene encoding a desired protein or nucleic acid sequence which upon transcription yields an acyltransferase protein useful to produce a compound of formula (I) or a mixture comprising a compound of formula (I) and one or more other compounds. The host cell may contain a recombinant gene which has been integrated into the nuclear or organelle genomes of the host cell. Alternatively, the host may contain the recombinant gene extra-chromosomally.

The host cell may be a prokaryotic, archaebacterial or eukaryotic cell.

A prokaryotic cell may be, but is not limited to, a bacterial cell. Bacterial cell may be Gram-negative or Gram-positive bacteria. Examples of bacteria include, but are not limited to, bacteria belonging to the genus Bacillus (e.g., B. subtilis, B. amyloliquefaciens, B. licheniformis, B. puntis, B. megaterium, B. halodurans, B. pumilus), Acinetobacter, Nocardia, Xanthobacter, Escherichia (e.g., E. coli), Streptomyces, Erwinia, Klebsiella, Serratia (e.g., S. marcessans), Pseudomonas (e.g., P. aeruginosa, P. fluorescens), Salmonella (e.g., S. typhimurium, S. typhi), Anabaena, Caulobactert, Gluconobacter, Rhodobacter, Paracoccus, Brevibacterium, Corynebacterium, Rhizobium (Sinorhizobium), Flavobacterium, Klebsiella, Enterobacter, Lactobacillus, Lactococcus, Methylobacterium, Staphylococcus. Bacteria also include, but are not limited to, photosynthetic bacteria (e.g., green non-sulfur bacteria green sulfur bacteria purple sulfur bacteria and purple non-sulfur bacteria.

A eukaryotic cell may be, but is not limited to, fungus (e.g. a yeast or a filamentous fungus), an algae, a plant cell, a cell line.

A eukaryotic cell may be a fungus, such as a filamentous fungus or yeast. Filamentous fungal strains include, but are not limited to, strains of Acremonium, Aspergillus (e.g. A. niger, A oryzae, A. nidulans), Agaricus, Aureobasidium, Coprinus, Cryptococcus, Corynascus, Chrysosporium, Filibasidium, Fusarium, Humicola, Magnaporthe, Monascus, Mucor, Myceliophthora, Mortierella, Neocallimastix, Neurospora, Paecilomyces, Penicillium (e.g. P. chrysogenum, P. camemberti), Piromyces, Phanerochaete Pleurotus, Podospora, Pycnoporus, Rhizopus, Schizophyllum, Sordaria, Talaromyces, Rasamsonia (e.g. Rasamsonia emersonii), Thermoascus, Thielavia, Tolypocladium, Trametes and Trichoderma.

Yeast cells may be selected from the genera: Saccharomyces (e.g., S. cerevisiae, S. bayanus, S. pastorianus, S. carlsbergensis), Kluyveromyces, Candida (e.g., C. rugosa, C. revkaufi, C. pulcherrima, C. tropicalis, C. utilis, C. krusei), Pichia (e.g., P. pastoris), Schizosaccharomyces, Issatchenkia (e.g. I. orientalis), Zygosaccharomyces, Hansenula, Kloeckera, Schwanniomyces, and Yarrowia (e.g., Y. lipolytica, formerly classified as Candida lipolytica).

The cell may be an algae, a microalgae or a marine eukaryote. The cell may be a Labyrinthulomycetes cell, preferably of the order Thraustochytriales, more preferably of the family Thraustochytriaceae, more preferably a member of a genus selected from the group consisting of Aurantiochytrium, Oblongichytrium, Schizochytrium, Thraustochytrium, and Ulkenia, even more preferably Schizochytrium sp. ATCC #20888.

Homologous sequences include orthologous or paralogous sequences. Methods of identifying orthologs or paralogs including phylogenetic methods, sequence similarity and hybridization methods are known in the art and are described herein.

Paralogs result from gene duplication that gives rise to two or more genes with similar sequences and similar functions. Paralogs typically cluster together and are formed by duplications of genes within related plant species. Paralogs are found in groups of similar genes using pair-wise Blast analysis or during phylogenetic analysis of gene families using programs such as CLUSTAL. In paralogs, consensus sequences can be identified characteristic to sequences within related genes and having similar functions of the genes.

Orthologs, or orthologous sequences, are sequences similar to each other because they are found in species that descended from a common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in host cells or organisms, such as plants, overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making the host cells, organism such as plants producing a compound of formula (I).

The term “selectable marker” refers to any gene which upon expression may be used to select a cell or cells that include the selectable marker. Examples of selectable markers are described below. The skilled artisan will know that different antibiotic, fungicide, auxotrophic or herbicide selectable markers are applicable to different target species.

The term “organism” refers to any non-human multicellular or unicellular organisms such as a plant, or a microorganism. Particularly, a microorganism is a bacterium, a archaebacterium, an algae or a fungus such as a yeast.

The term “plant” is used interchangeably to include plant cells including plant protoplasts, plant tissues, plant cell tissue cultures giving rise to regenerated plants, or parts of plants, or plant organs such as roots, stems, leaves, flowers, pollen, ovules, embryos, fruits and the like. Any plant can be used to carry out the methods of an embodiment herein.

As used herein, the term “phenylalanine ammonia lyase” and “PAL” refer to an encoding nucleic acid and phenylalanine ammonia lyase enzyme. Phenylalanine ammonia lyase (EC 4.3.1.24) catalyzes the conversion of L-phenylalanine to trans-cinnamic acid. An example of PAL sequence is provided in GenBank Accession No. AY303128. This term also includes enzymes of the class EC 4.3.1.25, which are bifunctional phenylalanine/tyrosine ammonia-lyases.

As used herein, the term “cinnamate-4-hydroxylase” and “C4H” refer to an encoding nucleic acid and cinnamate-4-hydroxylase enzyme. Cinnamate-4-hydroxylase (EC 1.14.14.91) catalyzes the conversion of trans-cinnamic acid to p-coumaric acid. C4H is a P450 monooxygenase that benefits from regeneration by a cytochrome P450 reductase. An example of C4H sequence is provided in GenBank Accession No. U71080.

As used herein, the term “cytochrome P450 reductase” and “CPR” refer to an encoding nucleic acid and cytochrome P450 reductase enzyme. Cytochrome P450 reductase (EC 1.6.2.4) is an enzyme required for electron transfer from NAD(P)H to cytochrome P450 monooxygenases including C4H and F3′H. An example of CPR sequence is provided in GenBank Accession No. X66017 and NM_119167.

As used herein, the term “tyrosine ammonia lyase” and “TAL” refer to an encoding nucleic acid and tyrosine ammonia lyase enzyme. Tyrosine ammonia lyase (EC 4.3.1.23) catalyzes the conversion of L-tyrosine into p-coumaric acid. An example of TAL sequence is provided in GenBank Accession No Q31WB0. This term also includes enzymes of the class EC 4.3.1.25, which are bifunctional phenylalanine/tyrosine ammonia-lyases.

As used herein, the term “4-coumarate-CoA ligase” and “4CL” refer to an encoding nucleic acid and 4-coumarate-CoA ligase enzyme. 4-coumarate-CoA ligase (EC 6.2.1.12) catalyzes the conversion of p-coumaric acid into p-coumaroyl-CoA. An example of 4CL sequence is provided in GenBank Accession No U18675.

As used herein, the term “chalcone synthase” and “CHS” refer to an encoding nucleic acid and chalcone synthase enzyme. Chalcone synthase (EC 2.3.1.74) catalyzes the condensation of p-coumaroyl-CoA with three molecules of malonyl-CoA to naringenin chalcone. An example of CHS sequence is provided in GenBank Accession No AF 112086.

As used herein, the term “chalcone isomerase” and “CHI” refer to an encoding nucleic acid and chalcone isomerase enzyme. Chalcone isomerase (EC 5.5.1.6) catalyzes the conversion of naringenin chalcone into naringenin. An example of CHI sequence is provided in GenBank Accession No M86358.

As used herein, the term “chalcone isomerase-like protein” and “CHIL” refer to an encoding nucleic acid and chalcone isomerase-like protein. Chalcone isomerase-like protein increases the activity of CHS. An example of CHIL sequence is provided in GenBank Accession No NP_850770.

As used herein, the term “flavanone-3-hydroxylase” and “F3H” refer to an encoding nucleic acid and flavanone-3-hydroxylase enzyme. Flavanone-3-hydroxylase (EC 1.14.11.9) catalyzes the conversion of naringenin into aromadendrin, eriodictyol into taxifolin, hesperetin into dihydrotamarixetin, homoeriodictyol into 3′-O-methyltaxifolin, pincembrin into pinobanksin, liquiritigenin into 5-deoxyaromadendin, 5-deoxyeriodictyol into 5-deoxytaxifolin, 5-deoxyhesperetin into 5-deoxydihydrotamarixetin, 5-deoxyhomoeriodictyol into 5-deoxy-3′-O-methyl-taxifolin or 5-deoxypinocembrin into 5-deoxypinobanksin. An example of F3H sequence is provided in GenBank Accession No U33932.

As used herein, the term “flavonoid 3-hydroxylase” and “F3′H” refer to an encoding nucleic acid and flavonoid 3-hydroxylase enzyme. Flavonoid 3-hydroxylase (EC 1.14.14.82) catalyzes the addition of an OH group to the 3′-position of flavanones such as naringenin or a dihydroflavonol, such as aromadendrin. F3′H is a P450 monooxygenase that benefits from regeneration by a cytochrome P450 reductase. An example of F3′H sequence is provided in GenBank Accession No AH009204.

As used herein, the term “3′-O-methyltransferase” and “3′-MT” refer to an encoding nucleic acid and 3′-O-methyltransferase enzyme. 3′-O-methyltransferase (EC 2.1.1) catalyzes the transfer of a methyl group to 3′-OH of flavanones, such as eriodictyol, or dihydroflavonols, such as taxifolin. An example of 3′-MT sequence is provided in GenBank accession No NP_200227.

As used herein, the term “4′-O-methyltransferase” and “4′-MT” refer to an encoding nucleic acid and 4′-O-methyltransferase enzyme. 4′-O-methyltransferase (EC 2.1.1) catalyzes the transfer of a methyl group to 4′-OH of flavanones, such as naringenin or eriodictyol, or dihydroflavonols, such as aromadendrin or taxifolin. An example of 4′-MT sequence is provided in GenBank accession No C6TAY1.

As used herein, the term “O-methyltransferase” and “OMT” refer to an encoding nucleic acid and O-methyltransferase enzyme. O-methyltransferase (EC 2.1.1) catalyzes the transfer of a methyl group to an OH of an acceptor molecule, such as tyrosine, (hydroxyphenyl)-2-propenoic acid such as coumaric or caffeic acid, flavanones, such as eriodictyol, or dihydroflavonols, such as taxifolin.

As used herein, the term “glycosyltransferase” and “GT” refer to an encoding nucleic acid and glycosyltransferase enzyme. Glycosyltransferase catalyzes the transfer of saccharide moieties from an activated nucleotide sugar to a nucleophilic glycosyl acceptor molecule, in this case dihydroflavonol-3-O-acetate such as aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, or 5-deoxy-3′-O-methyltaxifolin-3-O-acetate or 5-deoxypinobanksin-3-O-acetate.

As used herein, the term glycosidase (glycoside hydrolase) refers to an encoding nucleic acid and glycosidase enzyme. Glycosidases (EC 3.2.1) catalyze the hydrolysis of glycosidic bonds in this case a glycosylated flavanone precursors, such as naringin or hesperidin to the corresponding aglycons such as naringenin and hesperetin.

As used herein, the term«polyketide reductase»and«PKR»refer to an encoding nucleic acid and polyketide reductase enzyme. Polyketide reductase (EC 2.3.1.170) coupled with a CHS catalyzes the reduction of a specific keto group of the tetraketide intermediate resulting in 6′-deoxychalcones. In some cases in the literature, the term chalcone reductase or CHR is also used. It is however discouraged as this term is misleading (Schröder, in Comprehensive Natural Product Chemistry, 1999, chapter 1.27.6.1). An example of PKR sequence is provided in GenBank Accession No. AB263016.

Other terms are defined in other portions of this description, even though not included in this subsection.

### Product and Precursor Compounds

The present invention concerns a process for making a compound of formula (I).

Compounds of formula (I):

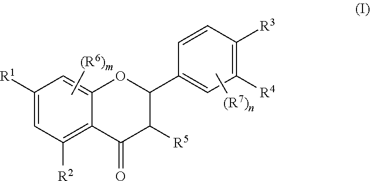

wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A}
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A};
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁵ is —O—C(O)—(C₁₋₂₄ alkyl);
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3.

The process of the invention comprises reacting a precursor compound of formula (Ia).

Compounds of formula (Ia):

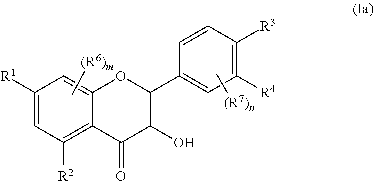

wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A}
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A}
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3.

R¹ can have any suitable value according to the parameters set forth above. In some embodiments, R¹ is H, —OH or —OCH₃. In some further embodiments, R¹ is —OH.

R² can have any suitable value according to the parameters set forth above. In some embodiments, R² is H, —OH or —OCH₃. In some further embodiments, R² is —OH. In some embodiments, R¹ and R² are —OH.

R³ can have any suitable value according to the parameters set forth above. In some embodiments, R³ is H, —OH or —OCH₃. In some further embodiments, R³ is H. In some further embodiments, R³ is —OH. In some other embodiments, R³ is —OCH₃.

R⁴ can have any suitable value according to the parameters set forth above. In some embodiments, R⁴ is H, —OH or —OCH₃. In some further embodiments, R⁴ is H. In some further embodiments, R⁴ is —OH. In some other embodiments, R⁴ is —OCH₃. In some embodiments, R³ and R⁴ are hydrogen atoms. In some embodiments, R³ is —OH and R⁴ is a hydrogen atom. In some embodiments, R³ and R⁴ are —OH. In some embodiments, R³ is —OH and R⁴ is —OCH₃. In some embodiments, R³ is —OCH₃ and R⁴ is —OH. In some embodiments, at least one of R³ and R⁴ is —OH.

R⁵ can have any suitable value according to the parameters set forth above. In some embodiments, R⁵ is —O—C(O)—(C₁₋₂₂ alkyl). In some embodiments, R⁵ is —O—C(O)—(C₁₋₁₈ alkyl). In some embodiments, R⁵ is —O—C(O)—(C₁₋₁₂ alkyl). In some embodiments, R⁵ is —O—C(O)—(C₁₋₈ alkyl). In some embodiments, R⁵ is —O—C(O)—(C₁₋₆ alkyl). In some embodiments, R⁵ is —O—C(O)—CH₃. In some embodiments, R⁵ is —O—C(O)—CH₂—CH₃. In some embodiments, R⁵ is —O—C(O)—CH(CH₃)₂. In some embodiments, R⁵ is —O—C(O)—(CH₂)₂—CH₃. In some embodiments, R⁵ is —O—C(O)—(CH₂)₃—CH₃. In some embodiments, R⁵ is —O—C(O)—(CH₂)₄—CH₃.

In some embodiments, R⁵ is —O—C(O)—(CH₂)₅—CH₃.

R⁶ and R⁷ can have any suitable value according to the parameters set forth above, and can be present any number of times according to the variables m and n. In some embodiments, R⁶ and R⁷ are independently —OH or —OCH₃. In some embodiments, m+n is 0, 1, or 2. In some embodiments, m+n is 0 or 1. In some embodiments, m is 0 and n is 0 or 1. In some embodiments, m and n are both 0.

A preferred embodiment of the invention is wherein the compound of formula (Ia) is aromadendrin, taxifolin, dihydrotamarixetin, 3′-O-methyltaxifolin, pinobanksin, 5-deoxyaromadendrin, 5-deoxytaxifolin, 5-deoxydihydrotamarixetin, 5-deoxy-3′-O-methyltaxifolin, or 5-deoxypinobanksin.

“Aromadendrin”, also known as dihydrokaempferol, is a compound known in the art. The preferred IUPAC name is (2R,3R)-3,5,7-trihydroxy-2-(4-hydroxyphenyl)-2,3-dihydrochromen-4-one.

“Taxifolin” is a compound known in the art. The preferred IUPAC name is (2R,3R)-3,5,7-trihydroxy-2-(3,4-dihydroxyphenyl)-2,3-dihydrochromen-4-one.

“Dihydrotamarixetin” is a compound known in the art. The IUPAC name is (2R,3R)-3,5,7-trihydroxy-2-(3-hydroxy-4-methoxyphenyl)-2,3-dihydrochromen-4-one.

“3′-O-methyltaxifolin” is a compound known in the art. The IUPAC name is (2R,3R)-3,5,7-trihydroxy-2-(4-hydroxy-3-methoxyphenyl)-2,3-dihydrochromen-4-one.

“Pinobanksin” is a compound known in the art. The IUPAC name is (2R,3R)-3,5,7-trihydroxy-2-phenyl-2,3-dihydrochromen-4-one.

“5-deoxyaromadendrin” is known in the art. The preferred IUPAC name is (2R,3R)-3,7-dihydroxy-2-(4-hydroxyphenyl)-2,3-dihydrochromen-4-one.

“5-deoxytaxifolin” is known in the art. The preferred IUPAC name is (2R,3R)-3,7-dihydroxy-2-(3,4-dihydroxyphenyl)-2,3-dihydrochromen-4-one.

“5-deoxydihydrotamarixetin” is known in the art. The preferred IUPAC name is (2R,3R)-3,7-dihydroxy-2-(3-hydroxy-4-methoxyphenyl)-2,3-dihydrochromen-4-one.

“5-deoxy-3′-O-methyltaxifolin” is known in the art. The preferred IUPAC name is (2R,3R)-3,7-dihydroxy-2-(4-hydroxy-3-methoxyphenyl)-2,3-dihydrochromen-4-one.

“5-deoxypinobanksin” is known in the art. The preferred IUPAC name is (2R,3R)-3,7-dihydroxy-2-phenyl-2,3-dihydrochromen-4-one.

A preferred embodiment of the invention is wherein the compound of formula (I) is aromadendrin-3-O-acetate, taxifolin-3-O-acetate, dihydrotamarixetin-3-O-actetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate.

“Aromadendrin-3-O-acetate” is a compound known in the art. The preferred IUPAC name is [(2R,3R)-5,7-dihydroxy-2-(4-hydroxyphenyl)-4-oxo-2,3-dihydrochromen-3-yl]acetate.

“Taxifolin-3-O-acetate” is a compound known in the art. The preferred IUPAC name is [(2R,3R)-5,7-dihydroxy-2-(3,4-dihydroxyphenyl)-4-oxo-2,3-dihydrochromen-3-yl]acetate.

“Dihydrotamarixetin-3-O-acetate” is a compound known in the art. The IUPAC name is [(2R,3R)-5,7-dihydroxy-2-(3-hydroxy-4-methoxyphenyl)-4-oxo-2,3-dihydrochromen-3-yl]acetate.

“3′-O-methyltaxifolin-3-O-acetate” is a compound known in the art. The IUPAC name is [(2R,3R)-5,7-dihydroxy-2-(4-hydroxy-3-methoxyphenyl)-4-oxo-2,3-dihydrochromen-3-yl]acetate.

“Pinobanksin-3-O-acetate” is a compound known in the art. The IUPAC name is [(2R,3R)-5,7-dihydroxy-2-phenyl-4-oxo-2,3-dihydrochromen-3-yl]acetate.

“5-deoxyaromadendrin-3-O-acetate” has the preferred IUPAC name [(2R,3R)-7-hydroxy-2-(4-hydroxyphenyl)-4-oxo-2,3-dihydrochromen-3-yl]acetate.

“5-deoxytaxifolin-3-O-acetate” has the preferred IUPAC name [(2R,3R)-7-hydroxy-2-(3,4-dihydroxyphenyl)-4-oxo-2,3-dihydrochromen-3-yl]acetate.

“5-deoxydihydrotamarixetin-3-O-acetate” has the preferred IUPAC name [(2R,3R)-7-hydroxy-2-(3-hydroxy-4-methoxyphenyl)-4-oxo-2,3-dihydrochromen-3-yl]acetate.

“5-deoxy-3′-O-methyltaxifolin-3-O-acetate” has the preferred IUPAC name [(2R,3R)-7-hydroxy-2-(4-hydroxy-3-methoxyphenyl)-4-oxo-2,3-dihydrochromen-3-yl]acetate.

“5-deoxypinobanksin-3-O-acetate” has the IUPAC name [(2R,3R)-7-hydroxy-2-phenyl-4-oxo-2,3-dihydrochromen-3-yl]acetate.

The compounds aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate and 5-deoxypinobanksin-3-O-acetate are prepared by the acetylation of the R5 position of the compound of formula (Ia).

Where the compounds disclosed herein have at least one chiral center that is not specifically indicated in the formula, they may exist as individual enantiomers and diastereomers or as mixtures of such isomers. In some embodiments, the sweet-enhancing compound has substantial enantiomeric purity.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Physiologically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in comestible food and/or beverage products.

Where the compounds disclosed herein have at least one chiral center, they may exist as individual enantiomers and diastereomers or as mixtures of such isomers. In some embodiments in connection with the second aspect, the sweet-enhancing compound has substantial enantiomeric purity.

For example, in some embodiments, a composition comprising the compound of formula (I or Ia), or a salt thereof, the (2R,3R) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound [i.e., amount of (2R,3R)+amount of (2S,3R)+amount of (2R,3S)+amount of (2S,3S)]. In some other embodiments, a composition comprising the compound of formula (I or Ia), or a salt thereof, the (2R,3S) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition. In some other embodiments, a composition comprising the compound of formula (I or Ia), or a salt thereof, the (2S,3R) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition. In some other embodiments, a composition comprising the compound of formula (I or Ia), or a salt thereof, the (2S,3S) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition.

In some other embodiments of any of the embodiments of the preceding paragraph, a composition comprising the compound of formula (I or Ia), or a salt thereof, the (2R,3R) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2R,3S) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2S,3R) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2S,3S) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition.

For example, in some embodiments, a composition comprising the compound of formula (I or Ia), or a salt thereof, the (2R,3R) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound [i.e., amount of (2R,3R)+amount of (2S,3R)+amount of (2R,3S)+amount of (2S,3S)] in the composition. In some other embodiments, a composition comprising the compound of formula (I or Ia), or a salt thereof, the (2R,3S) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition. In some other embodiments, a composition comprising the compound of formula (I or Ia), or a salt thereof, the (2S,3R) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition. In some other embodiments, a composition comprising the compound of formula (I or Ia), or a salt thereof, the (2S,3S) enantiomer makes up at least 50% by weight, or at least 60% by weight, or at least 70% by weight, or at least 80% by weight, or at least 90% by weight, or at least 95% by weight, or at least 97% by weight, or at least 99% by weight, of the amount of compound present in the composition, based on the total weight of the compound in the composition.

In some other embodiments of any of the embodiments of the preceding paragraph, a composition comprising the compound of formula (I or Ia), or a salt thereof, the (2R,3R) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2R,3S) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2S,3R) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition. In some other embodiments of any of the embodiments of the preceding paragraph, a composition comprising the compound of formula (I or Ia), or a salt thereof, the (2S,3S) enantiomer makes up no more than 50% by weight, or no more than 40% by weight, or no more than 30% by weight, or no more than 20% by weight, or no more than 10% by weight, or no more than 5% by weight, or no more than 3% by weight, or no more than 1% by weight, of the amount of compound present in the composition, based on the total weight of the compound in the composition.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

### Polypeptides of the Invention

The present invention concerns a process of acylating a precursor compound of formula (Ia) to make a compound of formula (I).

As can be appreciated, such a reaction could be performed with known chemical reactions. However, a disadvantage of this approach is that the precursor compounds can be acylated at a number of different locations in the structure. In addition, due to chemical reaction conditions, unwanted side product formation and isomerization can be observed. This is not desirable since such molecules may not function as sweetener enhancers, which is the application of the compounds of formula (I).

The inventors therefore sought to identify enzymes that can be used in the process for making a compound of formula (I). They identified several different acyltransferases which can be used for this purpose provided herein as SEQ ID NOs: 1 to 7, 31 to 36 and 55 to 68. These acyltransferases when prepared using recombinant expression technologies are considered polypeptides of the invention.

An acyltransferase is an enzyme that catalyzes the transfer of an acyl group to the oxygen molecule of an acceptor molecule. They constitute a large and very diverse class of enzymes and are involved in numerous metabolic pathways in cells.

The present inventors sought to identify whether acyltransferase enzymes could be used in a process to prepare a compound of formula (I) from a compound of formula (Ia). Surprisingly they identified several such enzymes which can be used for this purpose, as shown in the accompanying examples. Moreover, the enzymes specifically acylate the compound of formula (Ia) at the R5 position and at no other positions in the structure.

Acyltransferases also have the advantage in that they are highly efficient in aqueous reaction conditions hence having good utility for in vivo reactions.

To the best knowledge of the inventors this is the first time that an acyltransferase has been used for the specific reaction at the R5 position in formula (Ia). Indeed, it has never been previously reported that acyltransferases accept flavanonols as acceptor molecules.

The transfer of an acyl group to the oxygen molecule of an acceptor molecule is preferably performed in the presence of a cofactor, preferably acyl-CoA. These cofactors are non-protein, chemical compounds which act as a catalyst for the transfer of acyl groups.

In a preferred embodiment of the invention, the acyltransferase enzyme is an acetyltransferase and the cofactor is acetyl-CoA. More preferably the acetyltransferase is an O-acetyltransferase.

In a further preferred embodiment of the invention the acetyltransferase enzyme comprises the amino acid sequence HXXXD (SEQ ID NO: 29) and/or amino acid sequence [DN]FGxG (SEQ ID NO:30) and/or amino acid sequence [ST]S[WL](SEQ ID NO: 94).

The HXXXD, [DN]FGxG and [ST]S[WL] motifs are in Prosite syntax, as defined in https://prosite.expasy.org/scanprosite/scanprosite_doc.html, wherein “X” or “x” denotes an arbitrary amino acid.

The HXXXD, the [DN]FGxG and the [ST]S[WL] motifs are amino acid motifs shared between the acetyltransferase enzymes demonstrated in the accompanying examples to have utility in the process of the first aspect of the invention. Accordingly therefore, the motifs define a collection of acetyltransferase enzymes which have been demonstrated to have function in the process of the invention and accordingly define a subgroup of these enzymes for utility in the process. The histidine (H) in HXXXD and the [ST] and [WL] of motif [ST]S[WL] are part of the enzyme's binding pocket.

Examples of acetyltransferase enzymes that may be used in the process of the invention include a polypeptide encoded by the amino acid sequence of any of SEQ ID NOs: 1 to 7, 31 to 36 and 55 to 68.

Hence a preferred embodiment of the process of the invention is wherein the acyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68.

SEQ ID NOs: 1, 4 and 5 encode acyltransferase enzymes isolated from Inula viscosa (Dittrichia viscosa), a highly branching perennial common throughout the Mediterranean basin. The present inventors are the first to identify and characterize these polypeptide sequences as enzymes having acyltransferase activity.

SEQ ID NOs: 2, 3 and 56 encode acyltransferase enzymes identified from Erigeron canadensis, an annual plant, tall with sparsely hairy stems which inhabits most of the temperate zone of Asia, Europe, North America and Australia. The present inventors are the first to identify and characterize these polypeptide sequences as enzymes having acyltransferase activity.

SEQ ID NOs: 6 and 7 encode acyltransferase enzymes identified from Pulicaria dysenterica, a species native to Europe and western Asia. The present inventors are the first to identify and characterize these polypeptide sequences as enzymes having acyltransferase activity.

SEQ ID NO: 31 encodes an acyltransferase enzyme identified from Cynara cardunculus var. scolymus, commonly called globe artichoke. The present inventors are the first to identify and characterize this polypeptide sequence as an enzyme having acyltransferase activity.

SEQ ID NOs: 32, 57 and 58 encodes acyltransferase enzymes identified from Helianthus annuus also called common sunflower. It is a large annual forb of the genus Helianthus grown as a crop for its edible oily seeds. The present inventors are the first to identify and characterize this polypeptide sequence as an enzyme having acyltransferase activity.

SEQ ID NO: 33 encodes an acyltransferase enzyme identified from Arctium lappa, also called greater burdock, and is a Eurasian species of plant in the family of Asteraceae and cultivated in gardens for its root used as a vegetable. The present inventors are the first to identify and characterize this polypeptide sequence as an enzyme having acyltransferase activity.

SEQ ID NOs: 34 and 60 encodes acyltransferase enzymes identified from Mikania micrantha, which is known as bitter vine, climbing hemp vine or American rope and belongs to the family of Asteraceae. It is native to the sub-tropical zones of North, Central and South America, but also found as weed in Asia. The present inventors are the first to identify and characterize this polypeptide sequence as an enzyme having acyltransferase activity.

SEQ ID NOs: 35 and 36 encode acyltransferase enzymes identified from Smallanthus sonchifolius, which is also called Yacon and belongs to the family of Asteraceae. It is a food plant traditionally grown in the Andes but can be found all around the world. The present inventors are the first to identify and characterize these polypeptide sequences as enzymes having acyltransferase activity.

SEQ ID NO: 55 encodes an acyltransferase enzyme identified from Artemisia annua, also known as sweet wormwood. Artemisia annua belongs to the family of Asteraceae. It is native to temperate Asia but naturalized in many countries around the globe. It is grown agriculturally for the extraction of artemisinin, which is a medication used to treat malaria.

SEQ ID NO: 59 encodes an acyltransferase enzyme identified from Daucus carota subsp. Sativus, also known as wild carrot. Daucus carota subsp. Sativus belongs to the Apiaceae family and can be found all over the world.

SEQ ID NO: 61 encodes an acyltransferase enzyme identified from Tanacetum cinerariifolium, also called Dalmatian chrysanthemum, which belongs to the Asteraceae family. It can be found in the Mediterranean. But it is grown all over the world as it is a natural source of an insecticide called pyrethrum.

SEQ ID NO: 62 is a variant of SEQ ID NO: 1 and comprises a P34A substitution.

SEQ ID NO: 63 is a variant of SEQ ID NO: 1 and comprises a P34H substitution.

SEQ ID NO: 64 is a variant of SEQ ID NO: 1 and comprises P34A and F354Y substitutions.

SEQ ID NO: 65 is a variant of SEQ ID NO: 1 and comprises P34A and F365Y substitutions.

SEQ ID NO: 66 is a variant of SEQ ID NO: 1 and comprises P34A, F354Y, and F365Y substitutions.

SEQ ID NO: 67 is a variant of SEQ ID NO: 1 and comprises a F358Y substitution.

SEQ ID NO: 68 is a variant of SEQ ID NO: 6 and comprises I302F, L304F, L362F, L403F, A400N substitutions.

The present inventors also examined the activity of the enzymes encoded by SEQ ID NOs: 1 to 4 with different acyl-CoA compounds. As shown in the accompanying examples, where the acyltransferase enzyme has the amino acid sequence shown in SEQ ID NO: 1, the cofactor is acetyl-CoA, propanoyl-CoA or butyryl-CoA, preferably acetyl-CoA.

Where the acyltransferase enzyme has the amino acid sequence shown in SEQ ID NO: 2, the cofactor is acetyl-CoA or propanoyl-CoA, preferably acetyl-CoA.

Where the acyltransferase enzyme has the amino acid sequence shown in SEQ ID NO: 3, the cofactor is acetyl-CoA or propanoyl-CoA, preferably acetyl-CoA.

Where the acyltransferase enzyme has the amino acid sequence shown in SEQ ID NO: 4, the cofactor is acetyl-CoA, butyryl-CoA, hexanoyl-CoA or octanoyl-CoA, preferably hexanoyl -CoA.

An alternative aspect of the invention is wherein the process for making a compound of formula (I) comprises acylating a precursor compound of formula (Ia) with a hydrolase enzyme.

Carboxylic ester hydrolase [EC 3.1.1] is a class of hydrolytic enzymes that are commonly used as biochemical catalysts which utilize water as a hydroxyl group donor during the substrate breakdown. In addition, they are known in the art to catalyze the synthesis of ester bonds most efficiently in the absence of water.

As shown in the accompanying examples, the present inventors have identified hydrolase enzymes including triacylglycerol lipase enzymes [EC 3.1.1.3] and cutinase enzymes [EC 3.1.1.74], that can be used in the process of the invention. This is the first time it has been demonstrated that a hydrolase can be used in conversion of a compound of formula (Ia) to a compound of formula (I), where the compound of formula (I) is aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate or 5-deoxypinobanksin-3-O-acetate.

Hence a further aspect of the invention provides a process for making a compound of formula (I) the process comprising reacting a precursor compound of formula (Ia) with a hydrolase enzyme for form a compound of formula (I).

Examples of hydrolases that may be used in this aspect of the invention include: IMMLIPX-COV-1, available from Chiralvision, using lipase Lipex 100L (Lipex 100L from Novozymes), covalent on IB-150A.

IMMAULI-COV-1, available from Chiralvision, using lipase from Bacillus subtilis (lipase from Aum Enzymes), covalent on IB-150A.

IMML51-COV-1, available from Chiralvision, using cutinase from Humicola insolens (NZ51032 from Novozymes), covalent on IB-150A.

IMMRES-COV-1, available from Chiralvision, using lipase from Aspergillus oryzae (Resinase HT from Novozymes), covalent on IB-150A.

IMMTLL-COV-1, available from Chiralvision, using lipase from Thermomyces lanuginosa (Lipolase from Novozymes), covalent on IB-150A.

IMMCALBY-COV-1, available from Chiralvision, using generic lipase B from Candida antarctica (CaLB) from c-Lecta, covalent on IB-150A.

IMMCALB-COV-1XL, available from Chiralvision, using lipase B from C. antarctica (CaLB) from Novozymes, covalent on IB-150A.

However, a preferred embodiment of the invention is wherein the lipase is IMMLIPX-COV-1.

As mentioned above, the present inventors sought to identify whether an acyltransferase could be used to acylate the compound of formula (Ia) to form a compound of formula (I). Several enzymes which can be used for this purpose, are disclosed in the accompanying examples.

Hence a further aspect of the invention provides a recombinant polypeptide having acyltransferase activity comprising an amino acid sequence having at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68 or comprising the amino acid sequence of any of SEQ ID NOs: 1 to 7, 31 to 36 and 55 to 68.

Further provided herein is a recombinant nucleic acid molecule comprising a nucleotide sequence encoding a polypeptide having acyltransferase activity and comprising an amino acid sequence having at least 50%, 55%, 60%, 65%, 70%, 75%, 80%, 85%, 90%, 95%, 96%, 97%, 98%, or 99% sequence identity to any of SEQ ID NOs: 1 to 7, 31 to 36 and 55 to 68 or comprising the amino acid sequence of any of SEQ ID NOs: 1 to 7, 31 to 36 and 55 to 68.

In addition to identifying polypeptides having acyltransferase activity as defined in any of SEQ ID NOs: 1 to 7, 31 to 36 and 55 to 68, the present inventors identified the native nucleic acid sequences for each enzyme of SEQ ID NOs: 1 to 7, 31 to 36 and 55 to 61. Furthermore, for all sequences including SEQ ID NO: 62 to 68, the inventors optimized the codon for each nucleic acid sequence such that it is suitable for expression in prokaryotic cells, preferably E. coli cells, and eukaryotic cells, preferably Saccharomyces cerevisiae.

Hence SEQ ID NO: 1 is encoded by its native nucleic acid sequence shown SEQ ID NO: 8. The nucleic acid sequence optimized for expression in eukaryotic cells is shown in SEQ ID NO: 9. The nucleic acid sequence optimized for expression in prokaryotic cells is shown in SEQ ID NO: 10.

Hence SEQ ID NO: 2 is encoded by its native nucleic acid sequence shown SEQ ID NO: 11. The nucleic acid sequence optimized for expression in eukaryotic cells is shown in SEQ ID NO: 12. The nucleic acid sequence optimized for expression in prokaryotic cells is shown in SEQ ID NO: 13.

Hence SEQ ID NO: 3 is encoded by its native nucleic acid sequence shown SEQ ID NO: 14. The nucleic acid sequence optimized for expression in eukaryotic cells is shown in SEQ ID NO: 15. The nucleic acid sequence optimized for expression in prokaryotic cells is shown in SEQ ID NO: 16.

Hence SEQ ID NO: 4 is encoded by its native nucleic acid sequence shown SEQ ID NO: 17. The nucleic acid sequence optimized for expression in eukaryotic cells is shown in SEQ ID NO: 18. The nucleic acid sequence optimized for expression in prokaryotic cells is shown in SEQ ID NO: 19.

Hence SEQ ID NO: 5 is encoded by its native nucleic acid sequence shown SEQ ID NO: 20. The nucleic acid sequence optimized for expression in eukaryotic cells is shown in SEQ ID NO: 21. The nucleic acid sequence optimized for expression in prokaryotic cells is shown in SEQ ID NO: 22.

Hence SEQ ID NO: 6 is encoded by its native nucleic acid sequence shown SEQ ID NO: 23. The nucleic acid sequence optimized for expression in eukaryotic cells is shown in SEQ ID NO: 24. The nucleic acid sequence optimized for expression in prokaryotic cells is shown in SEQ ID NO: 25.

Hence SEQ ID NO: 7 is encoded by its native nucleic acid sequence shown SEQ ID NO: 26. The nucleic acid sequence optimized for expression in eukaryotic cells is shown in SEQ ID NO: 27. The nucleic acid sequence optimized for expression in prokaryotic cells is shown in SEQ ID NO: 28.

Hence SEQ ID NO: 31 is encoded by its native nucleic acid sequence shown SEQ ID NO: 37. The nucleic acid sequence optimized for expression in eukaryotic cells is shown in SEQ ID NO: 38. The nucleic acid sequence optimized for expression in prokaryotic cells is shown in SEQ ID NO: 39.

Hence SEQ ID NO: 32 is encoded by its native nucleic acid sequence shown SEQ ID NO: 40. The nucleic acid sequence optimized for expression in eukaryotic cells is shown in SEQ ID NO: 41. The nucleic acid sequence optimized for expression in prokaryotic cells is shown in SEQ ID NO: 42.

Hence SEQ ID NO: 33 is encoded by its native nucleic acid sequence shown SEQ ID NO: 43. The nucleic acid sequence optimized for expression in eukaryotic cells is shown in SEQ ID NO: 44. The nucleic acid sequence optimized for expression in prokaryotic cells is shown in SEQ ID NO: 45.

Hence SEQ ID NO: 34 is encoded by its native nucleic acid sequence shown SEQ ID NO: 46. The nucleic acid sequence optimized for expression in eukaryotic cells is shown in SEQ ID NO: 47. The nucleic acid sequence optimized for expression in prokaryotic cells is shown in SEQ ID NO: 48.

Hence SEQ ID NO: 35 is encoded by its native nucleic acid sequence shown SEQ ID NO: 49. The nucleic acid sequence optimized for expression in eukaryotic cells is shown in SEQ ID NO: 50. The nucleic acid sequence optimized for expression in prokaryotic cells is shown in SEQ ID NO: 51.

Hence SEQ ID NO: 36 is encoded by its native nucleic acid sequence shown SEQ ID NO: 52. The nucleic acid sequence optimized for expression in eukaryotic cells is shown in SEQ ID NO: 53. The nucleic acid sequence optimized for expression in prokaryotic cells is shown in SEQ ID NO: 54.

Hence SEQ ID NO: 55 is encoded by its native nucleic acid sequence shown SEQ ID NO: 69. The nucleic acid sequence optimized for expression in eukaryotic cells is shown in SEQ ID NO: 70.

Hence SEQ ID NO: 56 is encoded by its native nucleic acid sequence shown SEQ ID NO: 71. The nucleic acid sequence optimized for expression in eukaryotic cells is shown in SEQ ID NO: 72.

Hence SEQ ID NO: 57 is encoded by its native nucleic acid sequence shown SEQ ID NO: 73. The nucleic acid sequence optimized for expression in eukaryotic cells is shown in SEQ ID NO: 74.

Hence SEQ ID NO: 58 is encoded by its native nucleic acid sequence shown SEQ ID NO: 75. The nucleic acid sequence optimized for expression in eukaryotic cells is shown in SEQ ID NO: 76.

Hence SEQ ID NO: 59 is encoded by its native nucleic acid sequence shown SEQ ID NO: 77. The nucleic acid sequence optimized for expression in eukaryotic cells is shown in SEQ ID NO: 78.

Hence SEQ ID NO: 60 is encoded by its native nucleic acid sequence shown SEQ ID NO: 79. The nucleic acid sequence optimized for expression in eukaryotic cells is shown in SEQ ID NO: 80.

Hence SEQ ID NO: 61 is encoded by its native nucleic acid sequence shown SEQ ID NO: 81. The nucleic acid sequence optimized for expression in eukaryotic cells is shown in SEQ ID NO: 82.

Hence SEQ ID NO: 62 is encoded by a nucleic acid sequence optimized for expression in eukaryotic cells as shown in SEQ ID NO: 83 or a nucleic acid sequence optimized for expression in prokaryotic cells as shown in SEQ ID NO: 84.

Hence SEQ ID NO: 63 is encoded by a nucleic acid sequence optimized for expression in eukaryotic cells as shown in SEQ ID NO: 85 or a nucleic acid sequence optimized for expression in prokaryotic cells as shown in SEQ ID NO: 86.

Hence SEQ ID NO: 64 is encoded by a nucleic acid sequence optimized for expression in eukaryotic cells as shown in SEQ ID NO: 87.

Hence SEQ ID NO: 65 is encoded by a nucleic acid sequence optimized for expression in eukaryotic cells as shown in SEQ ID NO: 88.

Hence SEQ ID NO: 66 is encoded by a nucleic acid sequence optimized for expression in eukaryotic cells as shown in SEQ ID NO: 89.

Hence SEQ ID NO: 67 is encoded by a nucleic acid sequence optimized for expression in eukaryotic cells as shown in SEQ ID NO: 90 or a nucleic acid sequence optimized for expression in prokaryotic cells as shown in SEQ ID NO: 91.

Hence SEQ ID NO: 68 is encoded by a nucleic acid sequence optimized for expression in eukaryotic cells as shown in SEQ ID NO: 92 or a nucleic acid sequence optimized for expression in prokaryotic cells as shown in SEQ ID NO: 93.

Accordingly therefore, further provided is an isolated nucleic acid comprising a nucleotide sequence having at least 50%, 55%, 60%, 65%, 70%, 75%, 80%, 85%, 90%, 95%, 96%, 97%, 98% or 99% sequence identity to any of SEQ ID NOs: 8 to 28, 37 to 54 and 69 to 93 or comprising the nucleotide sequence of any of SEQ ID NOs: 8 to 28, 37 to 54 and 69 to 93 or the reverse complement thereof.

Further provided is an isolated nucleic acid molecule encoding a recombinant polypeptide provided herein.

In one aspect provided herein is a vector comprising the nucleic acid molecules described herein. In another aspect, the vector is an expression vector. In a further aspect, the vector is a prokaryotic vector, viral vector or an eukaryotic vector.

Also provided is a non-human host organism or a host cell comprising (1) a nucleic acid molecule described above, or (2) an expression vector comprising said nucleic acid molecule. In one aspect the non-human organism or host cell is a prokaryotic or eukaryotic cell. In another aspect the host cell is a bacterial, archaebacterial, fungal such as yeast, algal or plant cell. In a further aspect, the bacterial cell is E. coli and the yeast cell is Saccharomyces cerevisiae.

Further provided is the use of a polypeptide described herein for producing a compound of formula (I).

Further provided is a nucleotide sequence obtained by modifying any of SEQ ID NOs: 8 to 28, 37 to 54 and 69 to 93 or the reverse complement thereof which encompasses any sequence that has been obtained by modifying the sequence of any of SEQ ID NOs: 8 to 28, 37 to 54 and 69 to 93 or of the reverse complement thereof using any method known in the art, for example, by introducing any type of mutations such as deletion, insertion and/or substitution mutations. The nucleic acids comprising a sequence obtained by mutation of any of SEQ ID NOs: 8 to 28, 37 to 54 and 69 to 93 or the reverse complement thereof are encompassed by an embodiment herein, provided that the sequences they comprise share at least the defined sequence identity of any of SEQ ID NOs: 8 to 28, 37 to 54 and 69 to 93 or the reverse complement thereof and provided that they encode a polypeptide having acyltransferase activity, as defined in any of the above embodiments. Mutations may be any kind of mutations of these nucleic acids, for example, point mutations, deletion mutations, insertion mutations and/or frame shift mutations of one or more nucleotides of the DNA sequence of any of SEQ ID NOs: 8 to 28, 37 to 54 and 69 to 93. In one embodiment, the nucleic acid of an embodiment herein may be truncated provided that it encodes a polypeptide as described herein.

A variant nucleic acid may be prepared in order to adapt its nucleotide sequence to a specific expression system. For example, bacterial and yeast expression systems are known to more efficiently express polypeptides if amino acids are encoded by particular codons.

Due to the degeneracy of the genetic code, more than one codon may encode the same amino acid sequence, multiple nucleic acid sequences can code for the same protein or polypeptide, all these DNA sequences being encompassed by an embodiment herein. Where appropriate, the nucleic acid sequences encoding the acyltransferase may be optimized for increased expression in the host cell. For example, nucleotides of an embodiment herein may be synthesized using codons particular to a host for improved expression.

In one embodiment provided herein is an isolated, recombinant or synthetic nucleic acid sequence of any of SEQ ID NOs: 8 to 28, 37 to 54 and 69 to 93 encoding for a polypeptide having acyltransferase activity comprising the amino acid sequence of SEQ ID NOs: 1 to 7, 31 to 36 and 55 to 68 or fragments thereof that catalyze production of a compound of formula (I):

Provided herein are also cDNA, genomic DNA and RNA sequences. Any nucleic acid sequence encoding the acyltransferase or variants thereof is also referred herein as a acyltransferase encoding sequence.

According to one embodiment, the nucleic acid of any of SEQ ID NOs: 8 to 28, 37 to 54 and 69 to 93 is the coding sequence of an acyltransferase gene encoding an acyltransferase obtained as described in the Examples.

A fragment of a polynucleotide of any of SEQ ID NOs: 8 to 28, 37 to 54 and 69 to 93 refers to contiguous nucleotides that is particularly at least 15 bp, at least 30 bp, at least 40 bp, at least 50 bp and/or at least 60 bp in length of the polynucleotide of an embodiment herein. Particularly the fragment of a polynucleotide comprises at least 25, more particularly at least 50, more particularly at least 75, more particularly at least 100, more particularly at least 150, more particularly at least 200, more particularly at least 300, more particularly at least 400, more particularly at least 500, more particularly at least 600, more particularly at least 700, more particularly at least 800, more particularly at least 900, more particularly at least 1000 contiguous nucleotides of the polynucleotide of an embodiment herein. Without being limited, the fragment of the polynucleotides herein may be used as a PCR primer, and/or as a probe, or for anti-sense gene silencing or RNAi.

It is clear to the person skilled in the art that genes, including the polynucleotides of an embodiment herein, can be cloned on basis of the available nucleotide sequence information, such as found in the attached sequence listing, by methods known in the art. These include e.g. the design of DNA primers representing the flanking sequences of such gene of which one is generated in sense orientations and which initiates synthesis of the sense strand and the other is created in reverse complementary fashion and generates the antisense strand. Thermo stable DNA polymerases such as those used in polymerase chain reaction are commonly used to carry out such experiments. Alternatively, DNA sequences representing genes can be chemically synthesized and subsequently introduced in DNA vector molecules that can be multiplied by e.g. compatible bacteria such as e.g. E. coli or a yeast cell.

In a related embodiment provided herein, PCR primers and/or probes for detecting nucleic acid sequences encoding an acyltransferase are provided. The skilled artisan will be aware of methods to synthesize degenerate or specific PCR primer pairs to amplify a nucleic acid sequence encoding the acyltransferase or fragments thereof, based on of any of SEQ ID NOs: 8 to 28, 37 to 54 and 69 to 93. A detection kit for nucleic acid sequences encoding the acyltransferase may include primers and/or probes specific for nucleic acid sequences encoding the acyltransferase, and an associated protocol to use the primers and/or probes to detect nucleic acid sequences encoding the acyltransferase in a sample. Such detection kits may be used to determine whether a plant, organism or cell has been modified, i.e., transformed with a sequence encoding the acyltransferase.

To test a function of variant DNA sequences according to an embodiment herein, the sequence of interest is tested in an activity assay, examples of which are provided in herein.

The skilled artisan will be aware of methods to identify homologous sequences in other organisms and methods to determine the percentage of sequence identity between homologous sequences. Such newly identified DNA molecules then can be sequenced and the sequence can be compared with the nucleic acid sequence of any of SEQ ID NOs: 8 to 28, 37 to 54 and 69 to 93.

The percentage of identity between two peptide or nucleotide sequences is a function of the number of amino acids or nucleotide residues that are identical in the two sequences when an alignment of these two sequences has been generated. Identical residues are defined as residues that are the same in the two sequences in a given position of the alignment. The percentage of sequence identity, as used herein, is calculated from the optimal alignment by taking the number of residues identical between two sequences dividing it by the total number of residues in the shortest sequence and multiplying by 100. The optimal alignment is the alignment in which the percentage of identity is the highest possible. Gaps may be introduced into one or both sequences in one or more positions of the alignment to obtain the optimal alignment. These gaps are then taken into account as non-identical residues for the calculation of the percentage of sequence identity. Alignment for the purpose of determining the percentage of amino acid or nucleic acid sequence identity can be achieved in various ways using computer programs and for instance publicly available computer programs available on the world wide web. Preferably, the BLAST program (Tatiana et al, FEMS Microbiol Lett., 1999, 174:247-250, 1999) set to the default parameters, available from the National Center for Biotechnology Information (NCBI) website at ncbi.nlm.nih.gov/BLAST/bl2seq/wblast2.cgi, can be used to obtain an optimal alignment of protein or nucleic acid sequences and to calculate the percentage of sequence identity.

A related embodiment provided herein provides a nucleic acid sequence which is complementary to the nucleic acid sequence according to of any of SEQ ID NOs: 8 to 28, 37 to 54 and 69 to 93 such as inhibitory RNAs, or nucleic acid sequence which hybridizes under stringent conditions to at least part of the nucleotide sequence according of any of SEQ ID NOs: 8 to 28, 37 to 54 and 69 to 93. An alternative embodiment of an embodiment herein provides a method to alter gene expression in a host cell. For instance, the polynucleotide of an embodiment herein may be enhanced or overexpressed or induced in certain contexts (e.g. upon exposure to a certain temperature or culture conditions) in a host cell or host organism.

Alteration of expression of a polynucleotide provided herein may also result in ectopic expression which is a different expression pattern in an altered and in a control or wild-type organism. Alteration of expression occurs from interactions of polypeptide of an embodiment herein with exogenous or endogenous modulators, or as a result of chemical modification of the polypeptide. The term also refers to an altered expression pattern of the polynucleotide of an embodiment herein which is altered below the detection level or completely suppressed activity.

In one embodiment, the at least one polypeptide having acyltransferase activity used in any of the herein-described embodiments or encoded by the nucleic acid used in any of the herein-described embodiments comprises an amino acid sequence that is a variant of any of SEQ ID NOs: 1 to 7, 31 to 36 and 55 to 68, obtained by genetic engineering. In one embodiment the polypeptide comprises an amino acid sequence encoded by a nucleotide sequence that has been obtained by modifying any of SEQ ID NOs: 8 to 28, 37 to 54 and 69 to 93 or the reverse complement thereof.

Polypeptides are also meant to include variants and truncated polypeptides provided that they have acyltransferase activity.

According to another embodiment, the at least one polypeptide having a acyltransferase activity used in any of the herein-described embodiments or encoded by the nucleic acid used in any of the herein-described embodiments comprises an amino acid sequence that is a variant of any of SEQ ID NOs: 1 to 7, 31 to 36 and 55 to 68, obtained by genetic engineering, provided that said variant has acyltransferase activity and has the required percentage of identity to any of SEQ ID NOs: 1 to 7, 31 to 36 and 55 to 68 as described herein.

According to another embodiment, the at least one polypeptide having a acyltransferase activity used in any of the herein-described embodiments or encoded by the nucleic acid used in any of the herein-described embodiments is a variant of any of SEQ ID NOs: 1 to 7, 31 to 36 and 55 to 68 that can be found naturally in other organisms provided that it has a acyltransferase activity. As used herein, the polypeptide includes a polypeptide or peptide fragment that encompasses the amino acid sequences identified herein, as well as truncated or variant polypeptides provided that they have acyltransferase activity and that they share at least the defined percentage of identity with the corresponding fragment of any of SEQ ID NOs: 1 to 7, 31 to 36 and 55 to 68.

Examples of variant polypeptides are naturally occurring proteins that result from alternate mRNA splicing events or from proteolytic cleavage of the polypeptides described herein. Variations attributable to proteolysis include, for example, differences in the N- or C-termini upon expression in different types of host cells, due to proteolytic removal of one or more terminal amino acids from the polypeptides of an embodiment herein. Polypeptides encoded by a nucleic acid obtained by natural or artificial mutation of a nucleic acid of an embodiment herein, as described thereafter, are also encompassed by an embodiment herein.

Polypeptide variants resulting from a fusion of additional peptide sequences at the amino and carboxyl terminal ends can also be used in the methods of an embodiment herein. In particular such a fusion can enhance expression of the polypeptides, be useful in the purification of the protein or improve the enzymatic activity of the polypeptide in a desired environment or expression system. Such additional peptide sequences may be signal peptides, for example. Another aspect encompasses methods using variant polypeptides, such as those obtained by fusion with other oligo- or polypeptides and/or those which are linked to signal peptides. Polypeptides resulting from a fusion with another functional protein, can also be advantageously used in the methods of an embodiment herein.

A variant may also differ from the polypeptide of an embodiment herein by attachment of modifying groups which are covalently or non-covalently linked to the polypeptide backbone. The variant also includes a polypeptide which differs from the polypeptide provided herein by introduced N-linked or O-linked glycosylation sites, and/or an addition of cysteine residues. The skilled artisan will recognize how to modify an amino acid sequence and preserve biological activity.

The present invention also relates to “functional equivalents” (also designated as “analogs” or “functional mutations”) of the polypeptides specifically described herein.

For example, “functional equivalents” refer to polypeptides which, in a test used for determining acyltransferase activity, display at least a 1 to 10%, or at least 20%, or at least 50%, or at least 75%, or at least 90% higher or lower acyltransferase activity, as that of the respective polypeptide specifically defined herein, as well as truncated or variant polypeptides provided that they have acyltransferase activity and that they share at least the defined percentage of identity with the corresponding fragment of any of SEQ ID NOs: 1 to 7, 31 to 36 and 55 to 68.

“Functional equivalents” may also be derived from helper polypeptides as described therein which assist in the functional expression of another, preferably enzymatically active, polypeptide, in particular the correct folding of said expressed polypeptide, as for example of a polypeptide with acyltransferase activity. Such modified helper polypeptide may still be regarded as functional, as long as it improves the correct expression or folding said enzymatically active polypeptide relative the expression of the same enzymatically active polypeptide under otherwise identical conditions but in the absence of such helper polypeptide.

In addition to the gene sequences shown in the sequences disclosed herein, it will be apparent for the person skilled in the art that DNA sequence polymorphisms may exist within a given population, which may lead to changes in the amino acid sequence of the polypeptides disclosed herein. Such genetic polymorphisms may exist in cells from different populations or within a population due to natural allelic variation. Allelic variants may also include functional equivalents.

Further embodiments also relate to the molecules derived by such sequence polymorphisms from the concretely disclosed nucleic acids. These natural variations usually bring about a variance of about 1 to 5% in the nucleotide sequence of a gene or in the amino acid sequence of the polypeptides disclosed herein. As mentioned above, the nucleic acid encoding the polypeptide or variants thereof of an embodiment herein is a useful tool to modify non-human host organisms or cells and to modify non-human host organisms or cells intended to be used in the methods described herein.

An embodiment provided herein provides amino acid sequences of acyltransferase proteins including orthologs and paralogs as well as methods for identifying and isolating orthologs and paralogs of the acyltransferase in other organisms. Particularly, so identified orthologs and paralogs of the acyltransferase and are capable of producing a compound of formula (I).

The acyltransferase polypeptide can be obtained by extraction from any organism expressing it, using standard protein or enzyme extraction technologies. If the host organism is an unicellular organism or cell releasing the polypeptide of an embodiment herein into the culture medium, the polypeptide may simply be collected from the culture medium, for example by centrifugation, optionally followed by washing steps and re-suspension in suitable buffer solutions. If the organism or cell accumulates the polypeptide within its cells, the polypeptide may be obtained by disruption or lysis of the cells and optionally further extraction of the polypeptide from the cell lysate.

According to another embodiment, the at least one polypeptide having a acyltransferase can be used in the processes of the invention.

The functionality or activity of any acyltransferase protein, variant or fragment, may be determined using various methods. For example, transient or stable overexpression in plant, bacterial or yeast cells can be used to test whether the protein has activity, i.e., produces a compound of formula (I). Acyltransferase activity may be assessed in assays described in the examples herein, indicating functionality. A variant or derivative of an acyltransferase polypeptide of an embodiment herein retains an ability to produce a compound of formula (I). Amino acid sequence variants of the acyltransferase provided herein may have additional desirable biological functions including, e.g., altered substrate utilization, reaction kinetics, product distribution or other alterations.

Further provided is at least one vector comprising the nucleic acid molecules described herein.

Also provided herein is a vector selected from the group of a prokaryotic vector, viral vector and a eukaryotic vector.

Further provided here is a vector that is an expression vector.

The nucleic acid sequences of an embodiment herein encoding acyltransferase proteins can be inserted in expression vectors and/or be contained in chimeric genes inserted in expression vectors, to produce acyltransferase proteins in a host cell or non-human host organism. The vectors for inserting transgenes into the genome of host cells are well known in the art and include plasmids, viruses, cosmids and artificial chromosomes. Binary or co-integration vectors into which a chimeric gene is inserted can also be used for transforming host cells. For the sake of clarity, multiple copies of a gene can be inserted into a host cell.

An embodiment provided herein provides recombinant expression vectors comprising a nucleic acid sequence of an acyltransferase gene, or a chimeric gene comprising a nucleic acid sequence of an acyltransferase gene, operably linked to associated nucleic acid sequences such as, for instance, promoter sequences. For example, a chimeric gene comprising a nucleic acid sequence of any of SEQ ID NOs: 8 to 28, 37 to 54 and 69 to 93 or a variant thereof may be operably linked to a promoter sequence suitable for expression in plant cells, bacterial cells or fungal cells, optionally linked to a 3′ non-translated nucleic acid sequence.

Alternatively, the promoter sequence may already be present in a vector so that the nucleic acid sequence which is to be transcribed is inserted into the vector downstream of the promoter sequence. Vectors can be engineered to have an origin of replication, a multiple cloning site, and a selectable marker.

In one embodiment, an expression vector comprising a nucleic acid as described herein can be used as a tool for transforming non-human host organisms or host cells suitable to carry out the method of an embodiment herein in vivo.

The expression vectors provided herein may be used in the methods for preparing a genetically transformed non-human host organism and/or host cell, in non-human host organisms and/or host cells harboring the nucleic acids of an embodiment herein and in the methods for making polypeptides having an acyltransferase activity, as described herein.

Recombinant non-human host organisms and host cells transformed to harbor at least one nucleic acid of an embodiment herein so that it heterologously expresses or over-expresses at least one polypeptide of an embodiment herein are also very useful tools to carry out the method of an embodiment herein. Such non-human host organisms and host cells are therefore provided herein.

In one embodiment is provided a host cell or non-human host organism comprising at least one of the nucleic acid molecules described herein or comprising at least one vector comprising at least one of the nucleic acid molecules.

A nucleic acid according to any of the above-described embodiments can be used to transform the non-human host organisms and cells and the expressed polypeptide can be any of the above-described polypeptides.

In one embodiment, the non-human host organism or host cell is a prokaryotic cell. In another embodiment, the non-human host organism or host cell is a bacterial cell. In a further embodiment, the non-human host organism or host cell is Escherichia coli.

In one embodiment, the non-human host organism or host cell is a eukaryotic cell. In another embodiment, the non-human host organism or host cell is a yeast cell. In a further embodiment, the non-human host organism or cell is Saccharomyces cerevisiae.

In one embodiment the non-human host organism or host cell expresses a polypeptide, provided that the organism or cell is transformed to harbor a nucleic acid encoding said polypeptide, this nucleic acid is transcribed to mRNA and the polypeptide is found in the host organism or cell.

Suitable methods to transform a non-human host organism or a host cell have been previously described and are also provided herein.

To carry out an embodiment herein in vivo, the host organism or host cell is cultivated under conditions conducive to the production of a compound of formula (I). If the host is a unicellular organism, conditions conducive to the production of a compound of formula (I) may comprise addition of suitable cofactors to the culture medium of the host. In addition, a culture medium may be selected, so as to maximize a compound of formula (I) synthesis. Examples of optimal culture conditions are described in a more detailed manner in the examples.

Non-human host organisms suitable to carry out the method of an embodiment herein in vivo may be any non-human multicellular or unicellular organisms. In one embodiment, the non-human host organism used to carry out an embodiment herein in vivo is a plant, a prokaryote or a fungus. Any plant, prokaryote or fungus can be used. In another embodiment the non-human host organism used to carry out the method of an embodiment herein in vivo is a microorganism. Any microorganism can be used, for example, the microorganism can be a bacteria or yeast, such as E. coli, Corynebacterium glutamicum, Pseudomonas putida, Streptomyces sp, Saccharomyces cerevisiae, Candida krusei, Issatchenkia orientalis, Pichia pastoris or Yarrowia lipolytica.

Isolated higher eukaryotic cells can also be used, instead of complete organisms, as hosts to carry out the method of an embodiment herein in vivo. Suitable eukaryotic cells may be any non-human cell, such as plant or fungal cells.

Further provided here is a method comprising transforming a host cell or a non-human host organism with a nucleic acid encoding a polypeptide having acyltransferase activity and comprising an amino acid sequence having at least 50%, 55%, 60%, 65%, 70%, 75%, 80%, 85%, 90%, 95%, 96%, 97%, 98%, or 99% sequence identity to any of SEQ ID NOs: 1 to 7, 31 to 36 and 55 to 68 or comprising the amino acid sequence of any of SEQ ID NOs: 1 to 7, 31 to 36 and 55 to 68.

In one embodiment, a method provided herein comprises cultivating a non-human host organism or a host cell transformed to express a polypeptide wherein the polypeptide comprises a sequence of amino acids that has at least 75%, 80%, 85%, 90%, 95%, 98%, 99% or 100% sequence identity to any of any of SEQ ID NOs: 1 to 7, 31 to 36 and 55 to 68 under conditions that allow for the production of the polypeptide.

### Recombinant Production of Polypeptides

The invention further relates to methods for recombinant production of polypeptides according to the invention or functional, biologically active fragments thereof, wherein a polypeptide-producing microorganism is cultured, optionally the expression of the polypeptides is induced by applying at least one inducer inducing gene expression and the expressed polypeptides are isolated from the culture. The polypeptides can also be produced in this way on an industrial scale, if desired.

The microorganisms produced according to the invention can be cultured continuously or discontinuously in the batch method or in the fed-batch method or repeated fed-batch method. A summary of known cultivation methods can be found in the textbook by Chmiel (Bioprozesstechnik 1. Einführung in die Bioverfahrenstechnik [Bioprocess technology 1. Introduction to bioprocess technology](Gustav Fischer Verlag, Stuttgart, 1991)) or in the textbook by Storhas (Bioreaktoren and periphere Einrichtungen [Bioreactors and peripheral equipment](Vieweg Verlag, Braunschweig/Wiesbaden, 1994))

The culture medium to be used must suitably meet the requirements of the respective strains. Descriptions of culture media for various microorganisms are given in the manual “Manual of Methods for General Bacteriology” of the American Society for Bacteriology (Washington D. C., USA, 1981).

These media usable according to the invention usually comprise one or more carbon sources, nitrogen sources, inorganic salts, vitamins and/or trace elements.

Preferred carbon sources are sugars, such as mono-, di- or polysaccharides. Very good carbon sources are for example glucose, fructose, mannose, galactose, ribose, sorbose, ribulose, lactose, maltose, sucrose, raffinose, starch or cellulose. Sugars can also be added to the media via complex compounds, such as molasses, or other by-products of sugar refining. It can also be advantageous to add mixtures of different carbon sources. Other possible carbon sources are oils and fats, for example soybean oil, sunflower oil, peanut oil and coconut oil, fatty acids, for example palmitic acid, stearic acid or linoleic acid, alcohols, for example glycerol, methanol or ethanol and organic acids, for example acetic acid or lactic acid.

Nitrogen sources are usually organic or inorganic nitrogen compounds or materials that contain these compounds. Examples of nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soya flour, soya protein, yeast extract, meat extract and others. The nitrogen sources can be used alone or as a mixture.

Inorganic salt compounds that can be present in the media comprise the chloride, phosphorus or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, as well as organic sulfur compounds, such as mercaptans and thiols, can be used as the sulfur source.

Phosphoric acid, potassium dihydrogen phosphate or dipotassium hydrogen phosphate or the corresponding sodium-containing salts can be used as the phosphorus source.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention usually also contain other growth factors, such as vitamins or growth promoters, which include for example biotin, riboflavin, thiamine, folic acid, nicotinic acid, pantothenate and pyridoxine. Growth factors and salts often originate from the components of complex media, such as yeast extract, molasses, corn-steep liquor and the like. Moreover, suitable precursors can be added to the culture medium. The exact composition of the compounds in the medium is strongly dependent on the respective experiment and is decided for each specific case individually. Information on media optimization can be found in the textbook “Applied Microbiol. Physiology, A Practical Approach” (Ed. P. M. Rhodes, P. F. Stanbury, IRL Press (1997) p. 53-73, ISBN 0 19 963577 3). Growth media can also be obtained from commercial suppliers, such as Standard 1 (Merck) or BHI (brain heart infusion, DIFCO) and the like.

All components of the medium are sterilized, either by heat (20 min at 1.5 bar and 121° C.) or by sterile filtration. The components can either be sterilized together, or separately if necessary. All components of the medium can be present at the start of culture or can be added either continuously or batchwise.

The culture temperature is normally between 15° C. and 45° C., preferably 25° C. to 40° C. and can be varied or kept constant during the experiment. The pH of the medium should be in the range from 5 to 8.5, preferably around 7.0. The pH for growing can be controlled during growing by adding basic compounds such as sodium hydroxide, potassium hydroxide, ammonia or ammonia water or acid compounds such as phosphoric acid or sulfuric acid. Antifoaming agents, for example fatty acid polyglycol esters, can be used for controlling foaming. To maintain the stability of plasmids, suitable selective substances, for example antibiotics, can be added to the medium. To maintain aerobic conditions, oxygen or oxygen-containing gas mixtures, for example ambient air, are fed into the culture. The temperature of the culture is normally in the range from 20° C. to 45° C. The culture is continued until a maximum of the desired product has formed. This target is normally reached within 10 hours to 160 hours.

The fermentation broth is then processed further. Depending on requirements, the biomass can be removed from the fermentation broth completely or partially by separation techniques, for example centrifugation, filtration, decanting or a combination of these methods or can be left in it completely.

If the polypeptides are not secreted in the culture medium, the cells can also be lysed and the product can be obtained from the lysate by known methods for isolation of proteins. The cells can optionally be disrupted with high-frequency ultrasound, high pressure, for example in a French press, by osmolysis, by the action of detergents, lytic enzymes or organic solvents, by means of homogenizers or by a combination of several of the aforementioned methods.

The polypeptides can be purified by known chromatographic techniques, such as molecular sieve chromatography (gel filtration), such as Q-sepharose chromatography, ion exchange chromatography and hydrophobic chromatography, and with other usual techniques such as ultrafiltration, crystallization, salting-out, dialysis and native gel electrophoresis. Suitable methods are described for example in Cooper, T. G., Biochemische Arbeitsmethoden [Biochemical processes], Verlag Walter de Gruyter, Berlin, N.Y. or in Scopes, R., Protein Purification, Springer Verlag, New York, Heidelberg, Berlin.

For isolating the recombinant protein, it can be advantageous to use vector systems or oligonucleotides, which lengthen the cDNA by defined nucleotide sequences and therefore code for altered polypeptides or fusion proteins, which for example serve for easier purification. Suitable modifications of this type are for example so-called “tags” functioning as anchors, for example the modification known as hexa-histidine anchor or epitopes that can be recognized as antigens of antibodies (described for example in Harlow, E. and Lane, D., 1988, Antibodies: A Laboratory Manual. Cold Spring Harbor (N.Y.) Press). These anchors can serve for attaching the proteins to a solid carrier, for example a polymer matrix, which can for example be used as packing in a chromatography column, or can be used on a microtiter plate or on some other carrier. At the same time these anchors can also be used for recognition of the proteins. For recognition of the proteins, it is moreover also possible to use usual markers, such as fluorescent dyes, enzyme markers, which form a detectable reaction product after reaction with a substrate, or radioactive markers, alone or in combination with the anchors for derivatization of the proteins.

### Polypeptide Immobilization

The enzymes or polypeptides according to the invention or for use in the processes of the invention can be used free or immobilized in the method described herein. An immobilized enzyme is an enzyme that is fixed to an inert carrier. Suitable carrier materials include for example clays, clay minerals, such as kaolinite, diatomaceous earth, perlite, silica, aluminum oxide, sodium carbonate, calcium carbonate, cellulose powder, anion exchanger materials, synthetic polymers, such as polystyrene, acrylic resins, phenol formaldehyde resins, polyurethanes and polyolefins, such as polyethylene and polypropylene. For making the supported enzymes, the carrier materials are usually employed in a finely-divided, particulate form, porous forms being preferred. The particle size of the carrier material is usually not more than 5 mm, in particular not more than 2 mm (particle-size distribution curve). Similarly, when using dehydrogenase as whole-cell catalyst, a free or immobilized form can be selected. Carrier materials are e.g. Ca-alginate, and carrageenan. Enzymes as well as cells can also be crosslinked directly with glutaraldehyde (cross-linking to CLEAs). Corresponding and other immobilization techniques are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim. Further information on biotransformations and bioreactors for carrying out methods according to the invention are also given for example in Rehm et al. (Ed.) Biotechnology, 2nd Edn, Vol 3, Chapter 17, VCH, Weinheim.

The present invention provides a process for making a compound of formula (I) as described comprising reacting a precursor compound of formula (Ia) as described herein with an acyltransferase enzyme to form a compound of formula (I).

The process of the invention can be performed as an in vitro or in vivo reaction under conditions conducive to the production of a compound of formula (I).

### Reaction Conditions for Biocatalytic Production Processes of the Invention

The at least one acyltransferase enzyme which is present during a process of the invention or an individual step of a multi-step method as defined herein, can be present in living cells naturally or recombinantly producing the enzyme or enzymes, in harvested cells, in dead cells, in permeabilized cells, in crude cell extracts, in purified extracts, or in essentially pure or completely pure form. The at least one enzyme may be present in solution or as an enzyme immobilized on a carrier or encapsulated. One or several enzymes may simultaneously be present in soluble and/or immobilized form.

The processes according to the invention can be performed in common reactors, which are known to those skilled in the art, and in different ranges of scale, e.g. from a laboratory scale (few milliliters to dozens of liters of reaction volume) to an industrial scale (several liters to thousands of cubic meters of reaction volume). If the enzyme is used in a form encapsulated by non-living, optionally permeabilized cells, in the form of a more or less purified cell extract or in purified form, a chemical reactor can be used. The chemical reactor usually allows controlling the amount of the at least one enzyme, the amount of the at least one substrate, the pH, the temperature and the circulation of the reaction medium. When the at least one polypeptide/enzyme is present in living cells, the process will be a fermentation. In this case the biocatalytic production will take place in a bioreactor (fermenter), where parameters necessary for suitable living conditions for the living cells (e.g. culture medium with nutrients, temperature, aeration, presence or absence of oxygen or other gases, antibiotics, and the like) can be controlled. Those skilled in the art are familiar with chemical reactors or bioreactors, e.g. with procedures for up-scaling chemical or biotechnological methods from laboratory scale to industrial scale, or for optimizing process parameters, which are also extensively described in the literature (for biotechnological methods see e.g. Crueger und Crueger, Biotechnologie—Lehrbuch der angewandten Mikrobiologie, 2. Ed., R. Oldenbourg Verlag, MOnchen, Wien, 1984).

Cells containing the at least one enzyme can be permeabilized by physical or mechanical means, such as ultrasound or radiofrequency pulses, French presses, or chemical means, such as hypotonic media, lytic enzymes and detergents present in the medium, or combination of such methods. Examples for detergents are digitonin, n-dodecylmaltoside, octylglycoside, Triton®X-100, Tween® 20, deoxycholate, CHAPS (3-[(3-Cholamidopropyl)dimethylammonio]-1-propansulfonate), Nonidet® P40 (Ethylphenolpoly(ethyleneglycolether), and the like.

Instead of living cells biomass of non-living cells containing the required biocatalyst(s) may be applied for the biotransformation reactions of the invention as well.

If the at least one enzyme is immobilized, it is attached to an inert carrier as described above.

The conversion reaction can be carried out batch wise, fed-batch, semi-batch wise or continuously. Reactants (and optionally nutrients) can be supplied at the start of reaction or can be supplied subsequently, either semi-continuously or continuously.

The reaction of the invention, depending on the particular reaction type, may be performed in an aqueous, aqueous-organic or non-aqueous reaction medium.

An aqueous or aqueous-organic medium may contain a suitable buffer in order to adjust the pH to a value in the range of 5 to 11, like 6 to 10.

In an aqueous-organic medium an organic solvent miscible, partly miscible or immiscible with water may be applied. Non-limiting examples of suitable organic solvents are listed below. Further examples are mono- or polyhydric, aromatic or aliphatic alcohols, in particular polyhydric aliphatic alcohols like glycerol.

The non-aqueous medium may be substantially free of water, i.e. may contain less that about 1 wt.-% or 0.5 wt.-% of water.

Biocatalytic methods may also be performed in an organic non-aqueous medium. A suitable organic solvents might be selected from aliphatic hydrocarbons having for example 5 to 8 carbon atoms, like pentane, cyclopentane, hexane, cyclohexane, heptane, octane or cyclooctane, chlorinated hydrocarbons, aromatic hydrocarbons like benzene, toluene, xylenes, chlorobenzene or dichlorobenzene, esters, such as ethylacetate, isopropylmyristate, ethers, like diethylether, methyl-tert.-butylether, ethyl-tert.-butylether, dipropylether, diisopropylether, dibutylether, tetrahydrofuran or 2-methyltetrahydrofuran, ketones and alcohols. Additional mediums include DMF, DMSO, deep eutectic solvent or ionic liquids. In the case of hydrolase-catalyzed ester synthesis, the acyl donor like ethyl acetate can be also used as medium.

The concentration of the reactants/substrates may be adapted to the optimum reaction conditions, which may depend on the specific enzyme applied. For example, the initial substrate concentration may be in the 0.001 to 1 M.

The reaction temperature may be adapted to the optimum reaction conditions, which may depend on the specific enzyme applied. For example, the reaction may be performed at a temperature in a range of from 0 to 70° C., as for example 20 to 50 or to 40° C. Examples for reaction temperatures are about 25° C., 28° C., 30° C., about 35° C., about 37° C., about 40° C., about 45° C., about 50° C., about 55° C. and about 60° C.

The process may proceed until equilibrium between the substrate and the product(s) is achieved, but may be stopped earlier. Usual process times are in the range from 10 minutes to 48 hours, in particular 1 hour to 24 hours, as for example in the range from 1 hour to 4 hours. These parameters are non-limiting examples of suitable process conditions.

The methodology of the present invention can further include a step of recovering an end or intermediate product, optionally in stereoisomerically or enantiomerically substantially pure form. The term “recovering” includes extracting, harvesting, isolating or purifying the compound from culture or reaction media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Identity and purity of the isolated product may be determined by known techniques, like High Performance Liquid Chromatography (HPLC), gas chromatography (GC), Spektroskopy (like IR, UV, NMR), Colouring methods, TLC, NIRS, enzymatic or microbial assays. (see for example: Patek et al. (1994) Appl. Environ. Microbiol. 60:133-140; Malakhova et al. (1996) Biotekhnologiya 11 27-32; und Schmidt et al. (1998) Bioprocess Engineer. 19:67-70. Ullmann's Encyclopedia of Industrial Chemistry (1996) Bd. A27, VCH: Weinheim, S. 89-90, S. 521-540, S. 540-547, S. 559-566, 575-581 und S. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Bd. 17.)

### Fermentative Production

The invention also relates to process for the fermentative production of a compound of formula (I).

A fermentation as used according to the present invention can, for example, be performed in stirred fermenters, bubble columns and loop reactors. A comprehensive overview of the possible method types including stirrer types and geometric designs can be found in “Chmiel: Bioprozesstechnik: Einfuhrung in die Bioverfahrenstechnik, Band 1”. In the process of the invention, typical variants available are the following variants known to those skilled in the art or explained, for example, in “Chmiel, Hammes and Bailey: Biochemical Engineering”, such as batch, fed-batch, repeated fed-batch or else continuous fermentation with and without recycling of the biomass. Depending on the production strain, sparging with air, oxygen, carbon dioxide, hydrogen, nitrogen or appropriate gas mixtures may be effected in order to achieve good yield (YP/S).

The culture medium that is to be used must satisfy the requirements of the particular strains in an appropriate manner.

These media that can be used according to the invention may comprise one or more sources of carbon, sources of nitrogen, inorganic salts, vitamins and/or trace elements.

Preferred sources of carbon are sugars, such as mono-, di- or polysaccharides. Very good sources of carbon are for example glucose, fructose, mannose, galactose, ribose, sorbose, ribulose, lactose, maltose, sucrose, raffinose, starch or cellulose.

Sugars can also be added to the media via complex compounds, such as molasses, or other by-products from sugar refining. It may also be advantageous to add mixtures of various sources of carbon. Other possible sources of carbon are oils and fats such as soybean oil, sunflower oil, peanut oil and coconut oil, fatty acids such as palmitic acid, stearic acid or linoleic acid, alcohols such as glycerol, methanol or ethanol and organic acids such as acetic acid or lactic acid.

Sources of nitrogen are usually organic or inorganic nitrogen compounds or materials containing these compounds. Examples of sources of nitrogen include ammonia gas or ammonium salts, such as ammonium sulfate, ammonium chloride, ammonium phosphate, ammonium carbonate or ammonium nitrate, nitrates, urea, amino acids or complex sources of nitrogen, such as corn-steep liquor, soybean flour, soy-bean protein, yeast extract, meat extract and others. The sources of nitrogen can be used separately or as a mixture.

Inorganic salt compounds that may be present in the media comprise the chloride, phosphate or sulfate salts of calcium, magnesium, sodium, cobalt, molybdenum, potassium, manganese, zinc, copper and iron.

Inorganic sulfur-containing compounds, for example sulfates, sulfites, dithionites, tetrathionates, thiosulfates, sulfides, but also organic sulfur compounds, such as mercaptans and thiols, can be used as sources of sulfur.

Phosphoric acid, potassium dihydrogen phosphate or dipotassium hydrogen phosphate or the corresponding sodium-containing salts can be used as sources of phosphorus.

Chelating agents can be added to the medium, in order to keep the metal ions in solution. Especially suitable chelating agents comprise dihydroxyphenols, such as catechol or protocatechuate, or organic acids, such as citric acid.

The fermentation media used according to the invention may also contain other growth factors, such as vitamins or growth promoters, which include for example biotin, riboflavin, thiamine, folic acid, nicotinic acid, pantothenate and pyridoxine. Growth factors and salts often come from complex components of the media, such as yeast extract, molasses, corn-steep liquor and the like. In addition, suitable precursors can be added to the culture medium. The precise composition of the compounds in the medium is strongly dependent on the particular experiment and must be decided individually for each specific case. Information on media optimization can be found in the textbook “Applied Microbiol. Physiology, A Practical Approach” (1997) Growing media can also be obtained from commercial suppliers, such as Standard 1 (Merck) or BHI (Brain heart infusion, DIFCO) etc.

All components of the medium are sterilized, either by heating (20 min at 1.5 bar and 121° C.) or by sterile filtration. The components can be sterilized either together, or if necessary separately. All the components of the medium can be present at the start of growing, or optionally can be added continuously or by batch feed.

In some embodiments, the fermentation process can include the use of an overlay of an organic liquid phase immiscible with aqueous phase.

In-situ liquid-liquid extraction (biphasic fermentation) is a strategy that can be employed in accordance with the present invention for physical separation of product from microorganisms via partitioning into the water immiscible organic liquid phase from an aqueous culture phase. The organic liquid phase or organic phase is present as either an overlay if its density is less than that of the aqueous phase, or an underlay if its density is greater than that of the aqueous phase.

In one embodiment, the organic phase immiscible with aqueous phase, or simply the organic phase, comprises an alkane, an alcohol with carbon number greater than 4, an ester (such as isopropyl myristate), a triglyceride (including commercially available vegetable oils such as sunflower oil, soybean oil, or olive oil), a diester (such as dialkyl malonate), a ketone, or a glyme. Other organic solvents immiscible with water or the aqueous phase employed can be utilized. In another embodiment, the organic phase comprises isopropyl myristate. Suitable solvents include without limitation, other esters, aromatic solvents, and the likes. In one embodiment, the organic phase comprises an aromatic solvent. Non limiting examples of aromatic hydrocarbon solvents include benzene, toluene, other alkylated benzenes, anisole and the likes, and mixtures thereof. In one embodiment, the organic phase comprises toluene. Further embodiments include hexane or dodecane.

The temperature of the culture is normally between 15° C. and 45° C., preferably 25° C. to 40° C. and can be kept constant or can be varied during the experiment. In certain embodiments, the cells are eukaryotic, e.g. yeast, and the temperature is preferably in the range from 28° C. to 34° C. In certain embodiments, the cells are prokaryotic, e.g. bacteria, and the temperature is preferably in the range from 30° C. to 40° C., for instance 37° C.

The pH value of the medium should be in the range from 4 to 8.5. In certain embodiments, the cells are eukaryotic, e.g. yeast, and the pH is preferably from about 4.0 to about 6.5. In certain embodiments, the cells are prokaryotic, e.g. bacteria, and the pH is from about 6.5 to about 7.5, e.g. about 7.0. The pH value for growing can be controlled during growing by adding basic compounds such as sodium hydroxide, potassium hydroxide, ammonia or ammonia water or acid compounds such as phosphoric acid or sulfuric acid. Antifoaming agents, e.g. fatty acid polyglycol esters, can be used for controlling foaming. To maintain the stability of plasmids, suitable substances with selective action, e.g. antibiotics, can be added to the medium. Oxygen or oxygen-containing gas mixtures, e.g. the ambient air, are fed into the culture in order to maintain aerobic conditions. The temperature of the culture is normally from 20° C. to 45° C. Culture is continued until a maximum of the desired product has formed. This is normally achieved within 1 hour to 160 hours.

The processes of the present invention can further include a step of recovering a compound of formula (I).

The term “recovering” includes extracting, harvesting, isolating or purifying the compound from culture media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like.

Before the intended isolation the biomass of the broth can be removed. Processes for removing the biomass are known to those skilled in the art, for example filtration, sedimentation and flotation. Consequently, the biomass can be removed, for example, with centrifuges, separators, decanters, filters or in flotation apparatus. For maximum recovery of the product of value, washing of the biomass is often advisable, for example in the form of a diafiltration. The selection of the method is dependent upon the biomass content in the fermenter broth and the properties of the biomass, and also the interaction of the biomass with the product of value.

In one embodiment, the fermentation broth can be sterilized or pasteurized. In a further embodiment, the fermentation broth is concentrated. Depending on the requirement, this concentration can be done batch wise or continuously. The pressure and temperature range should be selected such that firstly no product damage occurs, and secondly minimal use of apparatus and energy is necessary. The skillful selection of pressure and temperature levels for a multistage evaporation in particular enables saving of energy.

### Embodiments of the Process of the Invention

A preferred embodiment of the process of the invention is wherein the compound of formula (Ia) is aromadendrin and the compound of formula (I) is aromadendrin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is aromadendrin which is a compound known in the art. The preferred IUPAC name is (2R,3R)-3,5,7-trihydroxy-2-(4-hydroxyphenyl)-2,3-dihydrochromen-4-one.

A further preferred embodiment of the process of the invention is wherein the compound of formula (Ia) is taxifolin and the compound of formula (I) is taxifolin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is taxifolin which is a compound known in the art. The preferred IUPAC name is (2R,3R)-2-(3,4-dihydroxyphenyl)-3,5,7-trihydroxy-2,3-dihydrochromen-4-one.

A further preferred embodiment of the process of the invention is wherein the compound of formula (Ia) is dihydrotamarixetin and the compound of formula (I) is dihydrotamarixetin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is dihydrotamarixetin which is a compound known in the art. The preferred chemical name is (2R,3R)-3,5,7-trihydroxy-2-(3-hydroxy-4-methoxyphenyl)-2,3-dihydrochromen-4-one.

A further preferred embodiment of the process of the invention is wherein the compound of formula (Ia) is 3′-O-methyltaxifolin and the compound of formula (I) is 3′-O-methyltaxifolin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 3′-O-methyltaxifolin which is a compound known in the art. The preferred chemical name is (2R,3R)-3,5,7-trihydroxy-2-(4-hydroxy-3-methoxyphenyl)-2,3-dihydrochromen-4-one.

A further preferred embodiment of the process of the invention is wherein the compound of formula (Ia) is pinobanksin and the compound of formula (I) is pinobanksin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is pinobanksin which is a compound known in the art. The preferred chemical name is (2R,3R)-3,5,7-trihydroxy-2-phenyl-2,3-dihydrochromen-4-one.

A further preferred embodiment of the process of the invention is wherein the compound of formula (Ia) is 5-deoxyaromadendrin and the compound of formula (I) is 5-deoxyaromadendrin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 5-deoxyaromadendrin which is a compound known in the art. The preferred IUPAC name is (2R,3R)-3,7-dihydroxy-2-(4-hydroxyphenyl)-2,3-dihydrochromen-4-one.

A further preferred embodiment of the process of the invention is wherein the compound of formula (Ia) is 5-deoxytaxifolin and the compound of formula (I) is 5-deoxytaxifolin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 5-deoxytaxifolin which is a compound known in the art. The preferred IUPAC name is (2R,3R)-3,7-dihydroxy-2-(3,4-dihydroxyphenyl)-2,3-dihydrochromen-4-one.

A further preferred embodiment of the process of the invention is wherein the compound of formula (Ia) is 5-deoxydihydrotamarixetin and the compound of formula (I) is 5-deoxydihydrotamarixetin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 5-deoxydihydrotamarixetin which is a compound known in the art. The preferred IUPAC name is (2R,3R)-3,7-dihydroxy-2-(3-hydroxy-4-methoxyphenyl)-2,3-dihydrochromen-4-one.

A further preferred embodiment of the process of the invention is wherein the compound of formula (Ia) is 5-deoxy-3′-O-methyltaxifolin and the compound of formula (I) is 5-deoxy-3′-O-methyltaxifolin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 5-deoxy-3′-O-methyltaxifolin which is a compound known in the art. The preferred IUPAC name is (2R,3R)-3,7-dihydroxy-2-(4-hydroxy-3-methoxyphenyl)-2,3-dihydrochromen-4-one.

A further preferred embodiment of the process of the invention is wherein the compound of formula (Ia) is 5-deoxypinobanksin and the compound of formula (I) is 5-deoxypinobanksin-3-O-acetate. Preferably the enzyme is an acetyltransferase and the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. In this embodiment the substrate is 5-deoxypinobanksin which is a compound known in the art. The preferred IUPAC name is (2R,3R)-3,7-dihydroxy-2-phenyl-2,3-dihydrochromen-4-one.

An embodiment of the invention is wherein the process is performed in the presence of acyl-CoA. More preferably the acyl-CoA is acetyl-CoA.

Acetyl-CoA is a well-known molecule that participates in many biochemical reactions. It can be commonly obtained from many reagent suppliers, for example Sigma. Where the process is an in vivo process then the acetyl-CoA can be produced by the cells.

An alternative aspect of the invention provides process for making a compound of formula (I) the process comprising reacting a precursor compound of formula (Ia) in the presence of a hydrolase. Preferably the hydrolase is a lipase, as described herein.

Where the enzyme is a hydrolase, suitable acetate esters to be used for acetylation of formula (Ia) are selected from either unsaturated, saturated or aromatic acetates including but not limited to vinyl acetate, phenylvinyl acetate, ethoxyvinyl acetate, isoprenyl acetate, isopropenyl acetate, or ethyl acetate, preferably ethyl acetate, then the source of the acetate molecule for the process is from ethylacetate, vinylacetate or other such molecules as can be appreciated by the skilled person.

Embodiments of this aspect of the invention include all the embodiments listed above where the enzyme is an acetyltransferase.

For the avoidance of doubt, the processes of the invention presented herein can make a compound of formula (I) and further reaction products which may be generated according to the substrate material and process conditions.

### Multi-Enzyme Process of the Invention

The flavonoid biosynthetic pathway in plants is known. The general upstream shikimate pathway converts chorismate to phenylalanine and L-tyrosine via several well characterized enzymes. Chorismate can be obtained from sugar such as glucose.

The flavonoid biosynthetic pathway starts with the conversion of L-phenylalanine into trans-cinnamic acid through the non-oxidative deamination by phenylalanine ammonia lyase (PAL). Next, trans-cinnamic acid is hydroxylated at the para position to p-coumaric acid (4-hydroxycinnamic acid) by cinnamate-4-hydroxylase (C4H). C4H is a cytochrome P450 monooxygenase, that benefits from regeneration by a cytochrome P450 reductase (CPR). As an alternative route, the amino acid L-tyrosine can be converted into p-coumaric acid by a tyrosine ammonia lyase (TAL). p-Coumaric acid is subsequently activated to p-coumaroyl-CoA by the 4-coumarate-CoA ligase (4CL). From here the biosynthetic pathway gives rise to many compounds, for example flavonoids, stilbenoids, curcuminoids and lignins.

In the case of flavonoids, a chalcone synthase (CHS) and a chalcone isomerase (CHI) catalyze the condensation of p-coumaroyl-CoA with three molecules of malonyl-CoA, resulting in the formation of naringenin chalcone and finally naringenin. Optionally, the expression of a chalcone isomerase-like (CHIL) protein has some positive effect on the CHS activity.

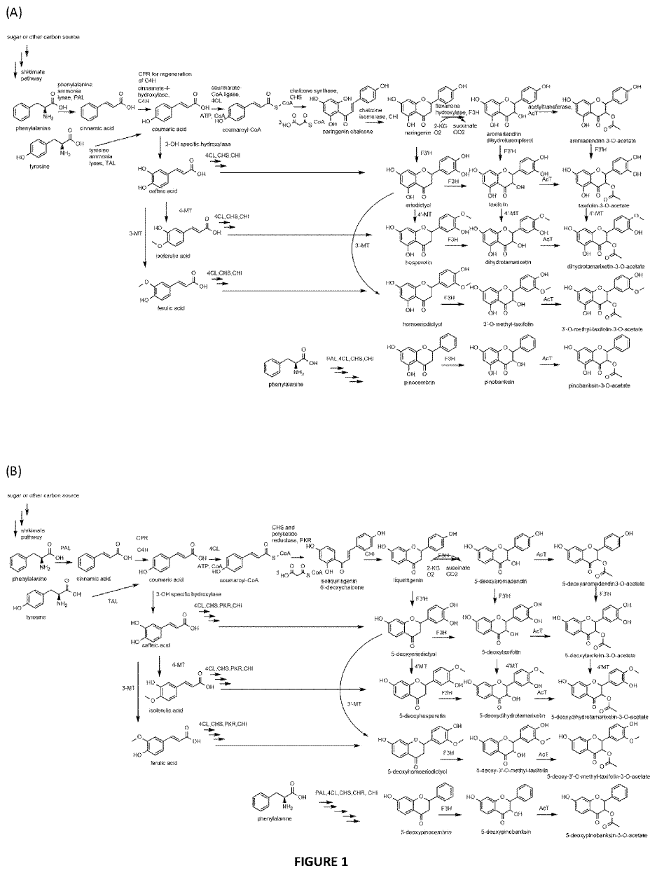

At this point, naringenin is subjected to 3-hydroxylation of naringenin to obtain aromadendrin (an example of a compound of formula (Ia) using a flavanone-3-hydroxylase (F3H) and subsequent O-acetylation at this position using the acyltransferase process of the current invention to obtain aromadendrin-3-acetate, an example of a compound of formula (I). A schematic of this process is shown in FIG. 1.

In addition to the central enzymes provide above, a flavonoid 3-hydroxylase (F3′H) enzyme, can add a hydroxy group to the 3′-position of naringenin, which in combination with F3H and the acetyltransferase used in the process of the invention, would give taxifolin-3-O-acetate, a compound of formula (I). F3′H is a P450 monooxygenase that benefits from regeneration by a cytochrome P450 reductase (CPR).

Alternatively in addition to the central enzymes provided above, a flavonoid 3′-hydroxylase (F3′H) enzyme, can add a hydroxy group to the 3′-position of aromadendrin, which in combination with the acetyltransferase used in the process of the invention would give taxifolin-3-O-acetate, a compound of formula (I). F3′H is a P450 monooxygenase that benefits from regeneration by a cytochrome P450 reductase (CPR).

Another possibility to obtain eriodictyol and taxifolin is the use of caffeic acid as starting molecule or by hydroxylating coumaric acid at position 3 by application of a 3-OH specific hydroxylase, such as coumaric acid 3-hydroxylase or 4-hydroxybenzoate-m-hydroxylase.

In addition to the enzymes mentioned above, a 4′-O-methyltransferase can add a methyl group to 4′—OH of eriodictyol or taxifolin, which in combination with F3H and the acetyltransferase used in the process of the invention, or the acetyltransferases used in the process of the invention, respectively, would give dihydrotamarixetin-3-O-acetate, a compound of formula (I). 4′-MT might be further engineered to improve its selectivity for the 4′—OH position. 4′-MT as most methyltransferases is S-adenosylmethionine (SAM) cofactor dependent and benefits from regeneration of SAM, which can be provided either in vivo by the (for this purpose engineered) cell metabolism or in vitro by the addition of multiple enzymes, such as SAH hydrolase (SAHH), methionine synthase (MS), methionine adenosyltransferase (MAT), adenosine kinase (ADK) and polyphosphate kinases (PPK2) I and II.

Another possibility to obtain hesperetin and dihydrotamarixetin is the use of isoferulic acid as starting material or by methylating caffeic acid at position 4-OH by application of a 4-O-methyltransferase or caffeoyl-CoA by a 4—OH specific caffeoyl-O-methyltransferase. Alternatively, coumaric acid can be methylated at position 4 to give 4-methoxy cinnamic acid, and subsequently hydroxylated at positions 3 by application of a 3-OH specific hydroxylase, such as coumaric acid 3-hydroxylase or 4-hydroxybenzoate-m-hydroxylase. Another possibility is the use of 4-methoxy cinnamic acid as starting material and hydroxylation at position 3 by application of a 3-OH specific hydroxylase, such as coumaric acid 3-hydroxylase or 4-hydroxybenzoate-m-hydroxylase. 4-MT might be further engineered to improve its selectivity for the 4—OH position. 4-MT as most methyltransferases is S-adenosylmethionine (SAM) cofactor dependent and benefits from regeneration of SAM, which can be provided either in vivo by the (for this purpose engineered) cell metabolism or in vitro by the addition of multiple enzymes, such as SAH hydrolase (SAHH), methionine synthase (MS), methionine adenosyltransferase (MAT), adenosine kinase (ADK) and polyphosphate kinases (PPK2) I and II. In addition to the enzymes mentioned above, a 3′-O-methyltransferase can add a methyl group to 3′-OH of eriodictyol or taxifolin, which in combination with F3H and the acetyltransferase used in the process of the invention, or the acetyltransferase used in the process of the invention, respectively, would give 3′-O-methyl-taxifolin-3-O-acetate, a compound of formula (I). 3′-MT might be further engineered to improve its selectivity for the 3′-OH position. 3′-MT as most methyltransferases is S-adenosylmethionine (SAM) cofactor dependent and benefits from regeneration of SAM, which can be provided either in vivo by the (for this purpose engineered) cell metabolism or in vitro by the addition of multiple enzymes, such as SAH hydrolase (SAHH), methionine synthase (MS), methionine adenosyltransferase (MAT), adenosine kinase (ADK) and polyphosphate kinases (PPK2) I and II.

Another possibility to obtain homoeriodictyol and 3′-O-methyltaxifolin is the use of ferulic acid as starting material or by methylating caffeic acid at position 3-OH by application of a 3-O-methyltransferase caffeoyl-CoA by a 3-OH specific caffeoyl-O-methyltransferase. 3-MT might be further engineered to improve its selectivity for the 3-OH position. 3-MT as most methyltransferases is S-adenosylmethionine (SAM) cofactor dependent and benefits from regeneration of SAM, which can be provided either in vivo by the (for this purpose engineered) cell metabolism or in vitro by the addition of multiple enzymes, such as SAH hydrolase (SAHH), methionine synthase (MS), methionine adenosyltransferase (MAT), adenosine kinase (ADK) and polyphosphate kinases (PPK2) I and II.

In the case of 5-deoxyflavonoids a polyketide reductase (PKR) is added to the pathway. A PKR coupled with a CHS catalyzes the reduction of a specific keto group of the tetraketide intermediate resulting in 6′-deoxychalcones. Spontaneous or CHI catalyzed ring closure results in 5-deoxyflavanones such as liquiritigenin (5-deoxynaringenin) and/or 5-deoxypinocembrin. 3-hydroxylation using a flavanone-3-hydroxylase (F3H) and subsequent O-acetylation at this position using the acyltransferase process of the current invention obtain 5-deoxyaromadendrin-3-O-acetate and 5-deoxypinobanksin-3-O-acetate, examples of a compound of formula (I). To obtain 5-deoxytaxifolin-3-O-acetate a hydroxylase is added to the pathway as described for taxifolin-3-O-acetate above.

To obtain 5-deoxydihydrotamarixetin-3-O-acetate and 5-deoxy-3′-O-methyltaxifolin-3-O-acetate further methyltransferases are added to the pathways as described for dihydrotamarixetin-3-O-acetate and 3′-O-methyltaxifolin-3-O-acetate.

There are further P450 monooxygenase enzymes which add OH groups to the intermediate compounds to the making of a compound of formula (I). Furthermore, various hydroxy groups on both aromatic rings can be modified by methyltransferases, and also glycosyltransferases, which may be used to make glycosylated derivatives of a compound of formula (I).

Hence as can be appreciated, by using the enzymes listed herein it is possible to convert phenylalanine, tyrosine, sugar and/or other carbon sources to a compound of formula (I), preferably aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate. This can be performed using a series of cascading enzyme reactions. Such methods can be performed using phenylalanine and/or tyrosine as an initial substrate, or carbon sources such as sugar and glycerol if appropriate upstream enzymes are used.

For the avoidance of doubt, glycerol can also be used in replacement or in combination with glucose or any other carbon source in the processes of the invention disclosed herein.

Hence a preferred embodiment of the invention is wherein the process of making a compound of formula (I) further comprises using one or more of the following enzyme(s):
(a) flavanone 3-hydroxylase (F3H),
(b) chalcone isomerase (CHI),
(c) Chalcone synthase (CHS),
(d) 4-coumarate-coenzyme A ligase (4CL),
(e) cytochrome P450 reductase (CPR),
(f) tyrosine ammonia lyase (TAL),
(g) chalcone isomerase-like (CHIL),
(h) cinnamate-4-hydroxylase (C4H),
(i) phenylalanine ammonia lyase (PAL),
(j) flavonoid 3-hydroxylase (F3′H),
(k) 3′-O-methyltransferase (3′-MT),
(l) 4′-O-methyltransferase (4′-MT),
(m) 3-O-methyltransferase (3-MT),
(n) 4-O-methyltransferase (4-MT),
(o) 3-OH specific P450 monooxygenase,
(p) glycosidase, and/or
(q) polyketide reductase (PKR)

In addition to the list of enzymes provided above, further hydroxylases e.g. P450 monooxygenases specific adding OH to other positions and methyltransferases specific for methylation of other positions can be identified and adopted by the skilled person to make modifications to the compounds which can be used in the process of the invention. Glycosyltransferases may be used to make glycosylated derivatives of a compound of formula (I).

Particular combinations of the enzymes listed here are preferred according to whether the process of the invention is performed (i) in vitro or in vivo, (ii) the case of in vivo the background genetics of the recombinant host strain used (iii) the starting material for the process, and (iv) the preferred compound of formula (I) to be made.

In one embodiment of the invention the process is an in vitro or in vivo process and the starting material is glucose (or another carbon source), phenylalanine and/or tyrosine and the compound of formula (I) is aromadendrin-3-O-acetate. In this embodiment of the invention the process comprises the following enzyme(s): phenylalanine ammonia lyase (PAL), cinnamate-4-hydroxylase (C4H), cytochrome P450 reductase (CPR), tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed process one herein.

In another embodiment of the invention the process is an in vivo process, the starting material is glucose (or another carbon source), phenylalanine and/or tyrosine and the compound of formula (I) is aromadendrin-3-O-acetate and the microbial host cell comprises a functional CPR, for example a yeast cell. In this embodiment of the invention the process comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed process two herein.

In another embodiment of the invention the process is an in vitro or in vivo process, the starting material is glucose (or another carbon source), phenylalanine and/or tyrosine and the compound of formula (I) is aromadendrin-3-O-acetate. Here the CHIL enzyme is omitted since this enzyme is not necessary for the performance of the process. In this embodiment of the invention the process comprises the following enzyme(s): phenylalanine ammonia lyase (PAL), cinnamate-4-hydroxylase (C4H), cytochrome P450 reductase (CPR), tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed process three herein.

In another embodiment of the invention the process is an in vivo process, the starting material is glucose (or another carbon source), phenylalanine and/or tyrosine and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the process comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed process four herein.

In another embodiment of the invention the process is an in vitro or in vivo process, the starting material is glucose (or another carbon source), phenylalanine and/or tyrosine and the compound of formula (I) is aromadendrin-3-O-acetate. Here the CHIL and CHI enzymes are omitted since these enzymes are not necessary for the performance of the process. In this embodiment of the invention the process comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), flavanone-3-hydroxylase (F3H) and an acetyltransferase. This embodiment is termed process five herein.

In another embodiment of the invention the process is an in vivo process, the starting material is glucose (or another carbon source), phenylalanine and/or tyrosine and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the process comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), flavanone-3-hydroxylase (F3H) and an acetyltransferase. This embodiment is termed process six herein.

In one embodiment of the invention the process is an in vitro or in vivo process and the starting material is glucose (or another carbon source) and/or phenylalanine and the compound of formula (I) is aromadendrin-3-O-acetate. In this embodiment of the invention the process comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed process seven herein.

In another embodiment of the invention the process is an in vivo process, the starting material is glucose (or another carbon source) and/or phenylalanine and the compound of formula (I) is aromadendrin-3-O-acetate and the microbial host cell comprises a functional CPR, for example a yeast cell. In this embodiment of the invention the process comprises the following enzyme(s): phenylalanine ammonia lyase (PAL), cinnamate-4-hydroxylase (C4H), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed process eight herein.

In another embodiment of the invention the process is an in vitro or in vivo process, the starting material is glucose (or another carbon source) and/or phenylalanine and the compound of formula (I) is aromadendrin-3-O-acetate. Here the CHIL enzyme is omitted since this enzyme is not necessary for the performance of the process. In this embodiment of the invention the process comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), chalcone isomerase (CHI), flavanone-3-hydroxylase (F3H) and an acetyltransferase. This embodiment is termed process nine herein.

In another embodiment of the invention the process is an in vivo process, the starting material is glucose (or another carbon source) and/or phenylalanine and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the process comprises the following enzyme(s): phenylalanine ammonia lyase (PAL), cinnamate-4-hydroxylase (C4H), 4-coumarate-CoA ligase (4CL), chalcone synthase (CHS), chalcone isomerase (CHI), flavanone-3-hydroxylase (F3H) and an acetyltransferase. This embodiment is termed process ten herein.

In another embodiment of the invention the process is an in vitro or in vivo process, the starting material is glucose (or another carbon source) and/or phenylalanine and the compound of formula (I) is aromadendrin-3-O-acetate. Here the CHIL and CHI enzymes are omitted since these enzymes are not necessary for the performance of the process. In this embodiment of the invention the process comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), flavanone-3-hydroxylase (F3H) and an acetyltransferase. This embodiment is termed process 11 herein.

In another embodiment of the invention the process is an in vivo process, the starting material is glucose (or another carbon source), and/or phenylalanine and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the process comprises the following enzyme(s): phenylalanine ammonia lyase (PAL), cinnamate-4-hydroxylase (C4H), 4-coumarate-CoA ligase (4CL), chalcone synthase (CHS), flavanone-3-hydroxylase (F3H) and an acetyltransferase. This embodiment is termed process 12 herein.

In one embodiment of the invention the process is an in vitro or in vivo process and the starting material is glucose (or another carbon source) and/or tyrosine and the compound of formula (I) is aromadendrin-3-O-acetate. In this embodiment of the invention the process comprises the following enzyme(s): tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed process 13 herein.

In one embodiment of the invention the process is an in vitro or in vivo process and the starting material is glucose (or another carbon source) and/or tyrosine and the compound of formula (I) is aromadendrin-3-O-acetate. Here the CHIL enzyme is omitted since this enzyme is not necessary for the performance of the process. In this embodiment of the invention the process comprises the following enzyme(s): tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed process 14 herein.

In one embodiment of the invention the process is an in vitro or in vivo process and the starting material is glucose (or another carbon source) and/or tyrosine and the compound of formula (I) is aromadendrin-3-O-acetate. Here the CHIL and CHI enzymes are omitted since these enzymes are not necessary for the performance of the process. In this embodiment of the invention the process comprises the following enzyme(s): tyrosine ammonia lyase (TAL), 4-coumarate-CoA ligase (4CL), chalcone synthase (CHS), flavanone-3-hydroxylase (F3H) and an acetyltransferase. This embodiment is termed process 15 herein.

In another embodiment of the invention the process is an in vitro or in vivo process, the starting material is cinnamic acid and the compound of formula (I) is aromadendrin-3-O-acetate. In this embodiment of the invention the process comprises the following enzyme(s): cinnamate-4-hydroxylase (C4H), cytochrome P450 reductase (CPR), 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed process 16 herein.

In another embodiment of the invention the process is an in vivo process, the starting material is cinnamic acid and the compound of formula (I) is aromadendrin-3-O-acetate and the microbial host cell comprises a functional CPR, for example a yeast cell. In this embodiment of the invention the process comprises the following enzyme(s): cinnamate-4-hydroxylase (C4H), 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed process 17 herein.

In one embodiment of the invention the process is an in vitro or in vivo process and the starting material is cinnamic acid and the compound of formula (I) is aromadendrin-3-O-acetate. Here the CHIL enzyme is omitted since this enzyme is not necessary for the performance of the process. In this embodiment of the invention the process comprises the following enzyme(s): cinnamate-4-hydroxylase (C4H), cytochrome P450 reductase (CPR), 4-coumarate-CoA ligase (4CL), chalcone synthase (CHS), chalcone isomerase (CHI), flavanone-3-hydroxylase (F3H) and acetyltransferase. This embodiment is termed process 18 herein.

In another embodiment of the invention the process is an in vivo process, the starting material is cinnamic acid and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the process comprises the following enzyme(s): cinnamate-4-hydroxylase (C4H), 4-coumarate-CoA ligase (4CL), chalcone synthase (CHS), chalcone isomerase (CHI), flavanone-3-hydroxylase (F3H) and acetyltransferase. This embodiment is termed process 19 herein.

In another embodiment of the invention the process is an in vitro or in vivo process and the starting material is cinnamic acid and the compound of formula (I) is aromadendrin-3-O-acetate. Here the CHIL and CHI enzymes are omitted since these enzymes are not necessary for the performance of the process. In this embodiment of the invention the process comprises the following enzyme(s): cinnamate-4-hydroxylase (C4H), cytochrome P450 reductase (CPR), 4-coumarate-CoA ligase (4CL), chalcone synthase (CHS), flavanone-3-hydroxylase (F3H) and acetyltransferase. This embodiment is termed process 20 herein.

In another embodiment of the invention the process is an in vivo process, the starting material is cinnamic acid and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the process comprises the following enzyme(s): cinnamate-4-hydroxylase (C4H), 4-coumarate-CoA ligase (4CL), chalcone synthase (CHS), flavanone-3-hydroxylase (F3H) and acetyltransferase. This embodiment is termed process 21 herein.

In another embodiment of the invention the process is an in vitro or in vivo process and the starting material is coumaric acid and the compound of formula (I) is aromadendrin-3-O-acetate. In this embodiment of the invention the process comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed process 22 herein.

In another embodiment of the invention the process is an in vitro or in vivo process and the starting material is coumaric acid and the compound of formula (I) is aromadendrin-3-O-acetate. Here the CHIL enzyme is omitted since this enzyme is not necessary for the performance of the process. In this embodiment of the invention the process comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed process 23 herein.

In another embodiment of the invention the process is an in vitro or in vivo process and the starting material is coumaric acid and the compound of formula (I) is aromadendrin-3-O-acetate. Here the CHIL and CHI enzymes are omitted since these enzymes are not necessary for the performance of the process. In this embodiment of the invention the process comprises the following enzyme(s): 4-coumarate-CoA ligase (4CL), chalcone synthase (CHS), flavanone-3-hydroxylase (F3H) and acetyltransferase. This embodiment is termed process 24 herein.

In another embodiment of the invention the process is an in vitro or in vivo process and the starting material is coumaroyl-CoA and the compound of formula (I) is aromadendrin-3-O-acetate. In this embodiment of the invention the process comprises the following enzyme(s): chalcone synthase (CHS), chalcone isomerase (CHI), chalcone isomerase-like protein (CHIL), flavanone-3-hydroxylase (F3H) and acetyltransferase. This embodiment is termed process 25 herein.

In another embodiment of the invention the process is an in vitro or in vivo process and the starting material is coumaroyl-CoA and the compound of formula (I) is aromadendrin-3-O-acetate. Here the CHIL enzyme is omitted since this enzyme is not necessary for the performance of the process. In this embodiment of the invention the process comprises the following enzyme(s): chalcone synthase (CHS), chalcone isomerase (CHI), flavanone-3-hydroxylase (F3H) and acetyltransferase. This embodiment is termed process 26 herein.

In another embodiment of the invention the process is an in vitro or in vivo process and the starting material is coumaroyl-CoA and the compound of formula (I) is aromadendrin-3-O-acetate. Here the CHIL and CHI enzymes are omitted since these enzymes are not necessary for the performance of the process. In this embodiment of the invention the process comprises the following enzyme(s): chalcone synthase (CHS), flavanone-3-hydroxylase (F3H) and acetyltransferase. This embodiment is termed process 27 herein.

In another embodiment of the invention the process is an in vitro or in vivo process and the starting material is naringenin and the compound of formula (I) is aromadendrin-3-O-acetate. In this embodiment of the invention the process comprises the following enzyme(s): flavanone-3-hydroxylase (F3H) and acetyltransferase. This embodiment is termed process 28 herein.

In another embodiment of the invention the process is an in vitro or in vivo process and the starting material is aromadendrin and the compound of formula (I) is aromadendrin-3-O-acetate. In this embodiment of the invention the process comprises the following enzyme(s): acetyltransferase. This embodiment is termed process 29 herein.

In another embodiment of the invention the process is an in vitro or in vivo process and the starting material is a glycosylated precursor such as for example naringin and the compound of formula (I) is aromadendrin-3-O-acetate. In this embodiment of the invention the process comprises the following enzyme(s): glycosidase (or alternatively a chemical hydrolysis step if performed in vitro), flavanone-3-hydroxylase (F3H) and acetyltransferase. This embodiment is termed process 30 herein.

In another embodiment of the invention the process is an in vitro or in vivo process and the starting material is a glycosylated precursor such as for example engeletin and the compound of formula (I) is aromadendrin-3-O-acetate. In this embodiment of the invention the process comprises the following enzyme(s): glycosidase (or alternatively a chemical hydrolysis step if performed in vitro), and acetyltransferase. This embodiment is termed process 31 herein.

In addition to the preparation of aromadendrin-3-O-acetate, the enzyme combinations used in the processes of the invention numbered 1 to 31 can also be used to prepare the taxifolin-3-O-acetate (a compound of formula (I)) with the use of the additional enzyme flavonoid 3-hydroxylase (F3′H). Hence further embodiments of the invention provide a process of preparing taxifolin-3-O-acetate comprising the enzymes listed in processes 1 to 31 and flavonoid 3-hydroxylase (F3′H). As can be appreciated by the skilled person, the addition of F3′H to any one of processes 1 to 31 results in an additional 31 processes. These are herein termed process 32 to 62. Hence for example, process 32 is that of process 1 with the addition of F3′H, and so on. Alternatively, final 3′-hydroxylation can also be obtained by hydroxylating position 3 of phenylalanine, tyrosine, cinnamic acid, coumaric acid or coumaroyl-CoA present as intermediate or starting material in processes 1 to 27 with the use of an additional enzyme coumaric acid 3-hydroxylase or 4-hydroxybenzoate-m-hydroxylase in process 1 to 27. The use of the additional enzyme coumaric acid 3-hydroxylase or 4-hydroxybenzoate-m-hydroxylase in any of process 1 to 27 results in an additional 27 processes. These are herein termed process 63 to 89. Hence for example, process 63 is that of process 1 with the addition of coumaric acid 3-hydroxylase or 4-hydroxybenzoate-m-hydroxylase, and so on.

In another embodiment of the invention the process is an in vitro or in vivo process and the starting material caffeic acid and the compound of formula (I) is taxifolin-3-O-acetate. In this embodiment of the invention the process comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed process 90 herein.

In another embodiment of the invention the process is an in vitro or in vivo process and the starting material caffeic acid and the compound of formula (I) is taxifolin-3-O-acetate. Here the CHIL enzyme is omitted since this enzyme is not necessary for the performance of the process. In this embodiment of the invention the process comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed process 91 herein.

In another embodiment of the invention the process is an in vitro or in vivo process and the starting material caffeic acid and the compound of formula (I) is taxifolin-3-O-acetate. Here the CHIL and CHI enzymes are omitted since these enzymes are not necessary for the performance of the process. In this embodiment of the invention the process comprises the following enzyme(s): 4-coumarate-CoA ligase (4CL), chalcone synthase (CHS), flavanone-3-hydroxylase (F3H) and acetyltransferase. This embodiment is termed process 92 herein.

In another embodiment of the invention the process is an in vitro or in vivo process and the starting material eriodictyol and the compound of formula (I) is taxifolin-3-O-acetate. In this embodiment of the invention the process comprises the following enzyme(s): flavanone-3-hydroxylase (F3H) and acetyltransferase. This embodiment is termed process 93 herein.

In another embodiment of the invention the process is an in vitro or in vivo process and the starting material taxifolin and the compound of formula (I) is taxifolin-3-O-acetate. In this embodiment of the invention the process comprises the following enzyme(s): acetyltransferase. This embodiment is termed process 94 herein.

In another embodiment of the invention the process is an in vitro or in vivo process and the starting material a glycosylated precursor such as eriocitrin and the compound of formula (I) is taxifolin-3-O-acetate. In this embodiment of the invention the process comprises the following enzyme(s): glycosidase (or alternatively a chemical hydrolysis step if performed in vitro), flavanone-3-hydroxylase (F3H) and acetyltransferase. This embodiment is termed process 95 herein.

In another embodiment of the invention the process is an in vitro or in vivo process and the starting material is a glycosylated precursor such as astilbin and the compound of formula (I) is taxifolin-3-O-acetate. In this embodiment of the invention the process comprises the following enzyme(s): glycosidase (or alternatively a chemical hydrolysis step if performed in vitro), and acetyltransferase. This embodiment is termed process 96 herein.

In another embodiment of the invention the process is an in vitro or in vivo process and the starting material is a glycosylated precursor such as a mixture of engeletin and astilbin in e.g. Engelhardia Roxburghiana extract and the compounds of formula (I) are aromadendrin-3-O-acetate and taxifolin-3-O-acetate. In this embodiment of the invention the process comprises the following enzyme(s): glycosidase (or alternatively a chemical hydrolysis step if performed in vitro) and acetyltransferase. This embodiment is termed process 97 herein.

In addition to the preparation of aromadendrin-3-O-acetate and taxifolin-3-O-acetate, the enzyme combinations used in the processes of the invention numbered 1 to 97 can also be used to prepare the dihydrotamarixetin-3-O-acetate (a compound of formula (I)) with the use of the additional methyltransferase enzyme specific for the 4′-position (4′-MT). Hence further embodiments of the invention provide a process of preparing dihydrotamarixetin-3-O-acetate comprising the enzymes listed in processes 1 to 97 and a methyltransferase, which is specific for 4′—OH. The use of the methyltransferase enzyme specific for the 4′-position (4′-MT) in any of process 1 to 97 results in an additional 97 processes. These are herein termed process 98 to 194. Hence for example, process 98 is that of process 1 with the addition of the methyltransferase enzyme specific for the 4′-position (4′-MT), and so on.

Alternatively, final 4′-O-methylation can also be obtained by methylating position 4 of tyrosine, coumaric acid, coumaroyl-CoA, caffeic acid or caffeoyl-CoA present as intermediate or starting material in processes 1 to 27 and 63 to 89 with the use of an additional enzyme 4-O-methyltransferase or 4-O-caffeoyl-methyltransferase in process 1 to 27 and 63 to 89. The use of the methyltransferase enzyme specific for the 4-position (4-MT) in any of process 1 to 27 and 63 to 89 results in an additional 54 processes. These are herein termed process 195 to 249. Hence for example, process 195 is that of process 1 with the addition of 4-O-methyltransferase or 4-O-caffeoyl-methyltransferase and so on.

In another embodiment of the invention the process is an in vitro or in vivo process and the starting material is isoferulic acid and the compound of formula (I) is dihydrotamarixetin-3-O-acetate. In this embodiment of the invention the process comprises the following enzyme(s): 4-coumarate-CoA ligase (4CL), chalcone synthase (CHS), chalcone isomerase (CHI), chalcone isomerase-like protein (CHIL) flavanone-3-hydroxylase (F3H) and acetyltransferase. This embodiment is termed process 250 herein.

In another embodiment of the invention the process is an in vitro or in vivo process and the starting material is isoferulic acid and the compound of formula (I) is dihydrotamarixetin-3-O-acetate. Here the CHIL enzyme is omitted since this enzyme is not necessary for the performance of the process. In this embodiment of the invention the process comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed process 251 herein.

In another embodiment of the invention the process is an in vitro or in vivo process and the starting material is isoferulic acid and the compound of formula (I) is dihydrotamarixetin-3-O-acetate. Here the CHIL and CHI enzymes are omitted since these enzymes are not necessary for the performance of the process. In this embodiment of the invention the process comprises the following enzyme(s): 4-coumarate-CoA ligase (4CL), chalcone synthase (CHS), flavanone-3-hydroxylase (F3H) and acetyltransferase. This embodiment is termed process 252 herein.

In another embodiment of the invention the process is an in vitro or in vivo process and the starting material is hesperetin and the compound of formula (I) is dihydrotamarixetin-3-O-acetate. In this embodiment of the invention the process comprises the following enzyme(s): flavanone-3-hydroxylase (F3H) and acetyltransferase. This embodiment is termed process 253 herein.

In another embodiment of the invention the process is an in vitro or in vivo process and the starting material is dihydrotamarixetin and the compound of formula (I) is dihydrotamarixetin-3-O-acetate. In this embodiment of the invention the process comprises the following enzyme(s): acetyltransferase. This embodiment is termed process 254 herein.

In another embodiment of the invention the process is an in vitro or in vivo process and the starting material is a glycosylated precursor such as hesperidin and the compound of formula (I) is dihydrotamarixetin-3-O-acetate. In this embodiment of the invention the process comprises the following enzyme(s): glycosidase or (or alternatively a chemical hydrolysis step if performed in vitro), flavanone-3-hydroxylase (F3H) and acetyltransferase. This embodiment is termed process 255 herein.

In another embodiment of the invention the process is an in vitro or in vivo process and the starting material is a glycosylated precursor and the compound of formula (I) is dihydrotamarixetin-3-O-acetate. In this embodiment of the invention the process comprises the following enzyme(s): glycosidase (or alternatively a chemical hydrolysis step if performed in vitro) and acetyltransferase. This embodiment is termed process 256 herein.

In addition to the preparation of aromadendrin-3-O-acetate and taxifolin-3-O-acetate, the enzyme combinations used in the processes of the invention numbered 1 to 97 can also be used to prepare the 3′-O-methyl-taxifolin-3-O-acetate (a compound of formula (I)) with the use of the additional methyltransferase enzyme specific for the 3′-position (3′-MT). Hence further embodiments of the invention provide a process of preparing 3′-O-methyl-taxifolin-3-O-acetate comprising the enzymes listed in processes 1 to 97 and a 3′-OH methyltransferase. The use of the methyltransferase enzyme specific for the 3′-position (3′-MT) in any of process 1 to 97 results in an additional 97 processes. These are herein termed process 257 to 353. Hence for example, process 257 is that of process 1 with the addition of the methyltransferase enzyme specific for the 3′-position (3′-MT), and so on.

Alternatively, final 3′-O-methylation can also be obtained by methylating position 3 of caffeic acid or caffeoyl-CoA present as intermediate or starting material in processes 63 to 89 with the use of an additional enzyme 3-O-methyltransferase or 3-O-caffeoyl-methyltransferase in process 63 to 89. The use of the methyltransferase enzyme specific for the 3-position (3-MT) in any of process 63 to 89 results in an additional 27 processes. These are herein termed process 354 to 380. Hence for example, process 354 is that of process 1 with the addition of the methyltransferase enzyme specific for the 3-position (3-MT), and so on.

In another embodiment of the invention the process is an in vitro or in vivo process and the starting material is ferulic acid and the compound of formula (I) is 3′-O-methyl-taxifolin-3-O-acetate. In this embodiment of the invention the process comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed process 381 herein.

In another embodiment of the invention the process is an in vitro or in vivo process and the starting material is ferulic acid and the compound of formula (I) is 3′-O-methyl-taxifolin-3-O-acetate. Here the CHIL enzyme is omitted since this enzyme is not necessary for the performance of the process In this embodiment of the invention the process comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed process 382 herein.

In another embodiment of the invention the process is an in vitro or in vivo process and the starting material is ferulic acid and the compound of formula (I) is 3′-O-methyl-taxifolin-3-O-acetate. Here the CHIL and CHI enzymes are omitted since these enzymes are not necessary for the performance of the process. In this embodiment of the invention the process comprises the following enzyme(s): 4-coumarate-CoA ligase (4CL), chalcone synthase (CHS), flavanone-3-hydroxylase (F3H) and acetyltransferase. This embodiment is termed process 383 herein.

In another embodiment of the invention the process is an in vitro or in vivo process and the starting material is homoeriodictyol and the compound of formula (I) is 3′-O-methyl-taxifolin-3-O-acetate. In this embodiment of the invention the process comprises the following enzyme(s): flavanone-3-hydroxylase (F3H) and acetyltransferase. This embodiment is termed process 384 herein.

In another embodiment of the invention the process is an in vitro or in vivo process and the starting material is 3′-O-methyl-taxifolin and the compound of formula (I) is 3′-O-methyl-taxifolin-3-O-acetate. In this embodiment of the invention the process comprises the following enzyme(s): acetyltransferase. This embodiment is termed process 385 herein.

In another embodiment of the invention the process is an in vitro or in vivo process and the starting material is a glycosylated precursor such as homoeriodictyol-7-O-glucoside and the compound of formula (I) is 3′-O-methyl-taxifolin-3-O-acetate. In this embodiment of the invention the process comprises the following enzyme(s): glycosidase (or alternatively a chemical hydrolysis step if performed in vitro), flavanone-3-hydroxylase (F3H) and acetyltransferase. This embodiment is termed process 386 herein.

In another embodiment of the invention the process is an in vitro or in vivo process and the starting material is a glycosylated precursor and the compound of formula (I) is 3′-O-methyl-taxifolin-3-O-acetate. In this embodiment of the invention the process comprises the following enzyme(s): glycosidase (or alternatively a chemical hydrolysis step if performed in vitro) and acetyltransferase. This embodiment is termed process 387 herein.

In another embodiment of the invention the process is an in vitro or in vivo process and the starting material is glucose (or another carbon source) and/or phenylalanine and the compound of formula (I) is pinobanksin-3-O-acetate. In this embodiment of the invention the process comprises enzymes as in process 7, 9, and 11, but omitting cinnamate-4-hydroxylase (C4H) and P450 reductase (CPR). These embodiments are termed process 388, 389 and 390.

In another embodiment of the invention the process is an in vitro or in vivo process and the starting material is cinnamic acid and the compound of formula (I) is pinobanksin-3-O-acetate. In this embodiment of the invention the process comprises enzymes as in process 16, 18, and 20, but omitting cinnamate-4-hydroxylase (C4H) and P450 reductase (CPR). These embodiments are termed process 391, 392 and 393.

In another embodiment of the invention the process is an in vitro or in vivo process and the starting material is pinocembrin and the compound of formula (I) is pinobanksin-3-O-acetate. In this embodiment of the invention the process comprises the following enzyme(s): flavanone-3-hydroxylase (F3H) and acetyltransferase. This embodiment is termed process 394 herein.

In another embodiment of the invention the process is an in vitro or in vivo process and the starting material is pinobanksin and the compound of formula (I) is pinobanksin-3-O-acetate. In this embodiment of the invention the process comprises the following enzyme(s): acetyltransferase. This embodiment is termed process 395 herein.

In another embodiment of the invention the process is an in vitro or in vivo process and the starting material is a glycosylated precursor such as pinocembrin-7-glucoside and the compound of formula (I) is pinobanksin-3-O-acetate. In this embodiment of the invention the process comprises the following enzyme(s): glycosidase (or alternatively a chemical hydrolysis step if performed in vitro), flavanone-3-hydroxylase (F3H) and acetyltransferase. This embodiment is termed process 396 herein.

In another embodiment of the invention the process is an in vitro or in vivo process and the starting material is a glycosylated precursor such as pinobanksin 5-galactosyl-(1-4)-glucoside and the compound of formula (I) is pinobanksin-3-O-acetate. In this embodiment of the invention the process comprises the following enzyme(s): glycosidase (or alternatively a chemical hydrolysis step if performed in vitro) and acetyltransferase. This embodiment is termed process 397 herein.

In addition to the preparation of aromadendrin-3-O-acetate, taxifolin-3-O-acetate, dihydrotamarixetin-3-O-acetate, 3′-O-methyltaxifolin-3-O-acetate or pinobanksin-3-O-acetate the enzyme combinations used in the processes of the invention numbered 1-27, 32-58, 63-92, 98-124, 129-155, 160-189, 195-252, 257-283, 288-314, 319-348, 354-383, 388-393 can also be used to prepare 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate (compounds of formula (I)) with the use of the additional polyketide reductase (PKR). Hence further embodiments of the invention provide processes of preparing 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate comprising the enzymes listed in processes 1-27, 32-58, 63-92, 98-124, 129-155, 160-189, 195-252, 257-283, 288-314, 319-348, 354-383, 388-393 and a polyketide reductase. The use of the polyketide reductase enzyme in any of the processes 1-27, 32-58, 63-92, 98-124, 129-155, 160-189, 195-252, 257-283, 288-314, 319-348, 354-383, 388-393 results in additional 345 processes. These are herein termed processes 398 to 742. Hence for example, process 398 is that of process 1 with the addition of the polyketide reductase, and so on.

In another embodiment of the invention the process is an in vitro or in vivo process and the starting material is isoliquiritigenin and the compound of formula (I) is 5-deoxyaromadendrin-3-O-acetate. In this embodiment of the invention the process comprises the following enzyme(s): chalcone isomerase (CHI), flavanone-3-hydroxylase (F3H) and acetyltransferase. This embodiment is termed process 743 herein.

In another embodiment of the invention the process is an in vitro or in vivo process and the starting material is liquiritigenin and the compound of formula (I) is 5-deoxyaromadendrin-3-O-acetate. In this embodiment of the invention the process comprises the following enzyme(s): flavanone-3-hydroxylase (F3H) and acetyltransferase. This embodiment is termed process 744 herein.

In another embodiment of the invention the process is an in vitro or in vivo process and the starting material is 5-deoxyaromadendrin and the compound of formula (I) is 5-deoxyaromadendrin-3-O-acetate. In this embodiment of the invention the process comprises the following enzyme(s): acetyltransferase. This embodiment is termed process 745 herein.

In another embodiment of the invention the process is an in vitro or in vivo process and the starting material is butein and the compound of formula (I) is 5-deoxytaxifolin-3-O-acetate. In this embodiment of the invention the process comprises the following enzyme(s): chalcone isomerase (CHI), flavanone-3-hydroxylase (F3H) and acetyltransferase. This embodiment is termed process 746 herein.

In another embodiment of the invention the process is an in vitro or in vivo process and the starting material is 5-deoxyeriodictyol and the compound of formula (I) is 5-deoxytaxifolin-3-O-acetate. In this embodiment of the invention the process comprises the following enzyme(s): flavanone-3-hydroxylase (F3H) and acetyltransferase. This embodiment is termed process 747 herein.

In another embodiment of the invention the process is an in vitro or in vivo process and the starting material is 5-deoxytaxifolin and the compound of formula (I) is 5-deoxytaxifolin-3-O-acetate. In this embodiment of the invention the process comprises the following enzyme(s): acetyltransferase. This embodiment is termed process 748 herein.

Further embodiments of the invention provide in vitro or in vivo processes of preparing 5-deoxytaxifolin-3-O-acetate comprising the enzymes listed in processes 743 to 745 and a flavonoid 3-hydroxylase (F3′H). As can be appreciated by the skilled person, the addition of F3′H to any one of processes 743 to 745 results in additional three processes. These are herein termed processes 749 to 751. Hence for example, process 749 is that of process 743 with the addition of F3′H, and so on.

In another embodiment of the invention the process is an in vitro or in vivo process and the starting material is 5-deoxyhesperetin chalcone and the compound of formula (I) is 5-deoxydihydrotamarixetin-3-O-acetate. In this embodiment of the invention the process comprises the following enzyme(s): chalcone isomerase (CHI), flavanone-3-hydroxylase (F3H) and acetyltransferase. This embodiment is termed process 752 herein.

In another embodiment of the invention the process is an in vitro or in vivo process and the starting material is 5-deoxyhesperetin and the compound of formula (I) is 5-deoxydihydrotamarixetin-3-O-acetate. In this embodiment of the invention the process comprises the following enzyme(s): flavanone-3-hydroxylase (F3H) and acetyltransferase. This embodiment is termed process 753 herein.

In another embodiment of the invention the process is an in vitro or in vivo process and the starting material is 5-deoxydihydrotamarixetin and the compound of formula (I) is 5-deoxydihydrotamarixetin-3-O-acetate. In this embodiment of the invention the process comprises the following enzyme(s): acetyltransferase. This embodiment is termed process 754 herein.

Further embodiments of the invention provide in vitro or in vivo processes of preparing 5-deoxydihydrotamarixetin-3-O-acetate comprising the enzymes listed in processes 746 to 751 and a 4′-O-methyltransferase (4′-MT). As can be appreciated by the skilled person, the addition of a 4′-MT to any one of processes 746 to 751 results in an additional six processes. These are herein termed processes 755 to 760. Hence for example, process 755 is that of process 746 with the addition of a 4′-MT, and so on.

In another embodiment of the invention the process is an in vitro or in vivo process and the starting material is 5-deoxyhomoeriodictyol chalcone and the compound of formula (I) is 5-deoxy-3′-O-methyl-taxifolin-3-O-acetate. In this embodiment of the invention the process comprises the following enzyme(s): chalcone isomerase (CHI), flavanone-3-hydroxylase (F3H) and acetyltransferase. This embodiment is termed process 761 herein.

In another embodiment of the invention the process is an in vitro or in vivo process and the starting material is 5-deoxyhomoeriodictyol and the compound of formula (I) is 5-deoxy-3′-O-methyl-taxifolin-3-O-acetate. In this embodiment of the invention the process comprises the following enzyme(s): flavanone-3-hydroxylase (F3H) and acetyltransferase. This embodiment is termed process 762 herein.

In another embodiment of the invention the process is an in vitro or in vivo process and the starting material is 5-deoxy-3′-O-methyl-taxifolin and the compound of formula (I) is 5-deoxy-3′-O-methyl-taxifolin-3-O-acetate. In this embodiment of the invention the process comprises the following enzyme(s): acetyltransferase. This embodiment is termed process 763 herein.

Further embodiments of the invention provide in vitro or in vivo processes of preparing 5-deoxydihydrotamarixetin-3-O-acetate comprising the enzymes listed in processes 746 to 751 and a 3′-O-methyltransferase (3′-MT). As can be appreciated by the skilled person, the addition of a 3′-MT to any one of processes 746 to 751 results in an additional six processes. These are herein termed processes 764 to 769. Hence for example, process 764 is that of process 746 with the addition of a 3′-MT, and so on.

In another embodiment of the invention the process is an in vitro or in vivo process and the starting material is 5-deoxypinocembrin (7-hydroxyflavanone) chalcone and the compound of formula (I) is 5-deoxypinobanksin-3-O-acetate. In this embodiment of the invention the process comprises the following enzyme(s): chalcone isomerase (CHI), flavanone-3-hydroxylase (F3H) and acetyltransferase. This embodiment is termed process 770 herein.

In another embodiment of the invention the process is an in vitro or in vivo process and the starting material is 5-deoxypinocembrin (7-hydroxyflavanone) and the compound of formula (I) is 5-deoxypinobanksin-3-O-acetate. In this embodiment of the invention the process comprises the following enzyme(s): flavanone-3-hydroxylase (F3H) and acetyltransferase. This embodiment is termed process 771 herein.

In another embodiment of the invention the process is an in vitro or in vivo process and the starting material is 5-deoxypinobanksin and the compound of formula (I) is 5-deoxypinobanksin-3-O-acetate. In this embodiment of the invention the process comprises the following enzyme(s): acetyltransferase. This embodiment is termed process 772 herein.

Further as can be appreciated by the skilled person, precursors of the starting material of processes 743 to 772 can be glycosylated. Hence further embodiments of the invention provide processes of preparing 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate comprising glycosylated precursors of the starting material of processes 743 to 772, the enzymes listed in processes 743 to 772 and a glycosidase. As can be appreciated by the skilled person, the use of a glycosylated precursor of the starting material and the addition of a glycosidase to any one of processes 743 to 772 results in an additional 30 processes. These are herein termed processes 773 to 802. Hence for example, process 773 is that of process 743 and the use of a glycosylated precursor of the starting material with the addition of a glycosidase, and so on.

As will be apparent to the skilled person, further acetylated molecules with additional hydroxy and methoxy groups can be prepared and used in the process of the invention by the use of specific P450 monooxygenases and optional P450 reductases, other type of hydroxylases and/or methyltransferases.

Further acylated molecules can be incorporated into the process of the invention using general acyltransferase enzymes.

Examples of such enzymes are well known in the art and may be readily used in the processes above. Each enzyme may also be codon optimized for expression is specific host cells as discussed herein.

As described herein, the process of the invention may be an in vitro or in vivo process. Where the process is in vivo, then examples of suitable host cells are provided in the accompanying description above.

Furthermore, as can be appreciated by the skilled person, such host cells can be optimized for use in the process of the invention by up or down-regulation, exchange and engineering, of certain genes to increase metabolic flux to flavonoid precursors and/or reducing carbon loss resulting from the production of unwanted products.

For example, by modifying the shikimate pathway, the acetyl-CoA and/or malonyl-CoA biosynthesis the metabolic flux to precursor molecules is increased.

In one embodiment, a cell engineered to produce a compound of formula (I) may be further engineered to increase the supply of precursor malonyl-CoA. One strategy for increasing malonyl-CoA includes increasing acetyl-CoA carboxylase (ACC) activity. In various embodiments, the ACC enzyme, which in most eukaryotes, including fungi, is a large single chain polypeptide, and in plant and bacteria such as E. co/i is a multi-subunit enzyme, is overexpressed in the host strain.

Also considered, in further embodiments, is an engineered host cell that overexpresses a gene encoding pyruvate dehydrogenase (PDH), which converts pyruvate to acetyl-CoA.

Alternatively, or in addition to strategies for increasing ACC activity and strategies for increasing acetyl-CoA, strategies for increasing malonyl-CoA by mechanisms that do not rely on the activity of an ACC can be employed.

In some embodiments, a cell engineered to produce a compound of formula (I) is further engineered to increase the cell's supply of malonyl-CoA and includes an exogenous nucleic acid sequence encoding a malonyl-CoA synthetase that generates malonyl-CoA from malonate. Malonate can optionally be added to the culture medium of a culture that includes a cell engineered to express a malonyl-CoA synthetase. An engineered cell that includes an exogenous gene encoding a malonyl-CoA synthetase can also include an exogenous nucleic acid sequence encoding a malonate transporter, such as a malonate transporter encoded by a matC gene.

In additional embodiments, a cell engineered to produce a compound of formula (I) is further engineered to include an exogenous nucleic acid sequence encoding malonate CoA-transferase that makes malonyl-CoA by direct transfer of the CoA from acetyl-CoA.

In some embodiments, a cell engineered to produce a compound of formula (I) is further engineered to increase the supply of coenzyme A (CoA) to increase its availability for producing acetyl-CoA, malonyl-CoA, and/or p-coumaroyl-CoA. Strategies for increasing CoA supply include upregulating endogenous pantothenate kinase (PanK) (EC2.7.1.33) that produces CoA from pantothenate. Alternatively, or in addition, a host cell can be engineered to include a nucleic acid sequence encoding type III pantothenate kinase that is not feedback inhibited by coenzyme A.

Additional strategies to increase malonyl-CoA flux to the flavonoid pathway include mutation or downregulation of one or more genes that function in fatty acid biosynthesis. Without limiting the embodiments to any particular mechanism, limiting fatty acid biosynthesis can increase the malonyl-CoA supply available for flavonoid biosynthesis. In some embodiments, the gene beta-ketoacyl-ACP synthase II can be disrupted to reduce fatty acid biosynthesis. Another example of a fatty acid biosynthesis gene of a host cell that may be mutated or downregulated is a gene encoding malonyl-CoA-ACP transacylase. Other fatty acid biosynthesis genes of the engineered host cell that can be downregulated include a beta-ketoacyl-ACP synthase I enzyme and acyl carrier protein.

Additional genetic modifications that may be present in a host cell engineered to produce a compound of formula (I) include downregulation, disruption, or deletion of genes encoding alcohol dehydrogenase, lactate dehydrogenase, pyruvate oxidase, acetyl phosphate transferase and acetate kinase.

Further, a cell engineered for the production of a compound of formula (I) can have one or more genes encoding thioesterases downregulated, disrupted, or deleted to prevent hydrolysis of precursors malonyl-CoA, acetyl-CoA, and/or p-coumaroyl-CoA.

Also considered, in further embodiments, is an engineered host cell for the production of a compound of formula (I) to upregulate the endogenous biosynthesis of the amino acids phenylalanine or tyrosine. Phenylalanine and tyrosine are precursors for the flavonoid biosynthesis. Phenylalanine and tyrosine are derived from the shikimate pathway. Strategies to increase phenylalanine and tyrosine production can include, without limitation, transcriptional deregulation, removing feedback inhibition, and overexpression of rate-limiting enzymes.

Alternatively the flux can be shifted towards tyrosine by deletion of the L-phenylalanine branch of the aromatic acid biosynthetic pathway.

Alternatively, or in addition, genes encoding enzymes of the tricarboxylic acid cycle (TCA) involved in the biosynthesis of alpha-ketoglutarate can be upregulated or overexpressed, while alpha-ketoglutarate dehydrogenase, which catalyzes the oxidative decarboxylation of alpha-ketoglutarate to succinyl-CoA in the TCA, can be disrupted or downregulated to increase alpha-ketoglutarate supply which serves as a cofactor for one or more of the flavonoid pathway enzymes. Succinate dehydrogenase can be modified to guarantee the flux towards ketoglutarate and avoid the accumulation of succinate. Alternatively, the transport of mitochondrial alpha-ketoglutarate to the cytosol can be improved by the overexpression of a transporter responsible for alpha-ketoglutarate transport from mitochondria to the cytosol. Alpha-ketoglutarate can optionally be added to the culture medium. An engineered cell can also include an alpha-ketoglutarate transporter.

Other TCA enzymes that can be modified include citrate synthase that converts acetyl-CoA to citrate.

Also considered, in further embodiments, is an engineered host cell for the production of a compound of formula (I) further engineered to upregulate the endogenous biosynthesis of the cofactor heme. Cytochrome P450 (CYPs), one of the exogenous genes in the engineered cells provided herein, contain heme as a cofactor. Improving heme supply can be an effective strategy to increase flavonoid biosynthesis. 5-aminolevulinic acid (ALA) is the first committed precursor to the heme pathway. Strategies to increase heme supply include overexpression of the genes that synthesize the precursor ALA. Further, one or more of the downstream genes that catalyze the synthesis of heme from ALA can be overexpressed to drive the flux from ALA to heme production.

Also considered in a further embodiment is an engineered host cell for the production of a compound of formula (I) further engineered for the regeneration of S-adenosylmethionine by the addition of multiple enzymes, such as S-adenosylmethionine synthetase (SAM) SAH hydrolase (SAHH), methionine synthase (MS), methionine adenosyltransferase (MAT), serine hydroxymethyltransferase, (SHM2), methylenetetrahydrofolate reductase (MTHFR), dihydrofolate reductase (DHFR), adenosine kinase (ADK) and polyphosphate kinases (PPK2) I and II. Alternatively these enzymes can be added to an in vitro reaction.

In addition, the host cell may comprise the deletion, down-regulation, exchange or engineering of some genes of the host organisms to guide the flux of precursors and intermediates towards the target pathway and avoid side product formation. For example, deletion, down-regulation, exchange or engineering of one or more double bond reductases, which reduce coumaroyl-CoA to 2,3-dihydrocoumaric acid, for example in S. cerevisiae called TSC13. Also included is where the host cell strain is such that the exchange or engineering of one or more aromatic aminotransferase to shift the equilibrium towards the aromatic amino acid, such as for example Aro8 in S. cerevisiae. Also included is where the host cell comprises the deletion, down-regulation, exchange or engineering of one or more 2-oxo acid decarboxylases such as for example Aro10, PDC1, PDC5 and PDC6 in S. cerevisiae to prevent the decarboxylation of phenylpyruvate. Further included is the deletion, down-regulation, exchange or engineering of one or more phenylacrylic acid decarboxylase and/or ferulic acid decarboxylase, such as for example PAD1 and FDC1 in S. cerevisiae and UbiX and UbiD in E. coli to prevent the decarboxylation of cinnamic acid and coumaric acid (or also caffeic acid, ferulic acid, and isoferulic acid, if they are used as starting material).

To avoid the accumulation of final product in the cell the export can also be optimized using appropriate transporter proteins. For example, any transporter protein which can bind and export flavonoids from cells can be used for this purpose. Examples of such transporter proteins may be found in plants, as can be appreciated by the skilled person.

For the avoidance of doubt, as discussed above the process of the invention can use a hydrolase in replacement of an acyltransferase.

Hence in each of the process of the invention listed as processes 1 to 802 provided above, the acetyltransferase enzyme can be substituted with a hydrolase enzyme.

### Recombinant Cells of the Invention

The present invention provides for the first time a recombinant cell comprising a compound of formula (I). This is of particular use in the preparation of specific compounds of formula (I), in particular aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate. Until the present invention it had not previously been able to prepare these compounds using in vivo methods involving the use to recombinant cells.

By synthesizing compounds of formula (I) using recombinant cell technology, it allows the production of compounds without synthetic chemistry thus providing a source of compounds of formula (I), in particular aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate, more acceptable for consumption by the user.

In some embodiments, a compound of formula (I) or (Ia) is produced by whole cell bioconversion. For whole cell bioconversion to occur, a host cell expressing one or more enzymes involved in the biosynthetic pathway takes up and modifies a precursor in the cell; following modification in vivo, a compound of formula (I) or (Ia) remains in the cell and/or is excreted into the culture medium.

Accordingly therefore an aspect of the present invention provides a recombinant cell comprising a compound of formula (I). Preferably the compound of formula (I), is aromadendrin-3-O-acetate, taxifolin-3-O-acetate, dihydrotamarixetin-3-O-acetate, 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate.

An embodiment of this aspect of the invention is wherein the recombinant cell further comprises a polypeptide capable of synthesizing a compound of formula (I) from a compound of formula (Ia). Preferably the polypeptide capable of synthesizing a compound of formula (I) from a compound of formula (Ia) is an acyltransferase or a hydrolase.

A preferred embodiment of the invention is wherein the recombinant cell comprises an acetyltransferase enzyme comprising the amino acid sequence HXXXD (SEQ ID NO: 29) and/or amino acid sequence [DN]FGxG (SEQ ID NO:30). Preferably, the acetyltransferase enzyme further comprises the amino acid sequence [ST]S[WL](SEQ ID NO: 94). Preferably, the acetyltransferase enzyme is a recombinant acetyltransferase enzyme. More preferably, the acetyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68. A further preferred embodiment of the invention is wherein the recombinant cell heterologously expresses or overexpresses said acetyltransferase enzyme.

A preferred embodiment of the invention is wherein the recombinant cell comprises a recombinant nucleic acid sequence encoding an acyltransferase enzyme, preferably an acetyltransferase enzyme. More preferably, the recombinant nucleic acid sequence encoding an acetyltransferase enzyme has at least 50%, 55%, 60%, 65%, 70%, 75%, 80%, 85%, 90%, 95%, 96%, 97%, 98% or 99% sequence identity to any of SEQ ID NOs: 8 to 28, 37 to 54 and 69 to 93 or comprises the nucleotide sequence of any of SEQ ID NOs: 8 to 28, 37 to 54 and 69 to 93 or the reverse complement thereof.

In some embodiments the recombinant cell of this aspect of the invention further comprises one or more of the following enzyme(s):
(a) flavanone 3-hydroxylase (F3H),
(b) chalcone isomerase (CHI),
(c) Chalcone synthase (CHS),
(d) 4-coumarate-coenzyme A ligase (4CL),
(e) cytochrome P450 reductase (CPR),
(f) tyrosine ammonia lyase (TAL),
(g) chalcone isomerase-like (CHIL),
(h) cinnamate-4-hydroxylase (C4H),
(i) phenylalanine ammonia lyase (PAL),
(j) flavonoid 3-hydroxylase (F3′H),
(k) 3′-O-methyltransferase (3′-MT),
(l) 4′-O-methyltransferase (4′-MT),
(m) 3-O-methyltransferase (3-MT),
(n) 4-O-methyltransferase (4-MT),
(o) 3-OH specific P450 monooxygenase,
(p) glycosidase, and/or
(q) polyketide reductase (PKR)

In one embodiment of the invention the recombinant cell is used in an in vitro or in vivo process and the starting material is glucose (or another carbon source), phenylalanine and/or tyrosine and the compound of formula (I) is aromadendrin-3-O-acetate. In this embodiment of the invention the recombinant cell comprises the following enzyme(s): phenylalanine ammonia lyase (PAL), cinnamate-4-hydroxylase (C4H), cytochrome P450 reductase (CPR), tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed recombinant cell one herein.

In another embodiment of the invention the recombinant cell is used in an in vivo process, the starting material is glucose (or another carbon source), phenylalanine and/or tyrosine and the compound of formula (I) is aromadendrin-3-O-acetate and the microbial host cell comprises a functional CPR, for example a yeast cell. In this embodiment of the invention the recombinant cell comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed recombinant cell two herein.

In another embodiment of the invention the recombinant cell is used in an in vitro or in vivo process, the starting material is glucose (or another carbon source), phenylalanine and/or tyrosine and the compound of formula (I) is aromadendrin-3-O-acetate. Here the CHIL enzyme is omitted since this enzyme is not necessary for the performance of the process. In this embodiment of the invention the recombinant cell comprises the following enzyme(s): phenylalanine ammonia lyase (PAL), cinnamate-4-hydroxylase (C4H), cytochrome P450 reductase (CPR), tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed recombinant cell three herein.

In another embodiment of the invention the recombinant cell is used in an in vivo process, the starting material is glucose (or another carbon source), phenylalanine and/or tyrosine and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the recombinant cell comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed recombinant cell four herein.

In another embodiment of the invention the recombinant cell is used in an in vitro or in vivo process, the starting material is glucose (or another carbon source), phenylalanine and/or tyrosine and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), flavanone-3-hydroxylase (F3H) and an acetyltransferase. This embodiment is termed recombinant cell five herein.

In another embodiment of the invention the recombinant cell is used in an in vivo process, the starting material is glucose (or another carbon source), phenylalanine and/or tyrosine and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the recombinant cell comprises the following enzyme(s): phenylalanine ammonia lyase (PAL), cinnamate-4-hydroxylase (C4H), tyrosine ammonia lyase (TAL), 4-coumarate-CoA ligase (4CL), chalcone synthase (CHS), flavanone-3-hydroxylase (F3H) and an acetyltransferase. This embodiment is termed recombinant cell six herein.

In one embodiment of the invention the recombinant cell is used in an in vitro or in vivo process and the starting material is glucose (or another carbon source) and/or phenylalanine and the compound of formula (I) is aromadendrin-3-O-acetate. In this embodiment of the invention the recombinant cell comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed recombinant cell seven herein.

In another embodiment of the invention the recombinant cell is used in an in vivo process, the starting material is glucose (or another carbon source) and/or phenylalanine and the compound of formula (I) is aromadendrin-3-O-acetate and the microbial host cell comprises a functional CPR, for example a yeast cell. In this embodiment of the invention the recombinant cell comprises the following enzyme(s): phenylalanine ammonia lyase (PAL), cinnamate-4-hydroxylase (C4H), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed recombinant cell eight herein.

In another embodiment of the invention the recombinant cell is used in an in vitro or in vivo process, the starting material is glucose (or another carbon source) and/or phenylalanine and the compound of formula (I) is aromadendrin-3-O-acetate. Here the CHIL enzyme is omitted since this enzyme is not necessary for the performance of the process. In this embodiment of the invention the recombinant cell comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), chalcone isomerase (CHI), flavanone-3-hydroxylase (F3H) and an acetyltransferase. This embodiment is termed recombinant cell nine herein.

In another embodiment of the invention the recombinant cell is used in an in vivo process, the starting material is glucose (or another carbon source) and/or phenylalanine and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the recombinant cell comprises the following enzyme(s): phenylalanine ammonia lyase (PAL), cinnamate-4-hydroxylase (C4H), 4-coumarate-CoA ligase (4CL), chalcone synthase (CHS), chalcone isomerase (CHI), flavanone-3-hydroxylase (F3H) and an acetyltransferase. This embodiment is termed recombinant cell ten herein.

In another embodiment of the invention the recombinant cell is used in an in vitro or in vivo process, the starting material is glucose (or another carbon source) and/or phenylalanine and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): phenylalanine ammonia lyase (PAL), cinnamate-4-hydroxylase (C4H), cytochrome P450 reductase (CPR), 4-coumarate-CoA ligase (4CL), chalcone synthase (CHS), flavanone-3-hydroxylase (F3H) and an acetyltransferase. This embodiment is termed recombinant cell 11 herein.

In another embodiment of the invention the recombinant cell is used in an in vivo process, the starting material is glucose (or another carbon source), and/or phenylalanine and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the recombinant cell comprises the following enzyme(s): phenylalanine ammonia lyase (PAL), cinnamate-4-hydroxylase (C4H), 4-coumarate-CoA ligase (4CL), chalcone synthase (CHS), flavanone-3-hydroxylase (F3H) and an acetyltransferase. This embodiment is termed recombinant cell 12 herein.

In one embodiment of the invention the recombinant cell is used in an in vitro or in vivo process and the starting material is glucose (or another carbon source) and/or tyrosine and the compound of formula (I) is aromadendrin-3-O-acetate. In this embodiment of the invention the recombinant cell comprises the following enzyme(s): tyrosine ammonia lyase (TAL), 4-coumarate-CoA ligase (4CL), chalcone synthase (CHS), chalcone isomerase (CHI), chalcone isomerase-like protein (CHIL), flavanone-3-hydroxylase (F3H) and an acetyltransferase. This embodiment is termed recombinant cell 13 herein.

In one embodiment of the invention the recombinant cell is used in an in vitro or in vivo process and the starting material is glucose (or another carbon source) and/or tyrosine and the compound of formula (I) is aromadendrin-3-O-acetate. Here the CHIL enzyme is omitted since this enzyme is not necessary for the performance of the process. In this embodiment of the invention the recombinant cell comprises the following enzyme(s): tyrosine ammonia lyase (TAL), 4-coumarate-CoA ligase (4CL), chalcone synthase (CHS), chalcone isomerase (CHI), flavanone-3-hydroxylase (F3H) and an acetyltransferase. This embodiment is termed recombinant cell 14 herein.

In one embodiment of the invention the recombinant cell is used in an in vitro or in vivo process and the starting material is glucose (or another carbon source) and/or tyrosine and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): tyrosine ammonia lyase (TAL), 4-coumarate-CoA ligase (4CL), chalcone synthase (CHS), flavanone-3-hydroxylase (F3H) and an acetyltransferase. This embodiment is termed recombinant cell 15 herein.

In another embodiment of the invention the recombinant cell is used in an in vitro or in vivo process, the starting material is cinnamic acid and the compound of formula (I) is aromadendrin-3-O-acetate. In this embodiment of the invention the recombinant cell comprises the following enzyme(s): cinnamate-4-hydroxylase (C4H), cytochrome P450 reductase (CPR), 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed recombinant cell 16 herein.

In another embodiment of the invention the recombinant cell is used in an in vivo process, the starting material is cinnamic acid and the compound of formula (I) is aromadendrin-3-O-acetate and the microbial host cell comprises a functional CPR, for example a yeast cell. In this embodiment of the invention the recombinant cell comprises the following enzyme(s): cinnamate-4-hydroxylase (C4H), 4-coumarate-CoA ligase (4CL), chalcone synthase (CHS), chalcone isomerase (CHI), chalcone isomerase-like protein (CHIL), flavanone-3-hydroxylase (F3H) and acetyltransferase.

This embodiment is termed recombinant cell 17 herein. In one embodiment of the invention the recombinant cell is used in an in vitro or in vivo process and the starting material is cinnamic acid and the compound of formula (I) is aromadendrin-3-O-acetate. Here the CHIL enzyme is omitted since this enzyme is not necessary for the performance of the process. In this embodiment of the invention the recombinant cell comprises the following enzyme(s): cinnamate-4-hydroxylase (C4H), cytochrome P450 reductase (CPR), 4-coumarate-CoA ligase (4CL), chalcone synthase (CHS), chalcone isomerase (CHI), flavanone-3-hydroxylase (F3H) and acetyltransferase. This embodiment is termed recombinant cell 18 herein.

In another embodiment of the invention the recombinant cell is used in an in vivo process, the starting material is cinnamic acid and the compound of formula (I) is aromadendrin-3-O-acetate and the microbial host cell comprises a functional CPR, for example a yeast cell. Here the CHIL enzyme is omitted since this enzyme is not necessary for the performance of the process. In this embodiment of the invention the recombinant cell comprises the following enzyme(s): cinnamate-4-hydroxylase (C4H), 4-coumarate-CoA ligase (4CL), chalcone synthase (CHS), chalcone isomerase (CHI), flavanone-3-hydroxylase (F3H) and acetyltransferase. This embodiment is termed recombinant cell 19 herein.

In another embodiment of the invention the recombinant cell is used in an in vitro or in vivo process and the starting material is cinnamic acid and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): cinnamate-4-hydroxylase (C4H), cytochrome P450 reductase (CPR), 4-coumarate-CoA ligase (4CL), chalcone synthase (CHS), flavanone-3-hydroxylase (F3H) and acetyltransferase. This embodiment is termed recombinant cell 20 herein.

In another embodiment of the invention the recombinant cell is used in an in vivo process, the starting material is cinnamic acid and the compound of formula (I) is aromadendrin-3-O-acetate and the microbial host cell comprises a functional CPR, for example a yeast cell. Here the CHIL and CHI enzymes are omitted since these enzymes are not necessary for the performance of the process. In this embodiment of the invention the recombinant cell comprises the following enzyme(s): cinnamate-4-hydroxylase (C4H), 4-coumarate-CoA ligase (4CL), chalcone synthase (CHS), flavanone-3-hydroxylase (F3H) and acetyltransferase. This embodiment is termed recombinant cell 21 herein.

In another embodiment of the invention the recombinant cell is used in an in vitro or in vivo process and the starting material is coumaric acid and the compound of formula (I) is aromadendrin-3-O-acetate. In this embodiment of the invention the recombinant cell comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed recombinant cell 22 herein.

In another embodiment of the invention the recombinant cell is used in an in vitro or in vivo process and the starting material is coumaric acid and the compound of formula (I) is aromadendrin-3-O-acetate. Here the CHIL enzyme is omitted since this enzyme is not necessary for the performance of the process. In this embodiment of the invention the recombinant cell comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed recombinant cell 23 herein.

In another embodiment of the invention the recombinant cell is used in an in vitro or in vivo process and the starting material is coumaric acid and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): 4-coumarate-CoA ligase (4CL), chalcone synthase (CHS), flavanone-3-hydroxylase (F3H) and acetyltransferase. This embodiment is termed recombinant cell 24 herein.

In another embodiment of the invention the recombinant cell is used in an in vitro or in vivo process and the starting material is coumaroyl-CoA and the compound of formula (I) is aromadendrin-3-O-acetate. In this embodiment of the invention the recombinant cell comprises the following enzyme(s): chalcone synthase (CHS), chalcone isomerase (CHI), chalcone isomerase-like protein (CHIL), flavanone-3-hydroxylase (F3H) and acetyltransferase. This embodiment is termed recombinant cell 25 herein.

In another embodiment of the invention the recombinant cell is used in an in vitro or in vivo process and the starting material is coumaroyl-CoA and the compound of formula (I) is aromadendrin-3-O-acetate. Here the CHIL enzyme is omitted since this enzyme is not necessary for the performance of the process. In this embodiment of the invention the recombinant cell comprises the following enzyme(s): chalcone synthase (CHS), chalcone isomerase (CHI), flavanone-3-hydroxylase (F3H) and acetyltransferase. This embodiment is termed recombinant cell 26 herein.

In another embodiment of the invention the recombinant cell is used in an in vitro or in vivo process and the starting material is coumaroyl-CoA and the compound of formula (I) is aromadendrin-3-O-acetate. Here the CHIL and CHI enzymes are omitted since these enzymes are not necessary for the performance of the process. In this embodiment of the invention the recombinant cell comprises the following enzyme(s): chalcone synthase (CHS), flavanone-3-hydroxylase (F3H) and acetyltransferase. This embodiment is termed recombinant cell 27 herein.

In another embodiment of the invention the recombinant cell is used in an in vitro or in vivo process and the starting material is naringenin and the compound of formula (I) is aromadendrin-3-O-acetate. In this embodiment of the invention the recombinant cell comprises the following enzyme(s): flavanone-3-hydroxylase (F3H) and acetyltransferase. This embodiment is termed recombinant cell 28 herein.

In another embodiment of the invention the recombinant cell is used in an in vitro or in vivo process and the starting material is aromadendrin and the compound of formula (I) is aromadendrin-3-O-acetate. In this embodiment of the invention the recombinant cell comprises the following enzyme(s): acetyltransferase. This embodiment is termed recombinant cell 29 herein.

In another embodiment of the invention the recombinant cell is used in an in vitro or in vivo process and the starting material is a glycosylated precursor such as for example naringin and the compound of formula (I) is aromadendrin-3-O-acetate. In this embodiment of the invention the recombinant cell comprises the following enzyme(s): glycosidase, flavanone-3-hydroxylase (F3H) and acetyltransferase. This embodiment is termed recombinant cell 30 herein.

In another embodiment of the invention the recombinant cell is used in an in vitro or in vivo process and the starting material is a glycosylated precursor such as for example engeletin and the compound of formula (I) is aromadendrin-3-O-acetate. In this embodiment of the invention the recombinant cell comprises the following enzyme(s): glycosidase, and acetyltransferase. This embodiment is termed recombinant cell 31 herein.

In addition to the preparation of aromadendrin-3-O-acetate, the enzyme combinations used in the processes of the invention numbered 1 to 31 can also be used to prepare the taxifolin-3-O-acetate (a compound of formula (I)) with the inclusion of the additional enzyme flavonoid 3-hydroxylase (F3′H). Hence further embodiments of the invention provide recombinant cells for preparing taxifolin-3-O-acetate comprising the enzymes listed in recombinant cells 1 to 31 and flavonoid 3′-hydroxylase (F3′H). As can be appreciated by the skilled person, the inclusion of F3′H to any one of recombinant cells 1 to 31 results in an additional 31 recombinant cells. These are herein termed recombinant cells 32 to 62. Hence for example, recombinant cell 32 is that of recombinant cell 1 with the addition of F3′H, and so on.

Alternatively, final 3′-hydroxylation can also be obtained by hydroxylating position 3 of phenylalanine, tyrosine, cinnamic acid, coumaric acid or coumaroyl-CoA present as intermediate or starting material in processes 1 to 27 with the inclusion of an additional enzyme coumaric acid 3-hydroxylase or 4-hydroxybenzoate-m-hydroxylase in the recombinant cells 1 to 27. The inclusion of the additional enzyme coumaric acid 3-hydroxylase or 4-hydroxybenzoate-m-hydroxylase in any of recombinant cells 1 to 27 results in an additional 27 recombinant cells. These are herein termed recombinant cells 63 to 89. Hence for example, recombinant cell 63 is that of recombinant cell 1 with the addition of coumaric acid 3-hydroxylase or 4-hydroxybenzoate-m-hydroxylase, and so on.

In another embodiment of the invention the recombinant cell is used in an in vitro or in vivo process and the starting material caffeic acid and the compound of formula (I) is taxifolin-3-O-acetate. In this embodiment of the invention the recombinant cell comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed recombinant cell 90 herein.

In another embodiment of the invention the recombinant cell is used in an in vitro or in vivo process and the starting material caffeic acid and the compound of formula (I) is taxifolin-3-O-acetate. Here the CHIL enzyme is omitted since this enzyme is not necessary for the performance of the process. In this embodiment of the invention the recombinant cell comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed recombinant cell 91 herein.

In another embodiment of the invention the recombinant cell is used in an in vitro or in vivo process and the starting material caffeic acid and the compound of formula (I) is taxifolin-3-O-acetate. Here the CHIL and CHI enzymes are omitted since these enzymes are not necessary for the performance of the process. In this embodiment of the invention the recombinant cell comprises the following enzyme(s): 4-coumarate-CoA ligase (4CL), chalcone synthase (CHS), flavanone-3-hydroxylase (F3H) and acetyltransferase. This embodiment is termed recombinant cell 92 herein.

In another embodiment of the invention the recombinant cell is used in an in vitro or in vivo process and the starting material eriodictyol and the compound of formula (I) is taxifolin-3-O-acetate. In this embodiment of the invention the recombinant cell comprises the following enzyme(s): flavanone-3-hydroxylase (F3H) and acetyltransferase. This embodiment is termed recombinant cell 93 herein.

In another embodiment of the invention the recombinant cell is used in an in vitro or in vivo process and the starting material taxifolin and the compound of formula (I) is taxifolin-3-O-acetate. In this embodiment of the invention the recombinant cell comprises the following enzyme(s): acetyltransferase. This embodiment is termed recombinant cell 94 herein.

In another embodiment of the invention the recombinant cell is used in an in vitro or in vivo process and the starting material a glycosylated precursor such as eriocitrin and the compound of formula (I) is taxifolin-3-O-acetate. In this embodiment of the invention the recombinant cell comprises the following enzyme(s): glycosidase, flavanone-3-hydroxylase (F3H) and acetyltransferase. This embodiment is termed recombinant cell 95 herein.

In another embodiment of the invention the recombinant cell is used in an in vitro or in vivo process and the starting material is a glycosylated precursor such as astilbin and the compound of formula (I) is taxifolin-3-O-acetate. In this embodiment of the invention the process comprises the following enzyme(s): glycosidase, and acetyltransferase. This embodiment is termed recombinant cell 96 herein.

In another embodiment of the invention the recombinant cell is used in an in vitro or in vivo process and the starting material is a glycosylated precursor such as a mixture of engeletin and astilbin in e.g. Engelhardia Roxburghiana extract and the compounds of formula (I) are aromadendrin-3-O-acetate and taxifolin-3-O-acetate. In this embodiment of the invention the recombinant cell comprises the following enzyme(s): glycosidase and acetyltransferase. This embodiment is termed recombinant cell 97 herein.

In addition to the preparation of aromadendrin-3-O-acetate and taxifolin-3-O-acetate, the enzyme combinations used in the recombinant cells of the invention numbered 1 to 97 can also be used to prepare the dihydrotamarixetin-3-O-acetate (a compound of formula (I)) with the inclusion of an additional methyltransferase enzyme specific for the 4′-position (4′-MT). Hence further embodiments of the invention provide recombinant cells for preparing dihydrotamarixetin-3-O-acetate comprising the enzymes listed in recombinant cells 1 to 97 and a methyltransferase, which is specific for 4′—OH. The inclusion of the methyltransferase enzyme specific for the 4′-position (4′-MT) in any of recombinant cells 1 to 97 results in an additional 97 recombinant cells. These are herein termed recombinant cells 98 to 194. Hence for example, recombinant cell 98 is that of recombinant cell 1 with the addition of the methyltransferase enzyme specific for the 4′-position (4′-MT), and so on.

Alternatively, final 4′-O-methylation can also be obtained by methylating position 4 of tyrosine, coumaric acid, coumaroyl-CoA, caffeic acid or caffeoyl-CoA present as intermediate or starting material using recombinant cells 1 to 27 and 63 to 89 with the inclusion of an additional enzyme 4-O-methyltransferase or 4-O-caffeoyl-methyltransferase in recombinant cells 1 to 27 and 63 to 89. The inclusion of the methyltransferase enzyme specific for the 4-position (4-MT) in any of recombinant cells 1 to 27 and 63 to 89 results in an additional 54 recombinant cells. These are herein termed recombinant cells 195 to 249. Hence for example, recombinant cell 195 is that of process 1 with the addition of 4-O-methyltransferase or 4-O-caffeoyl-methyltransferase and so on.

In another embodiment of the invention the recombinant cell is used in an in vitro or in vivo process and the starting material is isoferulic acid and the compound of formula (I) is dihydrotamarixetin-3-O-acetate. In this embodiment of the invention the recombinant cell comprises the following enzyme(s): 4-coumarate-CoA ligase (4CL), chalcone synthase (CHS), chalcone isomerase (CHI), chalcone isomerase-like protein (CHIL) flavanone-3-hydroxylase (F3H) and acetyltransferase. This embodiment is termed recombinant cell 250 herein.

In another embodiment of the invention the recombinant cell is used in an in vitro or in vivo process and the starting material is isoferulic acid and the compound of formula (I) is dihydrotamarixetin-3-O-acetate. Here the CHIL enzyme is omitted since this enzyme is not necessary for the performance of the process. In this embodiment of the invention the recombinant cell comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed recombinant cell 251 herein.

In another embodiment of the invention the recombinant cell is used in an in vitro or in vivo process and the starting material is isoferulic acid and the compound of formula (I) is dihydrotamarixetin-3-O-acetate. Here the CHIL and CHI enzymes are omitted since these enzymes are not necessary for the performance of the process. In this embodiment of the invention the recombinant cell comprises the following enzyme(s): 4-coumarate-CoA ligase (4CL), chalcone synthase (CHS), flavanone-3-hydroxylase (F3H) and acetyltransferase. This embodiment is termed recombinant cell 252 herein.

In another embodiment of the invention the recombinant cell is used in an in vitro or in vivo process and the starting material is hesperetin and the compound of formula (I) is dihydrotamarixetin-3-O-acetate. In this embodiment of the invention the recombinant cell comprises the following enzyme(s): flavanone-3-hydroxylase (F3H) and acetyltransferase. This embodiment is termed recombinant cell 253 herein.

In another embodiment of the invention the recombinant cell is used in an in vitro or in vivo process and the starting material is dihydrotamarixetin and the compound of formula (I) is dihydrotamarixetin-3-O-acetate. In this embodiment of the invention the recombinant cell comprises the following enzyme(s): acetyltransferase. This embodiment is termed recombinant cell 254 herein.

In another embodiment of the invention the recombinant cell is used in an in vitro or in vivo process and the starting material is a glycosylated precursor such as hesperidin and the compound of formula (I) is dihydrotamarixetin-3-O-acetate. In this embodiment of the invention the recombinant cell comprises the following enzyme(s): glycosidase, flavanone-3-hydroxylase (F3H) and acetyltransferase. This embodiment is termed recombinant cell 255 herein.

In another embodiment of the invention the recombinant cell is used in an in vitro or in vivo process and the starting material is a glycosylated precursor and the compound of formula (I) is dihydrotamarixetin-3-O-acetate. In this embodiment of the invention the recombinant cell comprises the following enzyme(s): glycosidase if performed in vitro) and acetyltransferase. This embodiment is termed recombinant cell 256 herein.

In addition to the preparation of aromadendrin-3-O-acetate and taxifolin-3-O-acetate, the enzyme combinations used in the recombinant cells of the invention numbered 1 to 97 can also be used to prepare the 3′-O-methyl-taxifolin-3-O-acetate (a compound of formula (I)) with the inclusion of the additional methyltransferase enzyme specific for the 3′-position (3′-MT). Hence further embodiments of the invention provide recombinant cells for preparing 3′-O-methyl-taxifolin-3-O-acetate comprising the enzymes listed in recombinant cell 1 to 97 and a 3′-OH methyltransferase. The inclusion of the methyltransferase enzyme specific for the 3′-position (3′-MT) in any of recombinant cells 1 to 97 results in an additional 97 recombinant cells. These are herein termed recombinant cells 257 to 353. Hence for example, recombinant cell 257 is that of process 1 with the addition of the methyltransferase enzyme specific for the 3′-position (3′-MT), and so on.

Alternatively, final 3′-O-methylation can also be obtained by methylating position 3 of caffeic acid or caffeoyl-CoA present as intermediate or starting material in processes 63 to 89 with the inclusion of an additional enzyme 3-O-methyltransferase or 3-O-caffeoyl-methyltransferase in recombinant cells 63 to 89. The inclusion of the methyltransferase enzyme specific for the 3-position (3-MT) in any of recombinant cells 63 to 89 results in an additional 27 recombinant cells. These are herein termed recombinant cells 354 to 380. Hence for example, recombinant cell 354 is that of recombinant cell 1 with the addition of the methyltransferase enzyme specific for the 3-position (3-MT), and so on.

In another embodiment of the invention the recombinant cell is used in an in vitro or in vivo process and the starting material is ferulic acid and the compound of formula (I) is 3′-O-methyl-taxifolin-3-O-acetate. In this embodiment of the invention the recombinant cell comprises the following enzyme(s): 4-coumarate-CoA ligase (4CL), chalcone synthase (CHS), chalcone isomerase (CHI), chalcone isomerase-like protein (CHIL), flavanone-3-hydroxylase (F3H) and acetyltransferase. This embodiment is termed recombinant cell 381 herein.

In another embodiment of the invention the recombinant cell is used in an in vitro or in vivo process and the starting material is ferulic acid and the compound of formula (I) is 3′-O-methyl-taxifolin-3-O-acetate. Here the CHIL enzyme is omitted since this enzyme is not necessary for the performance of the process. In this embodiment of the invention the recombinant cell comprises the following enzyme(s): 4-coumarate-CoA ligase (4CL), chalcone synthase (CHS), chalcone isomerase (CHI), flavanone-3-hydroxylase (F3H) and acetyltransferase. This embodiment is termed recombinant cell 382 herein.

In another embodiment of the invention the recombinant cell is used in an in vitro or in vivo process and the starting material is ferulic acid and the compound of formula (I) is 3′-O-methyl-taxifolin-3-O-acetate. Here the CHIL and CHI enzymes are omitted since these enzymes are not necessary for the performance of the process. In this embodiment of the invention the recombinant cell comprises the following enzyme(s): 4-coumarate-CoA ligase (4CL), chalcone synthase (CHS), flavanone-3-hydroxylase (F3H) and acetyltransferase. This embodiment is termed recombinant cell 383 herein.

In another embodiment of the invention the recombinant cell is used in an in vitro or in vivo process and the starting material is homoeriodictyol and the compound of formula (I) is 3′-O-methyl-taxifolin-3-O-acetate. In this embodiment of the invention the recombinant cell comprises the following enzyme(s): flavanone-3-hydroxylase (F3H) and acetyltransferase. This embodiment is termed recombinant cell 384 herein.

In another embodiment of the invention the recombinant cell is used in an in vitro or in vivo process and the starting material is 3′-O-methyl-taxifolin and the compound of formula (I) is 3′-O-methyl-taxifolin-3-O-acetate. In this embodiment of the invention the recombinant cell comprises the following enzyme(s): acetyltransferase. This embodiment is termed recombinant cell 385 herein.

In another embodiment of the invention the recombinant cell is used in an in vitro or in vivo process and the starting material is a glycosylated precursor such as homoeriodictyol-7-O-glucoside and the compound of formula (I) is 3′-O-methyl-taxifolin-3-O-acetate. In this embodiment of the invention the recombinant cell comprises the following enzyme(s): glycosidase, flavanone-3-hydroxylase (F3H) and acetyltransferase. This embodiment is termed recombinant cell 386 herein.

In another embodiment of the invention the recombinant cell is used in an in vitro or in vivo process and the starting material is a glycosylated precursor and the compound of formula (I) is 3′-O-methyl-taxifolin-3-O-acetate. In this embodiment of the invention the recombinant cell comprises the following enzyme(s): glycosidase and acetyltransferase. This embodiment is termed recombinant cell 387 herein.

In another embodiment of the invention the recombinant cell is used in an in vitro or in vivo process and the starting material is glucose (or another carbon source) and/or phenylalanine and the compound of formula (I) is pinobanksin-3-O-acetate. In this embodiment of the invention the recombinant cells comprise enzymes as in recombinant cell 7, 9, and 11, but omitting cinnamate-4-hydroxylase (C4H) and P450 reductase (CPR). These embodiments are termed recombinant cells 388, 389 and 390.

In another embodiment of the invention the recombinant cell is used in an in vitro or in vivo process and the starting material is cinnamic acid and the compound of formula (I) is pinobanksin-3-O-acetate. In this embodiment of the invention the recombinant cells comprise enzymes as in recombinant cells 16, 18, and 20, but omitting cinnamate-4-hydroxylase (C4H) and P450 reductase (CPR). These embodiments are termed recombinant cells 391, 392 and 393.

In another embodiment of the invention the recombinant cell is used in an in vitro or in vivo process and the starting material is pinocembrin and the compound of formula (I) is pinobanksin-3-O-acetate. In this embodiment of the invention the recombinant cell comprises the following enzyme(s): flavanone-3-hydroxylase (F3H) and acetyltransferase. This embodiment is termed recombinant cell 394 herein.

In another embodiment of the invention the recombinant cell is used in an in vitro or in vivo process and the starting material is pinobanksin and the compound of formula (I) is pinobanksin-3-O-acetate. In this embodiment of the invention the recombinant cell comprises the following enzyme(s): acetyltransferase. This embodiment is termed recombinant cell 395 herein.

In another embodiment of the invention the recombinant cell is used in an in vitro or in vivo process and the starting material is a glycosylated precursor such as pinocembrin-7-glucoside and the compound of formula (I) is pinobanksin-3-O-acetate. In this embodiment of the invention the recombinant cell comprises the following enzyme(s): glycosidase, flavanone-3-hydroxylase (F3H) and acetyltransferase. This embodiment is termed recombinant cell 396 herein.

In another embodiment of the invention the recombinant cell is used in an in vitro or in vivo process and the starting material is a glycosylated precursor such as pinobanksin 5-galactosyl-(1-4)-glucoside and the compound of formula (I) is pinobanksin-3-O-acetate. In this embodiment of the invention the recombinant cell comprises the following enzyme(s): glycosidase and acetyltransferase. This embodiment is termed recombinant cell 397 herein.

In addition to the preparation of aromadendrin-3-O-acetate, taxifolin-3-O-acetate, dihydrotamarixetin-3-O-acetate, 3′-O-methyltaxifolin-3-O-acetate or pinobanksin-3-O-acetate the enzyme combinations used in the recombinant cells of the invention numbered 1-27, 32-58, 63-92, 98-124, 129-155, 160-189, 195-252, 257-283, 288-314, 319-348, 354-383, 388-393 can also be used to prepare 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate (compounds of formula (I)) with the use of the additional polyketide reductase (PKR). Hence further embodiments of the invention provide recombinant cells for preparing 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate comprising the enzymes listed in any of the recombinant cells 1-27, 32-58, 63-92, 98-124, 129-155, 160-189, 195-252, 257-283, 288-314, 319-348, 354-383, 388-393 and a polyketide reductase. The use of the polyketide reductase enzyme in any of the recombinant cells 1-27, 32-58, 63-92, 98-124, 129-155, 160-189, 195-252, 257-283, 288-314, 319-348, 354-383, 388-393 results in additional 345 recombinant cells. These are herein termed recombinant cells 398 to 742. Hence for example, recombinant cell 398 is that of recombinant cell 1 with the addition of the polyketide reductase, and so on.

In another embodiment of the invention the recombinant cell is used in an in vitro or in vivo process and the starting material is isoliquiritigenin and the compound of formula (I) is 5-deoxyaromadendrin-3-O-acetate. In this embodiment of the invention the recombinant cell comprises the following enzyme(s): chalcone isomerase (CHI), flavanone-3-hydroxylase (F3H) and acetyltransferase. This embodiment is termed recombinant cell 743 herein.

In another embodiment of the invention the recombinant cell is used in an in vitro or in vivo process and the starting material is liquiritigenin and the compound of formula (I) is 5-deoxyaromadendrin-3-O-acetate. In this embodiment of the invention the recombinant cell comprises the following enzyme(s): flavanone-3-hydroxylase (F3H) and acetyltransferase. This embodiment is termed recombinant cell 744 herein.

In another embodiment of the invention the recombinant cell is used in an in vitro or in vivo process and the starting material is 5-deoxyaromadendrin and the compound of formula (I) is 5-deoxyaromadendrin-3-O-acetate. In this embodiment of the invention the recombinant cell comprises the following enzyme(s): acetyltransferase. This embodiment is termed recombinant cell 745 herein.

In another embodiment of the invention the recombinant cell is used in an in vitro or in vivo process and the starting material is butein and the compound of formula (I) is 5-deoxytaxifolin-3-O-acetate. In this embodiment of the invention the recombinant cell comprises the following enzyme(s): chalcone isomerase (CHI), flavanone-3-hydroxylase (F3H) and acetyltransferase. This embodiment is termed recombinant cell 746 herein.

In another embodiment of the invention the recombinant cell is used in an in vitro or in vivo process and the starting material is 5-deoxyeriodictyol and the compound of formula (I) is 5-deoxytaxifolin-3-O-acetate. In this embodiment of the invention the recombinant cell comprises the following enzyme(s): flavanone-3-hydroxylase (F3H) and acetyltransferase. This embodiment is termed recombinant cell 747 herein.

In another embodiment of the invention the recombinant cell is used in an in vitro or in vivo process and the starting material is 5-deoxytaxifolin and the compound of formula (I) is 5-deoxytaxifolin-3-O-acetate. In this embodiment of the invention the recombinant cell comprises the following enzyme(s): acetyltransferase. This embodiment is termed recombinant cell 748 herein.

Further embodiments of the invention provide recombinant cells used in in vitro or in vivo processes of preparing 5-deoxytaxifolin-3-O-acetate comprising the enzymes listed in the recombinant cells 743 to 745 and a flavonoid 3′-hydroxylase (F3′H). As can be appreciated by the skilled person, the addition of a F3′H to any one of the recombinant cells 743 to 745 results in an additional three recombinant cells. These are herein termed recombinant cells 749 to 751. Hence for example, recombinant cell 749 is that of recombinant cell 743 with the addition of a F3′H, and so on.

In another embodiment of the invention the recombinant cell is used in an in vitro or in vivo process and the starting material is 5-deoxyhesperetin chalcone and the compound of formula (I) is 5-deoxydihydrotamarixetin-3-O-acetate. In this embodiment of the invention the recombinant cell comprises the following enzyme(s): chalcone isomerase (CHI), flavanone-3-hydroxylase (F3H) and acetyltransferase. This embodiment is termed recombinant cell 752 herein.

In another embodiment of the invention the recombinant cell is used in an in vitro or in vivo process and the starting material is 5-deoxyhesperetin and the compound of formula (I) is 5-deoxydihydrotamarixetin-3-O-acetate. In this embodiment of the invention the recombinant cell comprises the following enzyme(s): flavanone-3-hydroxylase (F3H) and acetyltransferase. This embodiment is termed recombinant cell 753 herein.

In another embodiment of the invention the recombinant cell is used in an in vitro or in vivo process and the starting material is 5-deoxydihydrotamarixetin and the compound of formula (I) is 5-deoxydihydrotamarixetin-3-O-acetate. In this embodiment of the invention the recombinant cell comprises the following enzyme(s): acetyltransferase. This embodiment is termed recombinant cell 754 herein.

Further embodiments of the invention provide recombinant cells for in vitro or in vivo processes of preparing 5-deoxydihydrotamarixetin-3-O-acetate comprising the enzymes listed in recombinant cells 746 to 751 and a 4′-O-methyltransferase (4′-MT). As can be appreciated by the skilled person, the addition of a 4′-MT to any one of the recombinant cells 746 to 751 results in an additional 6 recombinant cells. These are herein termed recombinant cell 755 to 760. Hence for example, recombinant cell 755 is that of recombinant cell 746 with the addition of a 4′-MT, and so on.

In another embodiment of the invention the recombinant cell is used in an in vitro or in vivo process and the starting material is 5-deoxyhomoeriodictyol chalcone and the compound of formula (I) is 5-deoxy-3′-O-methyl-taxifolin-3-O-acetate. In this embodiment of the invention the recombinant cell comprises the following enzyme(s): chalcone isomerase (CHI), flavanone-3-hydroxylase (F3H) and acetyltransferase. This embodiment is termed recombinant cell 761 herein.

In another embodiment of the invention the recombinant cell is used in an in vitro or in vivo process and the starting material is 5-deoxyhomoeriodictyol and the compound of formula (I) is 5-deoxy-3′-O-methyl-taxifolin-3-O-acetate. In this embodiment of the invention the recombinant cell comprises the following enzyme(s): flavanone-3-hydroxylase (F3H) and acetyltransferase. This embodiment is termed recombinant cell 762 herein.

In another embodiment of the invention the recombinant cell is used in an in vitro or in vivo process and the starting material is 5-deoxy-3′-O-methyl-taxifolin and the compound of formula (I) is 5-deoxy-3′-O-methyl-taxifolin-3-O-acetate. In this embodiment of the invention the recombinant cell comprises the following enzyme(s): acetyltransferase. This embodiment is termed recombinant cell 763 herein.

Further embodiments of the invention provide recombinant cells used in in vitro or in vivo processes of preparing 5-deoxydihydrotamarixetin-3-O-acetate comprising the enzymes listed in recombinant cells 746 to 751 and a 3′-O-methyltransferase (3′-MT). As can be appreciated by the skilled person, the addition of a 3′-MT to any one of the recombinant cells 746 to 751 results in an additional 6 recombinant cells. These are herein termed recombinant cells 764 to 769. Hence for example, recombinant cell 764 is that of recombinant cell 746 with the addition of a 3′-MT, and so on.

In another embodiment of the invention the recombinant cell is used in an in vitro or in vivo process and the starting material is 5-deoxypinocembrin (7-hydroxyflavanone) chalcone and the compound of formula (I) is 5-deoxypinobanksin-3-O-acetate. In this embodiment of the invention the recombinant cell comprises the following enzyme(s): chalcone isomerase (CHI), flavanone-3-hydroxylase (F3H) and acetyltransferase. This embodiment is termed recombinant cell 770 herein.

In another embodiment of the invention the recombinant cell is used in an in vitro or in vivo process and the starting material is 5-deoxypinocembrin (7-hydroxyflavanone) and the compound of formula (I) is 5-deoxypinobanksin-3-O-acetate. In this embodiment of the invention the recombinant cell comprises the following enzyme(s): flavanone-3-hydroxylase (F3H) and acetyltransferase. This embodiment is termed recombinant cell 771 herein.

In another embodiment of the invention the recombinant cell is used in an in vitro or in vivo process and the starting material is 5-deoxypinobanksin and the compound of formula (I) is 5-deoxypinobanksin-3-O-acetate. In this embodiment of the invention the recombinant cell comprises the following enzyme(s): acetyltransferase. This embodiment is termed recombinant cell 772 herein.

Further embodiments of the invention provide recombinant cells used in in vitro or in vivo processes for preparing 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate, or 5-deoxypinobanksin-3-O-acetate from glycosylated precursors of the starting material of processes 743 to 772. In these embodiments of the invention the recombinant cells comprise the enzymes listed in recombinant cells 743 to 772 and a glycosidase. As can be appreciated by the skilled person, the addition of a glycosidase to any one of the recombinant cells 743 to 772 results in an additional 30 recombinant cells. These are herein termed recombinant cells 773 to 802. Hence for example, recombinant cell 773 is that of recombinant cell 743 with the addition of a glycosidase, and so on.

Example sequences for the enzymes listed in the above embodiments of the invention are provided in the sections above.

For the avoidance of doubt, as discussed above the process of the invention can use a hydrolase in replacement of an acetyltransferase.

Hence in each of the recombinant cells of the invention listed above, the acetyltransferase enzyme can be substituted with a hydrolase enzyme.

Furthermore, as can be appreciated by the skilled person, such host cells can be optimized for use in the process of the invention by up or down-regulation, exchange and engineering, of certain genes to increase metabolic flux to flavonoid precursors and/or reducing carbon loss resulting from the production of unwanted products. Examples of such host cells are provided herein in the above section relating to processes of the invention.

### Cell Cultures and Cell Lysates

The present invention provides for the first time a process for the in vivo preparation of a compound of formula (I).

Accordingly further aspect of the invention provides a cell culture comprising any of the recombinant host cells of the invention, and one or more compounds of formula (I).

A further aspect of the invention provides a cell lysate from the recombinant host cells of the invention grown in the cell culture, wherein the cell lysate comprises one or more compounds of formula (I).

The cell culture and cell lysates of the invention may also comprise one or more compounds of formula (Ia).

The cell culture and cell lysates of the invention may also comprise one or more of the starting materials used in any of the processes of the invention described herein.

The cell culture and cell lysates of the invention may also comprise supplemental nutrients comprising trace metals, vitamins, salts, YNB, and/or amino acids, as is commonly used for the growth of microbial cells.

The cell culture medium includes at least one carbon source that is also an energy source. Exemplary carbon sources include glucose, glycerol, sucrose, fructose, and xylose. Such carbon sources may be purified or crude, including a biomass comprising glycerol, for example, crude glycerol produced as a byproduct of biodiesel production from com waste. In addition, the culture medium can include one or more other carbon sources or compounds to increase precursor generation or cofactor supply or substrate such as, without limitation, tyrosine, phenylalanine, cinnamic acid, coumaric acid, caffeic acid, ferulic acid, isoferulic acid, acetate, malonate, succinate, glycine, bicarbonate, biotin, naringenin, eriodictyol, hesperetin, homoeriodictyol, pinocembrin, aromadendrin, taxifolin, dihydrotamarixetin, 3′-methyltaxifolin, pinobanksin, isoliquiritigenin, butein, 5-deoxyhesperetin chalcone, 5-deoxyhomoeriodictyol chalcone, 5-deoxypinocembrin chalcone, liquiritigenin, 5-deoxyeriodictyol, 5-deoxyhesperetin, 5-deoxyhomoeriodictyol, 5-deoxypinocembrin, 5-deoxyaromadendin, 5-deoxytaxifolin, 5-deoxydihydrotamarixetin, 5-deoxy-3′-O-methyl-taxifolin, 5-deoxypinobanksin, glycosylated flavanones, glycosylated dihydroflavonols, 5-aminolevulinic acid, thiamine, pantothenate, alpha-ketoglutarate, and ascorbate.

### Uses, Methods, and Formulations

In another aspect, the disclosure provides uses of any flavor-modifying compounds of the foregoing aspects, including any embodiments or combination of embodiments thereof, as set forth above. In certain related aspects, the disclosure provides uses of any flavor-modifying compounds of the foregoing aspects, including any embodiments or combination of embodiments thereof, as set forth above, to enhance the sweetness of an ingestible composition. In some embodiments thereof, the ingestible composition comprises a sweetener, such as a caloric sweetener. In certain other related aspects, the disclosure provides uses of any flavor-modifying compounds of the foregoing aspects, including any embodiments or combination of embodiments thereof, as set forth above, to reduce the sourness of an ingestible composition. In another related aspect, the disclosure provides uses of any flavor-modifying compounds of the foregoing aspects, including any embodiments or combination of embodiments thereof, as set forth above, to reduce the bitterness of an ingestible composition. In certain other related aspects, the disclosure provides uses of any flavor-modifying compounds of the foregoing aspects, including any embodiments or combination of embodiments thereof, as set forth above, in the manufacture of an ingestible composition to enhance the sweetness of the ingestible composition. In some embodiments thereof, the ingestible composition comprises a caloric sweetener. In another related aspect, the disclosure provides uses of any flavor-modifying compounds of foregoing aspects, including any embodiments or combination of embodiments thereof, as set forth above, in the manufacture of an ingestible composition to reduce the sourness of the ingestible composition. In another related aspect, the disclosure provides uses of any flavor-modifying compounds of the foregoing aspects, including any embodiments or combination of embodiments thereof, as set forth above, in the manufacture of an ingestible composition to reduce the bitterness of the ingestible composition.

The following examples are illustrative only and are not intended to limit the scope of the claims and embodiments described herein.

### EXAMPLES

### Analytical Method

### HPLC

Samples of the Examples were analyzed by HPLC using the column and conditions described herein below.

Column: InfinityLab Poroshell 120 EC-C18, 3.0×150 mm, 2.7 μm, injection volume: 2 μL, A: 100% water with 0.1% formic acid, B: acetonitrile with 0.1% formic acid. Pressure: 800 bar, flow 1 mL/min.

TABLE 1
HPLC conditions

| time | A % | B % |
|---|---|---|
| start | 90 | 10 |
| 9.5 | 45 | 55 |
| 12 | 5 | 95 |
| 13 | 5 | 95 |
| 13.5 | 90 | 10 |
| 15 | 90 | 10 |

### Example 1: Analysis of Transcriptome Data of Solidago canadensis

O-acetyltransferases constitute a genetically diverse class of enzymes with more than 35,000 known representatives (PFAM database: PF02458 transferase family), of which only 145 are reviewed entries. Although the repertoire of molecules accepted as substrates by acetyltransferases is vast, none of them has been reported to accept flavanonols as substrates.

In house transcriptome data from Solidago canadensis, which according to literature contains (2R,3R)-Pinobanksin-3-O-acetate, (Wu, M., Ge, Y., Xu, C., and Wang, J. (2020) Metabolome and Transcriptome Analysis of Hexaploid Solidago canadensis Roots Reveals its Invasive Capacity Related to Polyploidy. Genes (Basel) 11), was analyzed by using a set of literature known acetyltransferases for BLAST search in Geneious Prime software. Mainly partial and very few full-length genes could be identified. The partial genes were used for a BLAST search in NCBI and it turned out that many hits belong to Erigeron canadensis, a plant, for which two genomes are deposited in the NCBI database (https://www.ncbi.nlm.nih.gov/genome/browse/#!/eukaryotes/12828/). However, no exact annotation was provided. Thus, in case that the genes of Solidago canadensis were not complete, the hits of Erigeron canadensis from the BLAST search (all above 90% seq ID), were used instead. All selected genes were codon-optimized for Saccharomyces cerevisiae and ordered as synthetic genes cloned into vector pF013 from Twist.

### Example 2: Plant Material of Inula viscosa and Isolation of mRNA

It has been reported in literature that Inula viscosa, also called Dittrichia viscosa, (Bohlmann, F., Czerson, H., and Schöneweiss, S. (1977) Neue Inhaltsstoffe aus Inula viscosa Ait. Chem. Ber. 110, 5; Wollenweber, E., Mayer, K., and Roitman, J. N. (1991) Exudate flavonoids of Inula viscosa. Phytochemistry 30, 2445-2446) contains flavanonols, which are acetylated at position 3.

Inula viscosa (Dittrichia viscosa) was obtained from plant shops. The presence of taxifolin-3-O-acetate, aromadendrin-3-O-acetate and dihydrotamarixetin-3-O-acetate in leaves was confirmed by HPLC-MS.

Young leaves (1 to 2 cm long) were collected, frozen in liquid nitrogen, and used for the extraction of RNA using the PureLink™ Plant RNA Reagent from Invitrogen according to the provided manual.

### Example 3: Sequencing, Assembly and Analysis of Inula Transcriptome

The isolated mRNA samples were sent for library preparation and Illumina sequencing to Fasteris Lifescience Services (Geneva, Switzerland). The reads were assembled de novo using Trinity (min. contig length=200, min. Kmer coverage=1, max. reads per graph=200,000, min. cluster size=25). The resulting transcriptome contained 231,718 contigs, of which 88,659 were predicted to encode a complete ORF. ORFs were extracted. The assembled transcriptome and ORFs were analyzed in the Geneious Prime software for the presence of acetyltransferases by using a set of literature known acetyltransferases with various substrate scope as search templates for BLAST searches. Full length candidates were chosen, codon-optimized for yeast and ordered as synthetic genes cloned into vector pF013 from Twist.

### Example 4: Heterologous Expression in Yeast and First Screening

SMM—Leu medium: 15 g/L of (NH₄)₂SO₄, 8 g/L of KH₂PO₄ and 6.15 g/L of MgSO₄ 7H₂O in water at pH 5.5. 12 mL of vitamin solution, 10 mL of trace element solution, 10 mL of histidine stock (12.5 g/L, 10 mL of tryptophan stock (7.5 g/L), 40 mL of uracil stock (3.75 g/L) and 100 mL of D-glucose at 20%. The vitamin stock solution contains 0.05 g/L D-biotin, 1 g/I Cₐ-D-pantothenate, 1 g/L nicotinic acid, 25 g/L myo-inositol, 1 g/L thiamine hydrochloride, 1 g/L pyridoxal hydrochloride and 0.2 g/L p-aminobenzoic acid in water at pH 6.5. The trace element stock solutions contain: 5.75 g/L ZnSO₄·7H₂O, 0.32 g/L MnCl₂·4H₂O, 0.32 g/L CuSO4, 0.47 g/L CoCl₂·6H₂O, 0.48 g/L Na₂MoO₄·2H₂O, 2.9 g/L CaCl₂·2H₂O, 2.8 g/L FeSO₄·7H₂O and 14.88 g/L EDTA in water at pH 4. In case of the seed medium 100 mL 0.5 M succinate at pH 5 are added. The Leu deficient yeast strain Saccharomyces cerevisiae CEN.PK2-1 D was transformed with the plasmids using a standard protocol on a robotic platform. Single colonies of transformed cells were used to inoculate selective seed medium without Leu. Each candidate was added to the screening plate in duplicate.

### Fermentation in DWP and In Vivo Addition of Substrate

500 μL/well of SMM—LEU medium were pipetted in the deep well plate (DWP) and inoculated with 20 μL from the glycerol stock DWP. The plates were closed with a membrane and incubated at 30° C. and 1000 rpm over-night. 20 μL of these precultures were used to inoculate 500 μL/well of SMM—LEU+2% Galactose. The plates were closed with a membrane and incubated at 30° C. and 1000 rpm for 24 h. 50 μL of 5 mg/mL aromadendrin in DMSO were added and the plates were closed with a membrane and incubated at 30° C. and 1000 rpm for three days. The samples were diluted with MeOH, filtered and analyzed by HPLC. Aromadendrin-3-O-acetate was used as references and the identity of the products was confirmed by HPLC-MS.

### Fermentation in DWP and Subsequent In Vitro Screening

500 μL/well of SMM—LEU medium were pipetted in the DWP and inoculated with 20 μL from the glycerol stock DWP. The plates were closed with a membrane and incubated at 30° C. and 1000 rpm over-night. 20 μL of these precultures were used to inoculate 500 μL/well of SMM—LEU+2% Galactose. The plates were closed with a membrane and incubated at 30° C. and 1000 rpm for three days, after which the plates were centrifuged and the pellets frozen. Cell disruption was done using 150 μL of YPER solution according to the manufacturer's protocol. The reaction solution contained (per well) 50 μL of 5 mg/mL aromadendrin in DMSO and 50 μL of 8 mg/mL acetyl-CoA stock in 250 μL 100 mM KPi buffer, pH 7, and was prepared as mastermix which was then added to the well containing lysed cells to obtain a final volume of 0.5 mL. The reaction was done at 30° C. and 1000 rpm for 24 h. The samples were diluted with MeOH, filtered and analyzed by HPLC. Aromadendrin-3-acetate was used as references and the identity of the products was confirmed by HPLC-MS.

Two candidates from Erigeron canadensis (EcaAcT13 and EcaAcT17) and three candidates from Inula viscosa (IviAcT36, IviAcT45 and IviAcT72) were identified, which produced aromadendrin-3-O-acetate.

IviAcT45 is the most active enzyme and gave almost full conversion in the in vitro screening.

Yeast cells harboring the five enzymes, which produced aromadendrin-3-O-acetate, were streaked on agar plates. Colony PCR was performed using standard conditions and the PCR products were sent for sequencing to confirm the gene sequence.

### Example 5: Rescreening of SEQ ID NOs: 1 to 5

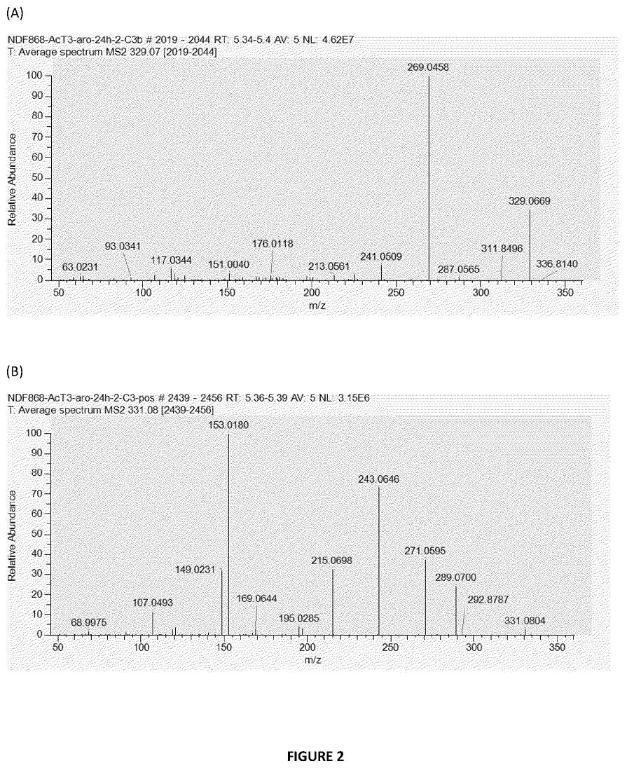

Yeast expressing the enzymes with SEQ ID NOs: 1 to 5 were grown on 20-100 mL scale in SMM-Leu+Gal medium. Lysed yeast cells expressing the five enzymes with SEQ ID NO: 1 to 5 were reacted with aromadendrin and acetyl-CoA in an in vitro assay in 100 mM phosphate buffer at pH 7 at 30° C. and 1000 rpm. The samples were diluted with MeOH, filtered and analyzed by HPLC. The in vitro rescreening confirmed the activity with aromadendrin, whereby EcaAcT13 and IviAcT36 showed only low activity of 2.5% and 9%, respectively, after 24 h using 0.36 g/L aromadendrin and 0.5 g/L acetyl-CoA. EcaAcT17 and IviAcT72 resulted in 20% aromadendrin-3-O-acetate and IviAcT45 gave 40% of aromadendrin-3-O-acetate. IviAcT45 was tested with higher aromadendrin concentrations and acetyl-CoA in molar excess. Using 5 g/L aromadendrin IviAcT45 could produce 120 mg/L/h of aromadendrin-3-O-acetate. The ESI-MS/MS spectra for the identification of aromadendrin-3-O-acetate are presented in FIG. 2.

### Example 6: Bioinformatics Analysis

The five sequences SEQ ID NOs: 1 to 5 were used for BLAST search in NCBI. Highest sequence similarities to the best hits are in the range of 60 to 71% for the different enzymes. All five enzymes show similarity to putative acetyltransferases and acyltransferases. However, many of the sequences identified from NCBI are not annotated or are annotated as predicted and putative acetyltransferases and acyltransferases and there is no data confirming activity.

The nucleic acid sequences encoding for SEQ ID NOs: 1 to 5 were also used in a BLAST search in NCBI. From this, two further sequences were identified as having homology. These sequences are part of a genome, which has not been translated or annotated. The polypeptide sequences for the two further nucleic acid sequences are given herein and named PdyAcT1 (SEQ ID NO: 6) and PdyAcT2 (SEQ ID NO: 7).

A further thirteen sequences were further identified as having homology to SEQ ID NOs: 1 to 7. They were not functionally annotated in any database.

All sequences SEQ ID NOs: 1 to 7, 31 to 36, and 55 to 61 contain a histidine residue, which is known to be highly conserved and important for the catalytic activity in acetyl/acyltransferases and is part of an HXXXD motif (motifs are in Prosite syntax, as defined in https://prosite.expasy.org/scanprosite/scanprosite_doc.html), wherein “X” denotes an arbitrary amino acid and with the histidine being part of the enzyme's binding pocket (Bontpart, T., Cheynier, V., Ageorges, A., and Terrier, N. (2015) BAHD or SCPL acyltransferase?What a dilemma for acylation in the world of plant phenolic compounds. New Phytol 208, 695-707), which is also present in all enzymes with SEQ ID NOs: 1 to 7, 31 to 36, and 55 to 61. By overlaying structural models of the enzymes with structures of literature known acetyltransferases, it can be seen that the location of this catalytic histidine is conserved. In addition, the less conserved DFGWG in the BAHD-AT family, is present exactly as DFGWG in IviAcT36, IviAcT45, IviAcT72, PdyAcT1, PdyAcT2, CcaAcT1, AIaAcT1, MmiAcT4, SsoAcT5, AanAcT3, HaAcT8, EcaAcT34 and DcaAcT2, and slightly modified to DFGFG in EcaAcT13, HaAcT11, HaAcT17 and MmiAcT9, and DFGLG in EcaAcT17, and DFGCG in SsoAcT4, and NFGLG in TciAcT5 indicating that enzymes with SEQ ID NOs: 1 to 7, 31 to 36, and 55 to 61 belong to the BAHD family of acyl/acetyltransferases. The BAHD-AT family takes its name from the first biochemically characterized enzymes, benzylalcohol O-acetyltransferase (BEAT), anthocyanin O-hydroxycinnamoyltransferase (AHCT), anthranilate N-hydroxycinnamoyl/benzoyltransferase (HCBT) and deacetylvindoline 4-O-acetyltransferase (DAT).

Structural models of SEQ ID NOs: 1 and 6 show that two amino acids form an oxyanion hole (T369 and W371 in SEQ ID NO: 1). These two amino acids are highly conserved in SEQ ID NOs: 1 to 7, 31 to 36, and 55 to 61, while they vary in the BAHD family of acyl/acetyltransferases as indicated in italic in Table 3. Motif [ST]SW is found in SEQ ID NOs: 1 to 3, 5 to 7, 31 to 36, and 55 to 61, motif SSL in SEQ ID NO: 4.

For the sake of clarity, the following sequence numbers were allocated to the enzymes.

TABLE 2
sequence number and enzyme name

| SEQ ID NO | Enzyme name |
|---|---|
| 1 | IviAcT45 |
| 2 | EcaAcT17 |
| 3 | EcaAcT13 |
| 4 | IviAcT36 |
| 5 | IviAcT72 |
| 6 | PdyAcT1 |
| 7 | PdyAcT2 |
| 31 | CcaAcT1 |
| 32 | HaAcT11 |
| 33 | AlaAcT1 |
| 34 | MmiAcT4 |
| 35 | SsoAcT4 |
| 36 | SsoAcT5 |
| 55 | AanAcT3 |
| 56 | EcaAcT34 |
| 57 | HaAcT8 |
| 58 | HaAcT17 |
| 59 | DcaAcT2 |
| 60 | MmiAcT9 |
| 61 | TciAcT5 |

### Example 7: Active Site Amino Acids were Chosen Based on the Structural Models of SEQ ID NOs: 1 to 7, 31 to 36, and 55 to 61

The present inventors reviewed the amino acid sequence of the enzymes of the present invention and also examined their 3D models using known protein modeling software. They have identified the amino acid residues in the table below as being suitable for modification to improve the activity of each of the enzymes.

TABLE 3
Amino acids in the potential aromadendrin binding cavity.

| EcaAcT13 | EcaAcT17 | IviAcT36 | IviAcT45 | IviAcT72 | PdyAcT1 | PdyAcT2 |
|---|---|---|---|---|---|---|
| A34 | A34 | G45 | P34 | P34 | P34 | P34 |
| S36 | I36 | S47 | I36 | G36 | I36 | I36 |
| I38 | T38 | S49 | T38 | I38 | T38 | T38 |
| N39 | N39 | P50 | P39 | N39 | P39 | P39 |
| H150 | H159 | H173 | H156 | H152 | H156 | H156 |
| A155 | M164 | G178 | M161 | T157 | M161 | M161 |
| S200 | S215 | E216 | S207 | S203 | S207 | S207 |
| V202 | R217 | L218 | R209 | N205 | R209 | R209 |
| A267 | Y285 | L292 | Q276 | Y271 | Q280 | Q280 |
| A269 | V287 | P294 | G278 | P273 | G282 | G282 |
| W289 | V307 | G316 | F298 | W293 | I302 | I302 |
| P291 | F309 | E318 | F300 | P295 | L304 | L304 |
| Y339 | Y354 | T363 | F351 | Y345 | F355 | F355 |
| V342 | L357 | E366 | F354 | A348 | F358 | F358 |
| L346 | L361 | K369 | F358 | L352 | L362 | F362 |
| F351 | I373 | Y374 | F365 | F357 | F369 | F369 |
| I353 | Y375 | I376 | C367 | L359 | S371 | S371 |
| T355 | T377 | S378 | T369 | T361 | T373 | T373 |
| W357 | W379 | L380 | W371 | W363 | W375 | W375 |
| R380 | F402 | T403 | Y394 | S386 | Y398 | Y398 |
| D383 | G404 | K405 | N396 | M388 | A400 | S400 |
| M384 | L407 | V407 | F399 | M391 | L403 | V403 |

| HaAcT11 | CcaAcT1 | AlaAcT1 | MmiAcT4 | SsoAcT4 | SsoAcT5 |
|---|---|---|---|---|---|
| P38 | A33 | S34 | V32 | R34 | E34 |
| H40 | S35 | S36 | M34 | M36 | V36 |
| T42 | I37 | I38 | V36 | V38 | V38 |
| G43 | N38 | N39 | P37 | P39 | P39 |
| H157 | H148 | H149 | H157 | H159 | H159 |
| A162 | T153 | A154 | M162 | M164 | M164 |
| F205 | Y197 | Y198 | K212 | D212 | H211 |
| F207 | L199 | L200 | L214 | I214 | L213 |
| I273 | A265 | A266 | H284 | S285 | G284 |
| A275 | A267 | A268 | P286 | A287 | A286 |
| C295 | W287 | W288 | F303 | I304 | S303 |
| A297 | P289 | P290 | A305 | L306 | F305 |
| I344 | Y337 | Y338 | F354 | Y355 | F354 |
| S347 | A340 | V341 | V357 | V358 | C357 |
| I351 | V344 | V345 | — | I362 | I361 |
| V357 | F349 | F350 | V364 | S368 | S368 |
| F359 | I351 | I352 | T366 | L370 | L370 |
| T361 | T353 | T354 | T368 | T372 | S372 |
| W363 | W355 | W356 | W370 | W374 | W374 |
| V386 | S378 | S379 | V393 | T397 | E397 |
| V388 | I380 | I381 | V395 | R399 | I399 |
| M391 | I382 | I383 | A398 | T402 | S402 |

| DcaAcT2 | AanAcT3 | EcaAcT34 | HaAcT8 | TciAcT5 | HaAc17 | MmiAcT9 |
|---|---|---|---|---|---|---|
| P34 | P34 | P34 | P34 | P36 | P38 | P38 |
| M36 | S36 | A36 | S36 | E38 | H40 | V40 |
| A38 | T38 | I38 | I38 | I40 | T42 | V42 |
| P39 | K39 | N39 | N39 | G41 | G43 | G43 |
| H161 | H150 | H150 | H150 | H155 | H157 | H158 |
| A166 | A155 | A155 | G155 | A160 | A162 | A163 |
| L213? | V198 | E198 | G198 | P202 | P204 | P211 |
| F215? | I200 | S200 | S200 | P204 | P205 | P213 |
| M282 | S266 | A268 | T267 | L275 | I273 | I273 |
| P284 | A268 | A270 | A269 | P277 | A275 | A275 |
| C305 | W288 | W290 | W289 | L297 | C295 | F295 |
| L307 | P290 | P292 | P291 | I299 | A297 | T297 |
| L351 | Y338 | Y340 | Y339 | Y346 | I344 | L344 |
| M354 | | | | N349 | S347 | S347 |
| Y361 | S350 | F352 | F351 | V358 | V357 | G356 |
| M363 | I352 | M354 | I353 | F360 | F359 | I358 |
| S365 | T354 | T356 | T355 | T362 | T361 | T360 |
| W367 | W356 | W358 | W357 | W364 | W363 | W362 |
| F390 | R379 | R381 | S380 | V387 | V386 | I385 |
| C392 | S380 | S382 | A381 | S389 | V388 | I387 |
| M395 | M383 | L385 | M384 | L392 | M391 | S390 |

### Example 8: Optimal pH of the Five Enzymes with SEQ ID NOs: 1 to 5

Lysed yeast cells expressing the five enzymes with SEQ ID NOs: 1 to 5 were reacted with 1 mg/mL aromadendrin and 2 mg/mL acetyl-CoA in an in vitro assay in 100 mM citrate/phosphate or phosphate buffer at pH 5, 6, 7, and 8 at 30° C. and 1000 rpm. The samples were diluted with MeOH, filtered and analyzed by HPLC. All enzymes were active at all four tested pHs. IviAcT45 was most active at pH 7 and gave full conversion. EcAcT13 and EcaAcT17 were also most active at pH 7, while IviAcT36 was most active at pH 5 and 6. IviAcT72 was most active at pH 5.

TABLE 4
Relative activity of acetyltransferases at different pHs, 100%
is referring to the activity at the preferred pH of each enzyme

| pH | EcaAcT13 | EcaAcT17 | IviAcT36 | IviAcT45 | IviAcT72 |
|---|---|---|---|---|---|
| 5 | 72.7 | 50.5 | 100.0 | 38.5 | 100.0 |
| 6 | 77.3 | 73.7 | 85.2 | 90.1 | 79.4 |
| 7 | 90.9 | 92.6 | 70.5 | 100.0 | 79.0 |
| 8 | 100.0 | 100.0 | 77.9 | 80.3 | 82.5 |

### Example 9: Activity of the Four Enzymes with SEQ ID NOs: 1 to 4 with Other Acyl-CoAs

Lysed yeast cells expressing the four enzymes with SEQ ID NOs: 1 to 4 were reacted with 1 mg/mL aromadendrin and 1 mg/mL of various acyl-CoAs in an in vitro assay at their preferred pH 6 or 7 in 100 mM phosphate buffer for 24 h at 30° C. and 1000 rpm. The samples were diluted with MeOH, filtered and analyzed by HPLC.

TABLE 5
Activity of enzymes with SEQ ID NOs: 1 to
4 with aromadendrin and various acyl-CoAs

| substrate | EcaAcT13 | EcaAcT17 | IviAcT36 | IviAcT45 |
|---|---|---|---|---|
| propanoyl-CoA | traces | traces | traces | Low active |
| Butyryl-CoA | no | no | active | traces |
| Hexanoyl-CoA | no | no | active | no |
| Octanoyl-CoA | no | no | Low active | no |

### Example 10: Preparation of taxifolin, dihydrotamarixetin, 3′-methyl-taxifolin, pinobanksin, 5-deoxyaromadendrin, 5-deoxytaxifolin and 5-deoxy-3′-methyltaxifolin by F3H enzyme

The gene (GenBank: U33932) encoding the flavanone-3-hydroxylase (F3H) from Arabidopsis thaliana was ordered codon-optimized for E. coli. E. coli BL21 (DE3) was transformed with the resulting construct. For the precultures LB medium containing kanamycin (50 μg/mL) was inoculated with E. coli BL21 (DE3) strains harboring the constructs and incubated at 37° C. and 200 rpm over-night. The preculture was used to inoculate the main culture of 400 mL LB medium containing kanamycin (50 μg/mL), which was incubated at 37° C. until the optical density at 600 nm (OD600) reached approximately 0.8. Expression of AthF3H was induced by 0.1 mM IPTG (isopropyl-D-thiogalactopyranoside) and the cultures were shaken at 20-25° C. for 20 h. The cells were harvested by centrifugation, and then resuspended in 100 mM potassium phosphate buffer (KPi, pH 7) to an OD of 50. Protein production was confirmed by SDS-PAGE, which showed that AthF3H was very well expressed as soluble protein.

Biotransformations were carried out with whole cells of E. coli BL21 (DE3)-AthF3H at OD10 in a total volume of 1 mL in 100 mM potassium phosphate buffer (KPi, pH 7) containing 10% DMSO, 1 g/L of eriodictyol, hesperetin, homoeriodictyol, pinocembrin, liquiritigenin or 5-deoxyeriodictyol, 10 mM α-ketoglutarate, 10 mM ascorbic acid and 0.25 mM FeSO₄ at 30° C. and 1000 rpm for 2 h. To obtain 5-deoxy-3′-methyltaxifolin a methyltransferase from Arabidopsis thaliana (accession number NP_200227) was used in combination with SAM factor using 5-deoxyeriodictyol as substrate before doing the F3H reaction. The reaction was extracted with EtOAc and the EtOAc was evaporated. The full conversion of the available (S)-enantiomer was confirmed by HPLC for all substrates.

The residue was used as substrate for acetyltransferases.

### Example 11: Activity of the Four Enzymes with SEQ ID NOs: 1 to 4 with Other Flavanonols

The substrates taxifolin, dihydrotamarixetin, 3′-methyl-taxifolin, pinobanksin, 5-deoxyaromadendrin, 5-deoxytaxifolin and 5-deoxy-3′-methyltaxifolin were either prepared as in example 10 or purchased.

Lysed yeast cells expressing the four enzymes with SEQ ID NOs: 1 to 4 were reacted with 0.5-1 mg/mL taxifolin, dihydrotamarixetin, 3′-methyl-taxifolin, pinobanksin, 5-deoxyaromadendrin, 5-deoxytaxifolin or 5-deoxy-3′-methyltaxifolin and 1-2 mg/mL of acetyl-CoA in an in vitro assay at their preferred pH 6 or 7 in 100 mM phosphate buffer at 30° C. and 1000 rpm for 24 h. The samples were diluted with MeOH, filtered and analyzed by HPLC.

TABLE 6
Activity of IviAcT45 (enzyme with SEQ ID NO:
1) with various flavanonols and acetyl-CoA.
% is referring to the formed product acetate.

| substrate | IviAcT45 |
|---|---|
| taxifolin | 43% |
| dihydrotamarixetin | 1% |
| 3′-methyl-taxifolin | 34% |
| pinobanksin | 24% |
| 5-deoxyaromadendrin | 59% |
| 5-deoxytaxifolin | 58% |
| 5-deoxy-3′-methyltaxifolin | 50% |

### Example 12: Activity of Enzymes with SEQ ID NOs: 1 to 4 Expressed in E. coli

The genes encoding the four enzymes with SEQ ID NOs: 1 to 4 were ordered codon-optimized for E. coli (SEQ ID NOs: 10, 13, 16, 19). E. coli BL21 (DE3) was transformed with the resulting constructs. For the precultures LB medium containing kanamycin (50 μg/mL) was inoculated with E. coli BL21 (DE3) strains harboring the constructs and incubated at 37° C. and 200 rpm over-night. The precultures were used to inoculate the main cultures of 400 mL LB medium containing kanamycin (50 μg/mL), which was incubated at 37° C. until the optical density at 600 nm (OD600) reached approximately 0.8. Expression was induced by 0.2 mM IPTG (isopropyl-D-thiogalactopyranoside) and the cultures were shaken at 20-25° C. for 20 h. The cells were harvested by centrifugation, and then resuspended in 100 mM potassium phosphate buffer (KPi, pH 7) to an OD of 50. Protein production was confirmed by SDS-PAGE.

Biotransformations were carried out with whole cells and the cells were reacted at OD5 with 1 mg/mL of aromadendrin and 4 mg/mL of acetyl-CoA in an in vitro assay at their preferred pH 6 or 7 in 100 mM phosphate buffer at 30° C. and 1000 rpm. The samples were diluted with MeOH, filtered and analyzed by HPLC.

While the activities of IviAcT36, EcaAcT13 and EcaAcT17 was in the low % range, IviAcT45 produced 40% of aromadendrin-3-O-acetate in 4 h.

### Example 13: Transesterification Using Hydrolases

The use of lipases, or more general hydrolases for ester formation is well known, however, for most lipases/hydrolases the reaction needs to be performed in water-free conditions to avoid hydrolysis of the newly formed product as hydrolysis is the natural reaction of these enzymes. Few cases of use of (engineered) lipases in aqueous media have been reported (Subileau, M., Jan, A. H., Drone, J., Rutyna, C., Perrier, V., and Dubreucq, E. (2017) What makes a lipase a valuable acyltransferase in water abundant medium?Catalysis Science & Technology 7, 2566-2578). In many cases the acyl/acetyl donor is vinylacetate/vinylacyate, as vinyls are very reactive. Also the released vinyl alcohol tautomerises to acetaldehyde, can be removed from the reaction by reduced pressure, and the equilibrium is shifted. Vinyl acetate is classified as extremely hazardous substance in the context of large-scale release, it is often produced by no longer acceptable environmentally unfriendly synthesis routes and is also known to cause enzyme stability issues. Alternatively, ethylacetate can be used as acetyl donor, but it is known to be less reactive.

20 mg commercial hydrolases (immobilized lipase kit from Chiralvision, which also contains a cutinase) prewashed with anhydrous EtOAc or heptane) or nothing (as control) were mixed with 50 μL of aromadendrin stock in anhydrous EtOAc and 450 μL of anhydrous EtOAc or heptane and incubated at different temperatures over-night at 800 rpm. The samples were diluted with MeOH, filtered and analyzed by HPLC. Several lipases and a cutinase showed some formation of aromadendrin-3-O-acetate, while the negative control did not contain any product. The reaction of the most promising lipase was further optimized by varying the temperature, the ratio of immobilized lipase to substate and the addition of a molecular sieve to further reduce the water content in the reaction.

100 mg commercial lipase (IMMLIPX-COV-1 from Chiralvision) prewashed with anhydrous EtOAc were mixed with molecular sieve (3 A) and with 0.25 mL of 10 g/L aromadendrin stock in anhydrous EtOAc and anhydrous EtOAc to a final volume of 5 mL and incubated at 60° C. over-night at 800 rpm. The samples were diluted with MeOH, filtered and analyzed by HPLC. 14.2% aromadendrin-3-O-acetate could be obtained after 24 h. The reaction continues for up to 96 h forming 30% of aromadendrin-3-O-acetate.

The lipases showed very poor activity and are considered not suitable for large-scale applications.

### Example 14—Natural Mixture Testing

Hydrolysis of Engelhardia Roxburghiana extract

In a 500 mL three necked round bottom flask, fitted with a mechanical stirrer and a condenser, 25 g of Engelhardia Roxburghiana extract (80% astilbin, supplier Xi'an Tuofeng Biotech Ltd.), 250 mL of 1 M citric acid (freshly prepared) and 250 mg of vitamin E were refluxed and stirred overnight night. After cooling, the reaction mixture was extracted 2×with ethyl acetate. Combined organic layers were washed 1×with deionized water, 1×with brine, dried over sodium sulfate, filtered and evaporated to afford 14.4 g of crude Engelhardia hydrolysate containing aromadendrin and taxifolin (88.3% yield).

### Enzymatic Acetylation of Engelhardia Hydrolysate Using Acetyltransferases

Lysed yeast cells expressing the four enzymes with SEQ ID NOs: 1 to 4 were reacted with 0.5-1 mg/mL Engelhardia hydrolysate and 1-2 mg/mL of acetyl-CoA in an in vitro assay at their preferred pH 6 or 7 in 100 mM phosphate buffer at 30° C. and 1000 rpm. The samples were diluted with MeOH, filtered and analyzed by HPLC. The results correspond very well to the results of the single molecules aromadendrin and taxifolin resulting in 30-40% product formation with IviAcT45 after 24 h.

### Enzymatic Acetylation of Engelhardia Hydrolysate Using Lipases

20 mg commercial lipases (immobilized lipase kit from Chiralvision) prewashed with anhydrous EtOAc or heptane) or nothing (as control) were mixed with 50 μL of Engelhardia hydrolysate at 10 mg/mL in anhydrous EtOAc and 450 μL of anhydrous EtOAc or heptane and incubated at different temperature overnight at 800 rpm. The samples were diluted with MeOH, filtered and analyzed by HPLC. The same lipases as in example 13 showed some very low % formation of aromadendrin-3-O-acetate and taxifolin-3-O-acetate, while the negative control did not contain any product.

### Example 15—Acetyltransferase Activity of SEQ ID NOs: 6, 7 and 31 to 36

Yeast expressing the enzymes with SEQ ID NOs: 6, 7 and 31 to 36 were grown on 20 mL scale in SMM-Leu+Gal medium. Lysed yeast cells expressing the enzymes with SEQ ID NOs: 6, 7 and 31 to 36 were reacted with 0.5 mg/mL aromadendrin and 1.7 mg/mL acetyl-CoA in an in vitro assay in 100 mM phosphate buffer at pH 7 at 30° C. and 1000 rpm for 24 h. The samples were diluted with MeOH, filtered and analyzed by HPLC. CcaAcT1 formed slightly above 60% aromadendrin-3-O-acetate, HaAcT11 and AlaAcT1 showed slightly above 40% aromadendrin-3-O-acetate formation, and PdyAcT1 resulted in about 18% aromadendrin-3-O-acetate formation. For PdyAcT2, MmiAcT4, SsoAcT4 and SsoAcT5, activity was also observed at a level less than 10% aromadendrin-3-O-acetate formation. The most active enzymes were also tested at pH 5 and 6. CcaAcT1, AlaAcT1 and HaAcT11 showed similar activity at pH 6 as at pH 7. HaAcT11 formed 20% of product at pH 5 and AlaAcT1 5%.

### Example 16—Acetyltransferase Activity of SEQ ID NOs: 55 to 61

Yeast expressing the enzymes with SEQ ID NOs: 55 to 61 were grown on 20 mL scale in SMM-Leu+Gal medium. Lysed yeast cells expressing the enzymes with SEQ ID NOs: 55 to 61 were reacted with 0.5 mg/mL aromadendrin and 1.7 mg/mL acetyl-CoA in an in vitro assay in 100 mM phosphate buffer at pH 7 at 30° C. and 1000 rpm for 24 h. The samples were diluted with MeOH, filtered and analyzed by HPLC. DcaAcT2 formed 44% aromadendrin-3-O-acetate, HaAcT17 and MmiAcT9 showed above 30% aromadendrin-3-O-acetate formation (31% and 38%, respectively), and TciAcT5 resulted in about 12% aromadendrin-3-O-acetate formation. For AanAcT3, EcaAcT34, and HaAcT8 activity was also observed at a level less than 5% aromadendrin-3-O-acetate formation. The most active enzymes were also tested at pH 5 and 6. DcaAcT2 resulted in 20% more product at pH 5 and 6 than at pH 7. HaAcT17 has the same activity at pH 6 as at pH 7, and at pH 5 about 10% of product was formed. MmiAcT9 prefers pH 7.

### Example 17—Protein Engineering of SEQ ID NO: 1

Based on a structural model of SEQ ID NO: 1, the amino acids of the active site in SEQ ID NO: 1 and as listed in Table 3 were substituted; thereby, resulting for example in variants represented by SEQ ID NO: 62 to 67. The respective nucleotide sequences were codon-optimized for yeast or E. coli and ordered as synthetic genes cloned into vector pF013 or pET29b, respectively, from Twist.

When compared with SEQ ID NO: 1, the variants comprise the following substitutions:
SEQ ID NO: 62 comprises a P34A substitution,
SEQ ID NO: 63 comprises a P34H substitution,
SEQ ID NO: 64 comprises P34A and F354Y substitutions,
SEQ ID NO: 65 comprises P34A and F365Y substitutions,
SEQ ID NO: 66 comprises P34A, F354Y, and F365Y substitutions,
SEQ ID NO: 67 comprises a F358Y substitution.

Yeast expressing the enzymes with SEQ ID NOs: 1 and 62 to 67 were grown on 20 mL scale in SMM-Leu+Gal medium. Lysed yeast cells expressing the enzymes with SEQ ID NOs: 1 and 62 to 67 were reacted with 0.1 mg/mL aromadendrin or taxifolin and 2 mg/mL acetyl-CoA in an in vitro assay with and without 10% DMSO in 50 mM phosphate buffer at pH 6 at 30° C. and 1000 rpm for 10, 30 and 60 min. Alternatively, lysed yeast cells expressing the enzymes with SEQ ID NOs: 1 and 62 to 67 were reacted with 0.4 mg/mL dihydrotamarixetin and 2 mg/mL acetyl-CoA in an in vitro assay with 10% DMSO in 50 mM phosphate buffer at pH 6 at 30° C. and 1000 rpm for 22 h. The samples were diluted with MeOH, filtered and analyzed by HPLC.

As shown in Table 7, 8 and 9, several variants were more active than the wildtype enzyme with aromadendrin, taxifolin and dihydrotamarixetin.

TABLE 7
Relative aromadendrin-3-acetate formation (%)

| | 10% DMSO | | | no DMSO | | |
|---|---|---|---|---|---|---|
| | 10 | 30 | 60 | 10 | 30 | 60 |
| Sequences | min | min | min | min | min | min |
| SEQ ID NO: 1 | 100.0 | 100.0 | 100.0 | 100.0 | 100.0 | 100.0 |
| SEQ ID NO: 62 | 130.5 | 135.6 | 132.6 | 389.4 | 341.9 | 273.2 |
| SEQ ID NO: 63 | 85.3 | 94.7 | 94.2 | 188.6 | 288.2 | 243.8 |
| SEQ ID NO: 64 | 236.2 | 193.8 | 155.7 | 728.0 | 514.2 | 335.8 |
| SEQ ID NO: 65 | 65.0 | 76.5 | 85.1 | 180.0 | 177.3 | 170.6 |
| SEQ ID NO: 66 | 103.7 | 108.3 | 88.0 | 452.4 | 385.3 | 293.5 |
| SEQ ID NO: 67 | 99.2 | 103.7 | 106.5 | 194.8 | 193.4 | 174.4 |

TABLE 8
Relative taxifolin-3-acetate formation (%)

| | 10% DMSO | | | no DMSO | | |
|---|---|---|---|---|---|---|
| | 10 | 30 | 60 | 10 | 30 | 60 |
| Sequences | min | min | min | min | min | min |
| SEQ ID NO: 1 | 100.0 | 100.0 | 100.0 | 100.0 | 100.0 | 100.0 |
| SEQ ID NO: 62 | 180.9 | 145.0 | 124.1 | 282.2 | 203.5 | 171.7 |
| SEQ ID NO: 63 | 112.0 | 110.5 | 106.1 | 156.4 | 143.8 | 141.4 |
| SEQ ID NO: 64 | 240.7 | 176.8 | 135.2 | 517.5 | 223.6 | 193.9 |
| SEQ ID NO: 65 | 105.7 | 104.0 | 102.3 | 170.0 | 149.7 | 143.8 |
| SEQ ID NO: 66 | 180.2 | 153.0 | 130.3 | 369.4 | 244.5 | 188.5 |
| SEQ ID NO: 67 | 146.2 | 135.6 | 121.6 | 147.4 | 136.7 | 139.1 |

TABLE 9
Relative dihydrotamarixetin-3-acetate formation (%)

| Sequences | 22 h, 10% DMSO |
|---|---|
| SEQ ID NO: 1 | 100.0 |
| SEQ ID NO: 62 | 874.2 |
| SEQ ID NO: 63 | 549.6 |
| SEQ ID NO: 64 | 1674.4 |
| SEQ ID NO: 65 | 840.1 |
| SEQ ID NO: 66 | 2420.3 |
| SEQ ID NO: 67 | 493.6 |

### Example 18—Protein Engineering of SEQ ID NO: 6

The two very similar enzymes IviAcT45 (SEQ ID NO: 1) and PdyAcT1 (SEQ ID NO: 6) have quite different enzyme activity. Structural models showed that several active site amino acids, in particular four phenylalanine residues and an asparagine residue, that might be important for substrate binding, are different in PdyAcT1 compared to IviAcT45. Substituting these amino acids (1302F, L304F, L362F, L403F, A400N) to the respective amino acids from IviAcT45, resulted in a significant increase in activity. Yeast expressing the enzymes with SEQ ID NO: 6 and 68 were grown on 20 mL scale in SMM-Leu+Gal medium. Lysed yeast cells expressing the enzymes with SEQ ID NOs: 6 and 68 were reacted with 0.1 mg/mL aromadendrin or taxifolin and 2 mg/mL acetyl-CoA in an in vitro assay with 10% DMSO in 50 mM phosphate buffer at pH 6 at 30° C. and 1000 rpm for 2, 6 and 24 h. The samples were diluted with MeOH, filtered and analyzed by HPLC.

PdyAcT1 comprising the 1302F, L304F, L362F, L403F, A400N substitutions (SEQ ID NO: 68) resulted in a five times improved activity compared to wildtype PdyAcT1 at all time points.

1. A process for making a compound of formula (I): 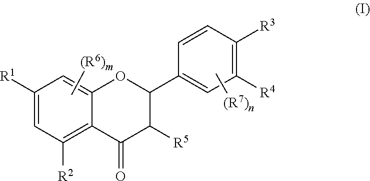
wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A};
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A};
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁵ is —O—C(O)—(C₁₋₂₄ alkyl);
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3;
the process comprising reacting a precursor compound of formula (Ia) 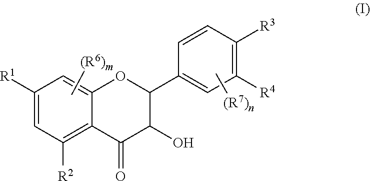
wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A};
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A};
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3;
with an acyltransferase enzyme to form a compound of formula (I).

2. The process of claim 1, wherein the process is performed in the presence of acyl-CoA.

3. The process of claim 1, wherein the acyltransferase is an acetyltransferase enzyme.

4. The process of claim 1, wherein the acyltransferase enzyme comprises the amino acid sequence HXXXD (SEQ ID NO: 29) and/or amino acid sequence [DN]FGxG (SEQ ID NO: 30).

5. The process of claim 1, wherein the acyltransferase enzyme comprises the amino acid sequence [ST]S[WL](SEQ ID NO: 94).

6. The process of claim 1, wherein the acyltransferase enzyme has at least 50%, 55%, 60%, 65%, 70%, 75%, 80%, 85%, 90%, 95%, 96%, 97%, 98% or 99% sequence identity to any of SEQ ID NOs: 1 to 7, 31 to 36 and 55 to 68.

7. The process of claim 1, wherein the compound of formula (Ia) is aromadendrin, taxifolin, dihydrotamarixetin, 3′-O-methyltaxifolin, pinobanksin, 5-deoxyaromadendrin, 5-deoxytaxifolin, 5-deoxydihydrotamarixetin, 5-deoxy-3′-O-methyltaxifolin or 5-deoxypinobanksin.

8. The process of claim 1, wherein the compound of formula (I) is aromadendrin-3-O-acetate, taxifolin-3-O-acetate, dihydrotamarixetin-3-O-acetate, or 3′-O-methyltaxifolin-3-O-acetate, pinobanksin-3-O-acetate, 5-deoxyaromadendrin-3-O-acetate, 5-deoxytaxifolin-3-O-acetate, 5-deoxydihydrotamarixetin-3-O-acetate, 5-deoxy-3′-O-methyltaxifolin-3-O-acetate or 5-deoxypinobanksin-3-O-acetate.

9. The process of claim 1, wherein the process is in vivo.

10. A recombinant polypeptide having acyltransferase activity and comprising an amino acid sequence having at least 70%, 75%, 80%, 85%, 90%, 95%, 96%, 97%, 98% or 99% sequence identity to any of SEQ ID NOs: 1 to 7, 31 to 36 and 55 to 68 or comprising the amino acid sequence of any of SEQ ID NOs: 1 to 7, 31 to 36 and 55 to 68.

11. A recombinant cell comprising a compound of formula (I); 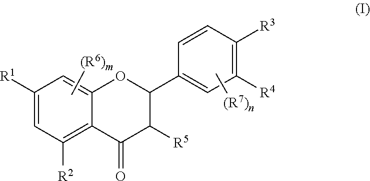
wherein:
R¹ is a hydrogen atom, —OH, or —O—R^{1A};
R^{1A} is C₁₋₆ alkyl, which is optionally substituted one or more times by substituents selected from the group consisting of —OH and C₁₋₆ alkoxy;
R² is a hydrogen atom, —OH, or —O—R^{2A};
R^{2A} is C₁₋₆ alkyl, which is optionally substituted one or more times by substituents selected from the group consisting of —OH and C₁₋₆ alkoxy;
R³ is a hydrogen atom, —OH or —O—R^{3A};
R^{3A} is C₁₋₆ alkyl, which is optionally substituted one or more times by substituents selected from the group consisting of —OH and C₁₋₆ alkoxy;
R⁴ is a hydrogen atom, —OH, or —O—R^{4A};
R^{4A} is C₁₋₆ alkyl, which is optionally substituted one or more times by substituents selected from the group consisting of —OH and C₁₋₆ alkoxy;
R⁵ is —O—C(O)—(C₁₋₂₄ alkyl);
R⁶ and R⁷ are independently selected from the group consisting of C₁₋₆ alkyl, —OH, C₁₋₆ alkoxy, and —O—(C₁₋₆ alkylene)-O—(C₁₋₆ alkyl);
m is 0, 1, or 2; and
n is 0, 1, 2, or 3.

12. The recombinant cell of claim 11 further comprising an acyltransferase enzyme.

13. The recombinant cell of claim 11 further comprising a recombinant nucleic acid sequence encoding an acyltransferase enzyme.

14. The recombinant cell of claim 11, wherein the cell further comprises one or more of the following enzyme(s):
(a) flavanone 3-hydroxylase, (F3H),
(b) chalcone isomerase (CHI),
(c) chalcone synthase (CHS),
(d) 4-coumarate-coenzyme A ligase (4CL),
(e) cytochrome P450 reductase (CPR),
(f) tyrosine ammonia lyase (TAL),
(g) chalcone isomerase-like (CHIL),
(h) cinnamate-4-hydroxylase (C4H),
(i) phenylalanine ammonia lyase (PAL),
(j) flavonoid 3-hydroxylase (F3′H),
(k) 3′-O-methyltransferase (3′-MT),
(l) 4′-O-methyltransferase (4′-MT),
(m) 3-O-methyltransferase (3-MT),
(n) 4-O-methyltransferase (4-MT),
(o) 3-OH specific P450 monooxygenase,
(p) glycosidase, and/or
(q) polyketide reductase (PKR).

15. The recombinant cell of claim 11, wherein the recombinant cell is a bacterial, archaebacterial, fungal, yeast, algal or plant cell.

16. A growth medium comprising the recombinant cell and the compound of formula (I) of claim 11.

17. A process for making a compound of formula (I) comprising growing the recombinant cell of claim 11 under growth conditions suitable for the production of the compound of formula (I).

18. A compound of formula (I) obtained by the process of claim 1.

19. (canceled)

20. A method of a) enhancing a sweet taste, (b) reducing a bitter taste, and/or (c) reducing a sour taste, of an ingestible composition of a product, the method comprising introducing to the product a compound of formula (I) obtained by the process of claim 1.
